(19) United States
(12) Patent Application Publication
(10) Pub. No.: US 2024/0247004 A1
(43) Pub. Date: Jul. 25, 2024

(54) NEW CERIUM (IV) COMPLEXES AND THEIR USE IN ORGANIC ELECTRONICS

(71) Applicant: CREDOXYS GmbH, Dresden (DE)

(72) Inventors: Sascha Dorok, Dresden (DE); Marcus Papmeyer, Nossen (DE); Leonard Eymann, Dresden (DE)

(21) Appl. No.: 18/549,034

(22) PCT Filed: Mar. 8, 2022
(86) PCT No.: PCT/EP2022/055890

§ 371 Date: Sep. 5, 2023

(30) Foreign Application Priority Data
Mar. 10, 2021 (EP) 21161845.9

(51) Int. Cl. C07F 5/00; C09K 9/02; C09K 11/06; H10K 30/85; H10K 30/86; H10K 50/15; H10K 50/16; H10K 50/17; H10K 85/30

(52) U.S. Cl. CPC C07F 5/003; C09K 9/02; C09K 11/06; H10K 85/351; C09K 2211/1007; C09K 2211/182; H10K 30/85; H10K 30/86; H10K 50/15; H10K 50/16; H10K 50/17

(57) ABSTRACT

The present invention relates to new cerium (IV) complexes. Further, the present invention relates to electronically doped semiconductor materials and an electronic component comprising cerium (IV) complexes. A further object of the invention is the use of the cerium (IV) complexes as electron acceptors, especially as p-dopants and electron transport materials in organic electronic components.

The present invention relates to new cerium (IV) complexes. Further, the present invention relates to electronically doped semiconductor materials and an electronic component comprising cerium (IV) complexes. A further object of the invention is the use of the cerium (IV) complexes as electron acceptors, especially as p-dopants and electron transport materials in organic electronic components.

### BACKGROUND OF THE INVENTION

Organic electronics focuses on the development, characterization and application of new materials, both based on small organic molecules and polymers with certain desired electronic properties for the production of electronic components. These comprise e.g. organic field effect transistors (OFETs), such as organic thin film transistors (OTFTs), organic electroluminescent devices, such as organic light emitting diodes (OLEDs), organic solar cells (OSCs), e.g. exciton solar cells, dye-sensitized solar cells (DSSCs) or perovskite solar cells, electrophotography, e.g. photoconductive materials in organic photoconductors (OPCs), organic optical detectors, organic photoreceptors, light-emitting electrochemical cells (LECs) and organic laser diodes.

It is known that organic semiconductor matrices can be heavily influenced regarding their electrical conductivity by doping. Such organic semiconductive matrix materials can be formed either from compounds with good electron donor properties (p-conductors) or from compounds with good electron acceptor properties (n-conductors). In contrast to inorganic semiconductors, organic semiconductors have a very low intrinsic charge carrier concentration. Organic semiconductor matrix materials are therefore usually doped in order to achieve good semiconductor properties. For n-doping strong electron donors (n dopants) are used, which transfer an electron to the LUMO of the semiconductor matrix (n-doping), resulting in a free electron on the matrix (SOMO). For p-doping strong electron acceptors (p-dopants) are used, which remove an electron from the HOMO of the semiconductor matrix (p-doping), resulting in a hole. In other words, for p-doping the LUMO of the dopant must be below the HOMO-energy of the matrix. The dopant acts as an acceptor and leaves a mobile hole (SOMO) in the matrix.

Known p-dopants for electron donor materials are electron acceptors such as tetracy-anoquinone methane (TCNQ), 2,3,5,6-tetrafluorotetracyano-1,4-benzoquinone methane (F4TCNQ), trinaphthylenes (HATNA), metal oxides such as MoO₃ or WO₃, or radialene compounds as e.g. described in EP 2180029. The acceptor molecules generate so-called holes in the semiconductor matrix materials (hole transport materials) by electron transfer processes, and the conductivity of the semiconductor matrix material (hole transport material) is more or less changed depending on the number and mobility of the holes.

However, the previously described compounds or compound classes have disadvantages for a technical use in the production of doped semiconductors or of corresponding electronic components with such doped layers. The compounds or compound classes mentioned are, for example, too volatile, have a too high absorption coefficient, have an unstable evaporation rate and/or show low thermostability. In addition, some of these compounds have very high production costs.

Thus, there is still a demand for compounds, which are easily available or producible, are suitable for doping electron donor materials and do not have the disadvantages described above.

Only a few cerium (IV) complexes of the class of diketonates are known. A few β-diketonate complexes of cerium (IV) are described in the literature. M. Ciampolini et al., J.C.S. Dalton, 1977, 1325; T. J. Pinnaviaia et al., Contribution from the department of Chemistry, Cornell University, Ithaca, New York, 1965, 233; I. Baxter et al., J. Chem. Cryst, Vol. 28, No 4, 1998, 267; N. A. Piro et al., Coord. Chem. Review, 260, 2014, 21, M. Delarosa et al., J. Coord. Chem., 55(7), 2002, 781; Jahr et al., Zeitschrift für Chemie, Bd. 15, 1975, S 280-281; Snezhko et al. Material Science and Engineering, Vol. 18, 1993, S. 230-231; Brill et al., Liebigs Annalen der Chemie, 1979, S. 803-810 and WO02/018394 describe cerium (IV) complexes.

WO 02/018394 relates to precursor source reagent of metal-organic compositions. The formation of cerium doped (Ca, Sr)Ga₂S₂ films with thio-containing solvent systems and deposition in the presence of hydrogen sulfide gas is described.

Kunkely et al., Journal of Photochemistry and Photobiology A, Vol 146, No 1-2, p. 63-66 describes cerium (IV) 2,2,6,6-tetramethyl-3,5-heptane-dionate anion. It is further described that this complex has luminescent properties and is also photoactive. These properties are irrelevant for a p-dopant or for redox doping pairs of transport layers. US 2010/0038632 describes a variety of complexes including cerium (IV) complexes. On the one hand explicit cerium (IV) complexes according to the invention are not mentioned. WO 2021/048044 relates to cerium (IV) complexes and their use in organic electronics. However, the compounds disclosed in this document differ from the compounds according to the invention.

U.S. Pat. No. 4,511,515 discloses a method for the synthesis of a specific cerium (IV) complex, namely [Ce(fod)₄], wherein fod is β-diketone 6,6,7,7,8,8,8-heptafluoro-2,2-dimetyl-3,5-octanedione. U.S. Pat. No. 4,511,515 does not even mention the use of this compound in organic electronics.

WO 00/32719 relates to metal complexes which form a film or layer on a substrate. The generic formulae to define the metal complexes is very broad. Thus the compounds according to the invention are distinct from the compounds disclosed in WO 00/32719.

On the other hand, the large band gaps in cerium complexes mentioned in this document are not relevant for a p-dopant.

Until now, it was unknown to use cerium (IV) complexes in organic semiconductor materials. In particular, it has not yet been described to use cerium (IV) complexes as p-dopants, as electron transport materials or as electron acceptors.

Surprisingly, it has now been found that cerium (IV) complexes can be advantageously used as p-dopants. Furthermore, it has been found that cerium (IV) complexes can be used as electron transport materials (ETM) in organic electronic components such as organic light emitting diodes (OLED), photovoltaic cells, organic solar cells (OPV), organic diodes or organic transistors.

Furthermore, many cerium (IV) diketonates can be evaporated very well under vacuum and occasionally exhibit high thermostability. Thus, they are basically suitable for both variants of processing of organicelectronic components, the vacuum coating (vapour deposition) and the solvent-based processing (solution processing).

### SUMMARY OF THE INVENTION

A first object of the invention is a of the general formula (I)

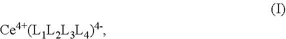

wherein
L₁; L₂; L₃; and L₄ are independently from each other selected from a bidentate ligand having the general formula (II)

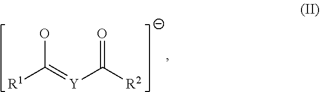

wherein
Y represents N or CR³;
R¹ represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
   cycloalkyl is unsubstituted or substituted with 1 to 13 radicals selected from fluorine and CF₃,
   phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
   naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂, pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂; or
R¹ and Y together with the C—O-group (in particular the carbon atom of the C—O-group) to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R² represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
   cycloalkyl is unsubstituted or substituted with 1 to 13 radicals selected from fluorine and CF₃,
   phenyl is substituted by at least one radical selected from NO₂, F, C, Br, CN, CF₃, SF₅, OCF₃, SCF₃, SO₂CF₃, N═C(CF₃)₂, and N(SO₂CF₃)₂,
   naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂, pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂; or
R² and Y together with the C—O-group (in particular the carbon atom of the C—O-group) to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R^{a} and R^{b} independently from each other represent halogen, CF₃, CN and C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN;
R³ represents hydrogen, F, C, CN, CF₃, OCF₃, SCF₃, methyl, ethyl, t-butyl, adamantyl, phenyl or hetaryl having 4 to 5 carbon atoms, wherein hetaryl has 1, 2 or 3 nitrogen atoms as ring members, wherein phenyl and hetaryl are unsubstituted or substituted by 1 or 2 identical or different radicals R⁵;
R⁴ represents CN, NO₂, SF₅, halogen, C₁-C₄-alkyl, C₁-C₄-haloalkyl, C₁-C₄-haloalkoxy, C₁-C₄-haloalkylsulfanyl, C₁-C₄-haloalkylsulfonyl, N═C(CF₃)₂ and N(SO₂CF₃)₂;
R⁵ represents CN, F, C, CF₃, OCF₃, SCF₃, SO₂CF₃, NO₂, N═C(CF₃)₂, SF₅, and N(SO₂CF₃)₂.

A further object of the invention is an electronic component comprising a at least one compound of the general formula (I)

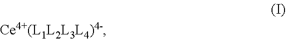

wherein
wherein
L₁; L₂; L₃; and L₄ are independently from each other selected from a bidentate ligand having the general formula (II)

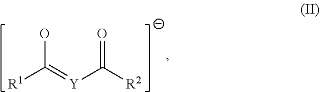

wherein
Y represents N or CR³;
R¹ represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
   cycloalkyl is unsubstituted or substituted with 1 to 13 radicals selected from fluorine and CF₃,
   phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
   naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂, pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂; or
R¹ and Y together with the C—O-group (in particular the carbon atom of the C—O-group) to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R² represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
   cycloalkyl is unsubstituted or substituted with 1 to 13 radicals selected from fluorine and CF₃,
   phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
   naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂, pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂; or
R² and Y together with the C—O-group (in particular the carbon atom of the C—O-group) to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R^{a} and R^{b} independently from each other represent halogen, CF₃, CN and C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN;
R³ represents hydrogen, F, Cl, CN, CF₃, OCF₃, SCF₃, methyl, ethyl, t-butyl, adamantyl, phenyl or hetaryl having 4 to 5 carbon atoms, wherein hetaryl has 1, 2 or 3 nitrogen atoms as ring members, wherein phenyl and hetaryl are unsubstituted or substituted by 1 or 2 identical or different radicals R⁵;
R⁴ represents CN, NO₂, SF₅, halogen, C₁-C₄-alkyl, C₁-C₄-haloalkyl, C₁-C₄-haloalkoxy, C₁-C₄-haloalkylsulfanyl, C₁-C₄-haloalkylsulfonyl, N═C(CF₃)₂ and N(SO₂CF₃)₂;
R⁵ represents CN, F, Cl, CF₃, OCF₃, SCF₃, SO₂CF₃, NO₂, N═C(CF₃)₂, SF₅, and N(SO₂CF₃)₂.

A further object of the invention is a doped semiconductor matrix material comprising at least one electron donor and at least one compound of formula (I), wherein the radicals Y, R¹ and R² have the meanings given above and as defined below.

Another object of the invention is the use of a compound (1) or mixtures thereof, wherein the radicals Y, R¹ and R² have the meanings defined before and as defined below,
as organic semiconductor,
as doping agent in organic semiconductor matrix materials, especially as p-dopant in hole transport layers,
as charge injector in a charge injection layer,
as cathode material in organic batteries,
as electrochromic material.

A further object of the invention is the use of Ce(Ill)-complex anions obtained by reduction of a compound (1) as defined above and below or of charge transfer complexes of a compound (1), as defined above and below, with electron donors as organic conductor, as electrochromic material or as ferrimagnets.

### DESCRIPTION OF THE INVENTION

The invention has the following advantages:
The cerium-(IV)-complexes according to the invention have only low production costs.
The cerium-(IV)-complexes according to the invention are advantageously suitable as electron acceptors for use as p-dopants and as electron transport materials in organic-electronic components.
The cerium-(IV)-complexes according to the invention exhibit better conductivity compared to known electron acceptors.
The cerium-(IV)-complexes according to the invention show an improved thermostability of the doped layers compared to the state of the art.
Furthermore, the cerium (IV) complexes according to the invention are characterized by a higher doping efficiency.
The cerium-(IV)-complexes and the corresponding reduced Cerium-(III)-species according to the invention show only a low absorption of the doped layer. Therefore, parasitic absorption and emissions can be reduced or even prevented.
The cerium-(IV)-complexes according to the invention are suitable for the production of organic and hybrid opto-electronic components, both by means of solvent processing and by vacuum reprocessing.

In the context of the invention, a bidentate ligand (also called didentate) is a ligand, which binds with two atoms to the metal atom (cerium atom).

In the context of the invention, a homoleptic cerium (IV) compound is a complex, wherein all ligands are identical.

In the context of the invention, a heteroleptic cerium (IV) compound is a complex, wherein the meaning of at least one ligand is different to the remaining ligands.

In the context of the invention, the prefix Cₙ-Cₘ indicates the number of carbon atoms that a molecule or residue designated thereby may contain.

In the context of the invention, the expression C₁-C₆-alkyl refers to unbranched or branched saturated hydrocarbon groups having 1 to 6 carbon atoms. C₁-C₆-alkyl are e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl. C₁-C₄-alkyl refers, e.g. to methyl, ethyl, propyl, 1-methylethyl, butyl, 1-methylpropyl, 2-methylpropyl or 1,1-dimethylethyl.

In the context of the invention the expression C₁-C₆-alkoxy refers to an unbranched or branched saturated C₁-C₆-alkyl group as defined above, which is bound via an oxygen atom. Alkoxy radicals with 1 to 4 carbon atoms are preferred, particularly preferred are 1 or 2 carbon atoms. C₁-C₂-alkoxy is methoxy or ethoxy. C₁-C₄-alkoxy is e.g. methoxy, ethoxy, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-alkoxy comprises the meanings given for C₁-C₄-alkoxy and additionally e.g. pentoxy, 1 methylbutoxy, 2-methylbutoxy, 3-methylbutoxy, 1,1-dimethylpropoxy, 1,2-dimethylpropoxy, 2,2-dimethylpropoxy, 1-ethylpropoxy, hexyloxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy and 3,3-dimethylbutoxy.

In the context of the invention the expression C₁-C₆-alkylsulfanyl refers to an unbranched or branched saturated C₁-C₆-alkyl group as defined above, which is bound via a sulfur atom. Alkylsulfanyl radicals with 1 to 4 carbon atoms are preferred, particularly preferred are 1 or 2 carbon atoms. C₁-C₂-alkylsulfanyl is methylsulfanyl or ethylsulfanyl. C₁-C₄-alkylsulfanyl is e.g. methylsulfanyl, ethylsulfanyl, n-propylsulfanyl, 1-methylethylsulfanyl (isopropylsulfanyl), butylsulfanyl, 1-methylpropylsulfanyl (sec-butylsulfanyl), 2-methylpropylsulfanyl (isobutylsulfanyl) or 1,1-dimethylethylsulfanyl (tert-butylsulfanyl). C₁-C₆-alkylthio comprises the meanings given for C₁-C₄-alkylsulfanyl and additionally also, e.g., pentylsulfanyl, 1-methylbutylsulfanyl, 2-methylbutylsulfanyl, 3-methylbutylsulfanyl, 1,1-dimethylpropylsulfanyl, 1,2-dimethylpropylsulfanyl, 2,2-dimethylpropylsulfanyl, 1-ethylpropylsulfanyl, hexylsulfanyl, 1-methylpentylsulfanyl, 2-methylpentylsulfanyl, 3-methylpentylsulfanyl, 4-methylpentylsulfanyl, 1,1-dimethylbutylsulfanyl, 1,2-dimethylbutylsulfanyl, 1,3-dimethylbutylsulfanyl, 2,2-dimethylbutylsulfanyl, 2,3-dimethylbutylsulfanyl, 3,3-dimethylbutylsulfanyl, 1-ethylbutylsulfanyl, 2-ethylbutylsulfanyl, 1,1,2-trimethylpropylsulfanyl, 1,2,2-trimethylpropylsulfanyl, 1-ethyl-1-methylpropylsulfanyl or 1-ethyl-2-methylpropylsulfanyl.

In the context of the invention the expressions haloalkyl, haloalkoxy and haloalkylsulfanyl refer to partially or fully halogenated alkyl, alkoxy or alkylsulfanyl. In other words, one or more hydrogen atoms, for example 1, 2, 3, 4 or 5 hydrogen atoms bonded to one or more carbon atoms of alkyl, alkoxy or alkylsulfanyl are replaced by a halogen atom, in particular by fluorine or chlorine.

The expression “halogen” denotes in each case fluorine, chlorine, bromine or iodine.

The expression “CN” denotes the cyano group (—C═N).

The expression “aryl” comprises in the context of the invention mono- or polynuclear aromatic hydrocarbon radicals with usually 6 to 14, especially preferably 6 to 10 carbon atoms. Examples of aryl are especially phenyl, naphthyl, indenyl, fluorenyl, anthracenyl, phenanthrenyl, naphthacenyl, chrysenyl, pyrenyl, etc. and especially phenyl or naphthyl.

The expression “hetaryl” comprises in the context of the invention mononuclear aromatic hydrocarbon radicals with 4 to 5 carbon atoms, wherein 1, 2 or 3 carbon atoms have been replaced by 1, 2 or 3 nitrogen atoms as ring members. The hetaryl group may be attached to the remainder of the molecule via a ring carbon or via a ring nitrogen. Examples of 5- or 6-membered aromatic heterocyclic rings (also called heteroaromatic rings or hetaryl) are 2-pyrrolyl, 3-pyrrolyl, 3-pyrazolyl, 4-pyrazolyl, 5-pyrazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

In the context of the invention the term “C₅-C₇-cycloalkyl” as used herein refers to a monocyclic membered saturated cycloaliphatic radicals, e.g. cyclopentyl, cyclohex-yl, cycloheptyl. The term C₅-C₇-halocycloalkyl as used herein, which is also expressed as “cycloalkyl which is partially or fully halogenated”, refers C₅-C₇-cycloalkyl as mentioned above, in which some or all of the hydrogen atoms are replaced by halogen atoms as mentioned above, in par-ticular fluorine.

When ˜ appears in a formula, showing a preferred substructure of a compound of the present invention, it denotes the bond to the rest of the molecule.

Cerium compounds of the formula (I)

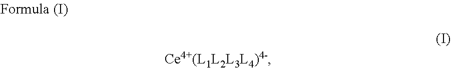

wherein
L₁; L₂; L₃; and L₄ are defined as above and below, encompasses compounds, wherein
all four ligands L₁, L₂, L₃ and L₄ have the same meaning,
three of the four ligands have the same meaning,
two of the four ligands have the same meaning,
all four ligands L₁, L₂, L₃ and L₄ have different meanings.

Preferred are compounds of formula (I) wherein L₁, L₂, L₃ and L₄ have the same meaning.

In the cerium compounds of formula (I) L₁, L₂, L₃ and L₄ are independently from each other selected from bidentate ligands having the general formula (II). In the following preferred embodiments of the compounds (1) are directly defined by preferred embodiments of their bidentate ligands (II).

In a first embodiment R¹ und R² have preferably different meanings. Preferably R¹ and R² are independently selected from CFR^{a}R^{b}, t-butyl, adamantyl, C₂F₅, n-C₃F₇, C₃-C₇-cycloalkyl, phenyl, naphthyl, pyridyl, pyrimidyl, triazinyl,
wherein
cycloalkyl is unsubstituted or substituted with 1 to 13 radicals selected from fluorine, CF₃, phenyl is substituted by at least one radical selected from NO₂, CN, F, Cl, Br, CF₃, SF₅, OCF₃, SCF₃, SO₂CF₃, N═C(CF₃)₂, and N(SO₂CF₃)₂, naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl, pyrimidyl and triazinyl are substituted by at least one radical selected from NO₂, CN, F, Cl, Br, CF₃, SF₅, OCF₃, SCF₃, SO₂CF₃, N═C(CF₃)₂, and N(SO₂CF₃)₂.

Preferably, R¹ is selected from from CFR^{a}R^{b}, t-butyl, adamantyl, C₂F₅, n-C₃F₇ and C₃-C₇-cycloalkyl, wherein cycloalkyl is unsubstituted or substituted with 1 to 13 radicals selected from F and CF₃.
and
R² is selected from CFR^{a}R^{b}, phenyl, naphthyl, pyridyl and pyrimidyl, wherein phenyl is substituted by at least one radical selected from NO₂, CN, F, Cl, Br, CF₃, SF₅, OCF₃, SCF₃, SO₂CF₃, N═C(CF₃)₂, and N(SO₂CF₃)₂,
naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl and pyrimidyl are substituted by at least one radical selected from NO₂, CN, F, Cl, Br, CF₃, SF₅, OCF₃, SCF₃, SO₂CF₃, N═C(CF₃)₂, and N(SO₂CF₃)₂.

In particular R¹ is selected from CFR^{a}R^{b}, t-butyl, adamantyl, C₂F₅, n-C₃F₇ and C₅-C₆-cycloalkyl, especially CFR^{a}R^{b}, t-butyl, adamantyl, C₂F₅, n-C₃F₇, 2,2,3,3,4,4,5,5-octafluoro-1-(trifluoromethyl)cyclopentyl, nonafluorocyclopentyl, 2,2,3,3,4,4,5,5,6,6-decafluoro-1-(trifluoromethyl)cyclohexyl and 1,2,2,3,3,4,4,5,5,6,6-undecafluorocyclohexyl.

In particular R² is selected from CFR^{a}R^{b}, phenyl, naphthyl, pyridyl and pyrimidyl, wherein phenyl is substituted by at least one radical selected from CN, F, C and CF₃, naphthyl is unsubstituted or substituted with 1 to 7 radicals selected from F, C, and CF₃, pyridyl and pyrimidyl are substituted by at least one radical selected from F, C and CF₃.

In a second embodiment R¹ und R² have the same meaning. Preferably R¹ und R² are selected from CFR^{a}R^{b} and phenyl, which is substituted by at least one radical selected from F, Cl, Br, CF₃, SF₅, OCF₃, SCF₃, SO₂CF₃, N═C(CF₃)₂, and N(SO₂CF₃)₂,
R^{a} is preferably F and R^{b} is preferably selected from C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN.

In a third embodiment R¹ and Y together with the C—O-group to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴, and
R² is selected from CFR^{a}R^{b}, t-butyl, adamantyl, C₂F₅, n-C₃F₇ and C₃-C₇-cycloalkyl, wherein cycloalkyl is unsubstituted or substituted with 1 to 13 radicals selected from F and CF₃.

Irrespective of its occurrence R³ is preferably selceted from H, CN, t-butyl, adamantyl, 3,5-triflouromethyl-phenyl, CF₃, OCF₃ and SCF₃, especially selceted from H, CN, t-butyl, adamantyl, 3,5-triflouromethyl-phenyl.

Irrespective of its occurrence R⁴ is preferably selcted from CN, NO₂, SF₅, halogen, C₁-C₄-alkyl, C₁-C₄-haloalkyl, C₁-C₄-alkoxy, C₁-C₄-haloalkoxy, C₁-C₄-alkylsulfanyl, C₁-C₄-haloalkylsulfanyl, C₁-C₄-alkylsulfonyl, C₁-C₄-haloalkylsulfonyl, N═C(CF₃)₂ and N(SO₂CF₃)₂ in particular F, CF₃, trifluoromethylsulfanyl, trifluoromethylsulfonyl, N═C(CF₃)₂, N(SO₂CF₃)₂, especially F and CF₃.

Irrespective of its occurrence R⁵ is preferably selcted F, C₁, CF₃.

Irrespective of its occurrence R^{a} and R^{b} independently form each other selected from F, CF₃ and a group A selected from A1 to A32

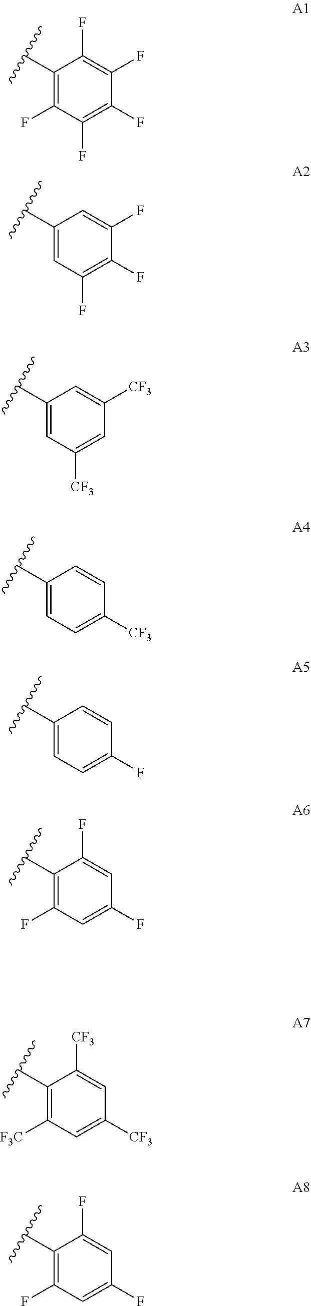

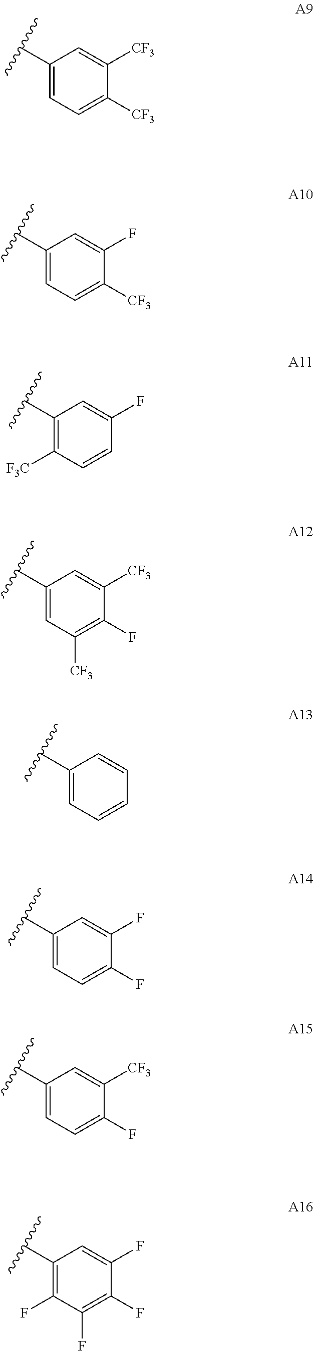

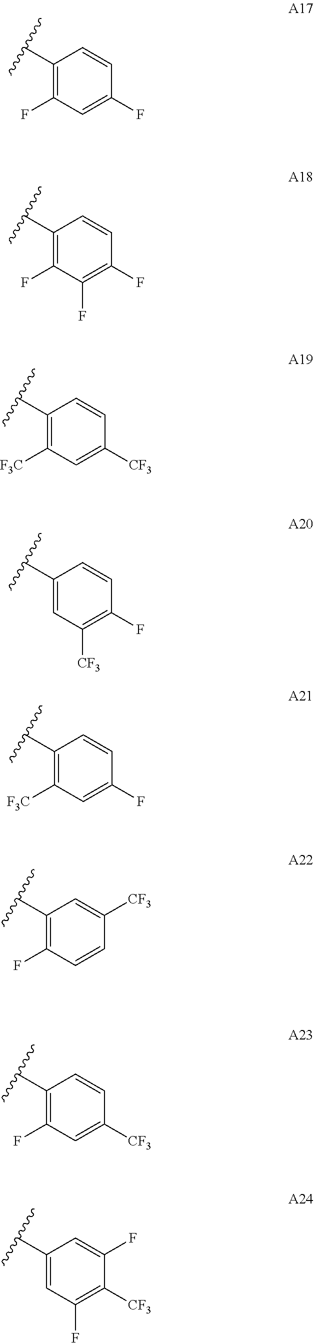

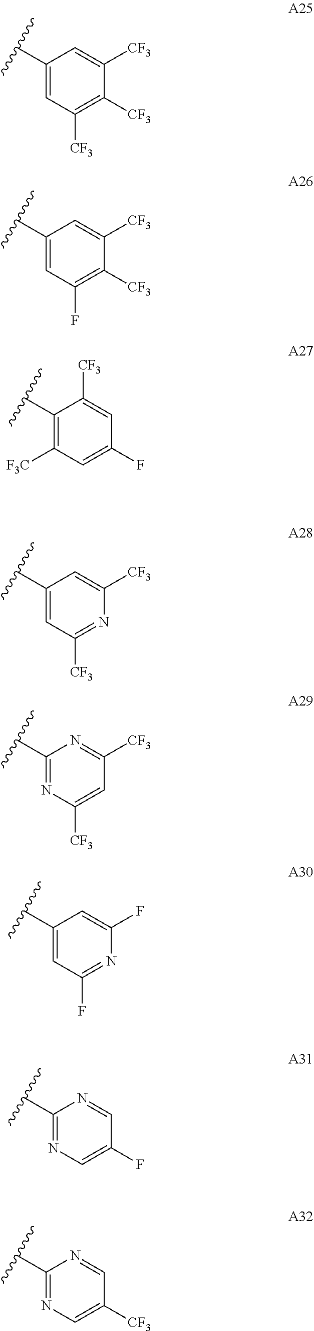

wherein ˜ denotes the bond to the remaining molecule.

In particular R^{a} is F, and R^{b} is a group A as defined above, or R^{a} and R^{b} are CF₃.

Preferably embodiment L₁, L₂, L₃, L₄ L₄ have the same meaning.

In another particular embodiment L₁, L₂, L₃, L₄ L₄ have the different meanings.

The following compounds of formula (I), wherein L₁, L₂, L₃, L₄ have the same meaning are selected from

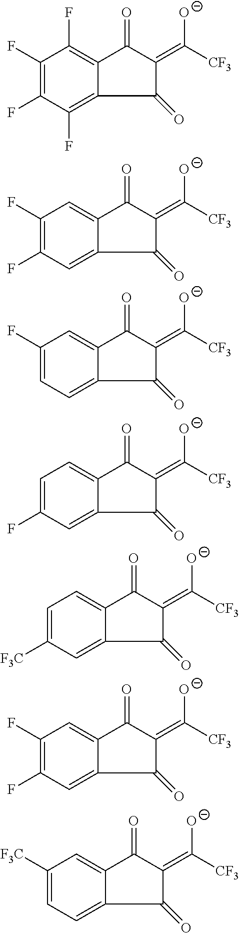

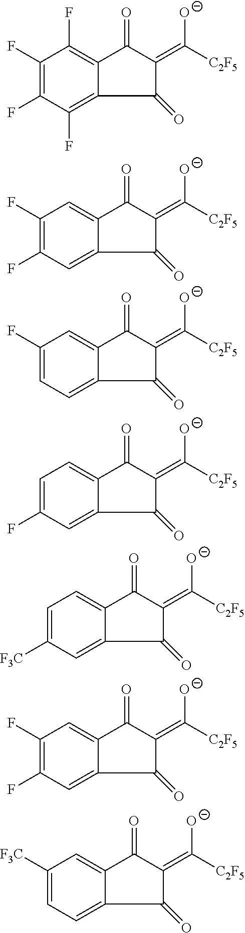

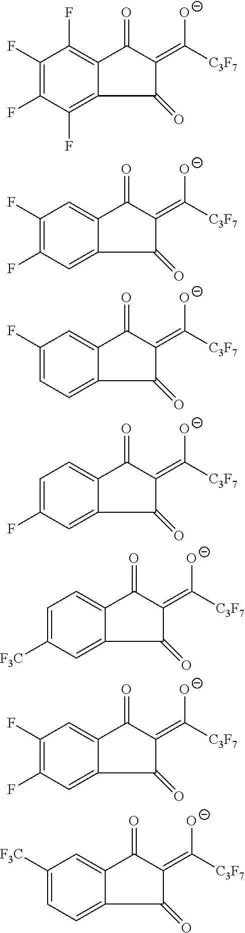

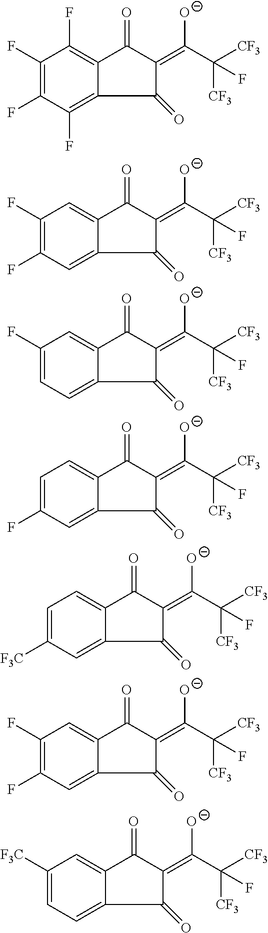

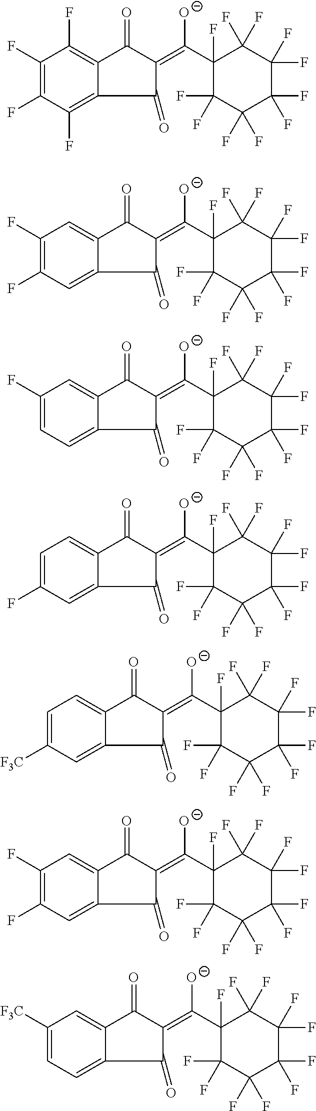

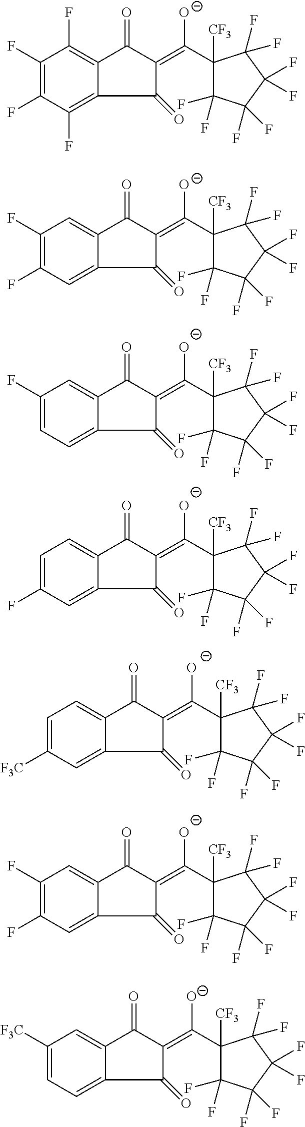

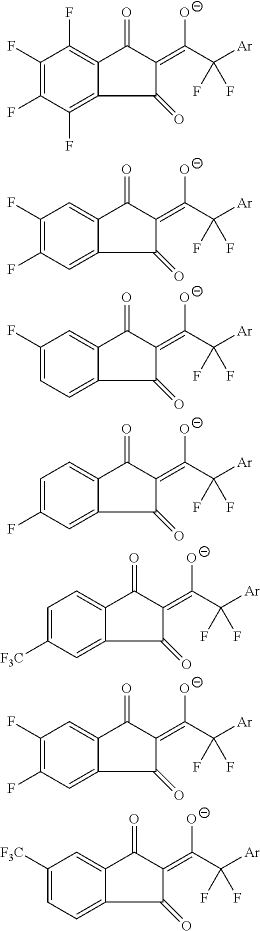

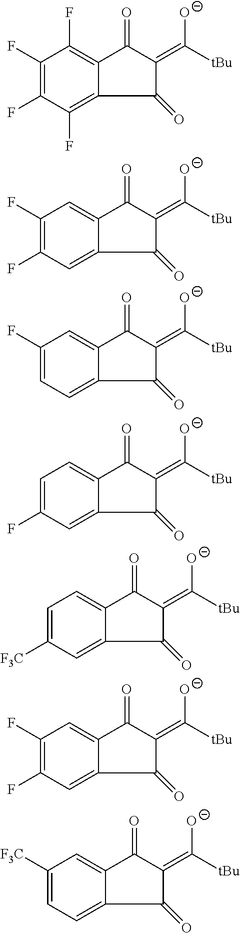

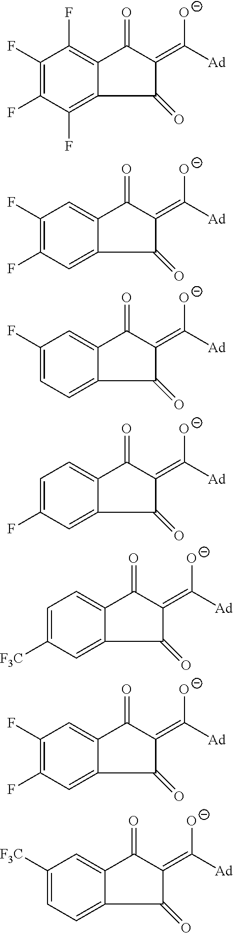

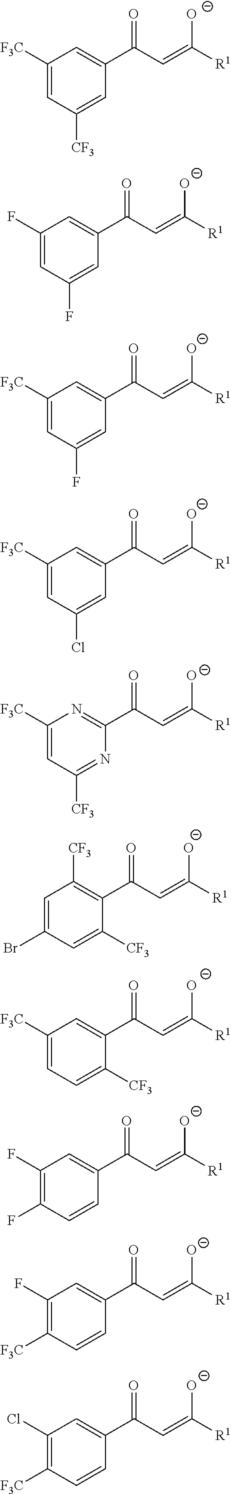

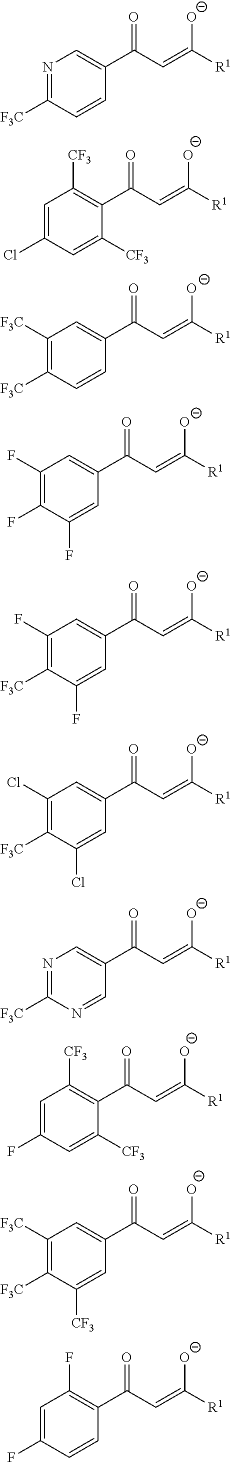

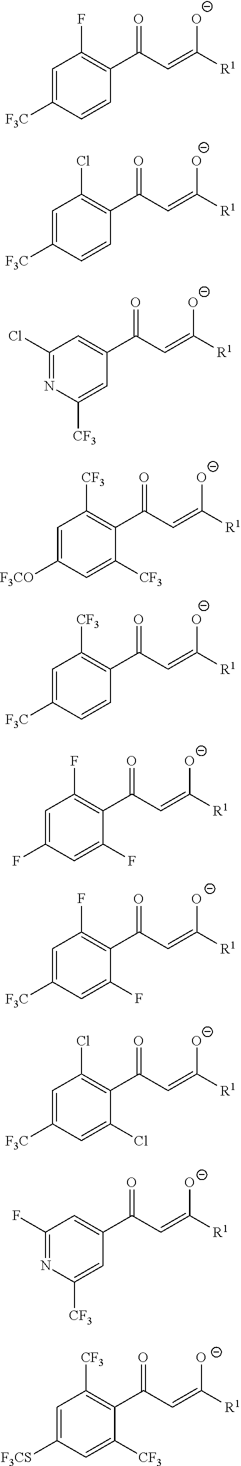

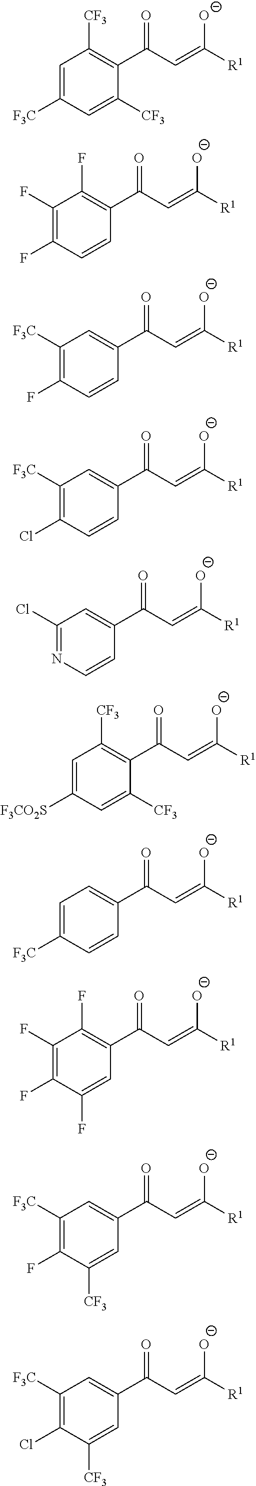

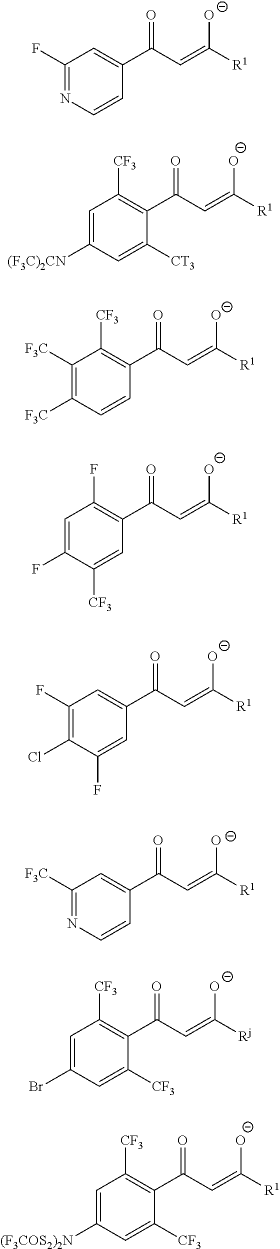

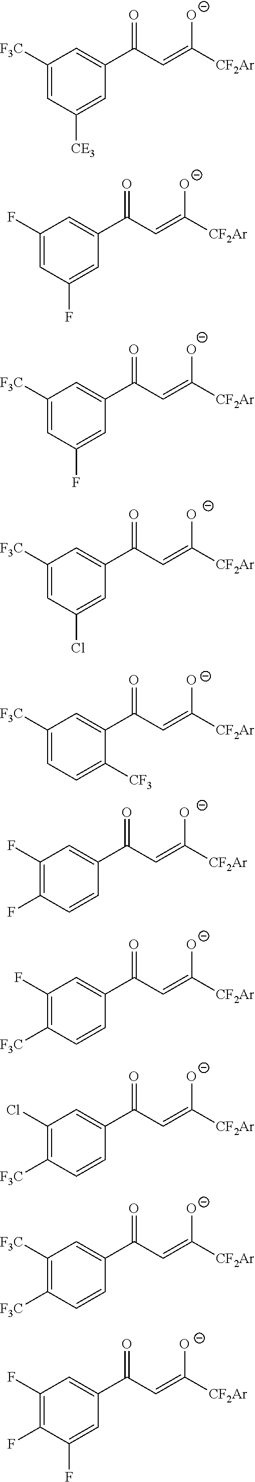

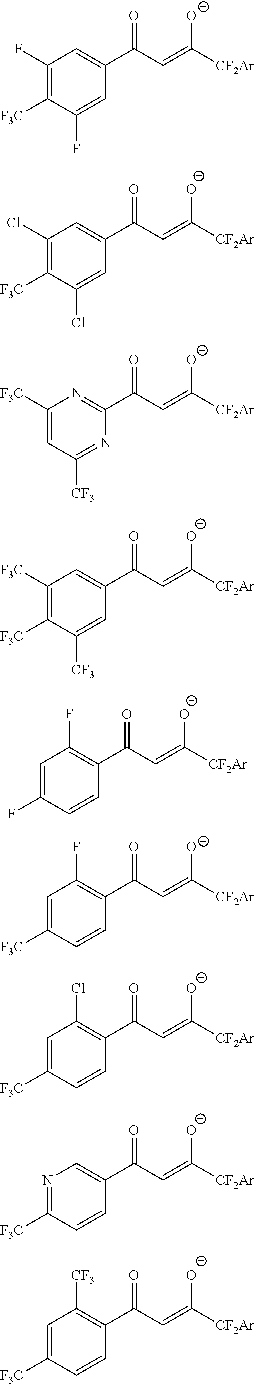

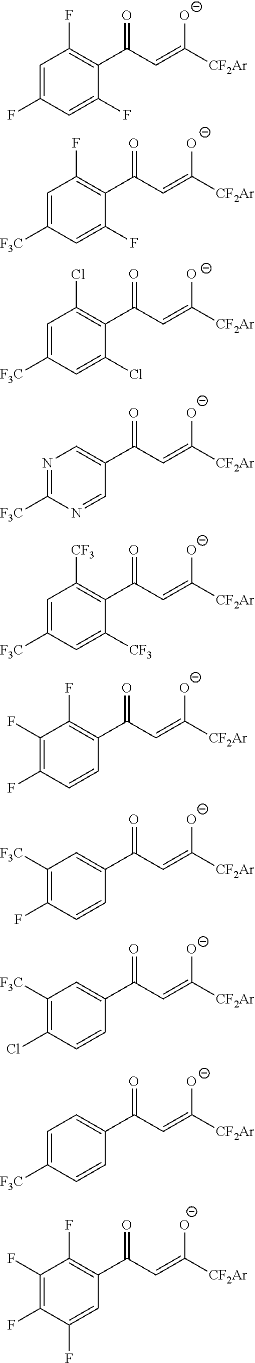

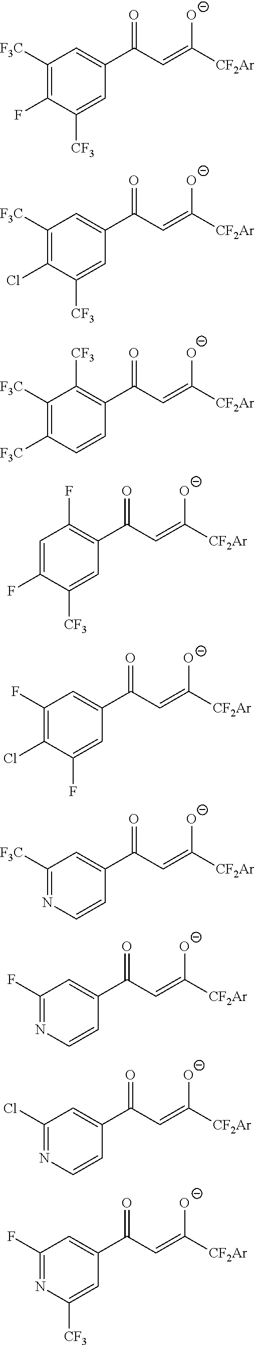

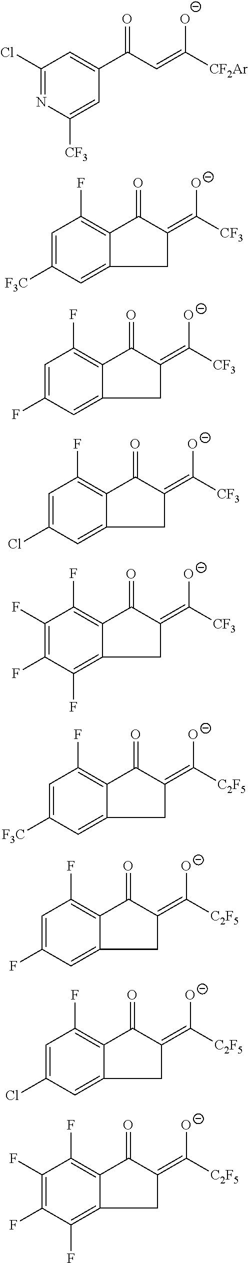

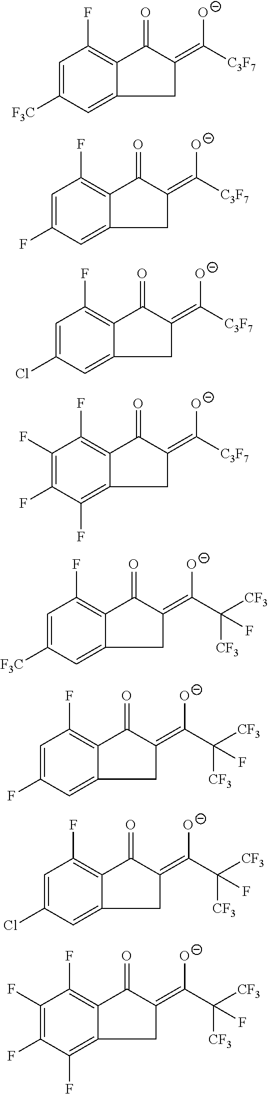

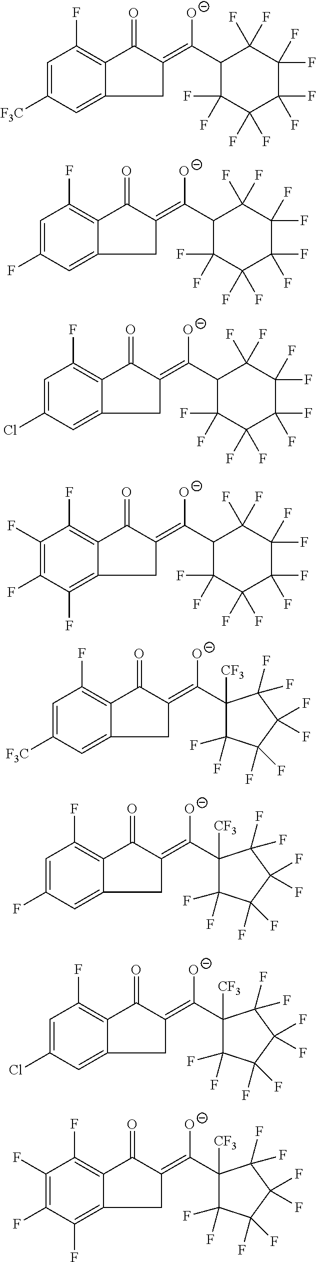

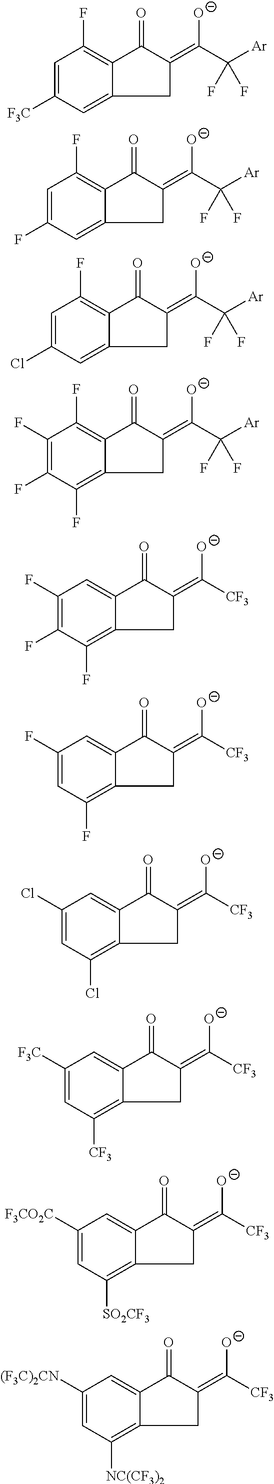

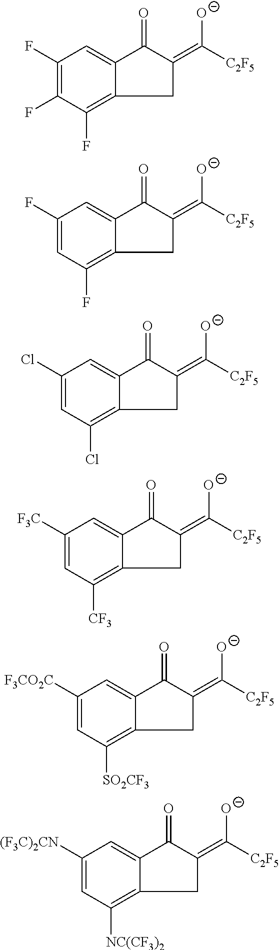

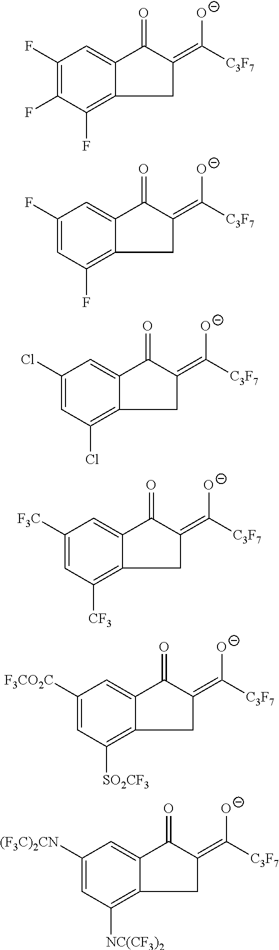

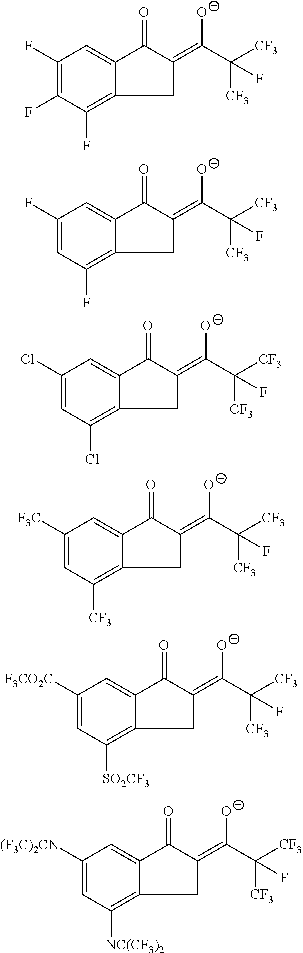

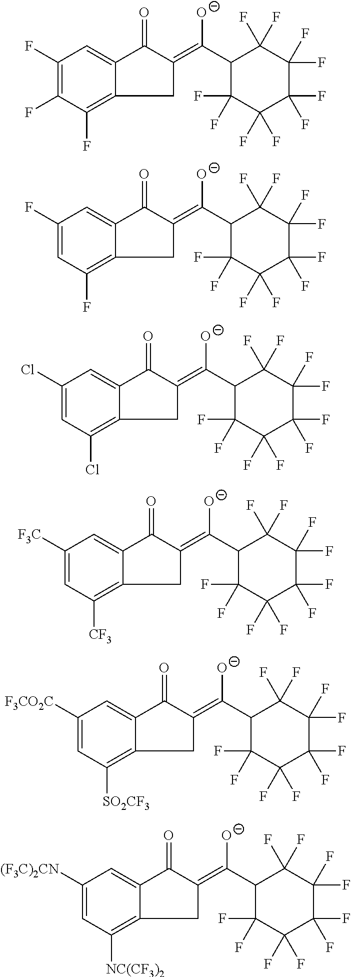

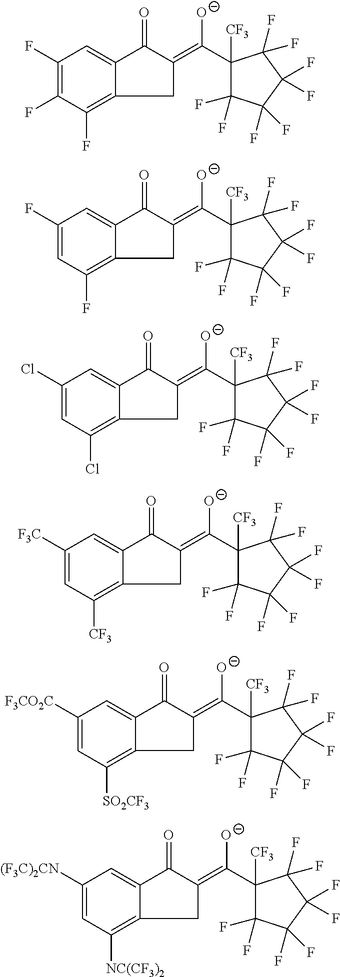

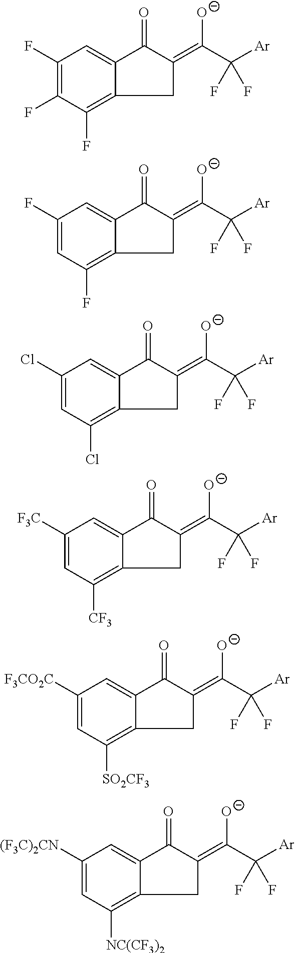

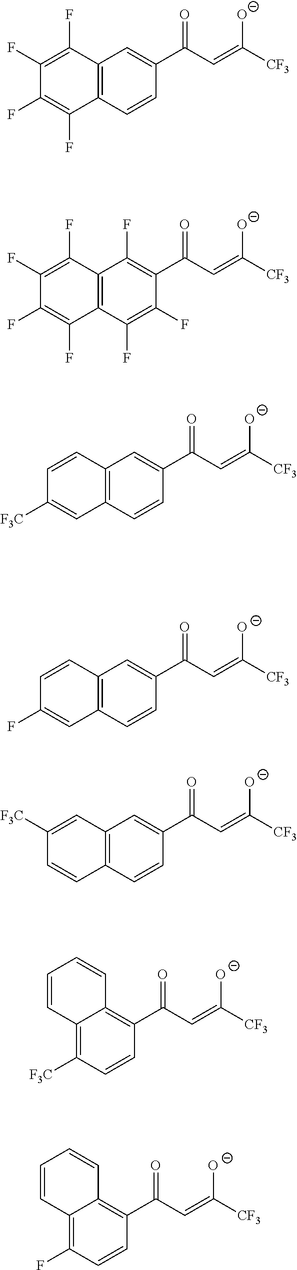

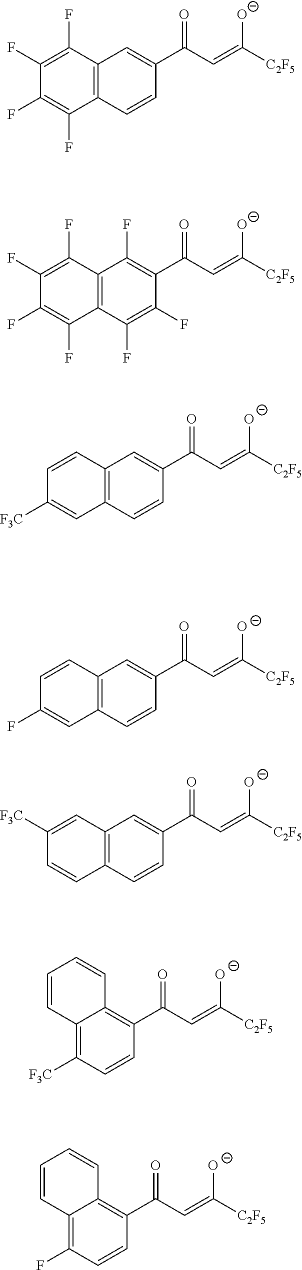

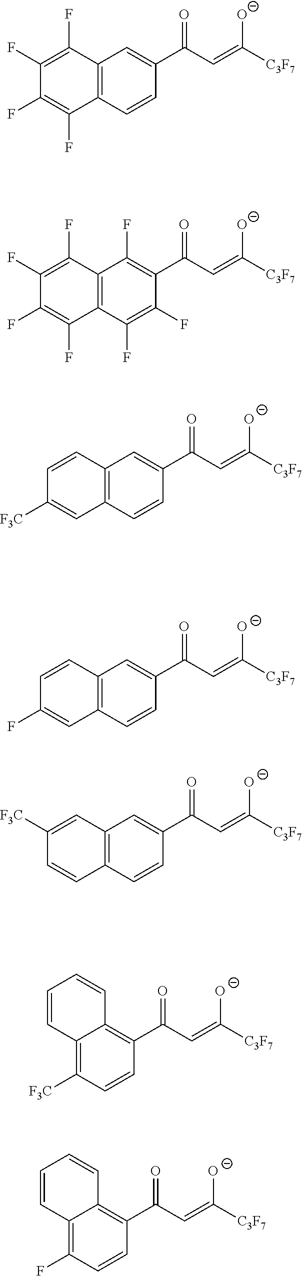

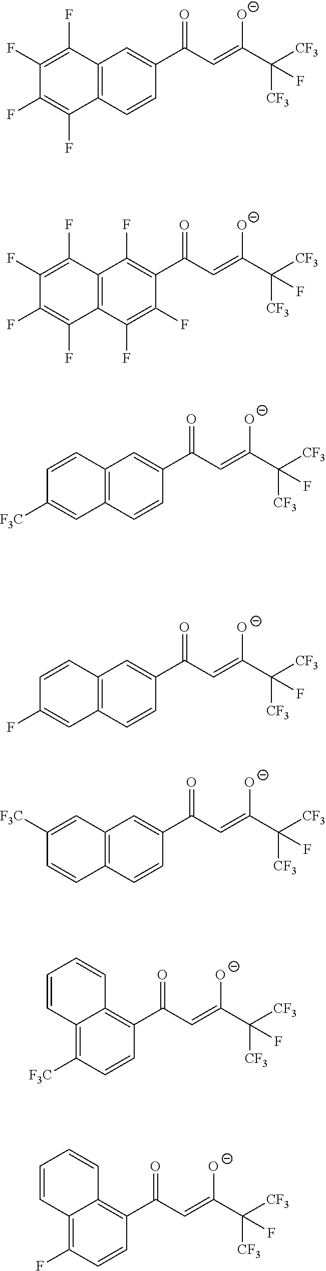

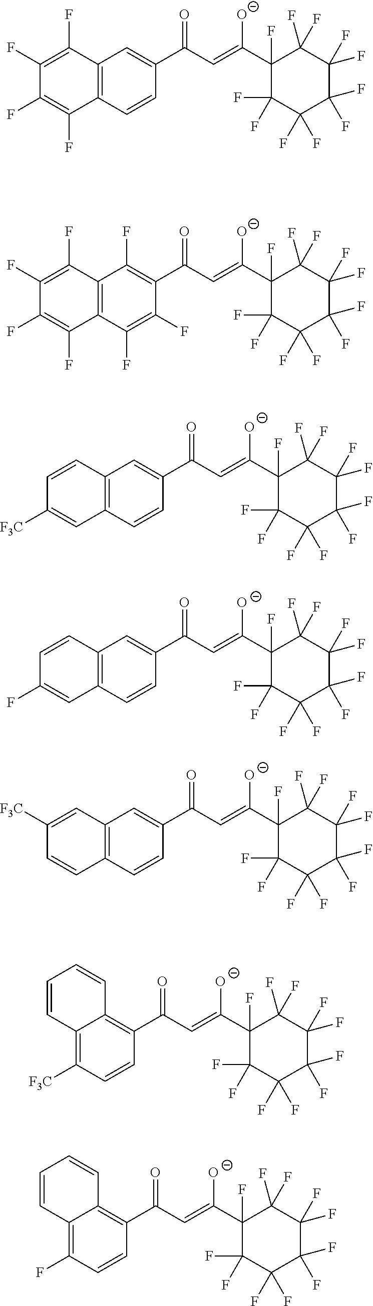

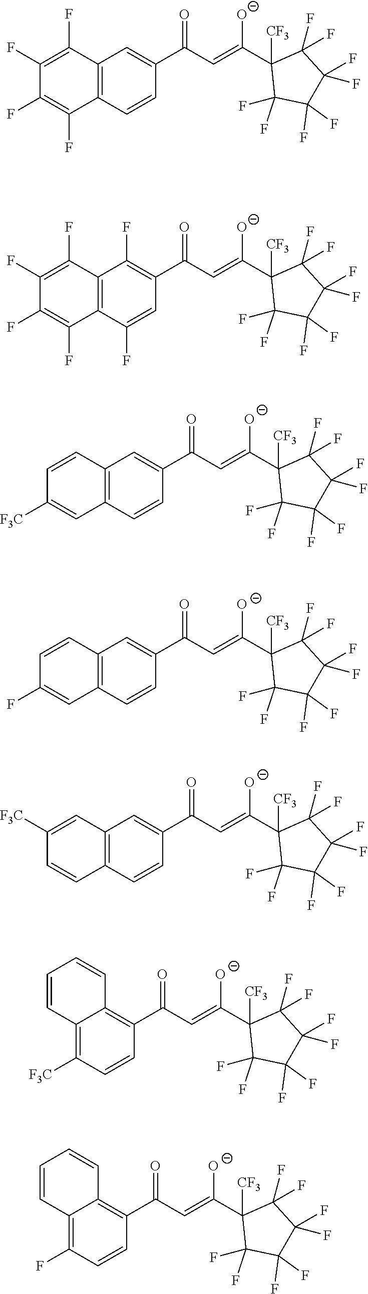

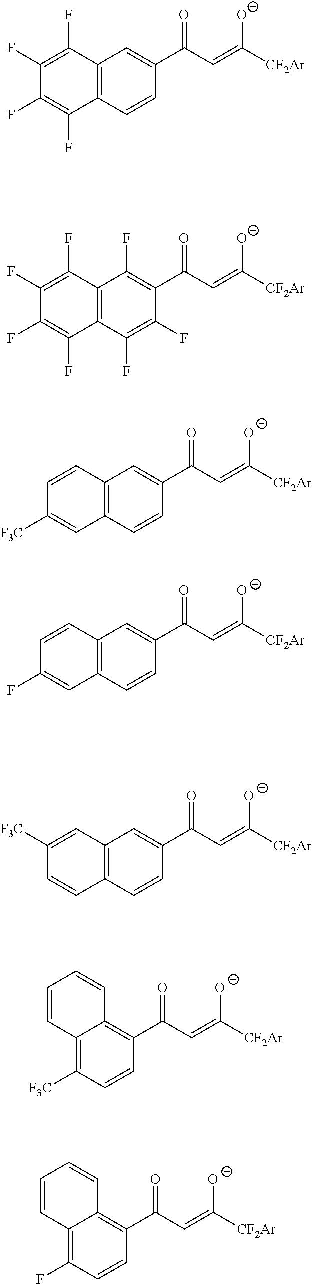

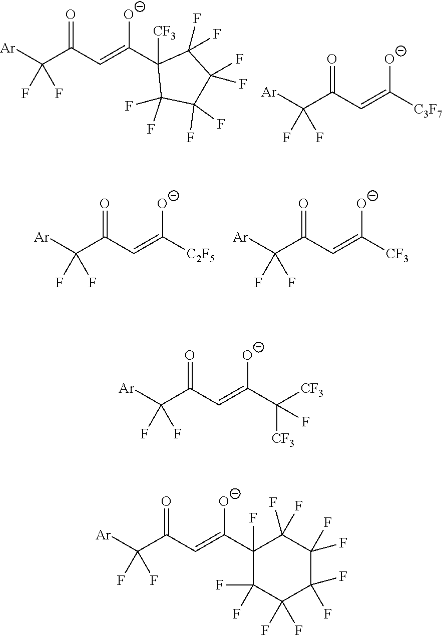

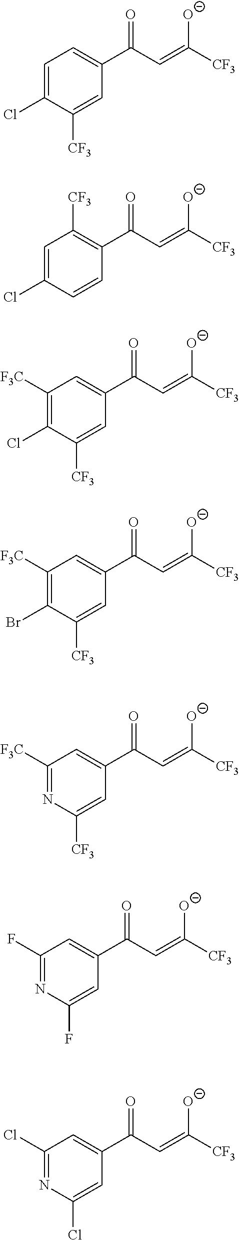

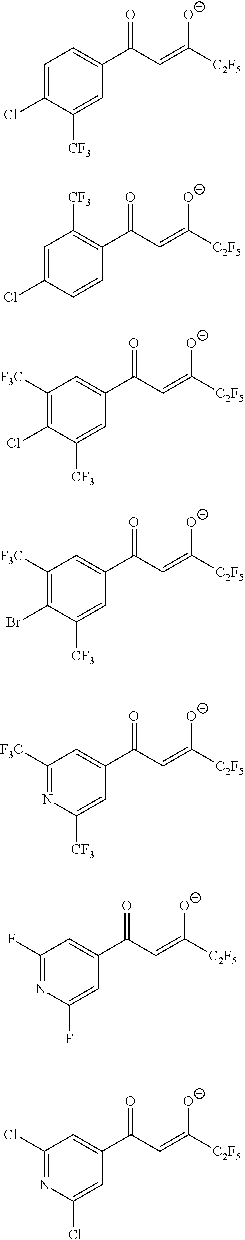

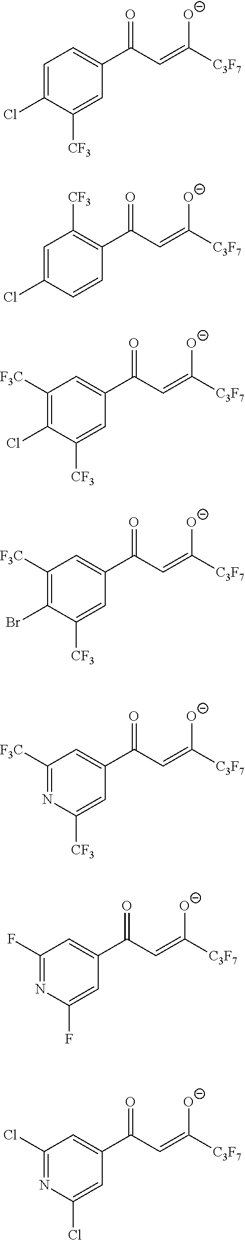

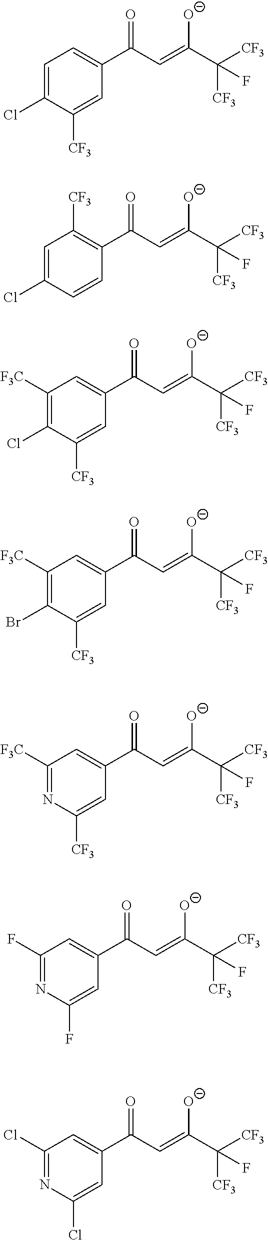

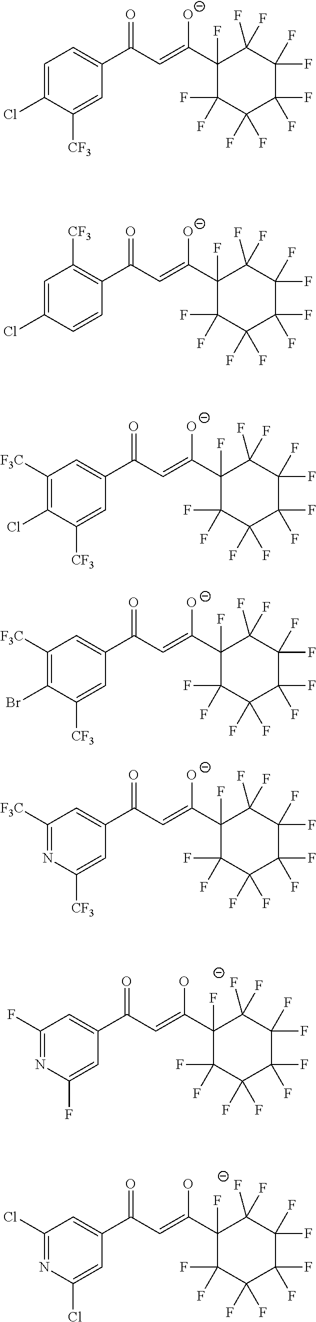

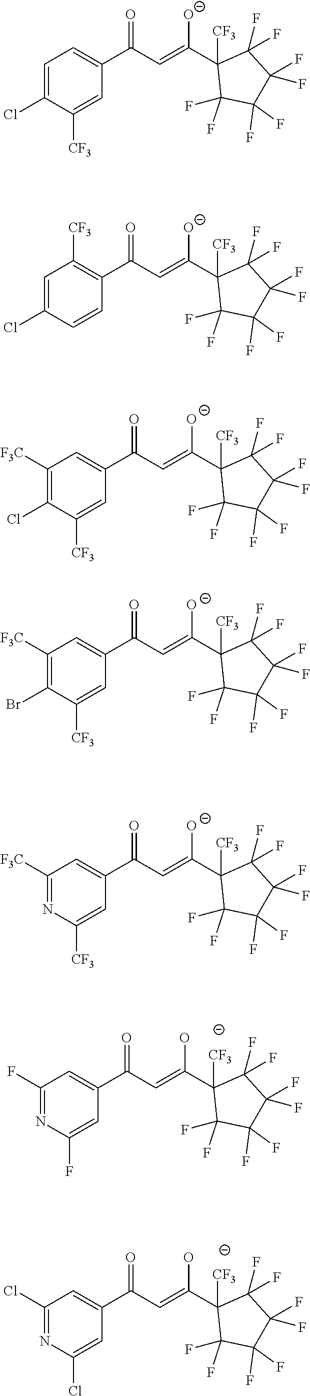

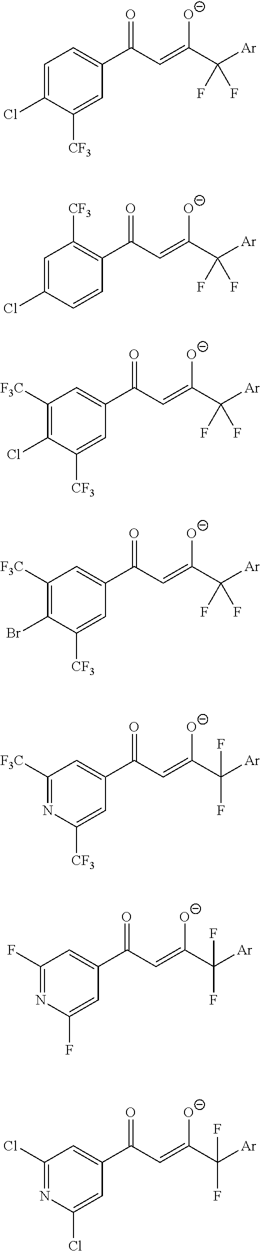

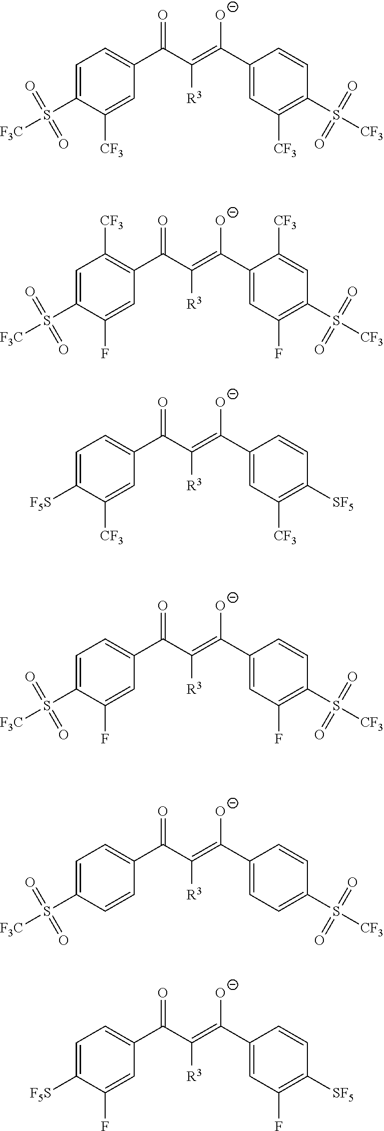

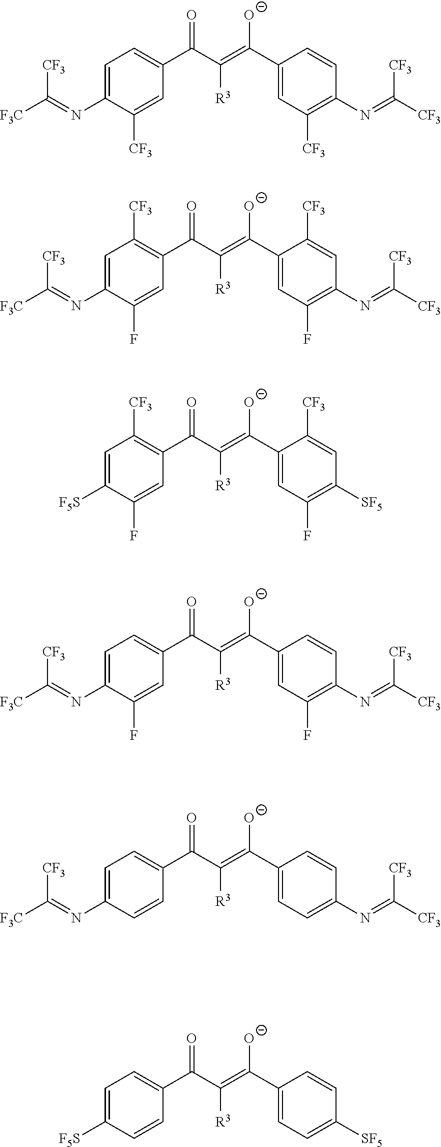

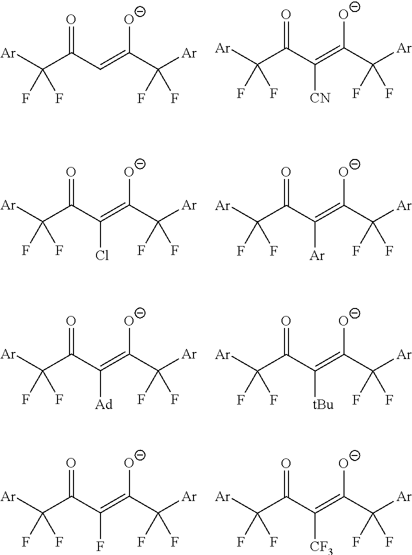

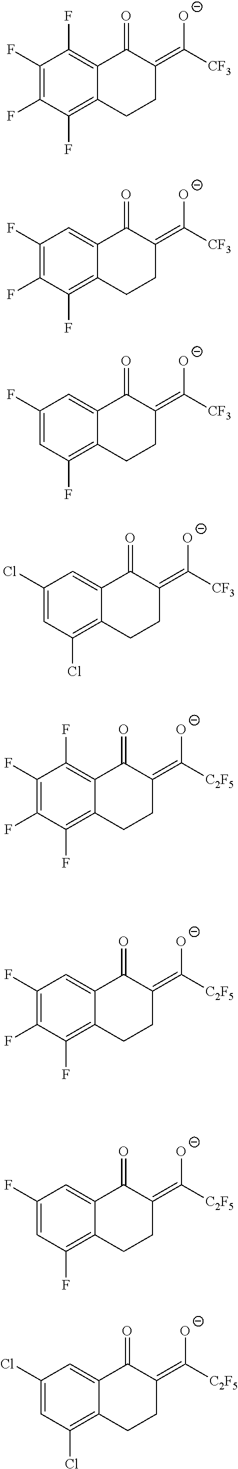

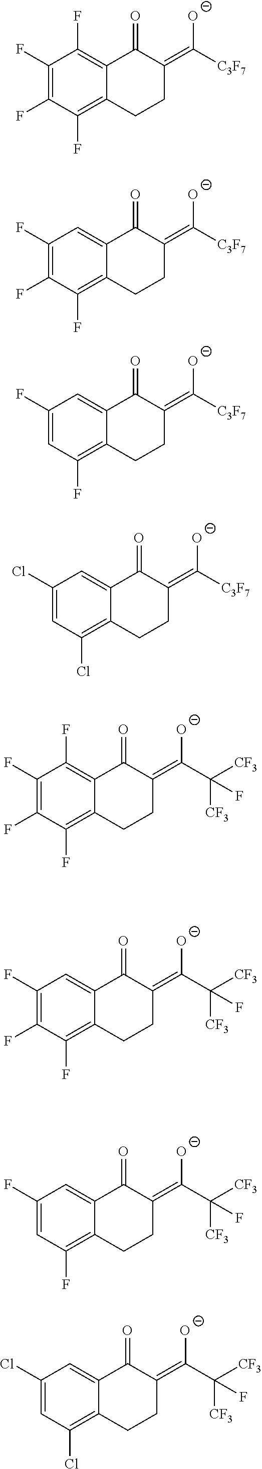

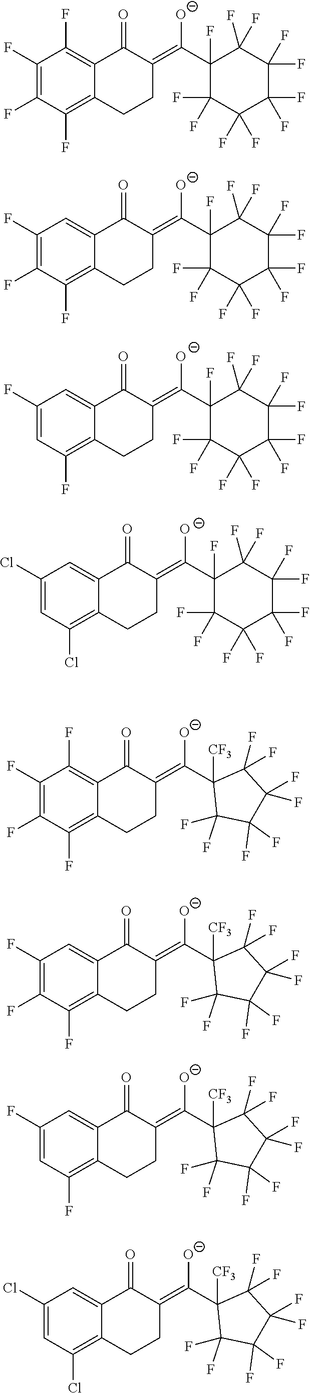

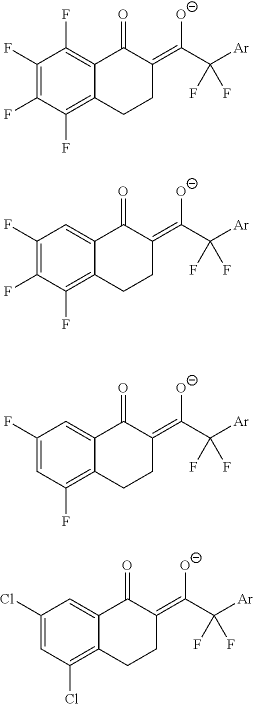

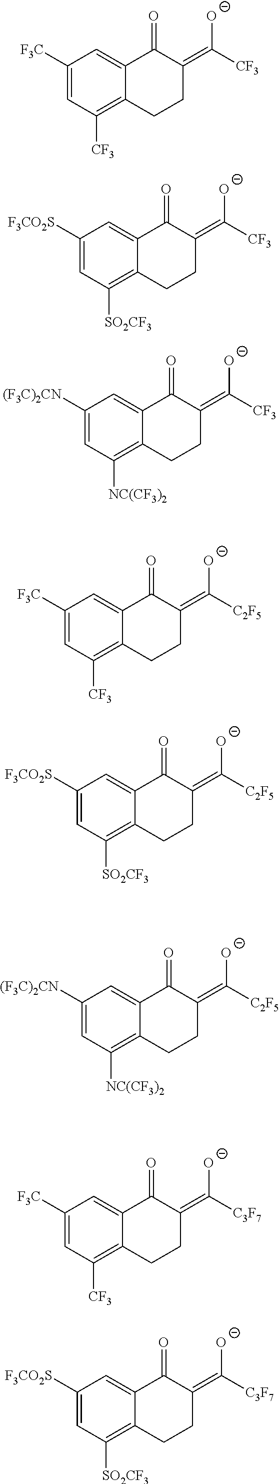

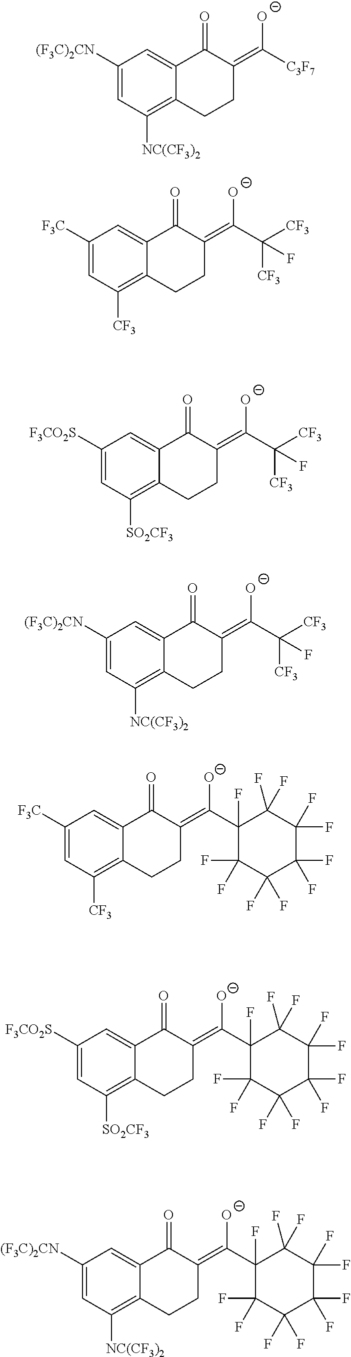

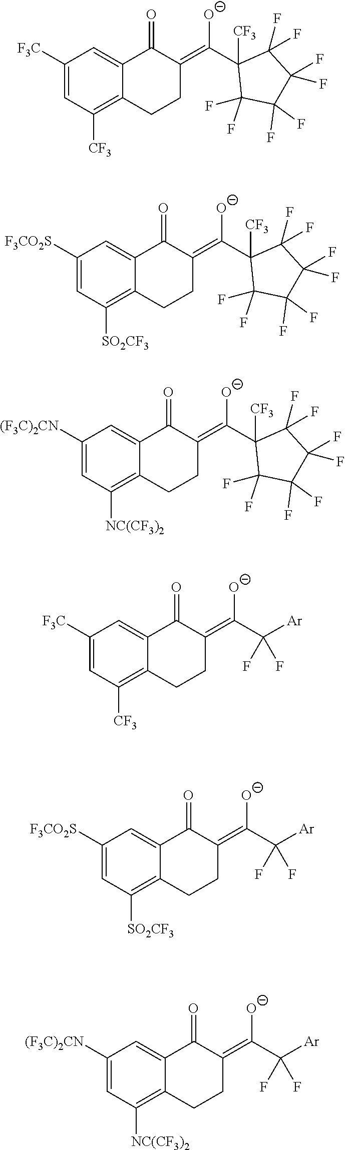

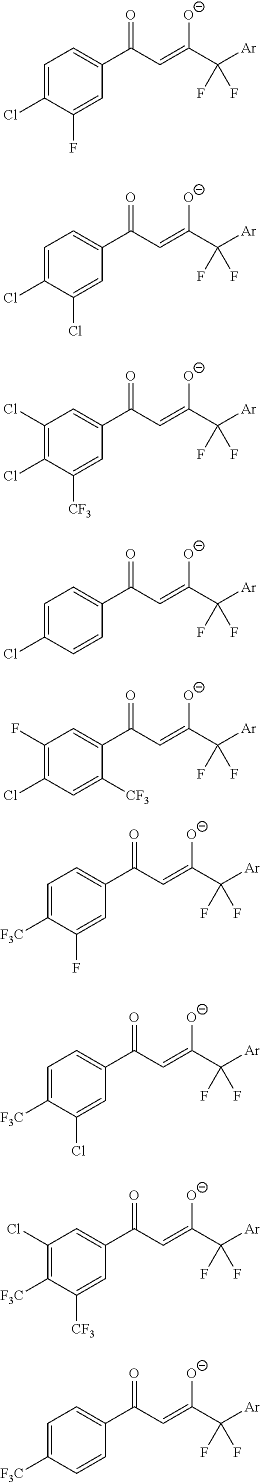

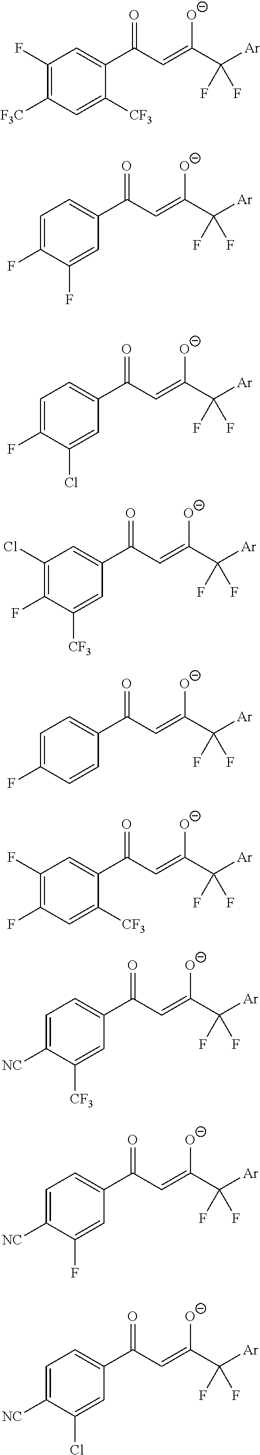

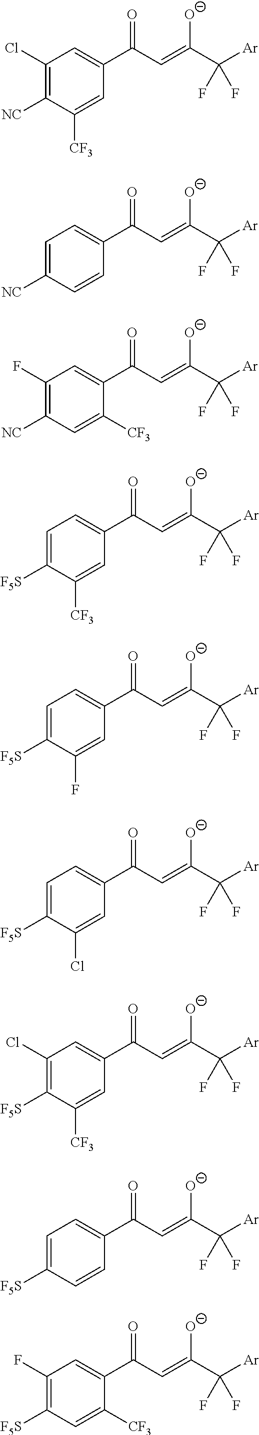

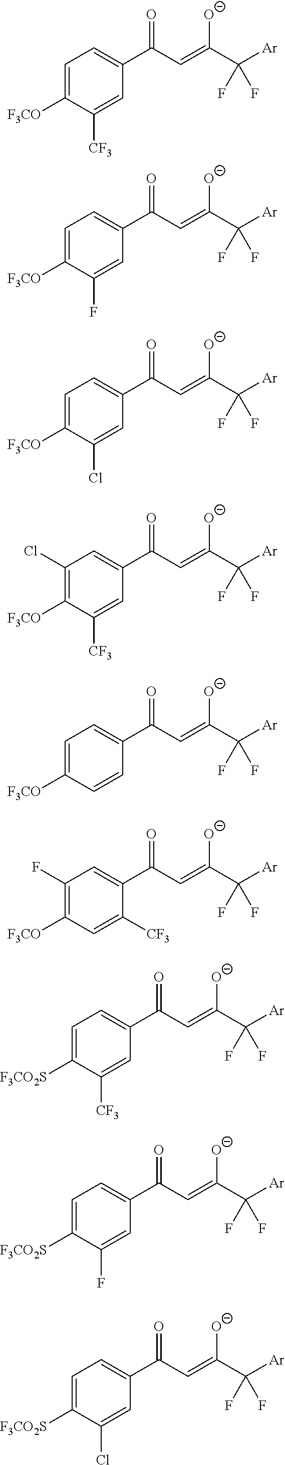

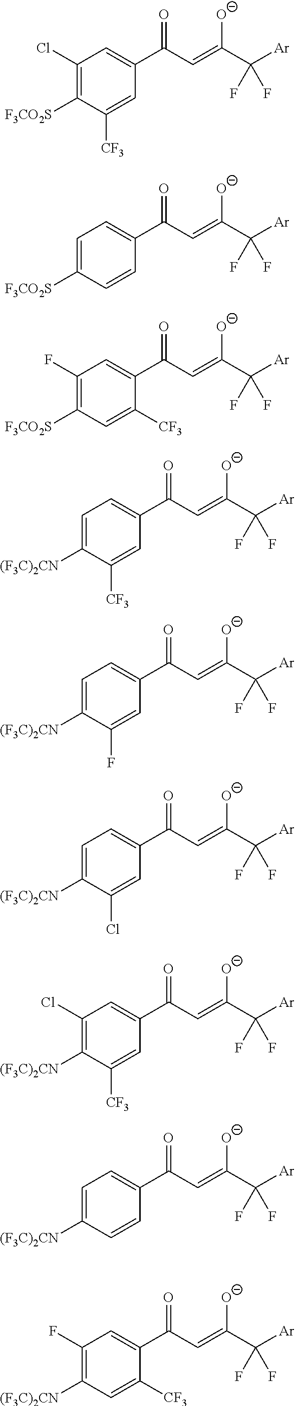

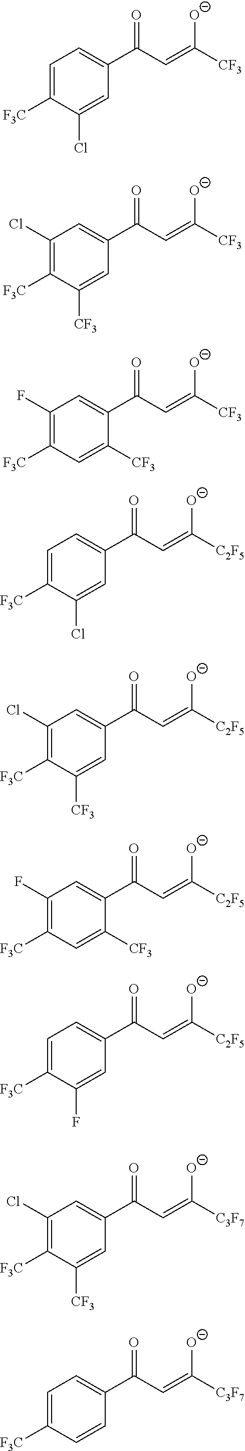

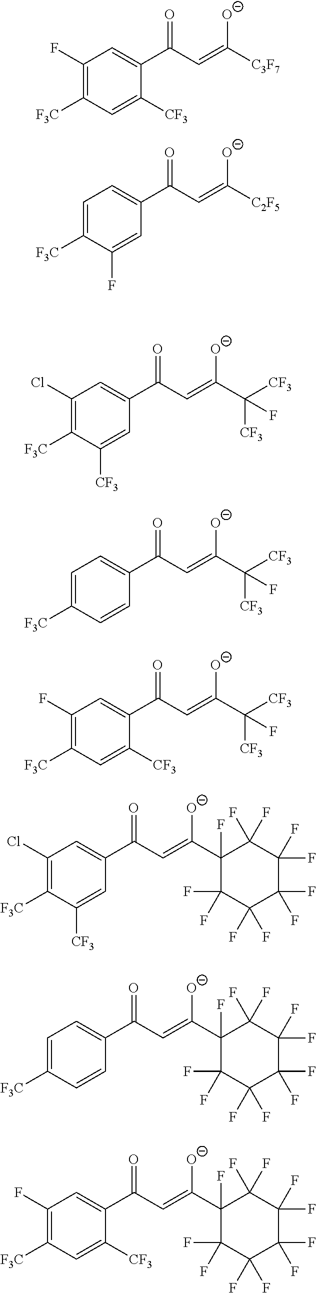

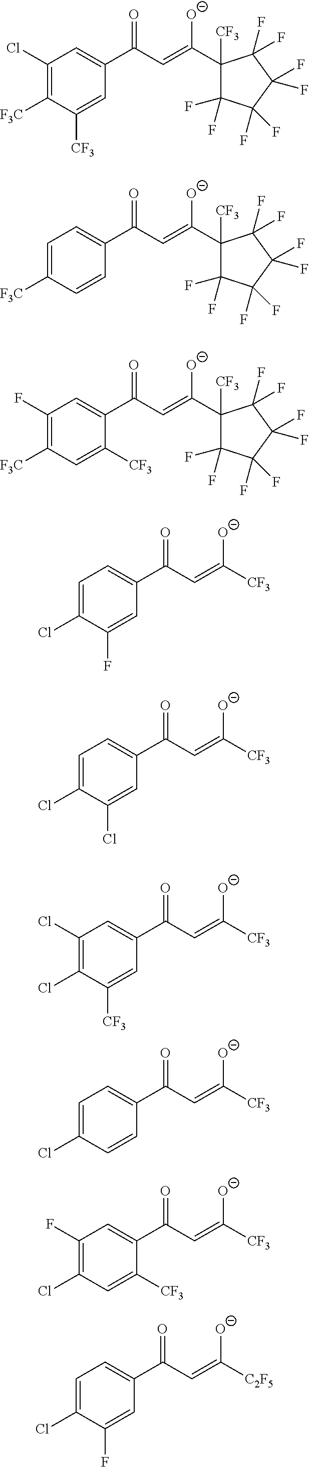

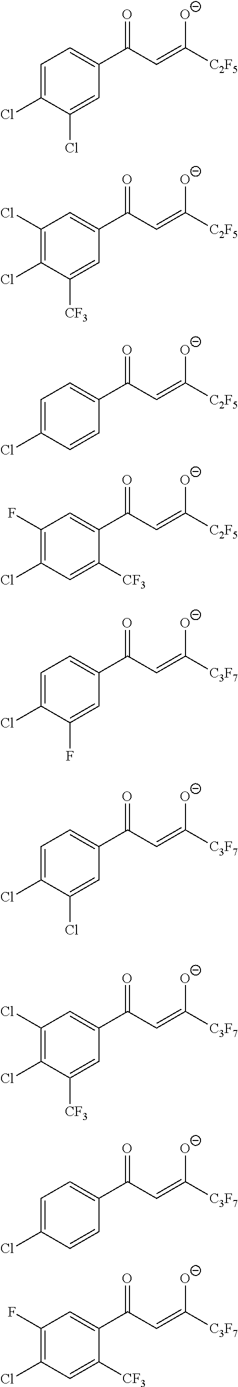

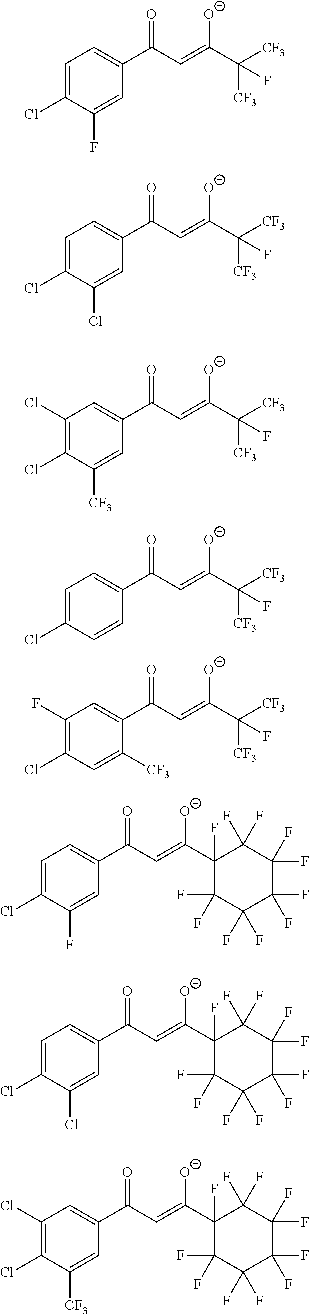

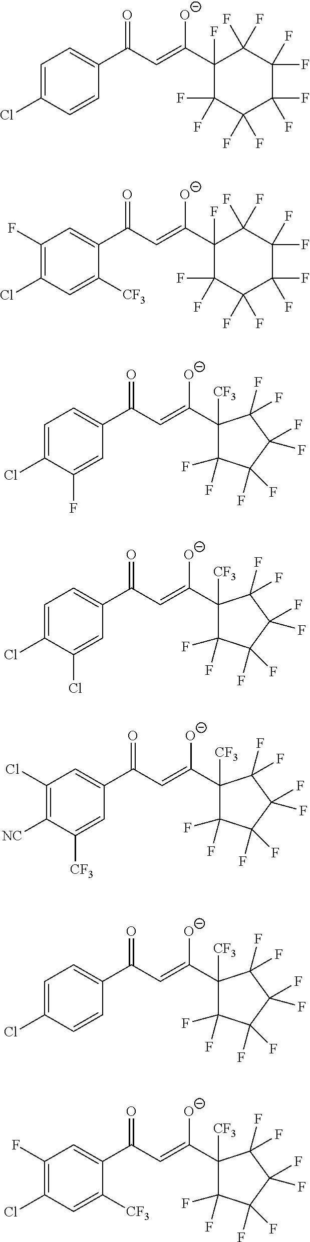

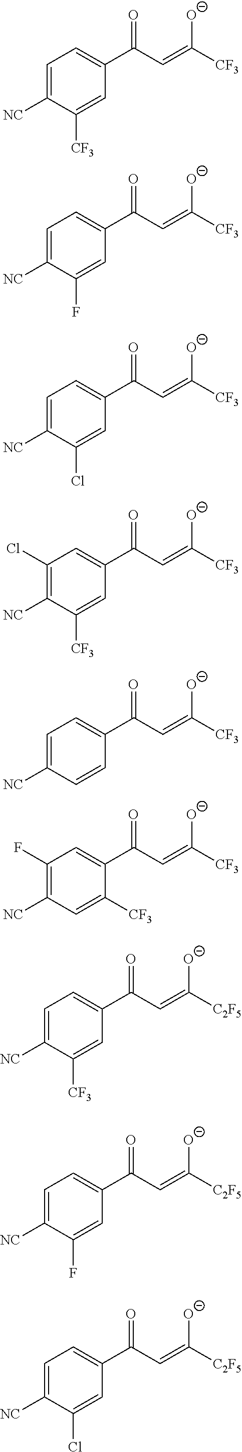

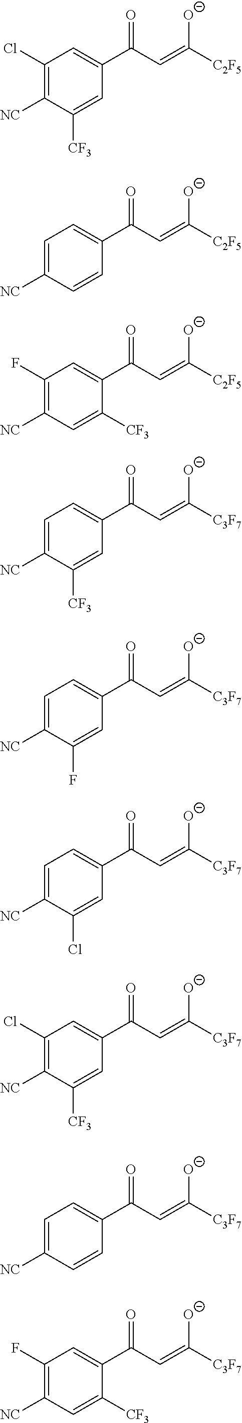

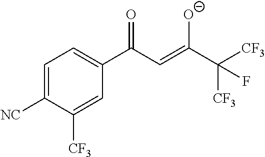 [SMILES: N#Cc1ccc(C(=O)/C=C(\[O-])C(F)(C(F)(F)F)C(F)(F)F)cc1C(F)(F)F]

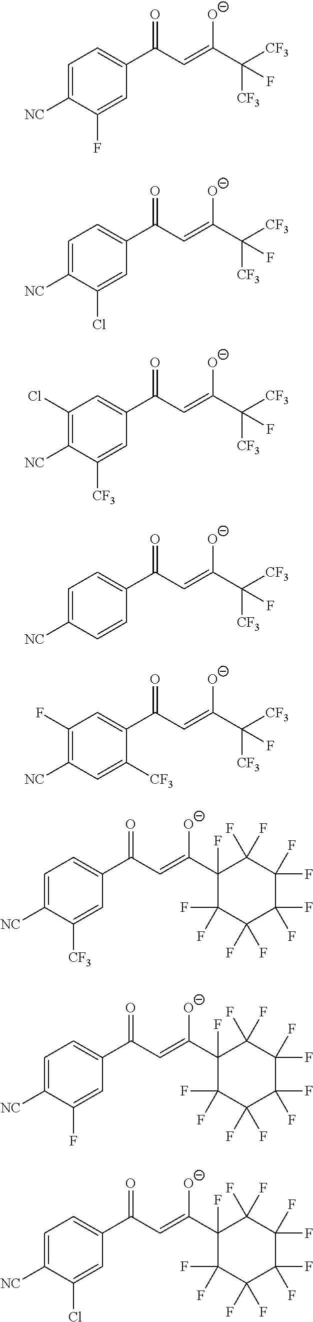

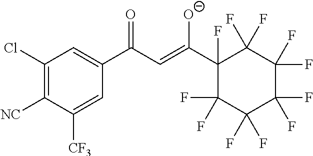 [SMILES: N#Cc1c(Cl)cc(C(=O)/C=C(\[O-])C2(F)C(F)(F)C(F)(F)C(F)(F)C(F)(F)C2(F)F)cc1C(F)(F)F]

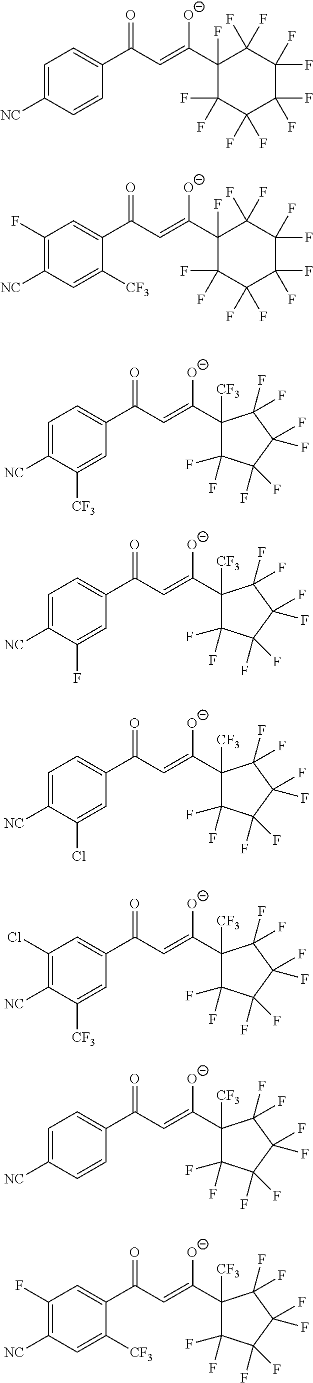

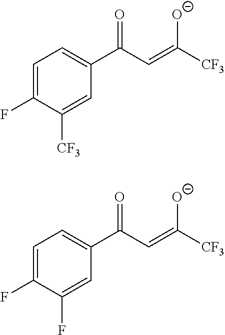 [SMILES: O=C(/C=C(\[O-])C(F)(F)F)c1ccc(F)c(C(F)(F)F)c1 ; O=C(/C=C(\[O-])C(F)(F)F)c1ccc(F)c(F)c1]

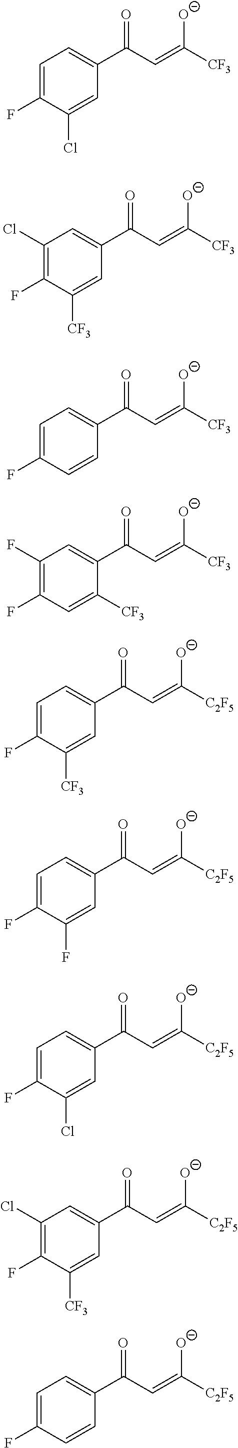

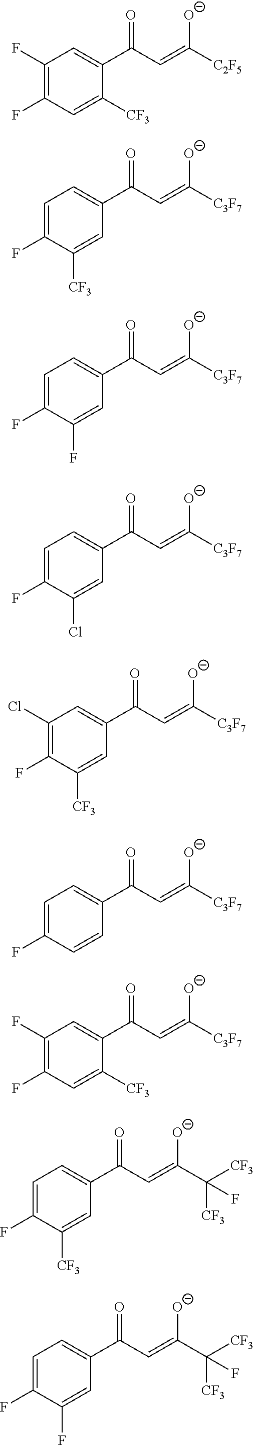

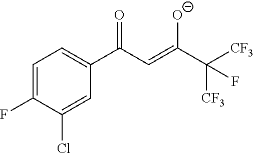 [SMILES: O=C(/C=C(\[O-])C(F)(C(F)(F)F)C(F)(F)F)c1ccc(F)c(Cl)c1]

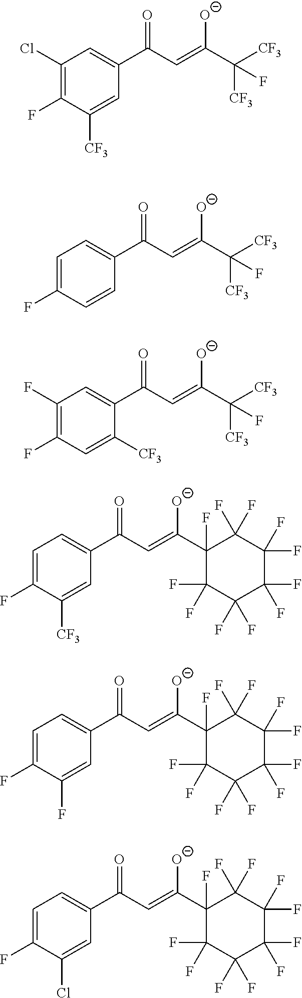 [SMILES: O=C(/C=C(\[O-])C(F)(C(F)(F)F)C(F)(F)F)c1cc(Cl)c(F)c(C(F)(F)F)c1 ; O=C(/C=C(\[O-])C(F)(C(F)(F)F)C(F)(F)F)c1ccc(F)cc1 ; O=C(/C=C(\[O-])C(F)(C(F)(F)F)C(F)(F)F)c1cc(F)c(F)cc1C(F)(F)F ; O=C(/C=C(\[O-])C1(F)C(F)(F)C(F)(F)C(F)(F)C(F)(F)C1(F)F)c1ccc(F)c(C(F)(F)F)c1 ; O=C(/C=C(\[O-])C1(F)C(F)(F)C(F)(F)C(F)(F)C(F)(F)C1(F)F)c1ccc(F)c(F)c1 ; O=C(/C=C(\[O-])C1(F)C(F)(F)C(F)(F)C(F)(F)C(F)(F)C1(F)F)c1ccc(F)c(Cl)c1]

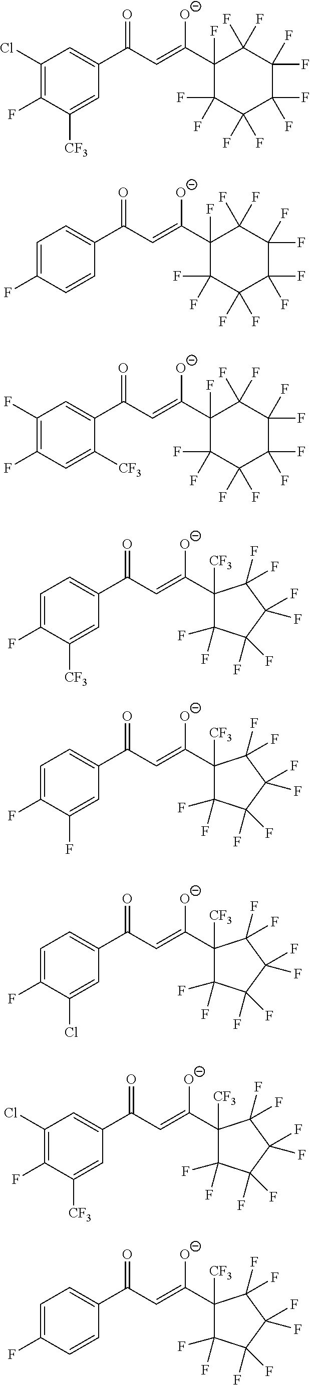

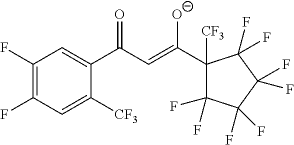 [SMILES: O=C(/C=C(\[O-])C1(C(F)(F)F)C(F)(F)C(F)(F)C(F)(F)C1(F)F)c1cc(F)c(F)cc1C(F)(F)F]

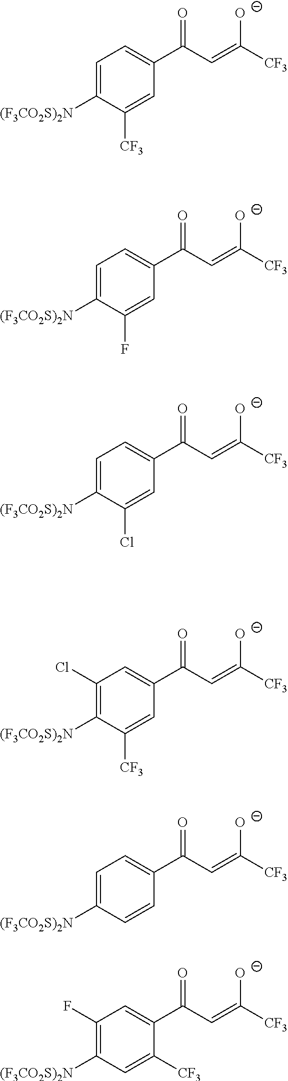

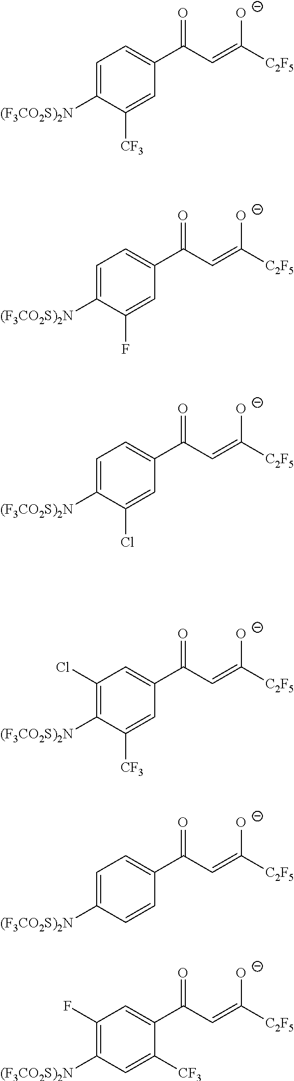

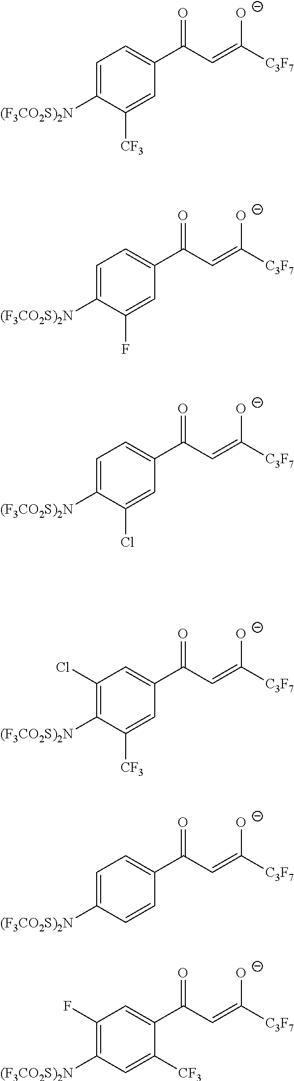

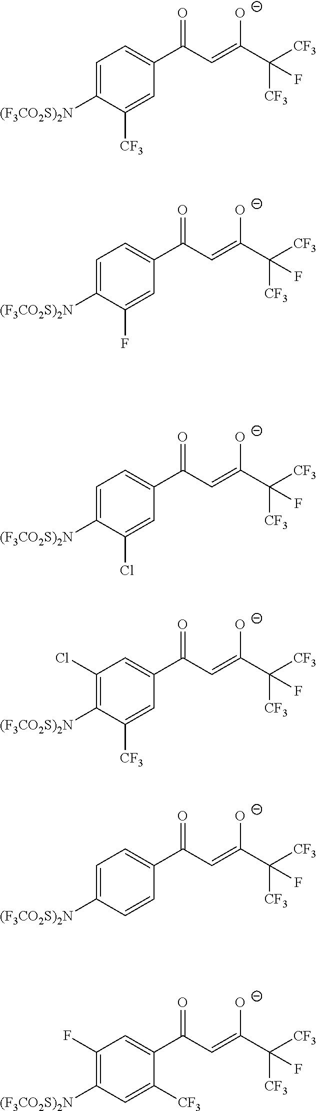

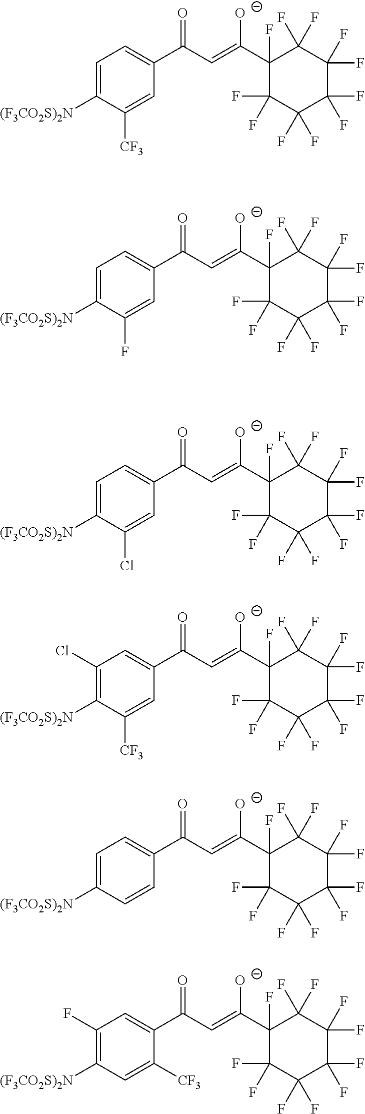

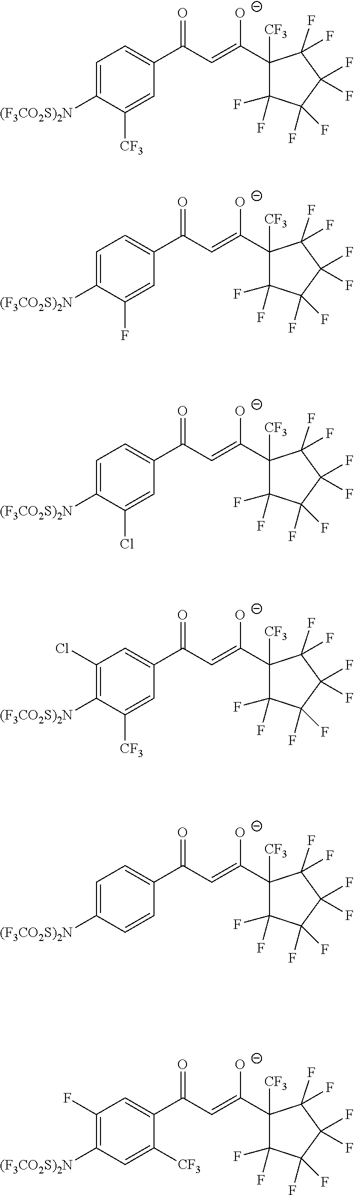

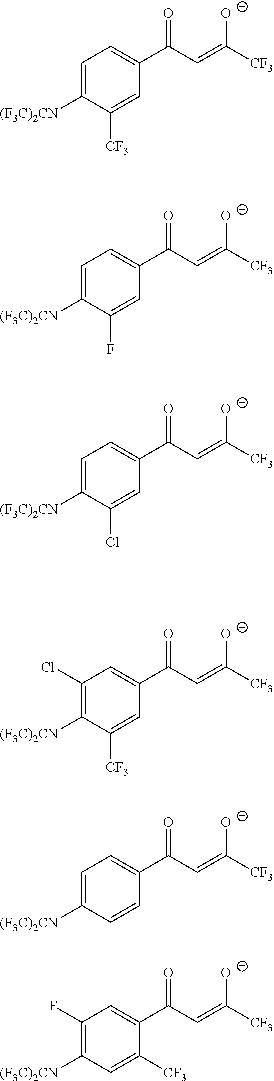

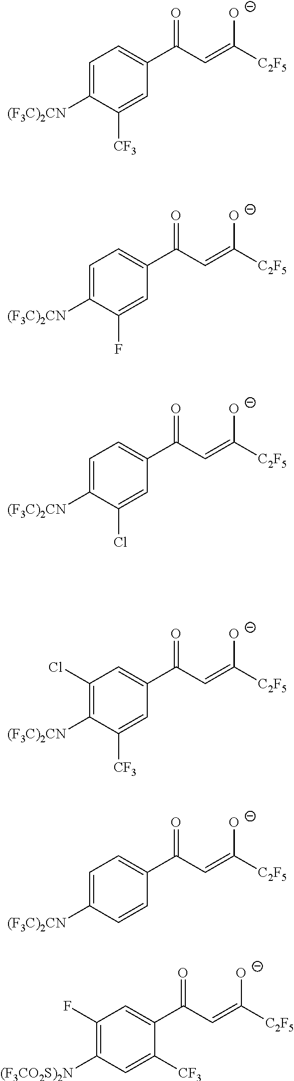

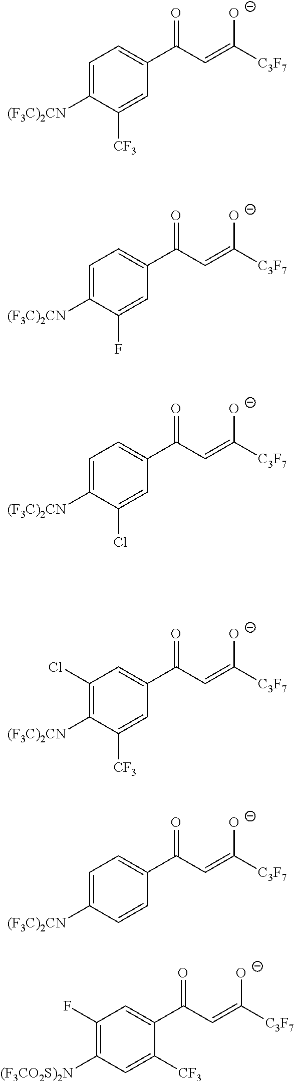

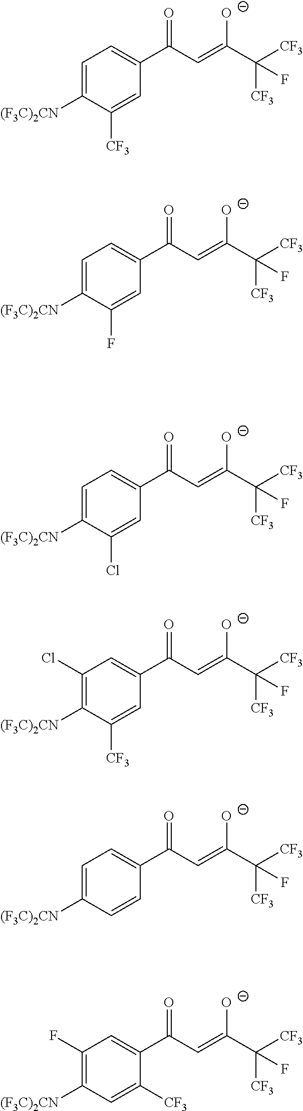

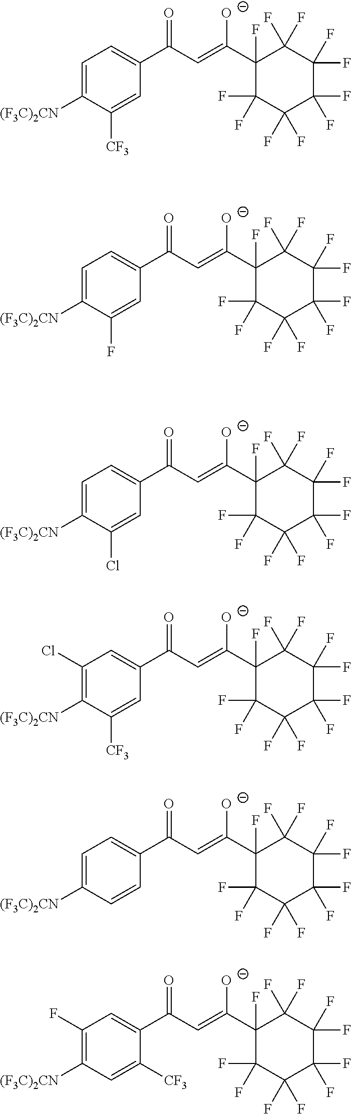

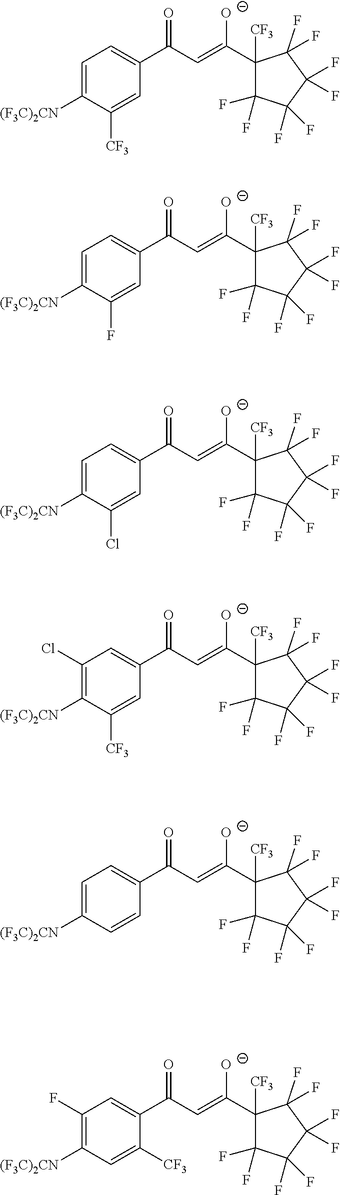

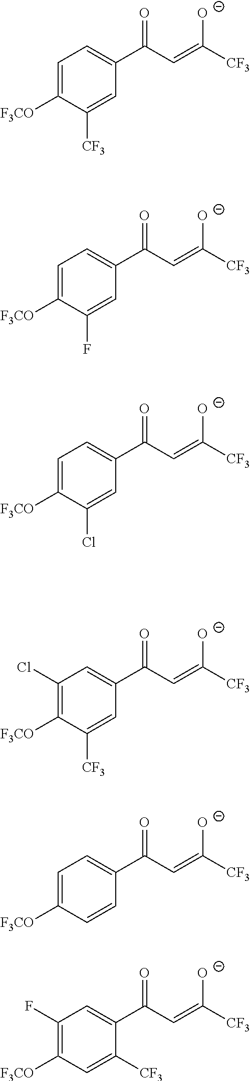 [SMILES: O=C(/C=C(\[O-])C(F)(F)F)c1ccc(OC(F)(F)F)c(C(F)(F)F)c1 ; O=C(/C=C(\[O-])C(F)(F)F)c1ccc(OC(F)(F)F)c(F)c1 ; O=C(/C=C(\[O-])C(F)(F)F)c1ccc(OC(F)(F)F)c(Cl)c1 ; O=C(/C=C(\[O-])C(F)(F)F)c1cc(Cl)c(OC(F)(F)F)c(C(F)(F)F)c1 ; O=C(/C=C(\[O-])C(F)(F)F)c1ccc(OC(F)(F)F)cc1 ; O=C(/C=C(\[O-])C(F)(F)F)c1cc(F)c(OC(F)(F)F)cc1C(F)(F)F]

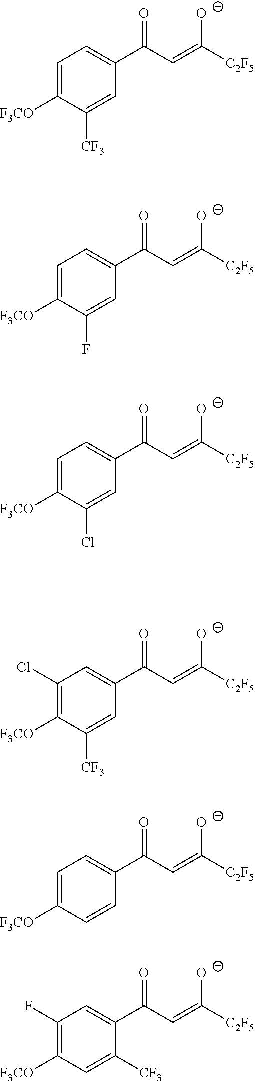

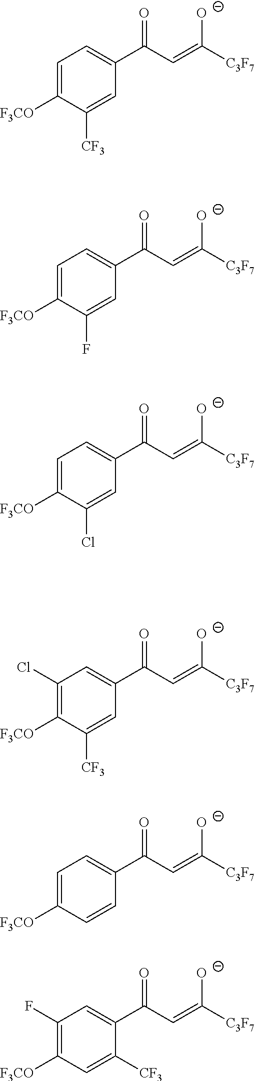

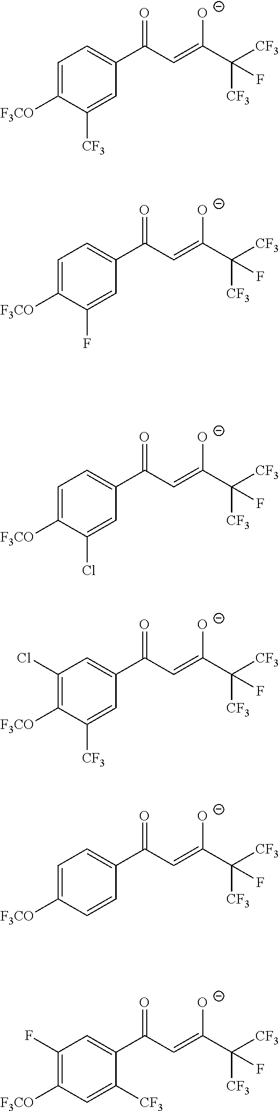 [SMILES: O=C(/C=C(\[O-])C(F)(C(F)(F)F)C(F)(F)F)c1ccc(OC(F)(F)F)c(C(F)(F)F)c1 ; O=C(/C=C(\[O-])C(F)(C(F)(F)F)C(F)(F)F)c1ccc(OC(F)(F)F)c(F)c1 ; O=C(/C=C(\[O-])C(F)(C(F)(F)F)C(F)(F)F)c1ccc(OC(F)(F)F)c(Cl)c1 ; O=C(/C=C(\[O-])C(F)(C(F)(F)F)C(F)(F)F)c1cc(Cl)c(OC(F)(F)F)c(C(F)(F)F)c1 ; O=C(/C=C(\[O-])C(F)(C(F)(F)F)C(F)(F)F)c1ccc(OC(F)(F)F)cc1 ; O=C(/C=C(\[O-])C(F)(C(F)(F)F)C(F)(F)F)c1cc(F)c(OC(F)(F)F)cc1C(F)(F)F]

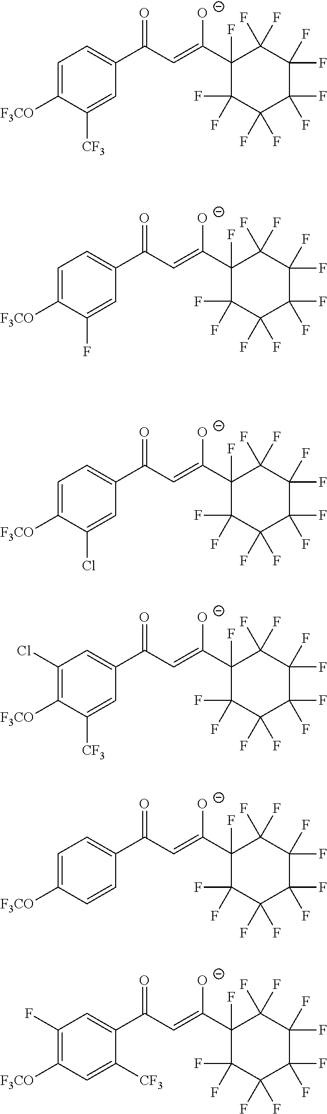

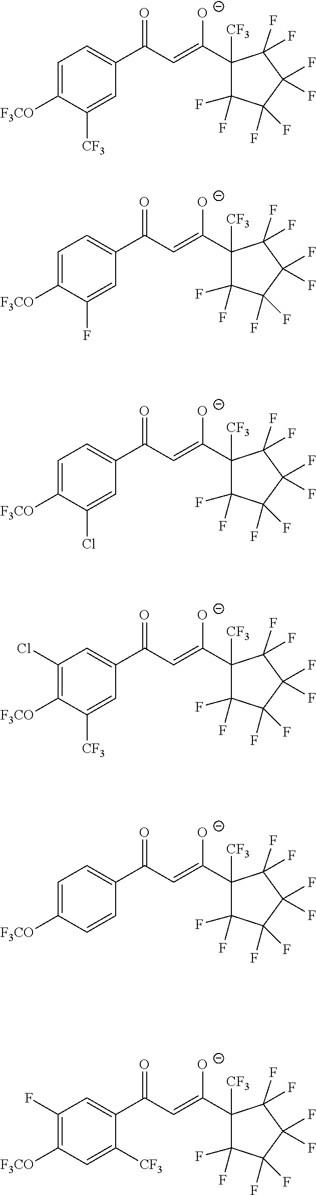

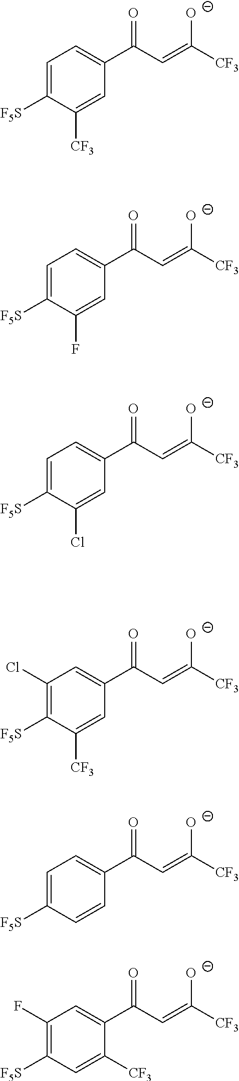

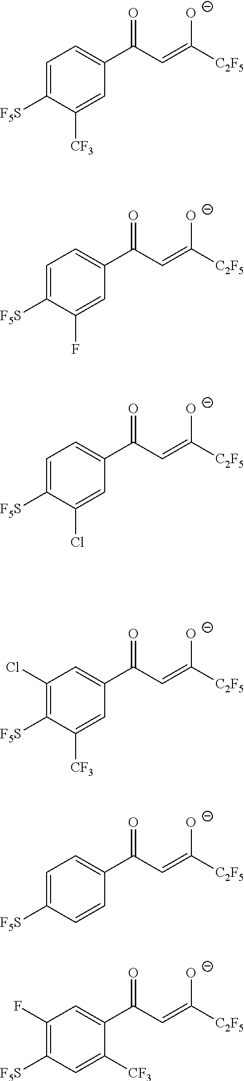

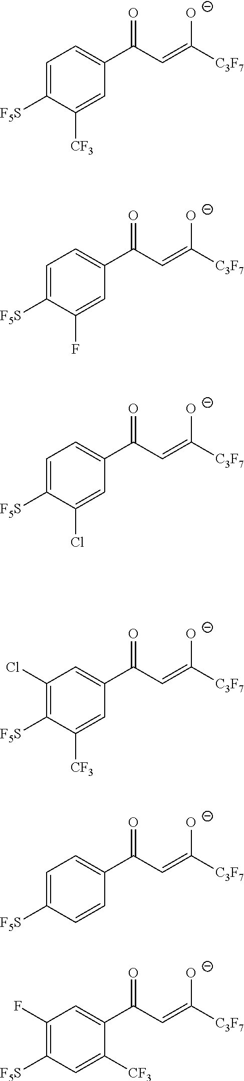

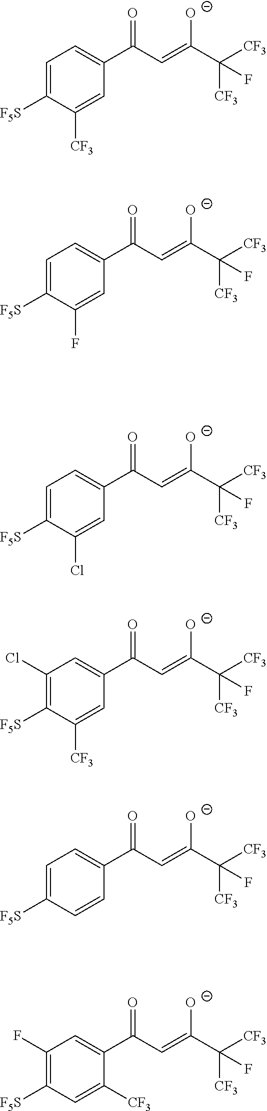

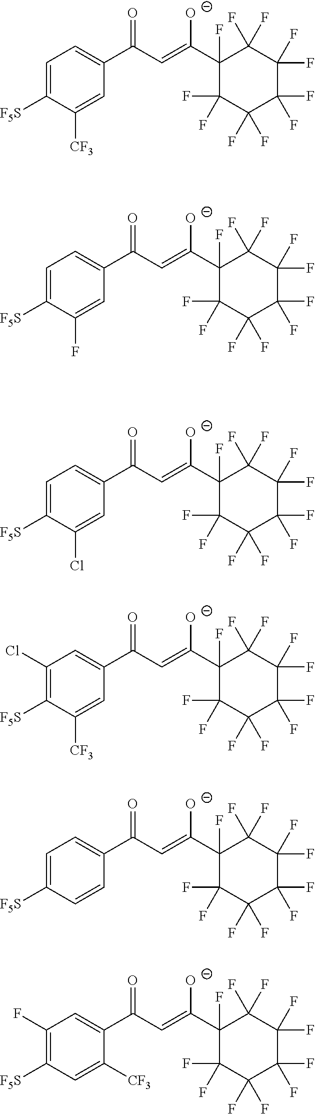

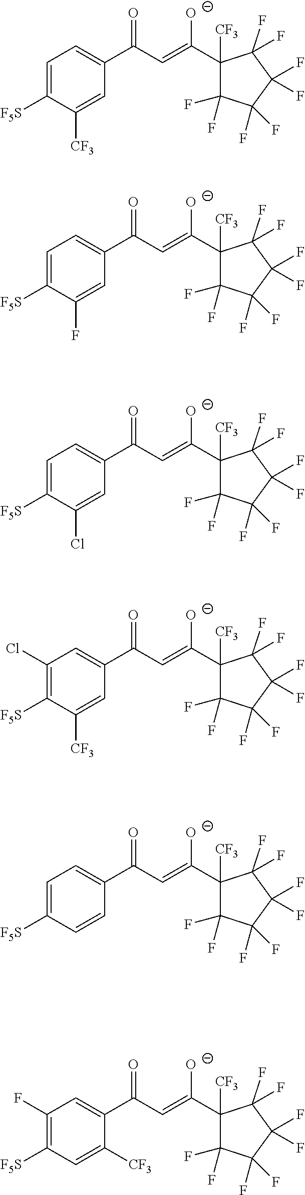

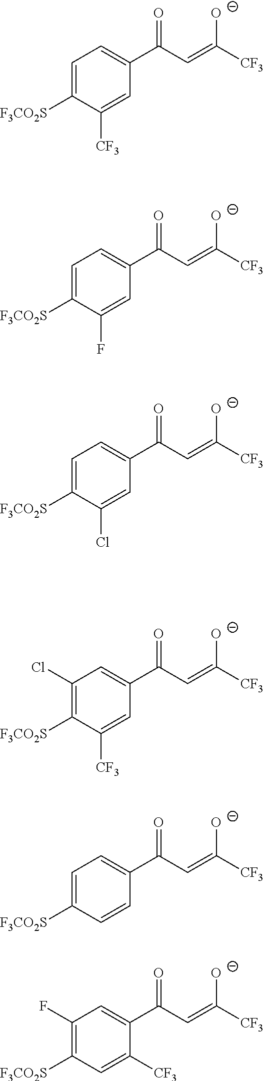

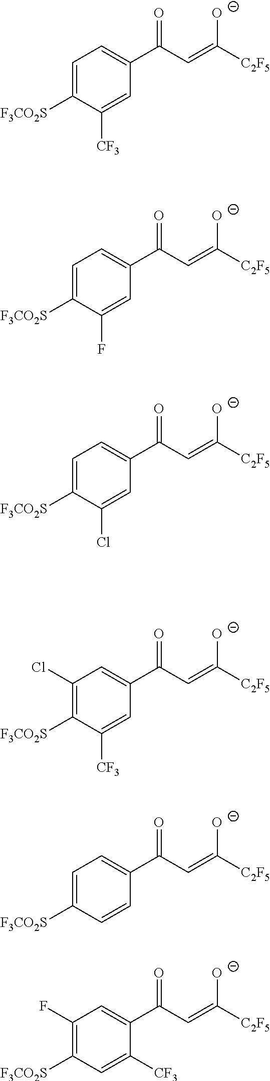

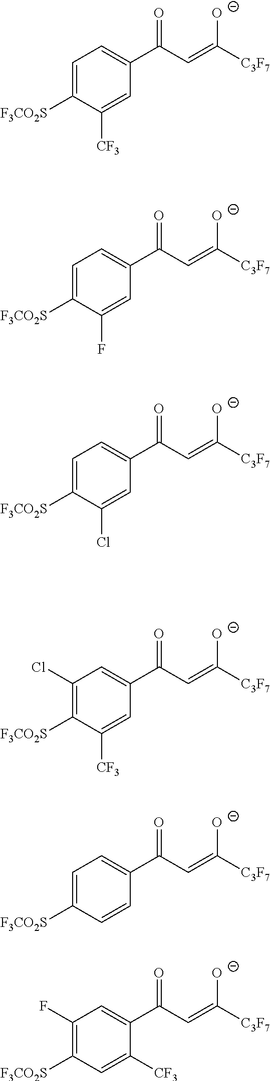

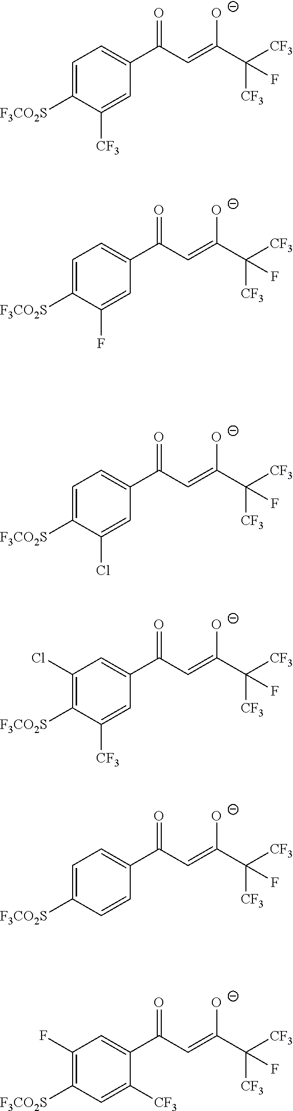

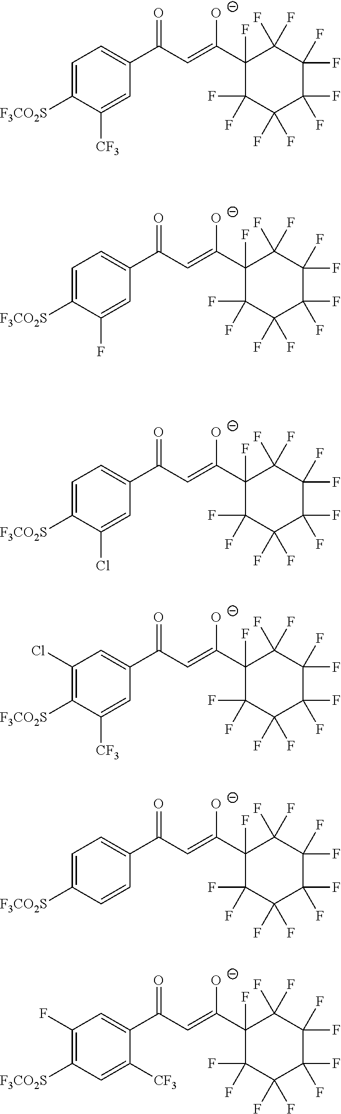

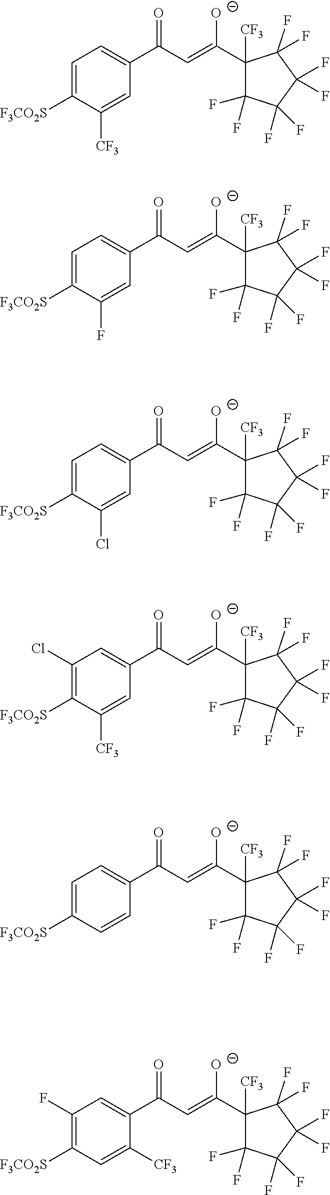

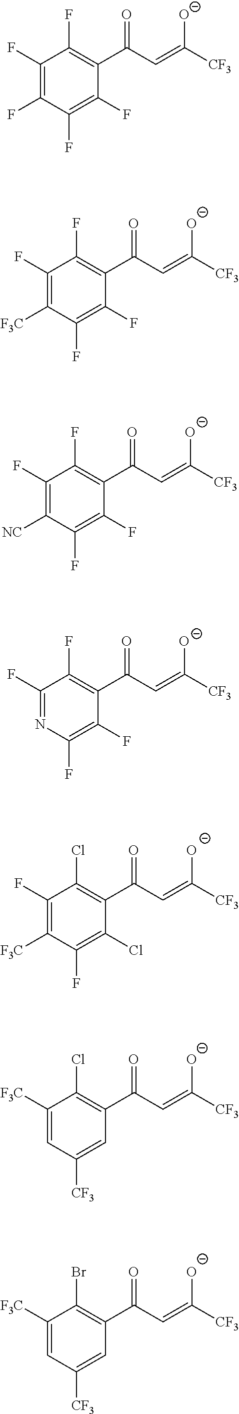

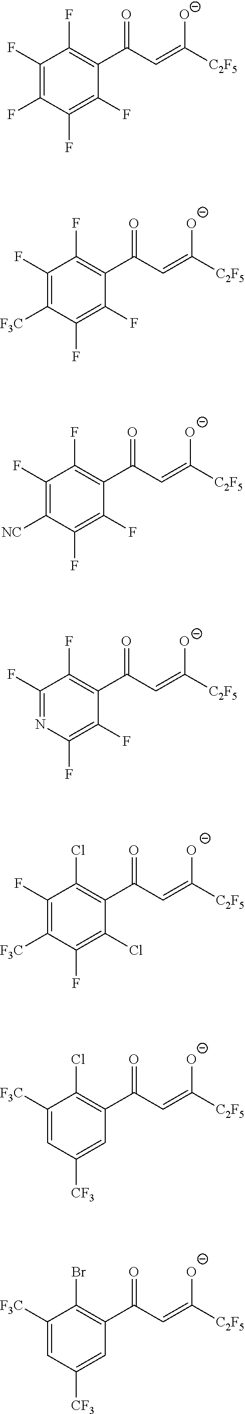

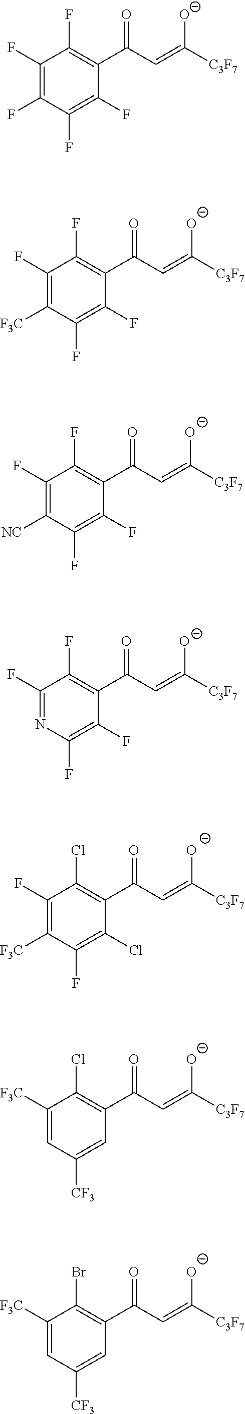

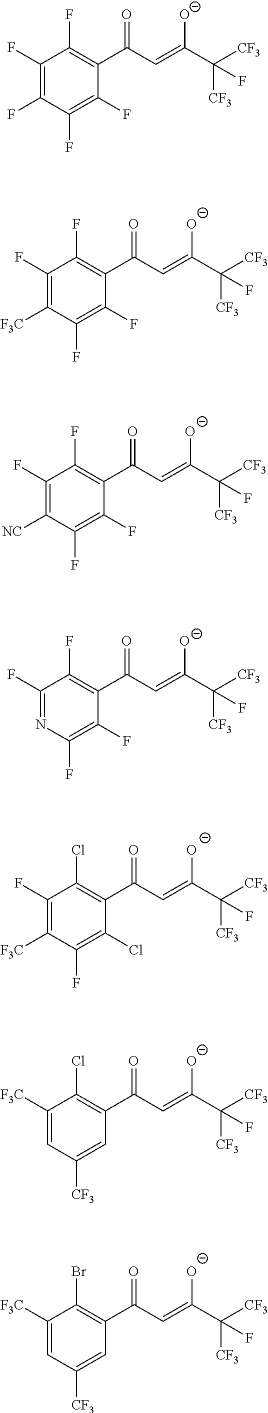

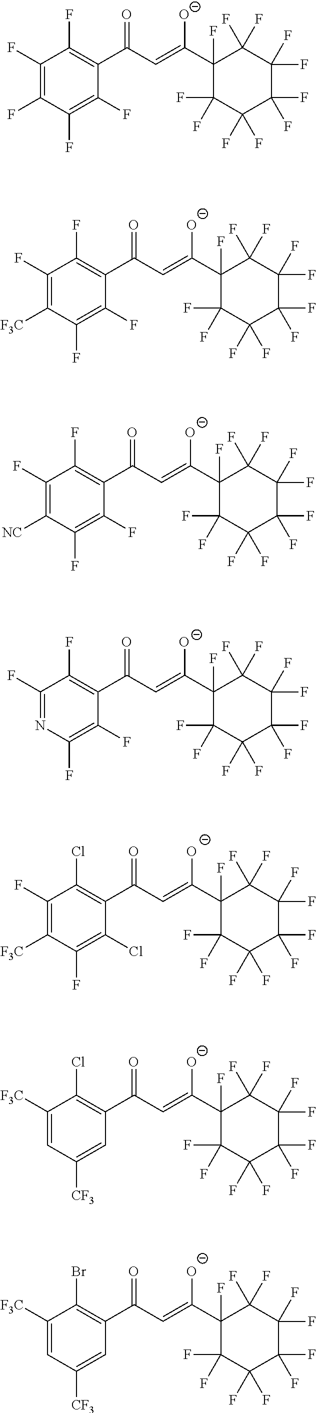

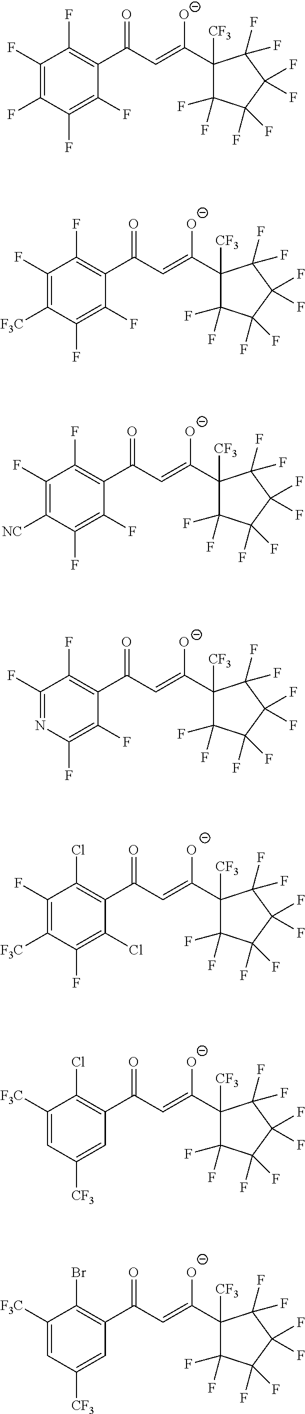

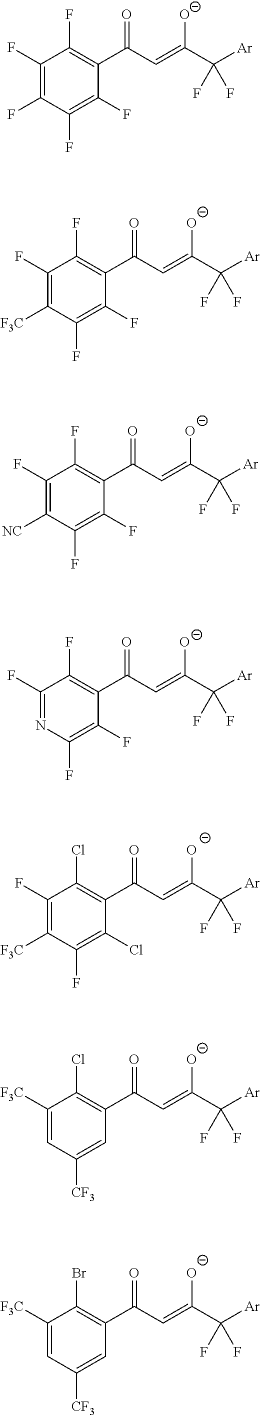

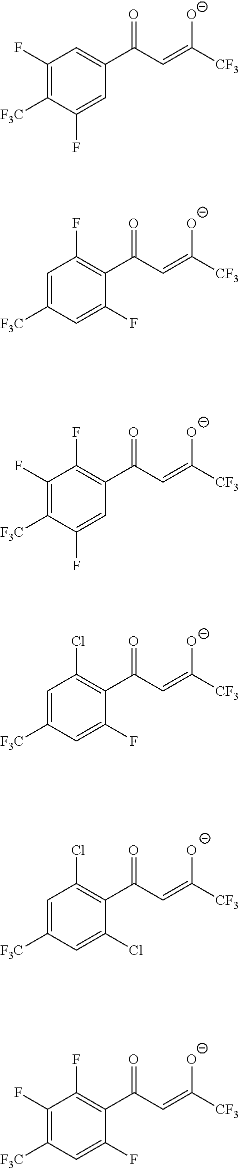 [SMILES: O=C(/C=C(\[O-])C(F)(F)F)c1cc(F)c(C(F)(F)F)c(F)c1 ; O=C(/C=C(\[O-])C(F)(F)F)c1c(F)cc(C(F)(F)F)cc1F ; O=C(/C=C(\[O-])C(F)(F)F)c1cc(F)c(C(F)(F)F)c(F)c1F ; O=C(/C=C(\[O-])C(F)(F)F)c1c(F)cc(C(F)(F)F)cc1Cl ; O=C(/C=C(\[O-])C(F)(F)F)c1c(Cl)cc(C(F)(F)F)cc1Cl ; O=C(/C=C(\[O-])C(F)(F)F)c1c(F)cc(C(F)(F)F)c(F)c1F]

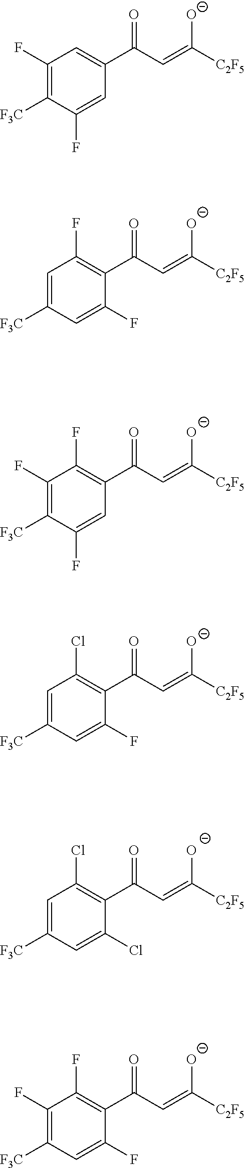

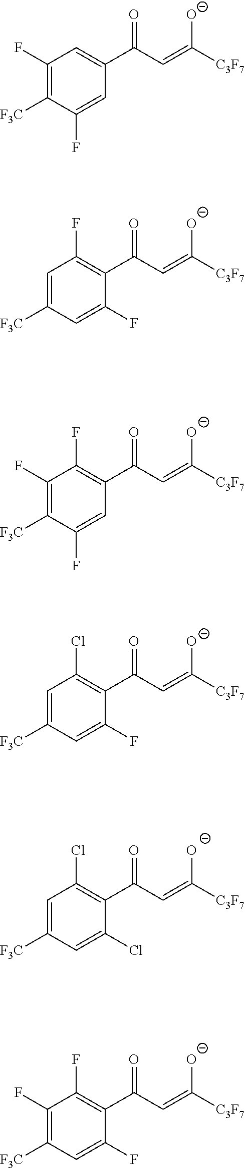

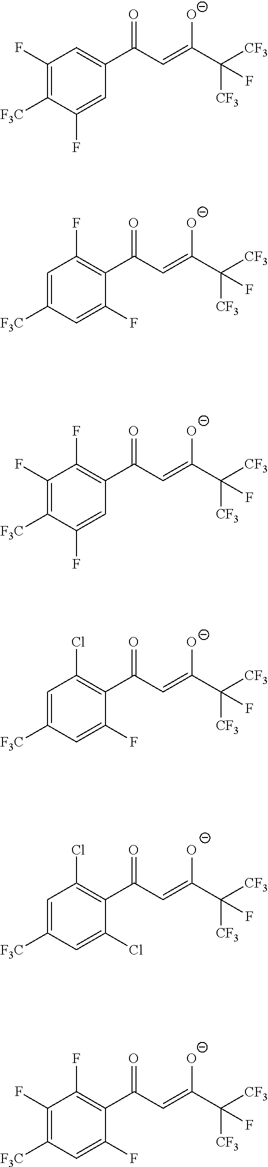 [SMILES: O=C(/C=C(\[O-])C(F)(C(F)(F)F)C(F)(F)F)c1cc(F)c(C(F)(F)F)c(F)c1 ; O=C(/C=C(\[O-])C(F)(C(F)(F)F)C(F)(F)F)c1c(F)cc(C(F)(F)F)cc1F ; O=C(/C=C(\[O-])C(F)(C(F)(F)F)C(F)(F)F)c1cc(F)c(C(F)(F)F)c(F)c1F ; O=C(/C=C(\[O-])C(F)(C(F)(F)F)C(F)(F)F)c1c(F)cc(C(F)(F)F)cc1Cl ; O=C(/C=C(\[O-])C(F)(C(F)(F)F)C(F)(F)F)c1c(Cl)cc(C(F)(F)F)cc1Cl ; O=C(/C=C(\[O-])C(F)(C(F)(F)F)C(F)(F)F)c1c(F)cc(C(F)(F)F)c(F)c1F]

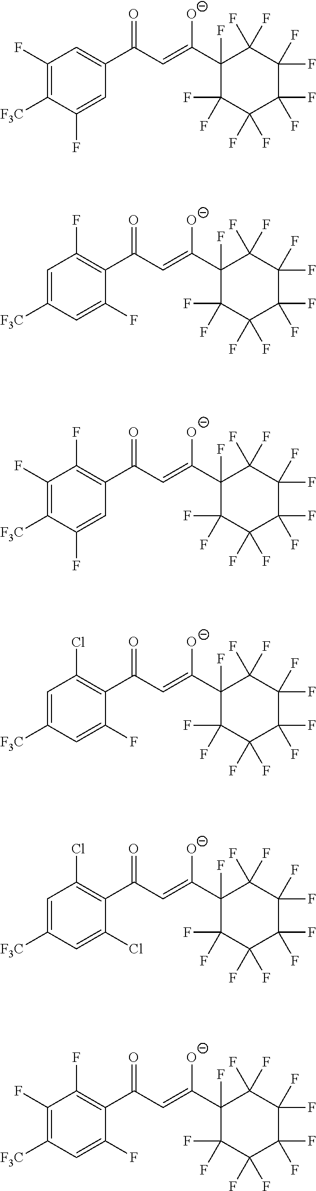 [SMILES: O=C(/C=C(\[O-])C1(F)C(F)(F)C(F)(F)C(F)(F)C(F)(F)C1(F)F)c1cc(F)c(C(F)(F)F)c(F)c1 ; O=C(/C=C(\[O-])C1(F)C(F)(F)C(F)(F)C(F)(F)C(F)(F)C1(F)F)c1c(F)cc(C(F)(F)F)cc1F ; O=C(/C=C(\[O-])C1(F)C(F)(F)C(F)(F)C(F)(F)C(F)(F)C1(F)F)c1cc(F)c(C(F)(F)F)c(F)c1F ; O=C(/C=C(\[O-])C1(F)C(F)(F)C(F)(F)C(F)(F)C(F)(F)C1(F)F)c1c(F)cc(C(F)(F)F)cc1Cl ; O=C(/C=C(\[O-])C1(F)C(F)(F)C(F)(F)C(F)(F)C(F)(F)C1(F)F)c1c(Cl)cc(C(F)(F)F)cc1Cl ; O=C(/C=C(\[O-])C1(F)C(F)(F)C(F)(F)C(F)(F)C(F)(F)C1(F)F)c1c(F)cc(C(F)(F)F)c(F)c1F]

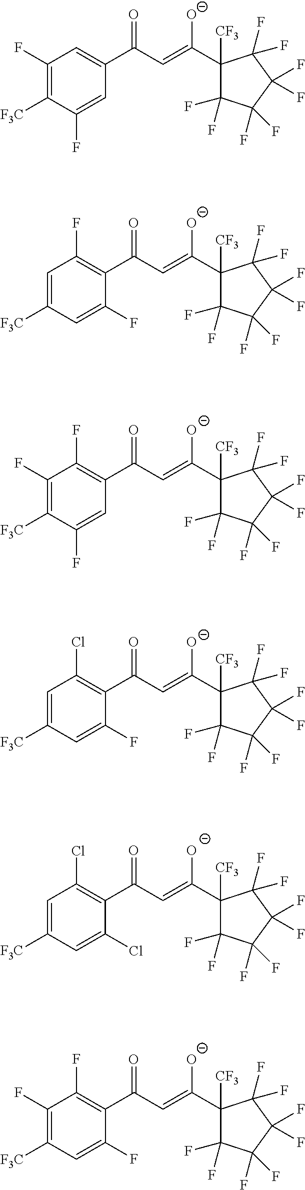 [SMILES: O=C(/C=C(\O)C1(C(F)(F)F)C(F)(F)C(F)(F)C(F)(F)C1(F)F)c1cc(F)c(C(F)(F)F)c(F)c1 ; O=C(/C=C(\O)C1(C(F)(F)F)C(F)(F)C(F)(F)C(F)(F)C1(F)F)c1c(F)cc(C(F)(F)F)cc1F ; O=C(/C=C(\[O-])C1(C(F)(F)F)C(F)(F)C(F)(F)C(F)(F)C1(F)F)c1cc(F)c(C(F)(F)F)c(F)c1F ; O=C(/C=C(\[O-])C1(C(F)(F)F)C(F)(F)C(F)(F)C(F)(F)C1(F)F)c1c(F)cc(C(F)(F)F)cc1Cl ; O=C(/C=C(\O)C1(C(F)(F)F)C(F)(F)C(F)(F)C(F)(F)C1(F)F)c1c(Cl)cc(C(F)(F)F)cc1Cl ; O=C(/C=C(\[O-])C1(C(F)(F)F)C(F)(F)C(F)(F)C(F)(F)C1(F)F)c1c(F)cc(C(F)(F)F)c(F)c1F]

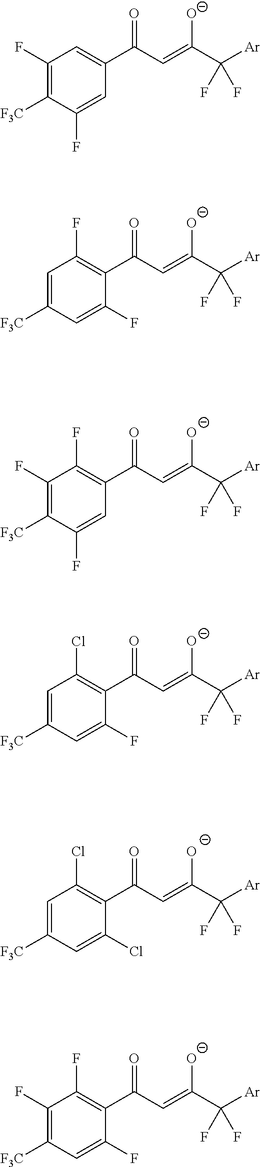

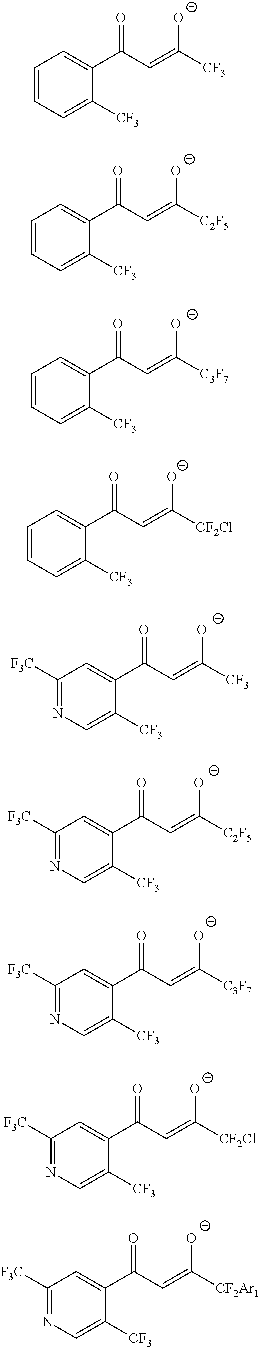

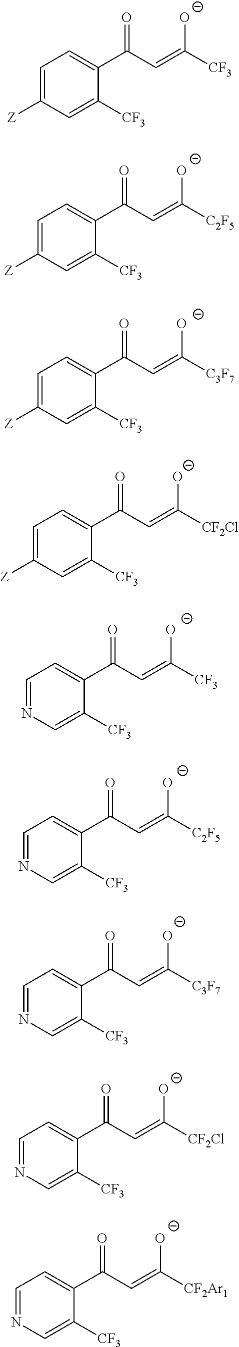

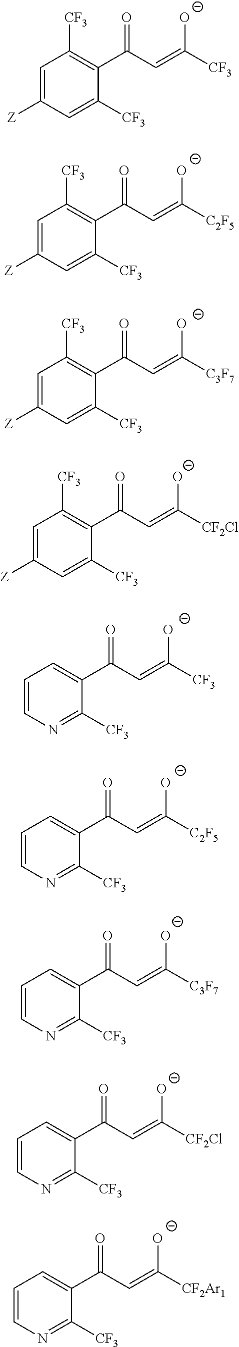

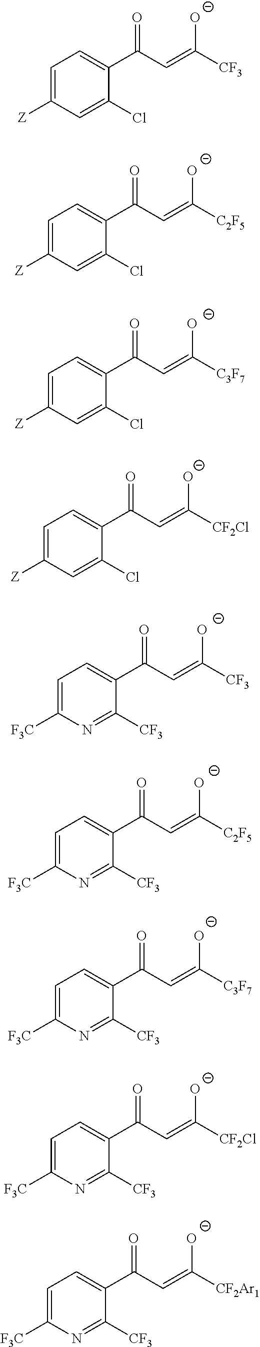

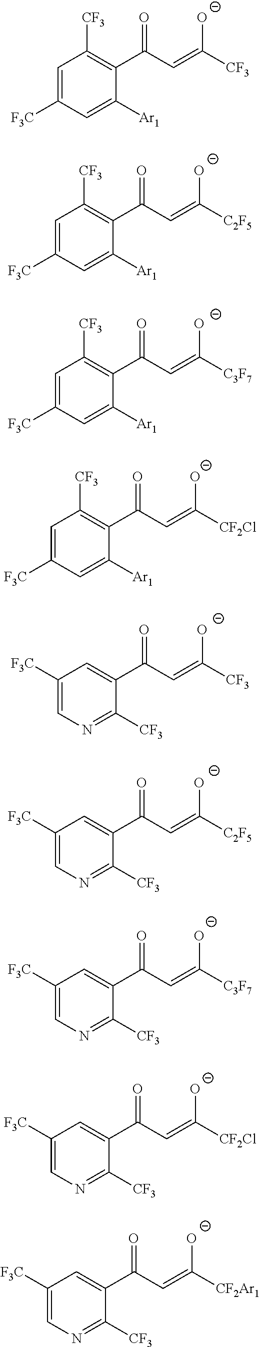

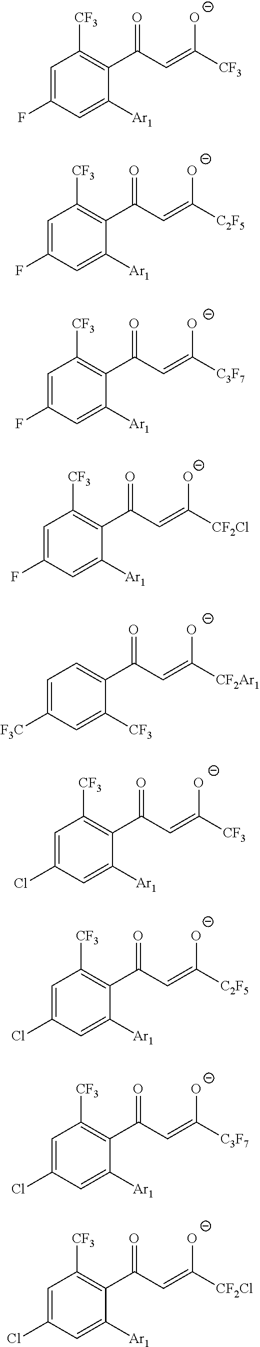

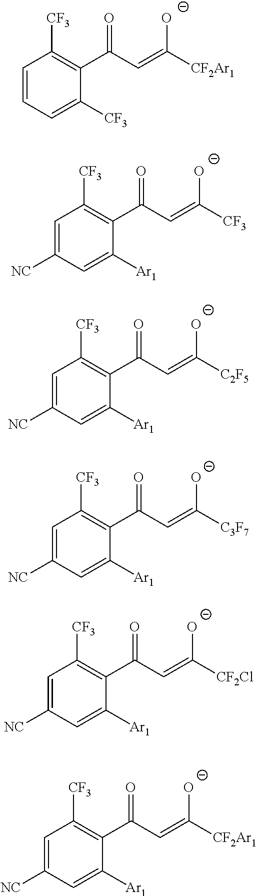

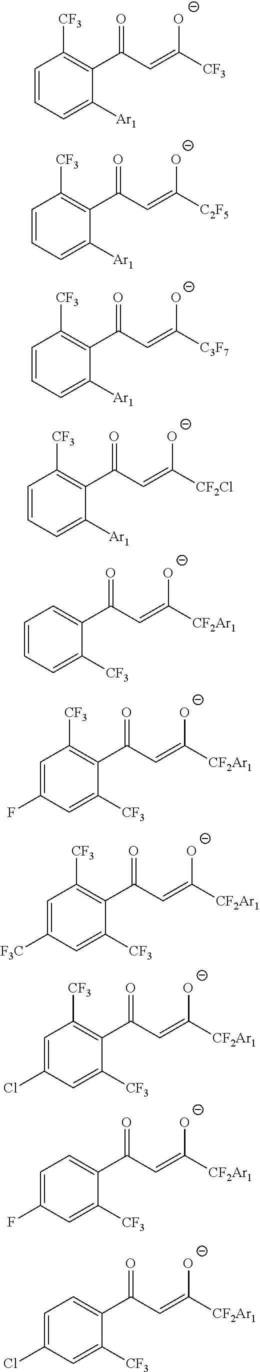

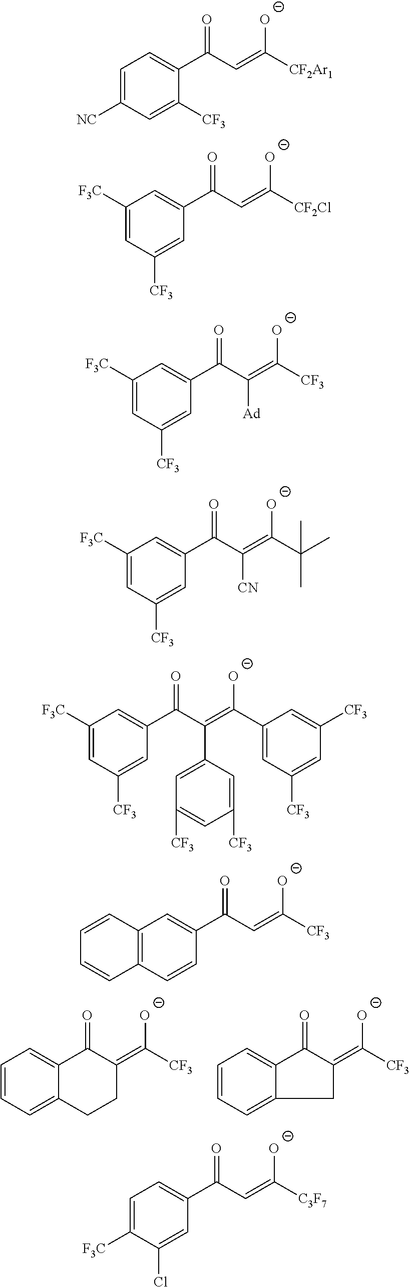

Ad is adamantyl,
R³ is CN, F, Cl, CF₃, Ar, t-butyl, adamantyl,
R¹ is CF(CF₃)₂, n-C₃F₇, 2,2,3,3,4,4,5,5-octafluoro-1-(trifluoromethyl)cyclopentyl, and 1,2,2,3,3,4,4,5,5,6,6-undecafluorocyclohexyl,
X is N(SO₂CF₃)₂, SF₅, OCF₃, SCF₃, CF(CF₃)₂,
Z is F, Cl, CF₃, CN,

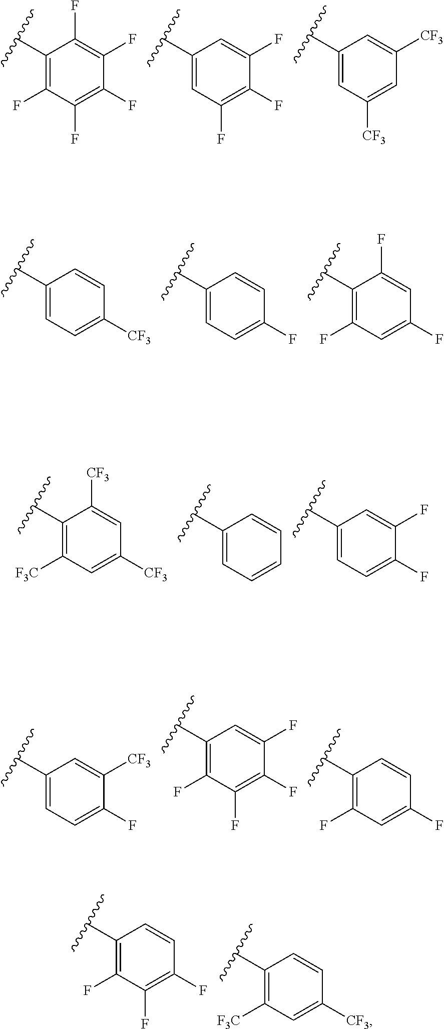

Ar is selected from A1, A2, A3, A4, A5, A6, A7, A8, A9, A10, A11, A12, At13, A14, A15, A16, A17, A18, A19, A20, A21, A22, A23, A24, A25, A26, A27, A28, A29, A30, A31 and A32

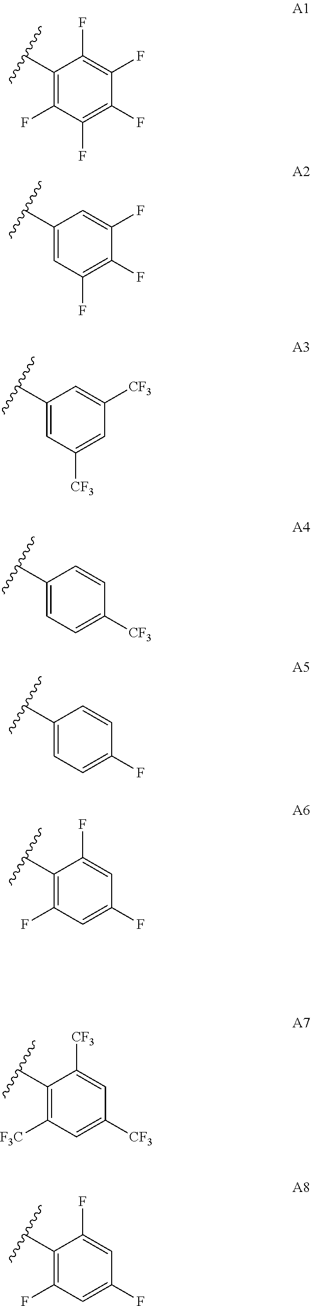

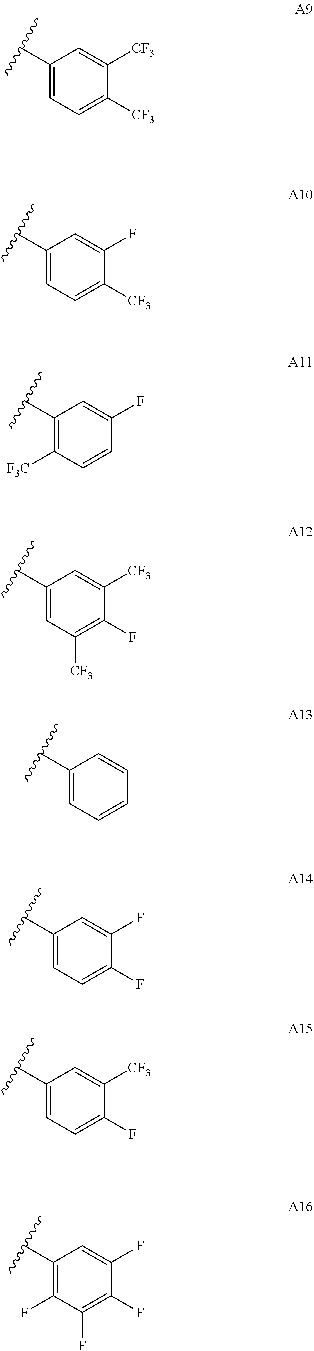

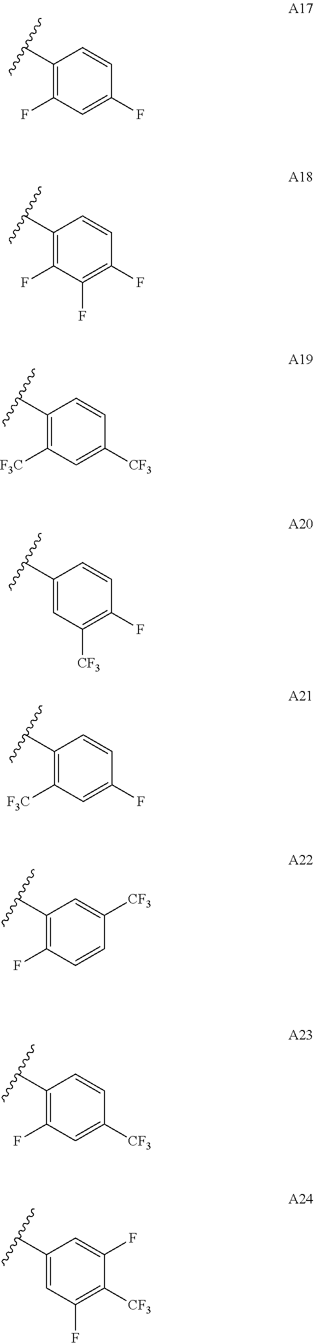

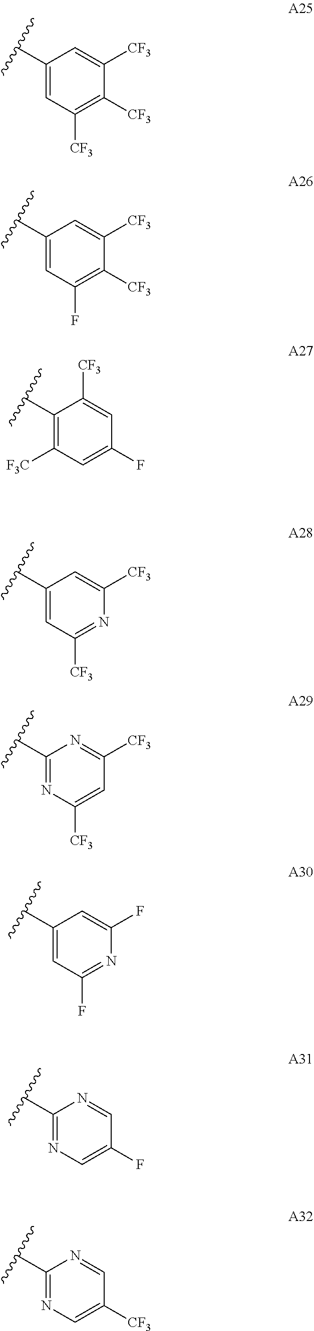

Ar₁ is

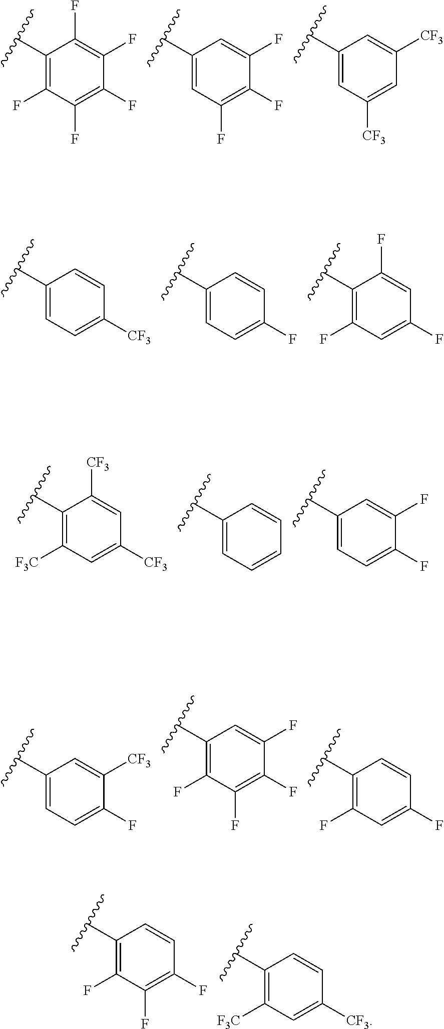

The homoleptic compounds of formula (I) are produced by reacting the β-diketone ligand with a ceric salt. Usually the ceric salt is soluble in the reaction medium. Suitable salts are ceric ammonium nitrate and ceric ammonium sulphate. The β-diketone ligands are either commercial available or they can be prepared by a synthesis known to a skilled person.

The heteroleptic compounds of formula (I) are produced by
mixing two different homoleptic cerium compounds in a suitable solvent,
mixing a homoleptic cerium compound with a ligand or its alkaline/earth alkaline salt different from the ligands of the compound,
vapor deposition of two different homoleptic cerium compounds,
vapor deposition (vapor co-condensation) of homoleptic cerium compounds with a ligand different from the ligands of the compound.

The compounds of formula (I.a),

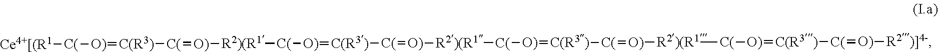

wherein
(R¹, R², R³), (R^{1′}, R^{2′}, R^{3′}), (R^{1″}, R^{2″}, R^{3″}) and (R^{1′″}, R^{2′″}, R^{3′″}) are each selected from the definition given in one line of the following table 1 are excluded from the invention:

TABLE 1

| | R¹, R^{1′}, | R², R^{2′}, | R³, R^{3′}, |
|---|---|---|---|
| | R^{1″}, R^{1′″} | R^{2″}, R^{2′″} | R^{3″}, R^{3′″} |
| 1. | trifluoromethyl | trifluoromethyl | H |
| 2. | trifluoromethyl | trifluoromethyl | CN |
| 3. | trifluoromethyl | trifluoromethyl | F |
| 4. | trifluoromethyl | trifluoromethyl | Cl |
| 5. | trifluoromethyl | trifluoromethyl | pentafluorophenyl |
| 6. | trifluoromethyl | trifluoromethyl | tetrafluoro-2- |
| | | | (trifluoromethyl)phenyl |
| 7. | trifluoromethyl | trifluoromethyl | tetrafluoro-3- |
| | | | (trifluoromethyl)phenyl |
| 8. | trifluoromethyl | trifluoromethyl | tetrafluoro-4- |
| | | | (trifluoromethyl)phenyl |
| 9. | trifluoromethyl | trifluoromethyl | tetrafluoro-2- |
| | | | pyridyl |
| 10. | trifluoromethyl | trifluoromethyl | tetrafluoro-3- |
| | | | pyridyl |
| 11. | trifluoromethyl | trifluoromethyl | tetrafluoro-4- |
| | | | pyridyl |
| 12. | trifluoromethyl | trifluoromethyl | tetrafluoro-2- |
| | | | benzonitrile |
| 13. | trifluoromethyl | trifluoromethyl | tetrafluoro-3- |
| | | | benzonitrile |
| 14. | trifluoromethyl | trifluoromethyl | tetrafluoro-4- |
| | | | benzonitrile |
| 15. | pentafluoroethyl | pentafluoroethyl | H |
| 16. | pentafluoroethyl | pentafluoroethyl | CN |
| 17. | pentafluoroethyl | pentafluoroethyl | F |
| 18. | pentafluoroethyl | pentafluoroethyl | Cl |
| 19. | pentafluoroethyl | pentafluoroethyl | pentafluorophenyl |
| 20. | pentafluoroethyl | pentafluoroethyl | tetrafluoro-2- |
| | | | (trifluoromethyl)phenyl |
| 21. | pentafluoroethyl | pentafluoroethyl | tetrafluoro-3- |
| | | | (trifluoromethyl)phenyl |
| 22. | pentafluoroethyl | pentafluoroethyl | tetrafluoro-4- |
| | | | (trifluoromethyl)phenyl |
| 23. | pentafluoroethyl | pentafluoroethyl | tetrafluoro-2- |
| | | | pyridyl |
| 24. | pentafluoroethyl | pentafluoroethyl | tetrafluoro-3- |
| | | | pyridyl |
| 25. | pentafluoroethyl | pentafluoroethyl | tetrafluoro-4- |
| | | | pyridyl |
| 26. | pentafluoroethyl | pentafluoroethyl | tetrafluoro-2- |
| | | | benzonitrile |
| 27. | pentafluoroethyl | pentafluoroethyl | tetrafluoro-3- |
| | | | benzonitrile |
| 28. | pentafluoroethyl | pentafluoroethyl | tetrafluoro-4- |
| | | | benzonitrile |
| 29. | heptafluoro-n- | heptafluoro-n- | H |
| | propyl | propyl | |
| 30. | heptafluoro-n- | heptafluoro-n- | CN |
| | propyl | propyl | |
| 31. | heptafluoro-n- | heptafluoro-n- | F |
| | propyl | propyl | |
| 32. | heptafluoro-n- | heptafluoro-n- | Cl |
| | propyl | propyl | |
| 33. | heptafluoro-n- | heptafluoro-n- | pentafluorophenyl |
| | propyl | propyl | |
| 34. | heptafluoro-n- | heptafluoro-n- | tetrafluoro-2- |
| | propyl | propyl | (trifluoromethyl)phenyl |
| 35. | heptafluoro-n- | heptafluoro-n- | tetrafluoro-3- |
| | propyl | propyl | (trifluoromethyl)phenyl |
| 36. | heptafluoro-n- | heptafluoro-n- | tetrafluoro-4- |
| | propyl | propyl | (trifluoromethyl)phenyl |
| 37. | heptafluoro-n- | heptafluoro-n- | tetrafluoro-2- |
| | propyl | propyl | pyridyl |
| 38. | heptafluoro-n- | heptafluoro-n- | tetrafluoro-3- |
| | propyl | propyl | pyridyl |
| 39. | heptafluoro-n- | heptafluoro-n- | tetrafluoro-4- |
| | propyl | propyl | pyridyl |
| 40. | heptafluoro-n- | heptafluoro-n- | tetrafluoro-2- |
| | propyl | propyl | benzonitrile |
| 41. | heptafluoro-n- | heptafluoro-n- | tetrafluoro-3- |
| | propyl | propyl | benzonitrile |
| 42. | heptafluoro-n- | heptafluoro-n- | tetrafluoro-4- |
| | propyl | propyl | benzonitrile |
| 43. | heptafluoro- | heptafluoro- | H |
| | isopropyl | isopropyl | |
| 44. | heptafluoro- | heptafluoro- | CN |
| | isopropyl | isopropyl | |
| 45. | heptafluoro- | heptafluoro- | F |
| | isopropyl | isopropyl | |
| 46. | heptafluoro- | heptafluoro- | Cl |
| | isopropyl | isopropyl | |
| 47. | heptafluor-iso- | heptafluoro- | pentafluorophenyl |
| | propyl | isopropyl | |
| 48. | heptafluor-iso- | heptafluoro- | tetrafluoro-2- |
| | propyl | isopropyl | (trifluoromethyl)phenyl |
| 49. | heptafluoro- | heptafluoro- | tetrafluoro-3- |
| | isopropyl | isopropyl | (trifluoromethyl)phenyl |
| 50. | heptafluoro- | heptafluoro- | tetrafluoro-4- |
| | isopropyl | isopropyl | (trifluoromethyl)phenyl |
| 51. | heptafluoro- | heptafluoro- | tetrafluoro-2- |
| | isopropyl | isopropyl | pyridyl |
| 52. | heptafluoro- | heptafluoro- | tetrafluoro-3- |
| | isopropyl | isopropyl | pyridyl |
| 53. | heptafluoro- | heptafluoro- | tetrafluoro-4- |
| | isopropyl | isopropyl | pyridyl |
| 54. | heptafluoro- | heptafluoro- | tetrafluoro-2- |
| | isopropyl | isopropyl | benzonitrile |
| 55. | heptafluoro- | heptafluoro- | tetrafluoro-3- |
| | isopropyl | isopropyl | benzonitrile |
| 56. | heptafluoro- | heptafluoro- | tetrafluoro-4- |
| | isopropyl | isopropyl | benzonitrile |
| 57. | tert-butyl | tert-butyl | H |
| 58. | tert-butyl | tert-butyl | CN |
| 59. | tert-butyl | tert-butyl | F |
| 60. | tert-butyl | tert-butyl | Cl |
| 61. | tert-butyl | tert-butyl | pentafluorophenyl |
| 62. | tert-butyl | tert-butyl | tetrafluoro-2- |
| | | | (trifluoromethyl)phenyl |
| 63. | tert-butyl | tert-butyl | tetrafluoro-3- |
| | | | (trifluoromethyl)phenyl |
| 64. | tert-butyl | tert-butyl | tetrafluoro-4- |
| | | | (trifluoromethyl)phenyl |
| 65. | tert-butyl | tert-butyl | tetrafluoro-2- |
| | | | pyridyl |
| 66. | tert-butyl | tert-butyl | tetrafluoro-3- |
| | | | pyridyl |
| 67. | tert-butyl | tert-butyl | tetrafluoro-4- |
| | | | pyridyl |
| 68. | tert-butyl | tert-butyl | tetrafluoro-2- |
| | | | benzonitrile |
| 69. | tert-butyl | tert-butyl | tetrafluoro-3- |
| | | | benzonitrile |
| 70. | tert-butyl | tert-butyl | tetrafluoro-4- |
| | | | benzonitrile |
| 71. | pentafluorophenyl | pentafluorophenyl | H |
| 72. | pentafluorophenyl | pentafluorophenyl | CN |
| 73. | pentafluorophenyl | pentafluorophenyl | F |
| 74. | pentafluorophenyl | pentafluorophenyl | Cl |
| 75. | pentafluorophenyl | pentafluorophenyl | pentafluorophenyl |
| 76. | pentafluorophenyl | pentafluorophenyl | tetrafluoro-2- |
| | | | (trifluoromethyl)phenyl |
| 77. | pentafluorophenyl | pentafluorophenyl | tetrafluoro-3- |
| | | | (trifluoromethyl)phenyl |
| 78. | pentafluorophenyl | pentafluorophenyl | tetrafluoro-4- |
| | | | (trifluoromethyl)phenyl |
| 79. | pentafluorophenyl | pentafluorophenyl | tetrafluoro-2- |
| | | | pyridyl |
| 80. | pentafluorophenyl | pentafluorophenyl | tetrafluoro-3- |
| | | | pyridyl |
| 81. | pentafluorophenyl | pentafluorophenyl | tetrafluoro-4- |
| | | | pyridyl |
| 82. | pentafluorophenyl | pentafluorophenyl | tetrafluoro-2- |
| | | | benzonitrile |
| 83. | pentafluorophenyl | pentafluorophenyl | tetrafluoro-3- |
| | | | benzonitrile |
| 84. | pentafluorophenyl | pentafluorophenyl | tetrafluoro-4- |
| | | | benzonitrile |
| 85. | 3,5- | 3,5- | H |
| | bis(trifluoromethyl)- | bis(trifluoromethyl)- | |
| | phenyl | phenyl | |
| 86. | 3,5- | trifluoromethyl | H |
| | bis(trifluoromethyl)- | | |
| | phenyl | | |
| 87. | 3,5- | pentafluoroethyl | H |
| | bis(trifluoromethyl)- | | |
| | phenyl | | |
| 88. | 4-fluoro-3- | pentafluoroethyl | H |
| | trifluoromethyl- | | |
| | phenyl | | |
| 89. | 4-fluoro-3- | 3,5- | H |
| | trifluoromethyl- | bis(trifluoromethyl)- | |
| | phenyl | phenyl | |
| 90. | 3,4,5- | 3,4,5- | H |
| | trifluorophenyl | trifluorophenyl | |
| 91. | 3,4,5- | pentafluoroethyl | H |
| | trifluorophenyl | | |
| 92. | 4- | 4- | H |
| | (trifluoromethyl)phenyl | (trifluoromethyl)phenyl | |
| 93. | 4- | pentafluoroethyl | H |
| | (trifluoromethyl)phenyl | | |
| 94. | 2,4,6- | 2,4,6- | H |
| | tris(trifluoromethyl)phenyl | tris(trifluoromethyl)phenyl | |
| 95. | 2,4,6- | Pentafluoroethyl | H |
| | tris(trifluoromethyl)phenyl | | |
| 96. | 3,5- | 3,5- | CN |
| | bis(trifluoromethyl)- | bis(trifluoromethyl)- | |
| | phenyl | phenyl | |
| 97. | 2,4,6- | 2,4,6- | CN |
| | tris(trifluoromethyl)phenyl | tris(trifluoromethyl)phenyl | |
| 98. | 3,5- | pentafluoroethyl | 3,5- |
| | bis(trifluoromethyl)- | | bis(trifluoromethyl)- |
| | phenyl | | phenyl |
| 99. | 3,4- | 3,4- | H |
| | bis(trifluoromethyl)- | bis(trifluoromethyl)- | |
| | phenyl | phenyl | |
| 100. | 3,4- | trifluoromethyl | H |
| | bis(trifluoromethyl)- | | |
| | phenyl | | |
| 101. | 3,4- | pentafluoroethyl | 3,5- |
| | bis(trifluoromethyl)- | | bis(trifluoromethyl)- |
| | phenyl | | phenyl |
| 102. | 3,4- | pentafluoroethyl | H |
| | bis(trifluoromethyl)- | | |
| | phenyl | | |
| 103. | 3,4- | 3,4- | CN |
| | bis(trifluoromethyl)- | bis(trifluoromethyl)- | |
| | phenyl | phenyl | |
| 104. | 4-cyano-3- | 3,5- | H |
| | (trifluoromethyl)- | bis(trifluoromethyl)- | |
| | phenyl | phenyl | |
| 105. | 4-cyano-3- | pentafluoroethyl | H |
| | (trifluoromethyl)- | | |
| | phenyl | | |
| 106. | 3,5- | pentafluoroethyl | CN |
| | bis(trifluoromethyl)- | | |
| | phenyl | | |
| 107. | 4-cyano-2,6- | 4-cyano-2,6- | H |
| | bis(trifluoromethyl)- | bis(trifluoromethyl)- | |
| | phenyl | phenyl | |
| 108. | 4-cyano-2,6- | pentafluoroethyl | H |
| | bis(trifluoromethyl)- | | |
| | phenyl | | |
| 109. | 2,4- | 2,6- | H |
| | bis(trifluoromethyl)- | bis(trifluoromethyl)- | |
| | phenyl | phenyl | |
| 110. | 2,4- | 2,6- | CN |
| | bis(trifluoromethyl)- | bis(trifluoromethyl)- | |
| | phenyl | phenyl | |
| 111. | 4-(3′,5′- | pentafluoroethyl | H |
| | bis(trifluoromethyl)phenyl)- | | |
| | 2,6- | | |
| | bis(trifluoromethyl)- | | |
| | phenyl | | |
| 112. | 3,5- | 3,5- | CF₃ |
| | bis(trifluoromethyl)- | bis(trifluoromethyl)- | |
| | phenyl | phenyl | |
| 113. | 3,5- | trifluoromethyl | CF₃ |
| | bis(trifluoromethyl)- | | |
| | phenyl | | |
| 114. | 3,5- | pentafluoroethyl | CF₃ |
| | bis(trifluoromethyl)- | | |
| | phenyl | | |
| 115. | 3,4,5- | 3,4,5- | CF₃ |
| | trifluorophenyl | trifluorophenyl | |
| 116. | 4- | 4- | CF₃ |
| | (trifluoromethyl)phenyl | (trifluoromethyl)phenyl | |
| 117. | tert-butyl | heptafluoro-n- | H |
| | | propyl | |
| 118. | tert-butyl | tert-butyl | tert-butyl |

A further object of the invention are an electronic component comprising at least one compound of the general formula (I)

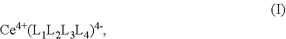

wherein
wherein
L₁; L₂; L₃; and L₄ are independently from each other selected from a bidentate ligand having the general formula (II)

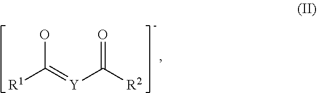

wherein
Y represents N or CR³;
R¹ represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
   cycloalkyl is unsubstituted or substituted with 1 to 13 radicals selected from fluorine and CF₃,
phenyl is substituted by at least one radical selected from NO₂, CN, F, Cl, Br, CF₃, SF₅, OCF₃, SCF₃, SO₂CF₃, N═C(CF₃)₂, and N(SO₂CF₃)₂,
naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl, pyrimidyl and triazinyl are substituted by at least one radical selected from NO₂, CN, F, Cl, Br, CF₃, SF₅, OCF₃, SCF₃, SO₂CF₃, N═C(CF₃)₂, and N(SO₂CF₃)₂; or
R¹ and Y together with the C—O-group (in particular the carbon atom of the C—O-group) to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R² represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
   cycloalkyl is unsubstituted or substituted with 1 to 13 radicals selected from fluorine and CF₃,
   phenyl is substituted by at least one radical selected from NO₂, CN, F, C, Br, CF₃, SF₅, OCF₃, SCF₃, SO₂CF₃, N═C(CF₃)₂, and N(SO₂CF₃)₂,
   naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂, pyridyl, pyrimidyl and triazinyl are substituted by at least one radical selected from NO₂, CN, F, C, Br, CF₃, SF₅, OCF₃, SCF₃, SO₂CF₃, N═C(CF₃)₂, and N(SO₂CF₃)₂; or
R² and Y together with the C—O-group (in particular the carbon atom of the C—O-group) to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R^{a} and R^{b} independently from each other represent halogen, CF₃, CN and C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN;
R³ represents hydrogen, F, Cl, CN, CF₃, OCF₃, SCF₃, methyl, ethyl, t-butyl, adamantyl, phenyl or hetaryl having 4 to 5 carbon atoms, wherein hetaryl has 1, 2 or 3 nitrogen atoms as ring members, wherein phenyl and hetaryl are unsubstituted or substituted by 1 or 2 identical or different radicals R⁵;
R⁴ represents CN, NO₂, SF₅, halogen, C₁-C₄-alkyl, C₁-C₄-haloalkyl, C₁-C₄-haloalkoxy, C₁-C₄-haloalkylsulfanyl, C₁-C₄-haloalkylsulfonyl, N═C(CF₃)₂ and N(SO₂CF₃)₂;
R⁵ represents CN, F, C₁, CF₃, OCF₃, SCF₃, SO₂CF₃, NO₂, N═C(CF₃)₂, SF₅, and N(SO₂CF₃)₂,
with the proviso that the electronic components comprising compounds of formula (I.a),

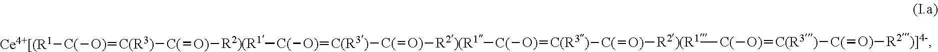

wherein
(R¹, R², R³), (R^{1′}, R^{2′}, R^{3′}), (R^{1″}, R^{2″}, R^{3″}) and (R^{1″′}, R^{2′″}, R^{3′″}) are defined in table 1 above are excluded.

### Component

In the context of the invention, an electronic component is understood to be a discrete or integrated electrical component, which uses the properties of compounds of the general formula (I) or semiconductor matrix materials containing a compound of the general formula (I). In a special embodiment, the electronic component has a layer structure comprising in particular 2, 3, 4, 5, 6, 7 or more layers, wherein at least one of the layers contains at least one compound of the general formula (I). Each of the layers may also contain inorganic materials, or the component may also comprise layers, which are composed entirely from inorganic materials.

Preferably, the electronic component is selected from organic field effect transistors (OFETs), organic electroluminescent devices, organic solar cells (OSCs), devices for electrophotography, organic optical detectors, organic photodetector organic photoreceptors, light-emitting electrochemical cells (LECs) and organic laser diodes. Organic field effect transistors (OFETs) are preferably organic thin film transistors (OTFTs). Organic electroluminescent devices are preferably organic light-emitting diodes (OLEDs). Organic solar cells are preferably exciton solar cells, dye sensitized solar cells (DSSCs) or perovskite solar cells. Devices for electrophotography are preferably photoconductive materials in organic photoconductors (OPC).

Preferably, the electronic component according to the invention is in the form of an organic light-emitting diode, an organic photodetectors, an organic solar cell, a photovoltaic cell, an organic diode or an organic transistor, preferably a field effect transistor or thin-film transistor or a Perovskite solar cell.

The electronic component may be preferably an organic electroluminescent device, in particular in the form of an organic light-emitting diode (OLED). An organic electroluminescent device comprises a cathode, an anode and at least one emitting layer. In addition to these layers, it may also comprise other layers, e.g. one or more hole injection layers, hole transport layers, hole blocking layers, electron transport layers, electron injection layers, exciton blocking layers, electron blocking layers and/or charge generation layers. Intermediate layers, which have e.g. an exciton-blocking-function can also be inserted between two emitting layers. Not all of these layers must necessarily be present.

A preferred embodiment is an electronic component, in particular in the form of an OLED, wherein the layer comprising the compound of formula (I) is a hole transport layer or a hole injection layer. Especially, the electronic component, in particular in the form of an OLED, wherein the layer comprising the compound of formula (I) is a hole transport layer, a hole injection layer or an electron blocking layer. Generally, a hole injection layer is a layer which facilitates electron injection from the anode into the organic semiconductor matrix material. The hole injection layer can be placed directly adjacent to the anode. A hole transport layer transports the holes from the anode to the emitting layer and is located between a hole injection layer and an emitting layer.

A preferred embodiment is an electronic component in the form of an organic photodetector cell. Generally organic photodetectors are layered and usually comprises at least the following layers: filter, anode, at least one photoactive layer and cathode. These layers are generally applied to a substrate commonly used for this purpose. The photoactive region of the photodetector may comprise two layers, each of which has a homogeneous composition and forms a flat donor-acceptor heterojunction. A photoactive region can also comprise a mixed layer and form a donor-acceptor heterojunction in the form of a donor-acceptor bulk heterojunction. In addition to these layers, the organic photodetector cell can also comprises other layers, e.g. selected from
layers with electron transport layer properties (electron transport layer, ETL),
layers comprising a hole-conducting material (hole transport layer, HTL), these do not have to absorb radiation.

A preferred embodiment is an electronic component in the form of an organic solar cell. Generally organic solar cells are layered and usually comprises at least the following layers: anode, at least one photoactive layer and cathode. These layers are generally applied to a substrate commonly used for this purpose. The photoactive region of the solar cell may comprise two layers, each of which has a homogeneous composition and forms a flat donor-acceptor heterojunction. A photoactive region can also comprise a mixed layer and form a donor-acceptor heterojunction in the form of a donor-acceptor bulk heterojunction. In addition to these layers, the organic solar cell can also comprises other layers, e.g. selected from
layers with electron transport layer properties (electron transport layer, ETL),
layers comprising a hole-conducting material (hole transport layer, HTL), these do not have to absorb radiation.

Another preferred embodiment is an electronic component in the form of an organic solar cell, wherein the layer, which comprises the compound of formula (I), has electron conductivity properties (electron transport layer, ETL).

A special embodiment is an electronic component, especially in the form of an organic solar cell, wherein the layer, which comprises at least one of the compounds of formula (I) is part of a pn-junction connecting a light absorbing unit to an additional light absorbing unit in a tandem device or in a multistacked device and/or a pn-junction connecting a cathode or an anode to a light absorbing unit.

### Semiconductor Matrix Materials

The compounds of formula (I) according to the invention and used according to the invention, as well as their charge transfer complexes, their reduction products, can be used as doping agents in organic semiconductor matrix materials, in particular as p-dopant in hole transport layers. The doped semiconductor matrix material, preferably comprising at least one electron donor and at least one compound of the formula (I), as defined above. The electron donor is preferably selected from
4,4′,4″-tris(N-(2-naphthyl)-N-phenyl-amino)triphenylamine (2-TNATA), 4,4′,4″-tris(N-3-methylphenyl-N-phenyl-amino)triphenylamine (m-MTDATA), N,N,N′,N′-tetrakis(4-methoxy-phenyl)benzidine (MeO-TPD), (2,2′,7,7′-tetrakis-(N,N-diphenylamino)-9,9′-spirobifluorene (spiro-TTB), N,N′-bis(naphthalen-1-yl)-N,N′-bis(phenyl)-benzidine, N,N′-bis(naphthalen-1-yl)-N,N′-bis(phenyl)-9,9-spiro-bifluorene, 9,9-bis[4-(N,N-bis-biphenyl-4-yl-amino)phenyl]-9H-fluorene, 2, 2′-bis[N,N-bis(biphenyl-4-yl)amino]-9,9-spiro-bifluorene, N,N′-((9H-fluorene-9,9-diyl)bis(4,1-phenylene))bis(N-([1,1′-biphenyl]-4-yl)-[1, 1-biphenyl]-4-amine) (BPAPF), N,N′-bis(phenanthren-9-yl)-N,N′-bis(phenyl)-benzidine, 1,3,5-tris{4-[bis(9,9-dimethyl-fluoren-2-yl)amino]phenyl}benzene, tri(terphenyl-4-yl)amine, diaminoterphenylene, diaminotrimethylphenylindanes, N,N′-bis(9,9-dimethyl-fluoren-2-yl)-N,N′-diphenyl-benzidine (BF-DPB), N,N′-((9H-fluorene-9,9-diyl)bis(4,1-phenylene))bis(N-([1,1′-biphenyl]-4-yl)-[1,1′-biphenyl]-4-amine) (BPAPF), N4,N4,N4′,N4′-tetrakis(9,9-dimethyl-9H-fluoren-2-yl)-[1,1′-biphenyl]-4,4′-diamine (TDMFB), N-([1,1′-biphenyl]-2-yl)-N-(9,9-dimethyl-9H-fluoren-2-yl)-9,9′-spirobi[fluoren]-2-amine, (2,7-bis[N,N-bis(4-methoxyphenyl)amino]-9,9-spirobi[9H-fluorene] (spiro-MeO-TPD), N-([1,1′-biphenyl]-4-yl)-9,9-dimethyl-N-(4-(9-phenyl-9H-carbazol-3-yl)phenyl)-9H-fluoren-2-amine and mixtures thereof.

Suitable diaminoterphenyls are described in DE 102012007795.

Diaminotrimethylphenylindanes are described in WO 2018/206769.

Di-, tri- and tetraphenylindane amine derivatives are described in WO2020094847.

In particular, the electron donors are selected from 4,4′,4″-tris(N-(2-naphthyl)-N-phenyl-amino)triphenylamine (2-TNATA), 4,4′,4″-tris(N-3-methylphenyl-N-phenyl-amino)triphenylamine (m-MTDATA), N,N,N′,N′-tetrakis(4-methoxy-phenyl)benzidine (MeO-TPD), (2,2′,7,7′-tetrakis-(N,N-diphenylamino)-9,9′-spirobifluorene (spiro-TTB), N,N′-bis(naphthalene-1-yl)-N,N′-bis(phenyl)-benzidine, N,N′-bis(naphthalene-1-yl)-N,N′-bis(phenyl)-9,9-spiro-bifluorene, 9,9-bis[4-(N,N-bis-biphenyl-4-yl-amino)phenyl]-9H-fluorene, 2,2′-bis[N,N-bis(biphenyl-4-yl)amino]-9,9-spiro-bifluorene, N,N′-((9H-fluorene-9,9-diyl)bis(4,1-phenylene))bis(N-([1,1′-biphenyl]-4-yl)-[1,1′-biphenyl]-4-amine) (BPAPF), N,N′-bis(phenanthrene-9-yl)-N,N′-bis(phenyl)-benzidine, 1,3,5-tris{4-[bis(9,9-dimethyl-fluorene-2-yl)amino]phenyl}benzene, tri(terphenyl-4-yl)amine, N-(4-(6-((9,9-dimethyl-9H-fluorene-2-yl)(6-methoxy-[1,1′-biphenyl]-3-yl)amino)-1,3,3-trimethyl-2,3-dihydro-1H-indene-1-yl)phenyl)-N-(6-methoxy-[1,1′-biphenyl]-3-yl)-9,9-dimethyl-9H-fluorene-2-amine, N-([1,1′-biphenyl]-4-yl)-N-(4-(6-([1,1′-biphenyl]-4-yl(9,9-dimethyl-9H-fluorene-2-yl)amino)-1,3,3-trimethyl-2,3-dihydro-1H-indene-1-yl)phenyl)-9,9-dimethyl-9H-fluorene-2-amine, N,N-di([1,1′-biphenyl]-4-yl)-3-(4-(di([1,1′-biphenyl]-4-yl)amino)phenyl)-1,1,3-trimethyl-2,3-dihydro-1H-indene-5-amine, N-(4-(6-(bis(9,9-dimethyl-9H-fluorene-2-yl)amino)-1,3,3-trimethyl-2,3-dihydro-1H-inden-1-yl)phenyl)-N-(9,9-dimethyl-9H-fluorene-2-yl)-9,9-dimethyl-9H-fluorene-2-amine, N-(4-(6-(9,9′-spirobi[fluorene]-2-yl(9,9-dimethyl-9H-fluorene-2-yl)amino)-1,3,3-trimethyl-2,3-dihydro-1H-inden-1-yl)phenyl)-N-(9,9-dimethyl-9H-fluoren-2-yl)-9,9′-spirobi[fluorene]-2-amine, N-(4-(6-(dibenzo[b,d]furane-2-yl(9,9-dimethyl-9H-fluorene-2-yl)amino)-1,3,3-trimethyl-2,3-dihydro-1H-indene-1-yl)phenyl)-N-(9,9-dimethyl-9H-fluorene-2-yl)dibenzo[b,d]furan-2-amine, 9-(4-(6-(9H-carbazol-9-yl)-1,3,3-trimethyl-2,3-dihydro-1H-inden-1-yl)phenyl)-9H-carbazole, N-([1,1′-biphenyl]-4-yl)-3-(4-([1,1′-biphenyl]-4-yl(4-methoxyphenyl)amino)phenyl)-N-(4-methoxyphenyl)-1,1,3-trimethyl-2,3-dihydro-1H-inden-5-amine, 3-(4-(bis(6-methoxy-[1,1′-biphenyl]-3-yl)amino)phenyl)-N,N-bis(6-methoxy-[1,1′-biphenyl]-3-yl)-1,1,3-trimethyl-2,3-dihydro-1H-indene-5-amine, N1-([1,1′-biphenyl]-4-yl)-N1-(4-(6-([1,1′-biphenyl]-4-yl(4-(diphenylamino)phenyl)amino)-1,3,3-trimethyl-2,3-dihydro-1H-inden-1-yl)phenyl)-N4, N4-diphenylbenzene-1,4-diamine, N,N-di([1,1′-biphenyl]-4-yl)-4′-(6-(4-(di([1,1′-biphenyl]-4-yl)amino)phenyl)-1,3,3-trimethyl-2,3-dihydro-1H-indene-1-yl)-[1,1′-biphenyl]-4-amine, N-(4-(5-(bis(9,9-dimethyl-9H-fluorene-2-yl)amino)-1,3,3-trimethyl-2,3-dihydro-1H-indene-1-yl)phenyl)-N-(9,9-dimethyl-9H-fluorene-2-yl)-9,9-dimethyl-9H-fluorene-2-amine, N-(4-(6-(bis(9,9-dimethyl-9H-fluorene-2-yl)amino)-1,3,3-trimethyl-2,3-dihydro-1H-indene-1-yl)-phenyl)-N-(9,9-dimethyl-9H-fluorene-2-yl)-9,9-dimethyl-9H-fluorene-2-amine, N,N′-bis(9,9-dimethyl-fluorene-2-yl)-N,N′-diphenyl-benzidine (BF-DPB), N,N′-((9H-fluorene-9,9-diyl)bis(4,1-phenylene))bis(N-([1,1′-biphenyl]-4-yl)-[1,1′-biphenyl]-4-amine) (BPAPF), N4, N4, N4′, N4′-tetrakis(9,9-dimethyl-9H-fluorene-2-yl)-[1,1′-biphenyl]-4,4′-diamine (TOMFB), N-([1,1′-biphenyl]-2-yl)-N-(9,9-dimethyl-9H-fluorene-2-yl)-9,9′-spirobi[fluorene]-2-amine, (2,7-bis[N,N-bis(4-methoxyphenyl)amino]-9,9-spirobi[9H-fluorene] (spiro-MeO-TPD), a mixture of N-(4-(5-(bis(9,9-dimethyl-9H-fluorene-2-yl)amino)-1,3,3-trimethyl-2,3-dihydro-1H-inden-1-yl)phenyl)-N-(9,9-dimethyl-9H-fluorene-2-yl)-9,9-dimethyl-9H-fluorene-2-amine and N-(4-(6-(bis(9,9-dimethyl-9H-fluorene-2-yl)amino)-1,3,3-trimethyl-2,3-dihydro-1H-inden-1-yl)phenyl)-N-(9,9-dimethyl-9H-fluorene-2-yl)-9,9-dimethyl-9H-fluorene-2-amine, N-([1,1-biphenyl]-4-yl)-9,9-dimethyl-N-(4-(9-phenyl-9H-carbazol-3-yl)phenyl)-9H-fluorene-2-amine and mixtures thereof.

Of course, other suitable organic semiconductor matrix materials, especially hole-conducting materials with semiconducting properties, can also be used.

### Doping

The doping can take place in particular in such a manner that the molar ratio of matrix molecule to compounds of formula (I) is 10000:1 to 1:1, preferably 1000:1 to 2:1, especially 5:1 to 100:1.

### Preparation of the Doped Semiconductor Matrix Material

The doping of the particular matrix material (in the following also indicated as hole-conducting matrix HT) with the compounds of the general formula (I) according to the invention and used according to the invention can be produced by one or a combination of the following processes:
a) Mixed evaporation in the vacuum with a source for HT and a source for at least one compound of the general formula (I).
b) Sequential deposition of HT and at least one compound of the general formula (I), with subsequent inward diffusion of the doping agent by thermal treatment.
c) Doping of an HT layer by a solution of at least one compound of the general formula (I), with subsequent evaporation of the solvent by thermal treatment.
d) Surface doping of an HT layer by a layer of at least one compound of the general formula (I) applied on either or both surfaces of the HT layer.
e) Preparation of a solution of host and at least one compound of the general formula (I) and forming a film from the solution e.g. by coating, casting or printing techniques or other film preparing techniques known to a person skilled in the art.

Another object of the invention is the use of a compound (I) or a mixture thereof as defined above
as organic semiconductor,
as redox doping agent in organic semiconductor matrix materials, especially as p-dopant in hole transport layers,
as electron transport material,
as charge injector in a charge injection layer,
as cathode material in organic batteries,
as electrochromic material.

A further object of the invention is the use of Ce(Ill) complex anions obtained by reduction of a compound (1) as defined above or of charge transfer complexes of a compound (1) as defined above with electron donors as organic semiconductor or as electrochromic material.

The following examples illustrate the invention without limiting it in any way.

### EXAMPLES

### Abbreviation:

TBME tert.-Butylmethylether
MTBE Methyl-tert-butylether
DCM Dichlormethane
DME 1,2-Dimethoxyethane
Spiro-TTB 2,2′,7,7′-tetrakis-(N,N-diphenylamino)-9,9′-spirobifluorene
MeO-TPD N,N,N′,N′-tetrakis(4-methoxy-phenyl)benzidine
Spiro-TAD 2,2′,7,7′-Tetrakis(N,N-diphenylamino)-2,7-diamino-9,9-spirobifluorene
BPAPF N,N′-((9H-fluorene-9,9-diyl)bis(4,1-phenylene))bis(N-([1,1′-biphenyl]-4-yl)-[1, 1′-biphenyl]-4-amine
Al Aluminium
BPhen 4,7-Diphenyl-1,10-phenanthrolin
Cs Caesium
BAlq₂ Bis-(8-hydroxy-2-methylchinolin)-(4-phenylphenoxy)-aluminium
TPBI 2,2′,2″-(1,3,5-Benzinetriyl)-tris(1-phenyl-1-H-benzimidazole)
ppy polypyrrole
acac acetylacetone
TAPC 1,1-Bis[(di-4-tolylamino)phenyl]cyclohexane
ITO Indium Tin Oxide
EQE External Quantum Efficiency
PE Power eFFICIENCY
n.d. not defines/measured

### Example 1

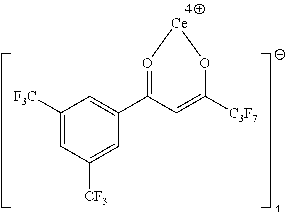

### Ligand:

NaOMe (4 g, 73.1 mmol) was suspended in TBME (50 ml) containing ethyl heptafluorobutyrate (20 g, 82.6 mmol) and cooled to 0° C. 1-(3,5-bis(trifluoromethyl)phenyl)ethan-1-one (16.3 g, 63.6 mmol) in TBME (20 ml) was added dropwise over 30 min. The mixture was warmed to room temperature and stirred overnight. HCl (1M, 80 ml) was added. The organic phase was separated and washed with saturated NaCl. The organic phase was dried over MgSO₄, filtered and the volatiles removed in vacuo. The crude diketone was distilled. The product was obtained as pale yellow liquid (23 g, 80%).

### Complex:

NaOMe (762 mg, 73.1 mmol) was added to a solution of 1-(3,5-bis(trifluoromethyl)phenyl)-6,6,6,6,6,6,6-heptafluoro-1,3-dione (6.4 g, 14.1 mmol) in EtOH (50 ml). The solution was stirred for 5 minutes, then a solution of ceric ammonium nitrate (1.93 g, 3.5 mmol) in EtOH (40 ml) was added. The dark red solution was stirred for 15 min, then water (250 ml) and diethyl ether (250 ml) were added. The organic phase was separated, dried over MgSO₄ and rotated off under reduced pressure. The red oily material was triturared in hexane (50 ml) were it crystallized in a form of a dark red microcrystalline material (4.79 g, 70%). APCI-MS: 1945 [M+H]
Melting point: 140° C. (peak) at 10 K/min determined with DSC.
Decomposition point: 261° C. (onset) at 10 K/min determined with DSC.
Cyclovoltametry in acetonitrile showed the following potential:
E_{1/2} (vs. Fc/Fc+ (MeCN): =+0.35 V

The compound 1 was co-evaporated with the hole transport material N,N′-((9H-fluorene-9,9-diyl)bis(4,1-phenylene))bis(N-([1,1′-biphenyl]-4-yl)-[1, 1′-biphenyl]-4-amine) (BPAPF).

At a doping concentration of 5 mol % a conductivity of 7.0·10⁻⁵ S/cm has been achieved.

### Example 2

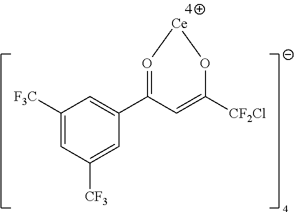

### Ligand:

NaOMe (3.9 g, 72.8 mmol) was suspended in TBME (100 ml) and cooled to 0° C. Ethyl 2-chloro-2,2-difluoroacetate (15 g, 94.6 mmol) and 1-(3,5-bis(trifluoromethyl)phenyl)ethan-1-one (18.6 g, 72.8 mmol) in TBME (100 ml) were added dropwise over 30 min. After 15 min stirring at 0° C., HCl (1M, 75 ml, 75 mmol) was added. The organic phase was separated and washed with saturated NaCl. The organic phase was dried (Na₂SO₄), filtered and the volatiles removed in vacuo. The residue was distilled (108° C., 8 mbar). The product was obtained as colorless liquid (16.0 g, 43.4 mmol). APCI-MS: 369 [M+H].

### Complex:

1-(3,5-bis(trifluoromethyl)phenyl)-4-chloro-4,4-difluorobutane-1,3-dione (15.9 g, 43.1 mmol) was dissolved in TBME (100 ml) and cooled to 0° C. NaH (0.93 g, 38.8 mmol) was added in small portions. A white precipitate formed and was filtered. 12.02 g white solid. The white solid was dissolved in acetonitrile (50 ml) and cooled to 0° C. Cerium ammonium nitrate (4.21 g, 7.68 mmol) was added and the suspension was stirred for 30 min. The suspension was filtered, washed with acetonitrile and the filtrate collected. The volatiles were removed in vacuo and the residue partitioned between n-octane and water. The suspension was filtered and the red crystalline solid recrystallized from acetonitrile (−20° C.). m.p. 149° C. APCI-MS: 1610 [M+].
Melting point: 148° C. (peak) at 10 K/min determined with DSC.
Decomposition point: 235° C. (onset) at 10 K/min determined with DSC.
Cyclovoltametry in acetonitrile showed the following potential:
E_{1/2} (vs. Fc/Fc+ (MeCN): =+0,39 V

The compound 2 was co-evaporated with the hole transport material N,N′-bis(9,9-dimethyl-fluoren-2-yl)-N,N′-diphenyl-benzidine (BF-DPB).

At a doping concentration of 2.5 mol % a conductivity of 2.1·10⁻⁵ S/cm has been achieved.

At a doping concentration of 5 mol % a conductivity of 4.1·10⁻⁵ S/cm has been achieved.

### Example 3

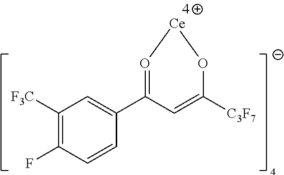

### Ligand:

NaOMe (1.23 g, 22.8 mmol) was suspended in TBME (25 ml) containing ethyl heptafluorobutyrate (6.3 g, 26 mmol) and cooled to 0° C. 1-(4-fluoro-3-(trifluoronethyl)phenyl)ethanone (4.1 g, 20 mmol) in TBME (25 ml) were added dropwise over 30 min. The mixture was warmed to room temperature and stirred overnight. HCl (1M, 25 ml) was added. The organic phase was separated and washed with saturated NaCl. The organic phase was dried over MgSO₄, filtered and the volatiles removed in vacuo. The crude diketone was distilled. The product was obtained as colorless liquid (7.6 g, 95%).

### Complex:

1-(4-fluoro-3-(trifluoromethyl)phenyl)-6,6,6,6,6,6,6-heptafluoro-1,3-dione (3.4 g, 8.6 mmol) was dissolved in EtOH (50 mL), then EtONa (590 mg, 8.6 mmol) was added. The mixture was stirred for 5 min then ceric ammonium nitrate (1.19 g, 2.15 mmol) was added. The dark red solution was stirred for 15 min. The volatiles were then removed in vacuo and the residue was redissolved in diethyl ether (100 ml) and washed with water (3×100 ml). The organic phase was then dried over MgSO₄ and evaporated under reduced pressure. The crude complex was recrystallized from hexane/chloroform (1:1). (2 g, 53%).
Melting point: 132° C. (peak) at 10 K/min determined with DSC.
Decomposition point: 267° C. (onset) at 10 K/min determined with DSC.
Cyclovoltametry in acetonitrile showed the following potential:
E_{1/2} (vs. Fc/Fc+(MeCN): =+0,35 V

The compound 3 was co-evaporated with the hole transport material N,N′-bis(9,9-dimethyl-fluoren-2-yl)-N,N′-diphenyl-benzidine (BF-DPB).

At a doping concentration of 2.1 mol % a conductivity of 4.1·10⁻⁵ S/cm has been achieved.

At a doping concentration of 5.5 mol % a conductivity of 7.0·10⁻⁵ S/cm has been achieved.

### Example 4

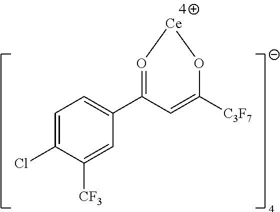

### Ligand:

Ethyl heptafluorobutynoate (8.5 g, 35.2 mmol) was dissolved in MTBE (30 ml) and cooled to 0° C. 1-(4-chloro-3-(trifluoromethyl)phenyl)ethan-1-one (6.02 g, 27.1 mmol) in MTBE (30 ml) was added over 30 min and stirring was continued for further 30 min. The reaction mixture was quenched with HCl (1 M, 40 ml, 40 mmol) and the organic phase was separated. The organic phase was washed with water, saturated NaCl and dried (MgSO₄), filtered and the volatiles were removed in vacuo. The residue was distilled (120° C., 18 mbar). Colorless liquid (8.4 g, 20.7 mmol). APCI-MS: 419 [M+H].

### Complex:

1-(4-chloro-3-(trifluoromethyl)phenyl)-heptafluoro-1,3-dione (4.00 g, 9.6 mmol) was dissolved in TBME (20 ml) and cooled to 0° C. NaH (0.23 g, 9.6 mmol) was added in small portions and the volatiles were removed in vacuo. The residue was dissolved in acetonitrile (20 ml), cooled to 0° C. and cerium ammonium nitrate (1.27 g, 2.33 mmol) was added. After 15 min, the suspension was filtered and the filtrate was collected. The volatiles were removed and the residue dissolved in hexane/TBME (5:1), washed with water and saturated NaCl. The organic phase was dried (MgSO₄), filtered, concentrated and cooled to −20° C. The red crystals were collected by filtration and recrystallized from hexane/toluene. Red powder, (1.8 g, 0.99 mmol) APCI-MS: 1814.
Melting point: 152° C. (peak) at 10 K/min determined with DSC.
Decomposition point: 240° C. (onset) at 10 K/min determined with DSC.
Cyclovoltametry in acetonitrile showed the following potential:
E1/2 (vs. Fc/Fc+(MeCN): =+0.32 V

The compound 4 was co-evaporated with the hole transport material N4,N4,N4′,N4′-tetrakis(9,9-dimethyl-9H-fluoren-2-yl)-[1,1′-biphenyl]-4,4′-diamine (TDMFB). At a doping concentration of 5.5 mol % a conductivity of 8.2·10⁻⁵ S/cm has been achieved.

### Example 5

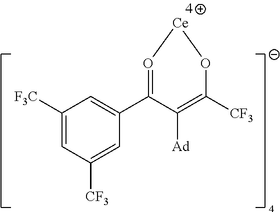

### Ligand:

1,3 dehydroadamantane (1.9 g, 14.7 mmol) was dissolved in diethyl ether (10 ml) and added to a solution of 4,4,5,5,5-pentafluoro-1-(4-(trifluoromethyl)phenyl)pentane-1,3-dione (4.92 g, 14.7 mmol) in diethyl ether (30 ml). The mixture was stirred overnight at room temperature. The solvent was then rotated off and the resulting solid was triturated in hexane (10 ml), followed by filtration. The microcrystalline material was dried under vacuum. (3.78, 55%)

### Complex:

NaH (120 mg, 5 mmol) was added to a solution of 2-((3r,5r,7r)-adamantan-1-yl)-4,4,5,5,5-pentafluoro-1-(4-(trifluoromethyl)phenyl)pentane-1,3-dione (2,34 g, 5 mmol) in TBME (15 ml). After 5 min, the solvent was rotated off and the oily residue was dissolved in MeCN (15 ml). Cerium ammonium nitrate (685 mg, 1.25 mmol) was added and the mixture was stirred for 30 minutes. The red solution was then filtered and the solvent was rotated off. The oily red solid was triturated in hexane (10 ml), filtered and rinsed with hexane (5 ml) and dried under vacuum. The product was obtained as a red powder (937 mg, 37%)
Melting point: 162° C. (peak) at 10 K/min determined with DSC.
Decomposition point: 180° C. (onset) at 10 K/min determined with DSC.
Cyclovoltametry in acetonitrile showed the following potential:
E_{1/2} (vs. Fc/Fc+(MeCN): =+0.30 V

### Example 6

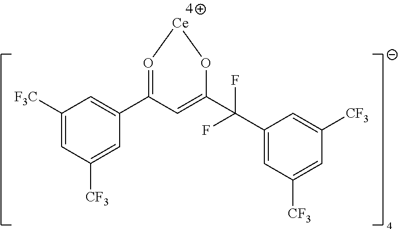

### Ligand:

Ethyl 2-(3,5-bis(trifluoromethyl)phenyl)-2,2-difluoroacetate was synthesized according to Angew.Chem. Int.Ed. 2018, 57,12819-12823.

Ethyl 2-(3,5-bis(trifluoromethyl)phenyl)-2,2-difluoroacetate (4.5 g, 13.4 mmol) was dissolved in TBME (25 ml) and the solution was cooled to 0° C. NaOMe (0.72 g, 13.4 mmol) was added and to the suspension 1-(3,5-bis(trifluoromethyl)phenyl)ethan-1-one (3.42 g, 13.4 mmol) dissolved in TBME (25 ml) was added over 30 min. Under a flow of N₂, the volatiles were evaporated and the residue suspended in HCl (1M, 200 ml, 200 mmol). The suspension was filtered and the white solid was washed with water and dried in vacuo. Off-white solid, 6.42 g. APCI-MS: 546 [M+].

### Complex:

1,4-Bis(3,5-bis(trifluoromethyl)phenyl)-4,4-difluorobutane-1,3-dione (3.00 g, 5.5 mmol) was dissolved in TBME (25 ml) and NaH (0.13 g, 5.5 mmol) was added. After the gas evolution ceased the volatiles were removed in vacuo and the residue was dissolved in acetonitrile (25 ml). Cerium ammonium nitrate (0.76 g, 1.38 mmol) was added and the suspemsion was stirred for 30 min. The suspension was filtered and the filtrate collected. The volatiles were removed in vacuo and the residue dissolved in hot petroleum ether and filtered. Upon cooling to −20° C. red crystals formed which were collected. 1.89 g.
Melting point: 102° C. (peak) at 10 K/min determined with DSC.
Decomposition point: 201° C. (onset) at 10 K/min determined with DSC.
Cyclovoltametry in acetonitrile showed the following potential:
E_{1/2} (vs. Fc/Fc+(MeCN): =+0.40 V

### Example 7

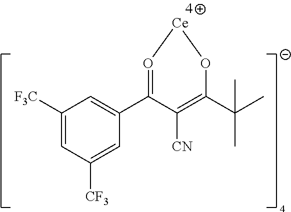

### Ligand:

Trimethylacetylacetonitrile (10 g, 80 mmol) in 20 mL toluene was added dropwise to a suspension of NaH (3.9 g, 160 mmol) in toluene (200 mL) at 0° C. After addition, the mixture allowed to warm up to room temperature and was stirred for 2 hours. 3′,5′ Bis(trifluoromethyl)benzoyl chloride (22 g, 80 mmol) was added and the mixture was stirred overnight. HCl (1M, 100 mL) was then added and the reaction mixture was extracted with 500 mL EtOAc. The organic phase was washed with water and brine, dried over MgSO₄ and evaporated under reduced pressure. The crude material was recrystallized from hot hexane (15.9 g, 42%). APCI-MS: 366 [M+H].

### Complex:

2-(3,5-bis(trifluoromethyl)benzoyl)-4,4-dimethyl-3-oxopentanenitrile (3.00 g, 8.22 mmol) was dissolved in TBME (30 ml) and NaH (0.197 g, 8.22 mmol) was added in small portions at 0° C. After 30 min of stirring, all volatiles were removed in vacuo. The oily residue was dissolved in acetonitrile (30 ml) and cerium ammonium nitrate (1.06 g, 1.93 mmol) was added at 0° C. After 30 min, the red solution was filtered and the filtrate was collected. The volatiles were removed in vacuo and the residue triturated with hot heptane and filtered while hot. Upon cooling to −20° C. dark red crystals were formed, which were collected by filtration. 1.54 g, m.p. 204° C. APCI-MS: 1596 [M+].
Melting point: 204° C. (peak) at 10 K/min determined with DSC.
Decomposition point: 229° C. (onset) at 10 K/min determined with DSC.
Cyclovoltametry in acetonitrile showed the following potential:
E_{1/2} (vs. Fc/Fc+(MeCN): =+0.31 V

### Example 8

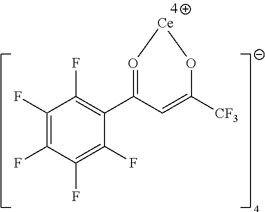

### Ligand:

NaOMe (3.5 g, 64.5 mmol) was suspended in TBME (50 ml) containing ethyl trifluoroacetate (10.3 g, 73 mmol) and cooled to 0° C. 3′,4′,5′,6′-Pentafluoroacetophenone (11.8 g, 56.1 mmol) in TBME (20 ml) was added dropwise over 30 min. The mixture was warmed to room temperature and stirred for 2 hours. HCl (1M,75 ml) was added. The organic phase was separated and washed with saturated NaCl. The organic phase was dried over MgSO₄, filtered and the volatiles removed in vacuo. The product was obtained as pale yellow liquid (8.4 g, 49%).

### Complex:

NaH (390 mg, 16.3 mmol) was added to a solution of 4,4,4-trifluoro-1-(2,3,4,5,6-pentafluorophenyl)butane-1,3-dione (5 g, 16.3 mmol) in 50 ml TBME. The mixture was stirred for 10 min and the solvent was then rotated off. The oily residue was redissolved in acetonitrile (40 ml) followed by the addition of cerium ammonium nitrate (2.2 g, 4 mmol). The mixture was stirred for 20 min, filtered and the solvent was rotated off under reduced pressure. The crude complex was extracted with hexane (20 mL) and rotated off. The product was obtained as a very viscous red liquid (4.8 g, 85%).

Cyclovoltametry in acetonitrile showed the following potential:
E_{1/2} (vs. Fc/Fc+(MeCN): =+0.51 V

### Example 9

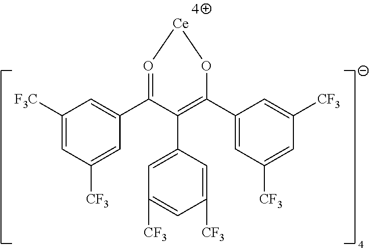

### Ligand:

1-(bromomethyl)-3,5-bis(trifluoromethyl)benzene (20 g, 72.3 mmol), Pd(PPh3)2Cl2 (1.53 g, 2.18 mmol) and activated Zn (9.46 g, 144.6 mmol) were suspended in DME and the suspension was cooled to 0° C. 3,5-bis(trifluoromethyl)benzoyl chloride (22.2 g, 72.3 mmol) dissolved in DME (100 ml) was over the course of 30 min and the solution was allowed to reach room temperature. Stirring was continued for 12 h and saturated NH₄Cl was added. The suspension was extracted with diethyl ether and the phases separated. The organic phase was washed with Na₂CO₃ (10% wt.), twice with water and saturated NaHCO₃. The organic phase was dried (Na₂SO₄), filtered and the volatiles were removed in vacuo. The residue was recrystallized from heptane. 6.1 g off-white crystals. APCI-MS: 469 [M+H].

Hexamethyldisilazane (2.1 g, 13.0 mmol) was dissolved in toluene (40 ml) and cooled to 0° C. n-BuLi in hexanes (1.6M, 12.8 mmol, 8 ml) was added dropwise and after complete addition 1,2-bis(3,5-bis(trifluoromethyl)phenyl)ethan-1-one (3 g, 4.3 mmol) was added. The solution was allowed to warm to room temperature and stirring was continued for 12 h. Acetic acid (20 ml) was added and the reaction mixture was diluted with water. The organic phase was separated and was washed twice with water. The organic phase was dried (MgSO₄), filtered and the volatiles were removed in vacuo. The residue was recrystallized from octane and a second time from EtOH:H2O 6:1. 2.60 g, 3.7 mmol. APCI-MS: 708 [M+].

### Complex:

1,2,3-tris(3,5-bis(trifluoromethyl)phenyl)propane-1,3-dione (2.43 g, 3.43 mmol) were dissolved in THF (25 ml), then NaH (123 mg, 5.14 mmol) were added. The solid was filtered off and to the filtrate was added hexane. All volatiles were removed in vacuo. The residue was dissolved in a minimum amount of diethyl ether and diluted with hexane. Upon concentration crystals formed which were collected by filtration (2.13 g). The white solid was dissolved in acetonitrile (20 ml) and cooled to 0° C. Cerium ammonium nitrate (372 mg, 0.678 mmol) was added and the mixture was cooled to −20° C. The suspension was filtered and the volatiles were removed in vacuo. Crystallization from toluene/hexane gave 25 mg of red crystals.
Melting point: 227° C. (peak) at 10 K/min determined with DSC.
Decomposition point: 271° C. (onset) at 10 K/min determined with DSC.
Cyclovoltametry in acetonitrile showed the following potential:
E_{1/2} (vs. Fc/Fc+(MeCN): =+0.38 V

### Synthesis of 10

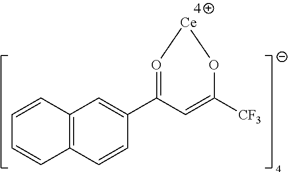

4,4,4-trifluoro-1-(naphthalen-2-yl)butane-1,3-dione is commercial available.

4,4,4-trifluoro-1-(2-naphthyl)-1,3-butanedione (2.5 g, 9.4 mmol) was dissolved in EtOH (50 mL), then NaOEt (640 mg, 9.4 mmol) was added. The mixture was stirred for 5 min, then ceric ammonium nitrate (1.28 g, 2.3 mmol) was added. The dark red solution was stirred for 15 min and filtered. The volatiles were removed in vacuo and the residue was redissolved in DCM (300 ml) and washed with water (100 ml). The organic phase was then dried over MgSO₄ and evaporated under reduced pressure. The crude complex was recrystallized from hexane/chloroform (1:1) at −20° C. (1.27 g, 45%). APCI-MS: 1197 [M+H].
Melting point: 133° C. (peak) at 10 K/min determined with DSC.
Decomposition point: 214° C. (onset) at 10 K/min determined with DSC.
Cyclovoltametry in acetonitrile showed the following potential:
E_{1/2} (vs. Fc/Fc+(MeCN): =+0.24 V

### Synthesis of 11

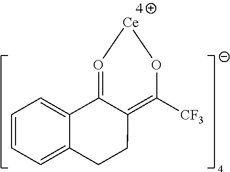

### Ligand:

Tetralone (10 g, 68.4 mmol) was dissolved in TBME and added to a solution of ethyl pentafluoropropanoate (17 g, 88.9 mmol) and NaOMe (3.7 g, 68.4 mmol) at 0° C. over the course of 45 min. Stirring was continued for 30 min, then HCl (1 M, 70 ml, 70 mmol) was added. The phases were separated and the organic phase washed with water and concentrated NaCl. The organic phase was dried (MgSO₄), filtered and the volatiles were removed in vacuo. The oily residue was crystallized from petroleum ether (−20° C.). 8.5 g white solid. M.p. 45° C. APCI-MS: 293 [M+H].

### Complex:

2-(pentafluoropropanoyl)-3,4-dihydronaphthalen-1(2H)-one (3 g, 10.3 mmol) was dissolved in TBME (30 ml) and cooled to 0° C. NaH (0.25 g, 10.3 mmol) was added in small portions and the volatiles removed in vacuo. The residue was dissolved in acetonitrile (30 ml), cooled to 0° C. and cerium ammonium nitrate (1.41 g, 2.58 mmol) was added. Stirring was continued for 1 h and the suspension was filtered. The filtrate was collected and the volatiles removed in vacuo. The residue was crystallized from n-octane/toluene. 1.30 g APCI-MS: 1304 [M+].
Melting point: 142° C. (peak) at 10 K/min determined with DSC.
Decomposition point: 220° C. (onset) at 10 K/min determined with DSC.
Cyclovoltametry in acetonitrile showed the following potential:
E_{1/2} (vs. Fc/Fc+(MeCN): =+0.18 V

### Example 12

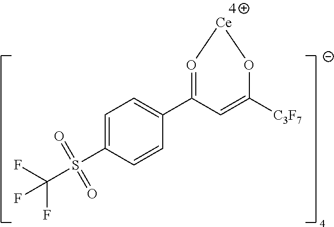

### Ligand:

Synthesis of 1-(4-(trifluoromethylsulfonyl)phenyl)ethan-1-one was carried out according to Journal of Organic Chemistry, 2015, vol. 80, 15, 7658-7665.

Ethyl heptafluorobutyrate (3.18 g, 13.2 mmol) and NaOMe (0.52 g, 9.64 mmol) were dissolved in TBME (11 ml) and the reaction mixture was cooled to 0° C. 1-(4-(trifluoromethylsulfonyl)phenyl)ethan-1-one (2.21 g, 8.77 mmol) was dissolved in TBME (11 ml) and added slowly. After 15 min the reaction mixture was diluted with 1M HCl (10 ml) and diluted with Et₂O and H₂O. The organic phase was separated and the organic phase was washed with H₂O and saturated NaCl. The organic phase was dried with MgSO₄, filtered, and the volatiles removed in vacuo. The residue was recrystallized from hexane to give the title product as yellow solid (3.00 g, 76%). APCI-MS 449 [M+H]+.

### Complex:

Heptafluoro-1-(4-((trifluoromethyl)sulfonyl)phenyl)-hex-4-yne-1,3-dione (3.00 g, 6.7 mmol) was dissolved in TBME (20 ml) and cooled to 0° C. and NaH (0.16 g, 6.7 mmol) was added. After the gas evolution ceased the volatiles were removed in vacuo. The residue was dissolved in acetonitrile (20 ml) and cerium ammonium nitrate (0.91 g, 1.7 mmol) was added. After 30 minutes, the suspension was filtered and the filtrate dissolved with toluene/hexane (1:1). The organic phase was washed with water (2×) and saturated NaCl. The organic phase was dried with MgSO₄, filtrered and the volatiles removed in vacuo. The residue was recrystallized from DCM/petroleum ether to give the title product as red crystalline solid (1.29 g, 40%). APCI-MS: 1930 [M+].

### Example 13

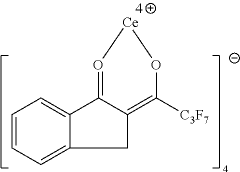

Ligand: 2-(4,4,4,4,4,4,4-heptafluoro-4λ⁸-but-2-ynoyl)-2,3-dihydro-1H-indenolate NaOMe (5.32 g, 98.5 mmol) was suspended in TBME (50 ml) containing ethyl heptafluorobutyrate (6.3 g, 26 mmol) and cooled to 0° C. 1-indanone (10 g, 75 mmol) in TBME (25 ml) was added dropwise over 30 min. The mixture was warmed to room temperature and stirred overnight. HCl (1M, 100 ml) was added. The organic phase was separated and washed with saturated NaCl. The organic phase was dried over MgSO₄, filtered and the volatiles removed in vacuo. The crude diketone was distilled twice. The product was obtained as colorless liquid (12.6 g, 51%).

### Sodium Diketonate:

NaH (0.66 g, 0.66 mmol) was added to a solution of 2-(4,4,4,4,4,4,4-heptafluoro-4λ⁸-but-2-ynoyl)-2,3-dihydro-1H-inden-1-one (7.5 g, 22.8 mmol) in TBME (50 ml) at 0° C. After the gas evolution ceased, the mixture was filtered. The filtrate was rotated off in vacuo and the oily residue was triturated in hexane. The product was obtained as white solid (7.8 g, 98%).

### Complex:

Sodium 2-(4,4,4,4,4,4,4-heptafluoro-4λ⁸-but-2-ynoyl)-2,3-dihydro-1H-indenolate (4 g, 11.4 mmol) was suspended in acetonitrile (50 ml) at 0° C. Cerium ammonium nitrate (1.56 g, 2.85 mmol) was added and the suspension was stirred for 30 min. The suspension was filtered and rinsed with water (50 ml) and hexane (50 ml). The dark purple crystalline solid was recrystallized from a mixture hexane/diethyl ether (1:1). 3.2 g, 77%).

### Example 14

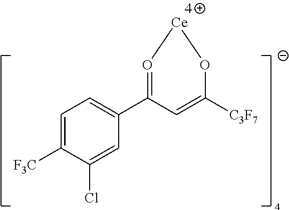

### Ligand:

1-(3-chloro-4-(trifluoromethyl)phenyl)-6,6,6,6,6,6,6-heptafluoro-6λ⁸-hex-4-yne-1,3-dione NaOMe (1.68 g, 30.5 mmol) was suspended in TBME (80 ml) containing ethyl heptafluorobutyrate (7.4 g, 30.5 mmol) and cooled to 0° C. 1-(3-chloro-4-(trifluoromethyl)phenyl)ethan-1-one (5.22 g, 23.5 mmol) in TBME (25 ml) were added dropwise over 30 min. The mixture was warmed to room temperature and stirred for 2 hours. HCl (1M, 30 ml) was added. The organic phase was separated and washed with saturated NaCl. The organic phase was dried over MgSO₄, filtered and the volatiles removed in vacuo. The crude diketone was distilled. The product was obtained as colorless liquid (7.88 g, 80%).

### Complex:

1-(3-chloro-4-(trifluoromethyl)phenyl)-6,6,6,6,6,6,6-heptafluoro-6λ⁸-hex-4-yne-1,3-dione (7.88 g, 18.85 mmol) was added to a cooled suspension of NaH (0.455 g, 18.9 mmol) in TBME (50 ml) at 0° C. After the gas evolution ceased the volatiles were removed in vacuo and the residue was dissolved in acetonitrile (20 ml). Cerium ammonium nitrate (2.58 g, 4.71 mmol) was added and the suspension was stirred for 30 min. The volatiles were removed and the residue dissolved in diethyl ether (100 ml), washed with water and saturated NaCl. The organic phase was dried (MgSO₄), filtered, and rotated off. The red material was recrystallized from hexane. (5.56 g, 65%).

### Example 15

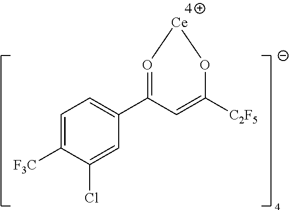

### Ligand:

1-(3-chloro-4-(trifluoromethyl)phenyl)-4,4,5,5,5-pentafluoropentane-1,3-dione NaOMe (2.1 g, 38.2 mmol) was suspended in TBME (50 ml) containing ethyl pentafluoropropionate (7.33 g, 38.2 mmol) and cooled to 0° C. 1-(3-chloro-4-(trifluoromethyl)phenyl)ethan-1-one (6.54 g, 29.5 mmol) in TBME (20 ml) were added dropwise over 30 min. The mixture was warmed to room temperature and stirred for 1 hour. HCl (1M, 40 ml) was added. The organic phase was separated and washed with saturated NaCl. The organic phase was dried over MgSO₄, filtered and the volatiles removed in vacuo. The crude diketone was distilled. The product was obtained as colorless liquid (10.42 g, 96%).

### Complex:

A solution of 1-(3-chloro-4-(trifluoromethyl)phenyl)-4,4,5,5,5-pentafluoropentane-1,3-dione (8 g, 21.7 mmol) in TBME (20 ml) was added to a cooled suspension of NaH (0.52 g, 21.7 mmol) in TBME (20 ml) at 0° C. After the gas evolution ceased the volatiles were removed in vacuo and the residue was dissolved in acetonitrile (50 ml). Cerium ammonium nitrate (2.97 g, 5.4 mmol) was added and the suspension was stirred for 30 min. The volatiles were removed and the residue dissolved in diethyl ether (100 ml), washed with water and saturated NaCl. The organic phase was dried (MgSO₄), filtered, and and rotated off. The red oily material was sonicated in hexane. The red solid obtained was recrystallizedfrom hexane. (3.4 g, 39%).

### Example 16

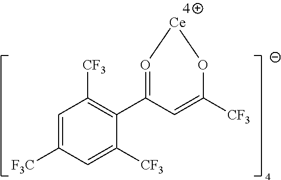

### Ligand:

4,4,4-trifluoro-1-(2,4,6-tris(trifluoromethyl)phenyl)butane-1,3-dione NaOMe (0.5 g, 9.4 mmol) was suspended in TBME (20 ml) containing ethyl trifluoroacetate (1.3 g, 9.4 mmol) and cooled to 0° C. 1-(2,4,6-tris(trifluoromethyl)phenyl)ethan-1-one (2.4 g, 7.4 mmol) in TBME (25 ml) were added dropwise over 30 min. The mixture was warmed to room temperature and stirred overnight. HCl (1M, 15 ml) was added. The organic phase was separated and washed with saturated NaCl. The organic phase was dried over MgSO₄, filtered and the volatiles removed in vacuo. The crude diketone was distilled. The product was obtained as colorless liquid (2.1 g, 67%).

### Complex:

4,4,4-trifluoro-1-(2,4,6-tris(trifluoromethyl)phenyl)butane-1,3-dione (2.1 g, 5 mmol) was added to a cooled suspension of NaH (0.12 g, 5 mmol) in TBME (20 ml) at 0° C. After the gas evolution ceased the volatiles were removed in vacuo and the residue was dissolved in acetonitrile (25 ml). Cerium ammonium nitrate (0.68 g, 1.25 mmol) was added and the suspension was stirred for 30 min. The suspension was filtered and rinsed with water (50 ml). The red crystalline solid was recrystallized mixture hexane/dichloromethane (9:1). 0.6 g, 30%).
Melting point: 148° C. (peak) at 10 K/min determined with DSC.
Decomposition point: 280° C. (onset) at 10 K/min determined with DSC.
Cyclovoltametry in acetonitrile showed the following potential:
E1/2 (vs. Fc/Fc+(MeCN): =+0,6 V

### Example 17

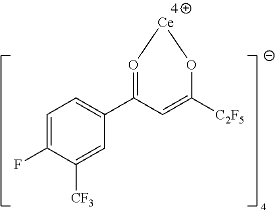

### Ligand:

4,4,5,5,5-pentafluoro-1-(4-fluoro-3-(trifluoromethyl)phenyl)pentane-1,3-dione NaOMe (1.4 g, 26 mmol) was suspended in TBME (50 ml) containing ethyl pentafluoropropionate (5 g, 26 mmol) and cooled to 0° C. 1-(3-chloro-4-(trifluoromethyl)phenyl)ethan-1-one (5.34 g, 26 mmol) in TBME (20 ml) were added dropwise over 30 min. The mixture was warmed to room temperature and stirred for 1 hour. HCl (1M, 25 ml) was added. The organic phase was separated and washed with saturated NaCl. The organic phase was dried over MgSO₄, filtered and the volatiles removed in vacuo. The product was obtained as pale yellow liquid (1.79 g, 20%).

### Complex:

4,4,5,5,5-pentafluoro-1-(4-fluoro-3-(trifluoromethyl)phenyl)pentane-1,3-dione (1.79 g, 5.1 mmol) was dissolved in EtOH (50 mL), then NaOH 1M in EtOH (5.1 ml, 5.1 mmol) was added. The mixture was stirred for 5 min then ceric ammonium nitrate (0,69 g, 1.27 mmol) was added. The dark red solution was stirred for 15 min. The volatiles were then removed in vacuo and the residue was redissolved in diethyl ether (100 ml) and washed with water (3×100 ml). The organic phase was then dried over MgSO₄ and evaporated under reduced pressure. The material was extracted in hexane, concentrated and cooled to −20° C. The red crystals were collected by filtration and dried under vacuum. (0,8 g, 41%).

### Sample Preparation

Thin films and OLEDs are prepared by thermal evaporation at room temperature under ultrahigh vacuum conditions (base pressure <5×10⁻⁷ mbar) by controlling the evaporation rates with quartz crystal microbalances (QCMs).

Doped layers for conductivity measurement are prepared by co-deposition of host and dopant by controlling the evaporation rates with two independent QCMs. 30-50 nm thick films with 10-20 wt % dopant were prepared on glass substrates with 50 nm thick gold electrodes. The channel length was 100 μm. Samples were encapsulated with cap glasses and getter.

Bottom-emitting OLEDs were prepared by subsequent deposition of an organic multi-layer stack (see page 5) on glass substrates with pre-patterned ITO electrodes. As top electrode 100 nm of aluminum was deposited.

### Measurement

Lateral conductivity was determined from current-voltage characteristics (−10V to 10 V). OLEDs were characterized in an integrating sphere using an SMU (Source Measure Unit) or current driving and voltage measurement and a photodiode and spectrometer to study emitting properties. The data are summarized in tabel 1.

TABLE 1
Conductivity Measurements

| | | Example 17 | | Example 14 | | Example 16 |
|---|---|---|---|---|---|---|
| | intrinsic | 10 wt % | 20 wt % | 10 wt % | 20 wt % | 10 wt % |
| Spiro- | 6E−9 | n.d. | n.d. | n.d. | n.d. | 3.0E−5 |
| TTB | | | | | | |
| MeO- | 7E−8* | n.d. | n.d. | 8.7E−5 | 2.3E−4 | n.d. |
| TPD | | | | | | |
| Spiro- | Not | n.d. | n.d. | 4.8E−5 | 8.0E−5 | n.d. |
| TAD | reported | | | | | |
| BPAPF | 5E−9 | 4.8E−7 | 1.8E−6 | 1.9E−5 | 3.1E−5 | 3.1E−5 |

*Snaith, H. J. (2021), Nature Materials 2021 20: 9, 20(9), 1248-1254.

### OLED Measurements

The parameters and conditions are listed in table 2.

TABLE 2

| Stack A | Stack B |
|---|---|
| AI (100 nm) | AI (100 nm) |
| BPhen:Cs (50 nm) | BPhen:Cs (50nm) |
| BAIq₂ (10 nm) | BAIq₂ (10 nm) |
| TPBI:Ir(ppy)₂(acac) (8 wt %, 12 nm) | TPBI:Ir(ppy)₂(acac) (8 wt %, 12 nm) |
| TCTA:Ir(ppy)₂(acac) (8 wt %, 8 nm) | TCTA:Ir(ppy)₂(acac) (8 wt %, 8 nm) |
| TAPC (10 nm) | TAPC (10 nm) |
| Spiro-TTB (40 nm) | BPAPF (40 nm) |
| Spiro-TTB:Example 14 (10 wt % 10 nm) | BPAPF:Example 14 (10 wt % 10 nm) |
| ITO (90 nm) | ITO (90 nm) |

Measurement was carried out at 10 mA/cm² in integrating sphere. The results are listed in table 3.

TABLE 3

| OLED | Voltage | Luminous | | PE | Emission |
|---|---|---|---|---|---|
| structure | (V) | flux (lm) | EQE | [lm/W] | max (nm) |
| A | 3.3 | 0.13 | 17.3% | 60.7 | 523.5 |
| B | 3.3 | 0.11 | 15.2% | 54.0 | 523.3 |

1. Compound of the general formula (I) 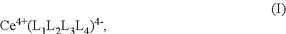
wherein
L₁; L₂; L₃; and L₄ are independently from each other selected from a bidentate ligand having the general formula (II) 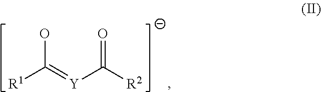
wherein
Y represents N or CR³;
R¹ represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein cycloalkyl is unsubstituted or substituted with 1 to 13 radicals selected from fluorine and CF₃,
phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂;
or
R¹ and Y together with the carbon atom of the C—O-group to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R² represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein cycloalkyl is unsubstituted or substituted with 1 to 13 radicals selected from fluorine and CF₃,
phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂;
or
R² and Y together with the carbon atom of the C—O-group to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R^{a} and R^{b} independently from each other represent halogen, CF₃, CN and C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN;
R³ represents hydrogen, F, Cl, CN, CF₃, OCF₃, SCF₃, methyl, ethyl, t-butyl, adamantyl, phenyl or hetaryl having 4 to 5 carbon atoms, wherein hetaryl has 1, 2 or 3 nitrogen atoms as ring members, wherein phenyl and hetaryl are unsubstituted or substituted by 1 or 2 identical or different radicals R⁵;
R⁴ represents CN, NO₂, SF₅, halogen, C₁-C₄-alkyl, C₁-C₄-haloalkyl, C₁-C₄-haloalkoxy, C₁-C₄-haloalkylsulfanyl, C₁-C₄-haloalkylsulfonyl, N═C(CF₃)₂ and N(SO₂CF₃)₂;
R⁵ represents CN, F, C₁, CF₃, OCF₃, SCF₃, SO₂CF₃, NO₂, N═C(CF₃)₂, SF₅, and N(SO₂CF₃)₂
with the proviso that the compounds of formula (I.a), 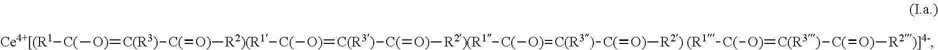
wherein
(R¹, R², R³), (R^{1′}, R^{2′}, R^{3′}), (R^{1″}, R^{2″}, R^{3″}) and (R^{1′″}, R^{2′″}, R^{3′″}) are each selected from the definition given in one line of the following table

| | R¹, R^{1′}, | R², R^{2′}, | R³, R^{3′}, |
|---|---|---|---|
| | R^{1″}, R^{1′″} | R^{2″}, R^{2′″} | R^{3″}, R^{3′″} |
| 1. | trifluoromethyl | trifluoromethyl | H |
| 2. | trifluoromethyl | trifluoromethyl | CN |
| 3. | trifluoromethyl | trifluoromethyl | F |
| 4. | trifluoromethyl | trifluoromethyl | Cl |
| 5. | trifluoromethyl | trifluoromethyl | pentafluorophenyl |
| 6. | trifluoromethyl | trifluoromethyl | tetrafluoro-2- |
| | | | (trifluoromethyl)phenyl |
| 7. | trifluoromethyl | trifluoromethyl | tetrafluoro-3- |
| | | | (trifluoromethyl)phenyl |
| 8. | trifluoromethyl | trifluoromethyl | tetrafluoro-4- |
| | | | (trifluoromethyl)phenyl |
| 9. | trifluoromethyl | trifluoromethyl | tetrafluoro-2- |
| | | | pyridyl |
| 10. | trifluoromethyl | trifluoromethyl | tetrafluoro-3- |
| | | | pyridyl |
| 11. | trifluoromethyl | trifluoromethyl | tetrafluoro-4- |
| | | | pyridyl |
| 12. | trifluoromethyl | trifluoromethyl | tetrafluoro-2- |
| | | | benzonitrile |
| 13. | trifluoromethyl | trifluoromethyl | tetrafluoro-3- |
| | | | benzonitrile |
| 14. | trifluoromethyl | trifluoromethyl | tetrafluoro-4- |
| | | | benzonitrile |
| 15. | pentafluoroethyl | pentafluoroethyl | H |
| 16. | pentafluoroethyl | pentafluoroethyl | CN |
| 17. | pentafluoroethyl | pentafluoroethyl | F |
| 18. | pentafluoroethyl | pentafluoroethyl | Cl |
| 19. | pentafluoroethyl | pentafluoroethyl | pentafluorophenyl |
| 20. | pentafluoroethyl | pentafluoroethyl | tetrafluoro-2- |
| | | | (trifluoromethyl)phenyl |
| 21. | pentafluoroethyl | pentafluoroethyl | tetrafluoro-3- |
| | | | (trifluoromethyl)phenyl |
| 22. | pentafluoroethyl | pentafluoroethyl | tetrafluoro-4- |
| | | | (trifluoromethyl)phenyl |
| 23. | pentafluoroethyl | pentafluoroethyl | tetrafluoro-2- |
| | | | pyridyl |
| 24. | pentafluoroethyl | pentafluoroethyl | tetrafluoro-3- |
| | | | pyridyl |
| 25. | pentafluoroethyl | pentafluoroethyl | tetrafluoro-4- |
| | | | pyridyl |
| 26. | pentafluoroethyl | pentafluoroethyl | tetrafluoro-2- |
| | | | benzonitrile |
| 27. | pentafluoroethyl | pentafluoroethyl | tetrafluoro-3- |
| | | | benzonitrile |
| 28. | pentafluoroethyl | pentafluoroethyl | tetrafluoro-4- |
| | | | benzonitrile |
| 29. | heptafluoro-n- | heptafluoro-n- | H |
| | propyl | propyl | |
| 30. | heptafluoro-n- | heptafluoro-n- | CN |
| | propyl | propyl | |
| 31. | heptafluoro-n- | heptafluoro-n- | F |
| | propyl | propyl | |
| 32. | heptafluoro-n- | heptafluoro-n- | Cl |
| | propyl | propyl | |
| 33. | heptafluoro-n- | heptafluoro-n- | pentafluorophenyl |
| | propyl | propyl | |
| 34. | heptafluoro-n- | heptafluoro-n- | tetrafluoro-2- |
| | propyl | propyl | (trifluoromethyl)phenyl |
| 35. | heptafluoro-n- | heptafluoro-n- | tetrafluoro-3- |
| | propyl | propyl | (trifluoromethyl)phenyl |
| 36. | heptafluoro-n- | heptafluoro-n- | tetrafluoro-4- |
| | propyl | propyl | (trifluoromethyl)phenyl |
| 37. | heptafluoro-n- | heptafluoro-n- | tetrafluoro-2- |
| | propyl | propyl | pyridyl |
| 38. | heptafluoro-n- | heptafluoro-n- | tetrafluoro-3- |
| | propyl | propyl | pyridyl |
| 39. | heptafluoro-n- | heptafluoro-n- | tetrafluoro-4- |
| | propyl | propyl | pyridyl |
| 40. | heptafluoro-n- | heptafluoro-n- | tetrafluoro-2- |
| | propyl | propyl | benzonitrile |
| 41. | heptafluoro-n- | heptafluoro-n- | tetrafluoro-3- |
| | propyl | propyl | benzonitrile |
| 42. | heptafluoro-n- | heptafluoro-n- | tetrafluoro-4- |
| | propyl | propyl | benzonitrile |
| 43. | heptafluoro- | heptafluoro- | H |
| | isopropyl | isopropyl | |
| 44. | heptafluoro- | heptafluoro- | CN |
| | isopropyl | isopropyl | |
| 45. | heptafluoro- | heptafluoro- | F |
| | isopropyl | isopropyl | |
| 46. | heptafluoro- | heptafluoro- | Cl |
| | isopropyl | isopropyl | |
| 47. | heptafluor-iso- | heptafluoro- | pentafluorophenyl |
| | propyl | isopropyl | |
| 48. | heptafluor-iso- | heptafluoro- | tetrafluoro-2- |
| | propyl | isopropyl | (trifluoromethyl)phenyl |
| 49. | heptafluoro- | heptafluoro- | tetrafluoro-3- |
| | isopropyl | isopropyl | (trifluoromethyl)phenyl |
| 50. | heptafluoro- | heptafluoro- | tetrafluoro-4- |
| | isopropyl | isopropyl | (trifluoromethyl)phenyl |
| 51. | heptafluoro- | heptafluoro- | tetrafluoro-2- |
| | isopropyl | isopropyl | pyridyl |
| 52. | heptafluoro- | heptafluoro- | tetrafluoro-3- |
| | isopropyl | isopropyl | pyridyl |
| 53. | heptafluoro- | heptafluoro- | tetrafluoro-4- |
| | isopropyl | isopropyl | pyridyl |
| 54. | heptafluoro- | heptafluoro- | tetrafluoro-2- |
| | isopropyl | isopropyl | benzonitrile |
| 55. | heptafluoro- | heptafluoro- | tetrafluoro-3- |
| | isopropyl | isopropyl | benzonitrile |
| 56. | heptafluoro- | heptafluoro- | tetrafluoro-4- |
| | isopropyl | isopropyl | benzonitrile |
| 57. | tert-butyl | tert-butyl | H |
| 58. | tert-butyl | tert-butyl | CN |
| 59. | tert-butyl | tert-butyl | F |
| 60. | tert-butyl | tert-butyl | Cl |
| 61. | tert-butyl | tert-butyl | pentafluorophenyl |
| 62. | tert-butyl | tert-butyl | tetrafluoro-2- |
| | | | (trifluoromethyl)phenyl |
| 63. | tert-butyl | tert-butyl | tetrafluoro-3- |
| | | | (trifluoromethyl)phenyl |
| 64. | tert-butyl | tert-butyl | tetrafluoro-4- |
| | | | (trifluoromethyl)phenyl |
| 65. | tert-butyl | tert-butyl | tetrafluoro-2- |
| | | | pyridyl |
| 66. | tert-butyl | tert-butyl | tetrafluoro-3- |
| | | | pyridyl |
| 67. | tert-butyl | tert-butyl | tetrafluoro-4- |
| | | | pyridyl |
| 68. | tert-butyl | tert-butyl | tetrafluoro-2- |
| | | | benzonitrile |
| 69. | tert-butyl | tert-butyl | tetrafluoro-3- |
| | | | benzonitrile |
| 70. | tert-butyl | tert-butyl | tetrafluoro-4- |
| | | | benzonitrile |
| 71. | pentafluorophenyl | pentafluorophenyl | H |
| 72. | pentafluorophenyl | pentafluorophenyl | CN |
| 73. | pentafluorophenyl | pentafluorophenyl | F |
| 74. | pentafluorophenyl | pentafluorophenyl | Cl |
| 75. | pentafluorophenyl | pentafluorophenyl | pentafluorophenyl |
| 76. | pentafluorophenyl | pentafluorophenyl | tetrafluoro-2- |
| | | | (trifluoromethyl)phenyl |
| 77. | pentafluorophenyl | pentafluorophenyl | tetrafluoro-3- |
| | | | (trifluoromethyl)phenyl |
| 78. | pentafluorophenyl | pentafluorophenyl | tetrafluoro-4- |
| | | | (trifluoromethyl)phenyl |
| 79. | pentafluorophenyl | pentafluorophenyl | tetrafluoro-2- |
| | | | pyridyl |
| 80. | pentafluorophenyl | pentafluorophenyl | tetrafluoro-3- |
| | | | pyridyl |
| 81. | pentafluorophenyl | pentafluorophenyl | tetrafluoro-4- |
| | | | pyridyl |
| 82. | pentafluorophenyl | pentafluorophenyl | tetrafluoro-2- |
| | | | benzonitrile |
| 83. | pentafluorophenyl | pentafluorophenyl | tetrafluoro-3- |
| | | | benzonitrile |
| 84. | pentafluorophenyl | pentafluorophenyl | tetrafluoro-4- |
| | | | benzonitrile |
| 85. | 3,5- | 3,5- | H |
| | bis(trifluoromethyl)- | bis(trifluoromethyl)- | |
| | phenyl | phenyl | |
| 86. | 3,5- | trifluoromethyl | H |
| | bis(trifluoromethyl)- | | |
| | phenyl | | |
| 87. | 3,5- | pentafluoroethyl | H |
| | bis(trifluoromethyl)- | | |
| | phenyl | | |
| 88. | 4-fluoro-3- | pentafluoroethyl | H |
| | trifluoromethyl- | | |
| | phenyl | | |
| 89. | 4-fluoro-3- | 3,5- | H |
| | trifluoromethyl- | bis(trifluoromethyl)- | |
| | phenyl | phenyl | |
| 90. | 3,4,5- | 3,4,5- | H |
| | trifluorophenyl | trifluorophenyl | |
| 91. | 3,4,5- | pentafluoroethyl | H |
| | trifluorophenyl | | |
| 92. | 4- | 4- | H |
| | (trifluoromethyl)phenyl | (trifluoromethyl)phenyl | |
| 93. | 4- | pentafluoroethyl | H |
| | (trifluoromethyl)phenyl | | |
| 94. | 2,4,6- | 2,4,6- | H |
| | tris(trifluoromethyl)phenyl | tris(trifluoromethyl)phenyl | |
| 95. | 2,4,6- | Pentafluoroethyl | H |
| | tris(trifluoromethyl)phenyl | | |
| 96. | 3,5- | 3,5- | CN |
| | bis(trifluoromethyl)- | bis(trifluoromethyl)- | |
| | phenyl | phenyl | |
| 97. | 2,4,6- | 2,4,6- | CN |
| | tris(trifluoromethyl)phenyl | tris(trifluoromethyl)phenyl | |
| 98. | 3,5- | pentafluoroethyl | 3,5- |
| | bis(trifluoromethyl)- | | bis(trifluoromethyl)- |
| | phenyl | | phenyl |
| 99. | 3,4- | 3,4- | H |
| | bis(trifluoromethyl)- | bis(trifluoromethyl)- | |
| | phenyl | phenyl | |
| 100. | 3,4- | trifluoromethyl | H |
| | bis(trifluoromethyl)- | | |
| | phenyl | | |
| 101. | 3,4- | pentafluoroethyl | 3,5- |
| | bis(trifluoromethyl)- | | bis(trifluoromethyl)- |
| | phenyl | | phenyl |
| 102. | 3,4- | pentafluoroethyl | H |
| | bis(trifluoromethyl)- | | |
| | phenyl | | |
| 103. | 3,4- | 3,4- | CN |
| | bis(trifluoromethyl)- | bis(trifluoromethyl)- | |
| | phenyl | phenyl | |
| 104. | 4-cyano-3- | 3,5- | H |
| | (trifluoromethyl)- | bis(trifluoromethyl)- | |
| | phenyl | phenyl | |
| 105. | 4-cyano-3- | pentafluoroethyl | H |
| | (trifluoromethyl)- | | |
| | phenyl | | |
| 106. | 3,5- | pentafluoroethyl | CN |
| | bis(trifluoromethyl)- | | |
| | phenyl | | |
| 107. | 4-cyano-2,6- | 4-cyano-2,6- | H |
| | bis(trifluoromethyl)- | bis(trifluoromethyl)- | |
| | phenyl | phenyl | |
| 108. | 4-cyano-2,6- | pentafluoroethyl | H |
| | bis(trifluoromethyl)- | | |
| | phenyl | | |
| 109. | 2,4- | 2,6- | H |
| | bis(trifluoromethyl)- | bis(trifluoromethyl)- | |
| | phenyl | phenyl | |
| 110. | 2,4- | 2,6- | CN |
| | bis(trifluoromethyl)- | bis(trifluoromethyl)- | |
| | phenyl | phenyl | |
| 111. | 4-(3′,5′- | pentafluoroethyl | H |
| | bis(trifluoromethyl)phenyl)- | | |
| | 2,6- | | |
| | bis(trifluoromethyl)- | | |
| | phenyl | | |
| 112. | 3,5- | 3,5- | CF₃ |
| | bis(trifluoromethyl)- | bis(trifluoromethyl)- | |
| | phenyl | phenyl | |
| 113. | 3,5- | trifluoromethyl | CF₃ |
| | bis(trifluoromethyl)- | | |
| | phenyl | | |
| 114. | 3,5- | pentafluoroethyl | CF₃ |
| | bis(trifluoromethyl)- | | |
| | phenyl | | |
| 115. | 3,4,5- | 3,4,5- | CF₃ |
| | trifluorophenyl | trifluorophenyl | |
| 116. | 4- | 4- | CF₃ |
| | (trifluoromethyl)phenyl | (trifluoromethyl)phenyl | |
| 117. | tert-butyl | heptafluoro-n- | H |
| | | propyl | |
| 118. | tert-butyl | tert-butyl | tert-butyl |

are excluded.

2. Compounds according to claim 1, wherein R¹ und R² have different meaning or
if R¹ und R² have the same meaning, then R¹ und R² are selected from CFR^{a}R^{b} and phenyl, which is substituted by at least one radical selected from F, Cl, Br, CN, CF₃, SF₅, OCF₃, SCF₃, SO₂CF₃, N═C(CF₃)₂, and N(SO₂CF₃)₂, wherein
R^{a} is F and R^{b} is selected from C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN.

3. Compounds according to claim 1, wherein
R¹ is selected from CFR^{a}R^{b}, t-butyl, adamantyl, C₂F₅, n-C₃F₇ and C₃-C₇-cycloalkyl, wherein cycloalkyl is unsubstituted or substituted with 1 to 13 radicals selected from F and CF₃,
and
R² is selected from CFR^{a}R^{b}, phenyl, naphthyl, pyridyl and pyrimidyl, wherein phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂, naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂, pyridyl and pyrimidyl are substituted by at least one radical selected from NO₂, F, Cl, Br, CN, CF₃, SF₅, OCF₃, SCF₃, SO₂CF₃, N═C(CF₃)₂, and N(SO₂CF₃)₂,
or
R¹ and Y together with the carbon atom of the C—O-group to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
and
R² is selected from CFR^{a}R^{b}, t-butyl, adamantyl, C₂F₅, n-C₃F₇ and C₃-C₇-cycloalkyl, wherein cycloalkyl is unsubstituted or substituted with 1 to 13 radicals selected from F and CF₃,
R^{a} and R^{b} independently from each other represent halogen, CF₃, CN and C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN;
R⁴ represents CN, NO₂, SF₅, halogen, C₁-C₄-alkyl, C₁-C₄-haloalkyl, C₁-C₄-alkoxy, C₁-C₄-haloalkoxy, C₁-C₄-alkylsulfanyl, C₁-C₄-haloalkylsulfanyl, C₁-C₄-alkylsulfonyl, C₁-C₄-haloalkylsulfonyl, N═C(CF₃)₂ and N(SO₂CF₃)₂.

4. Compounds according to claim 1, wherein R¹ and R² are independently from each other selected from CFR^{a}R^{b} and phenyl which is substituted by at least one radical selected from NO₂, F, Cl, Br, CN, CF₃, SF₅, OCF₃, SCF₃, SO₂CF₃, N═C(CF₃)₂ and N(SO₂CF₃)₂,
R^{a} and R^{b} have one of the meanings as defined in claims 1 to 3.

5. Compound according to claim 1, wherein R^{a} and R^{b} independently from each other represent F, CF₃ and a group A selected from A1 to A32 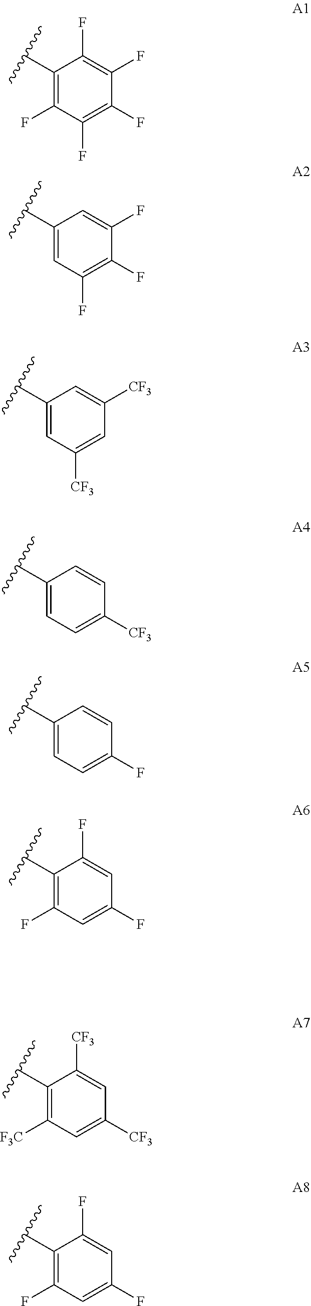 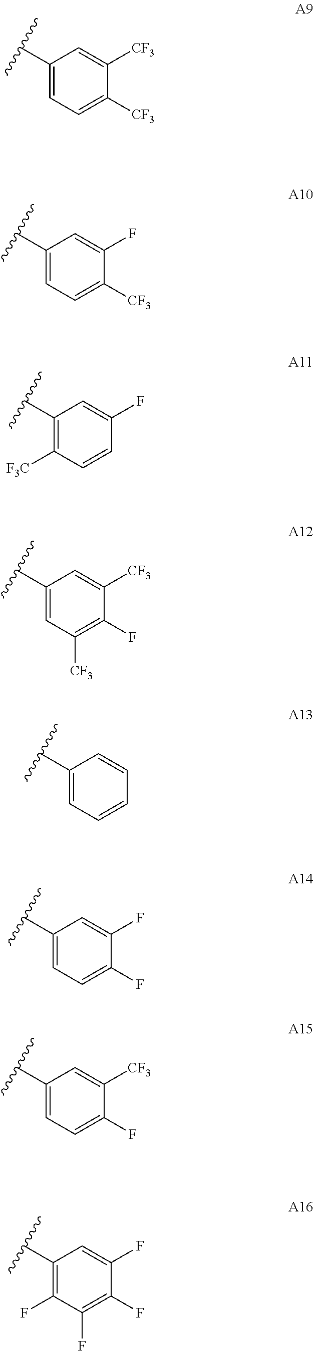 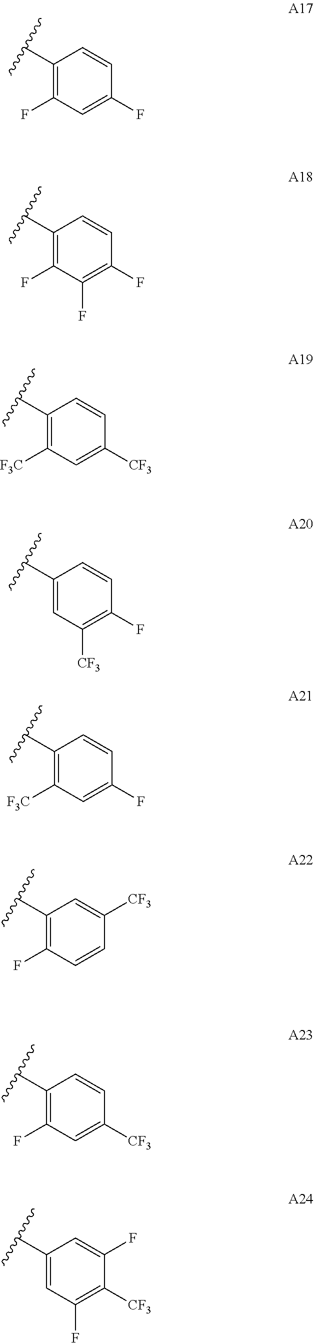 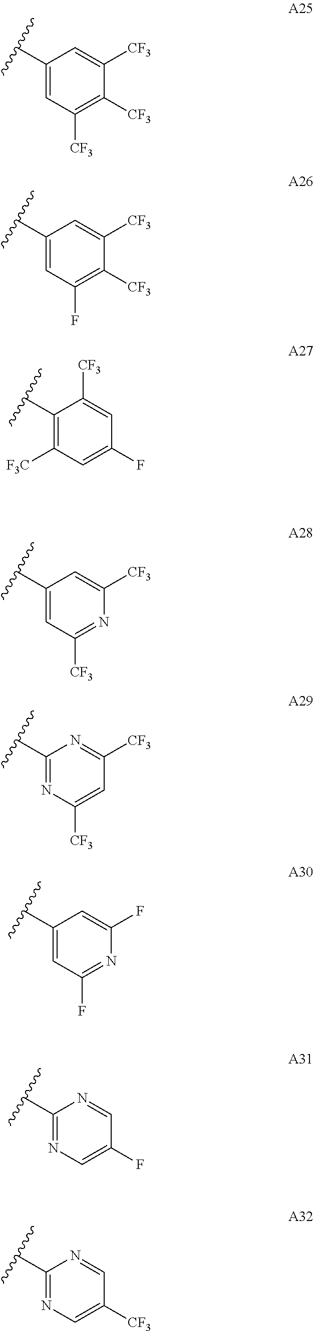
wherein ˜ denotes the bond to the remaining molecule.

6. Compounds according to claim 1, wherein L₁, L₂, L₃, L₄ have the same meaning and are selected from 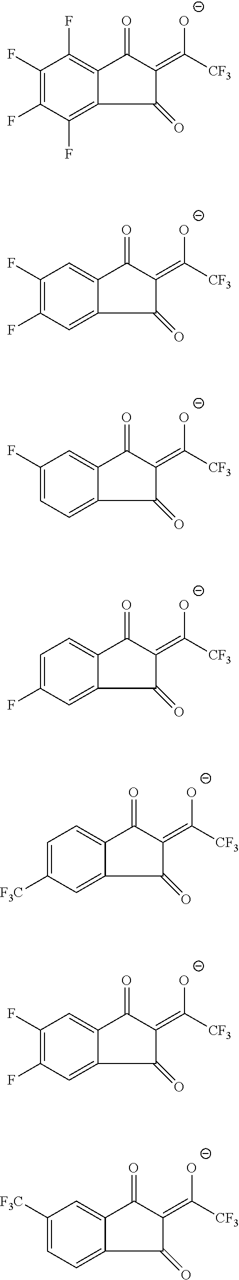 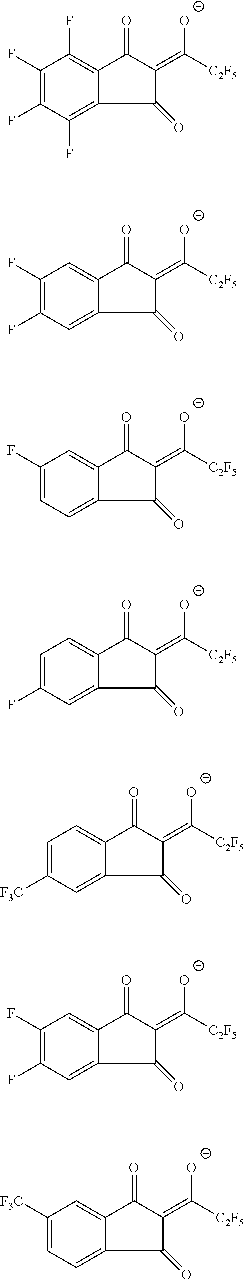 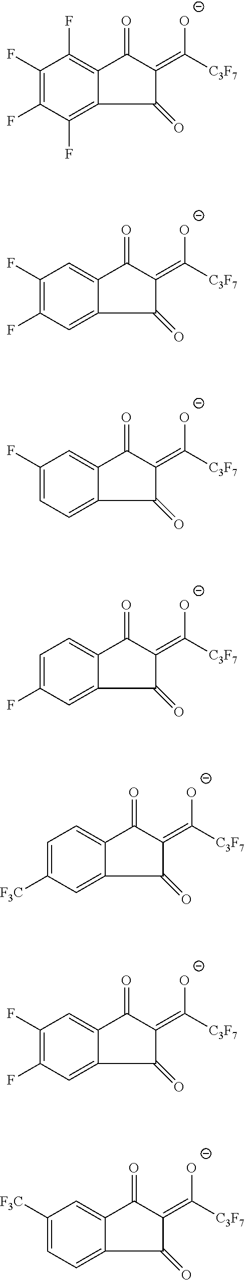 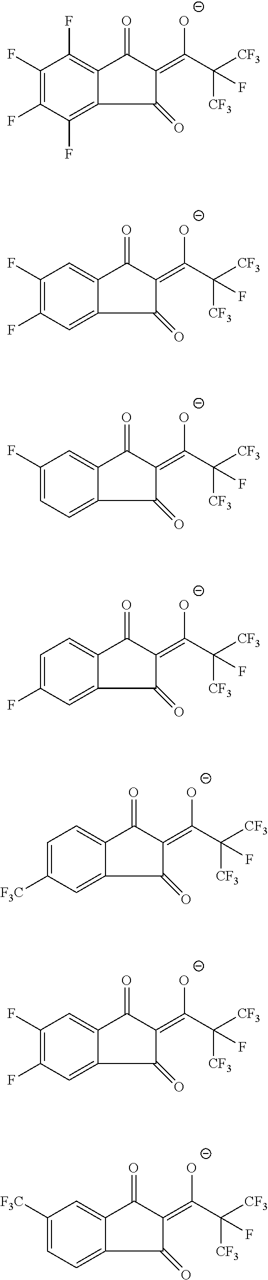 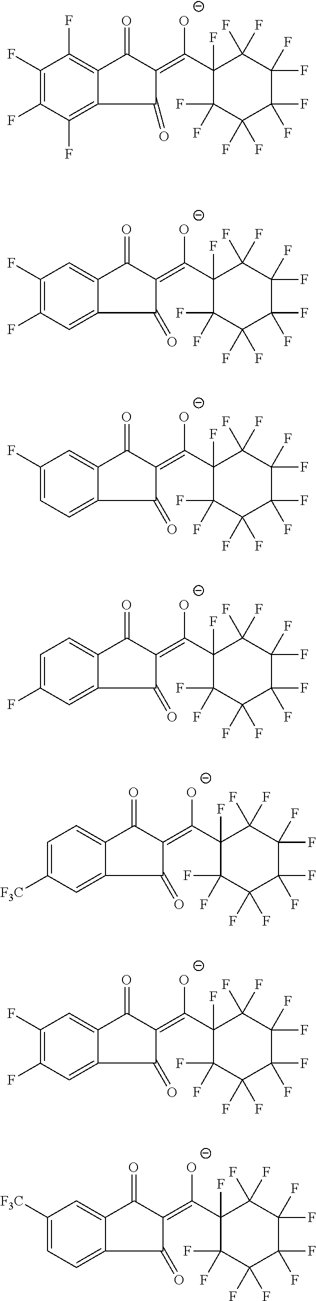 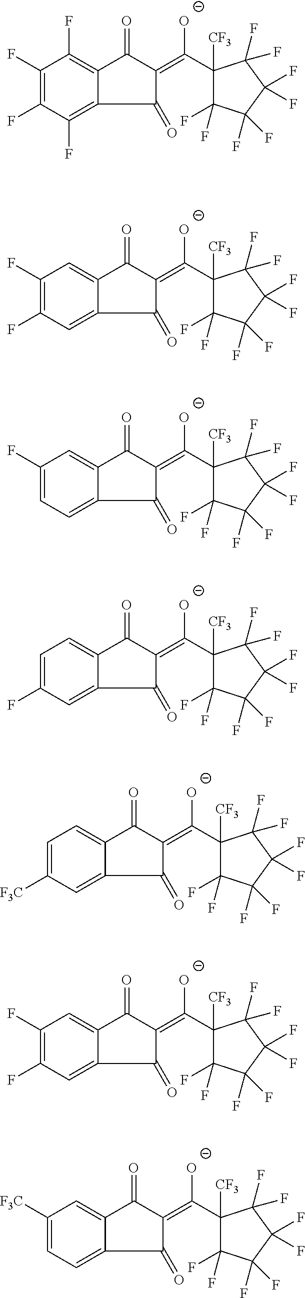 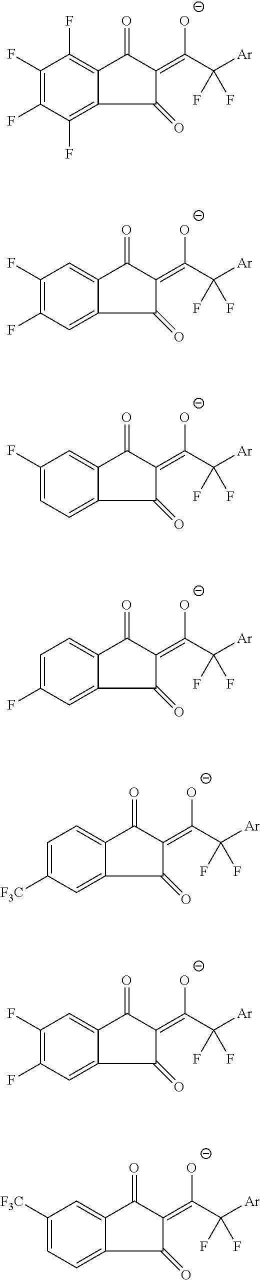 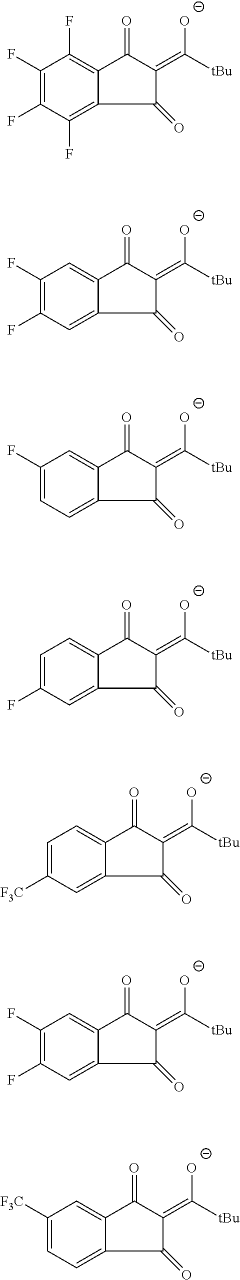 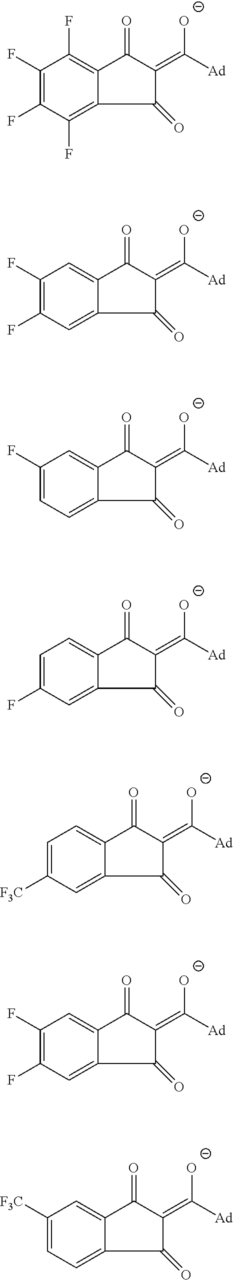 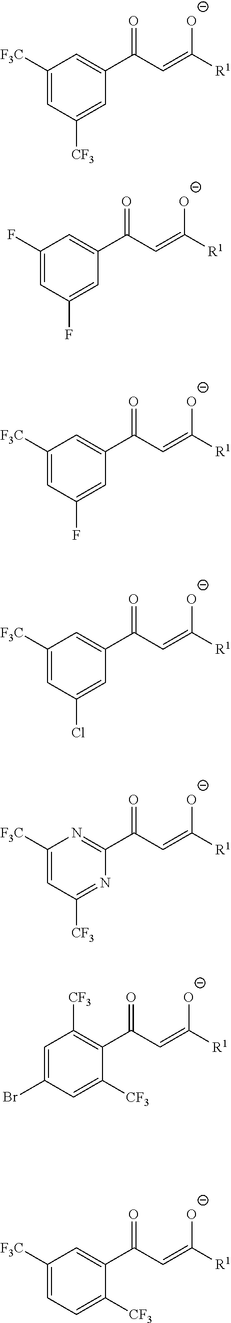 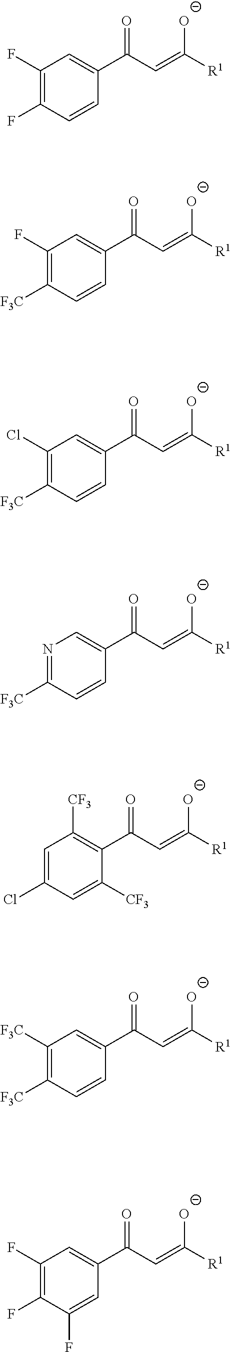 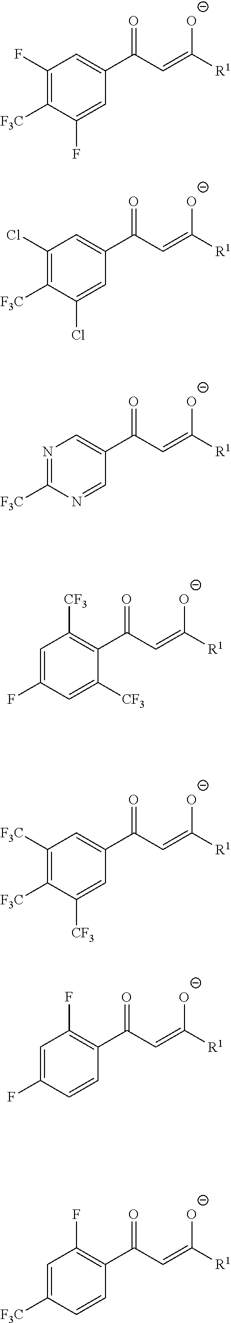 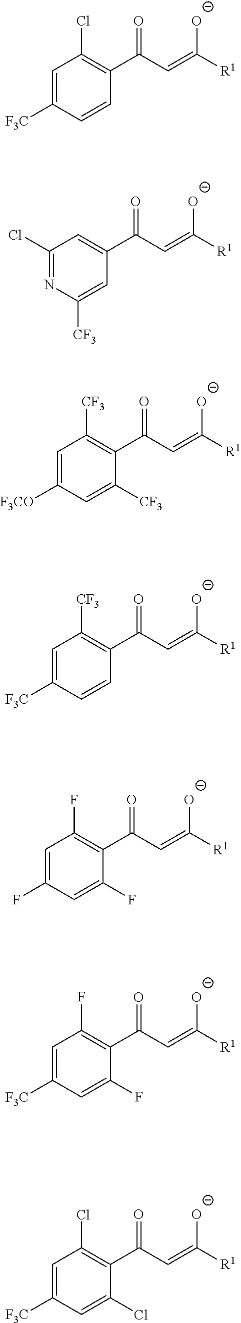 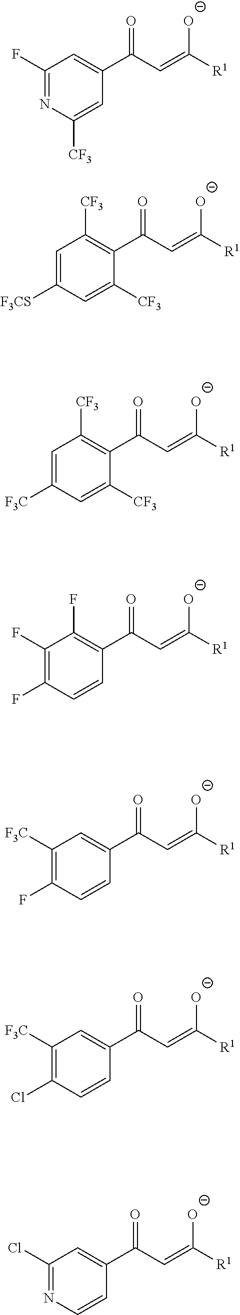 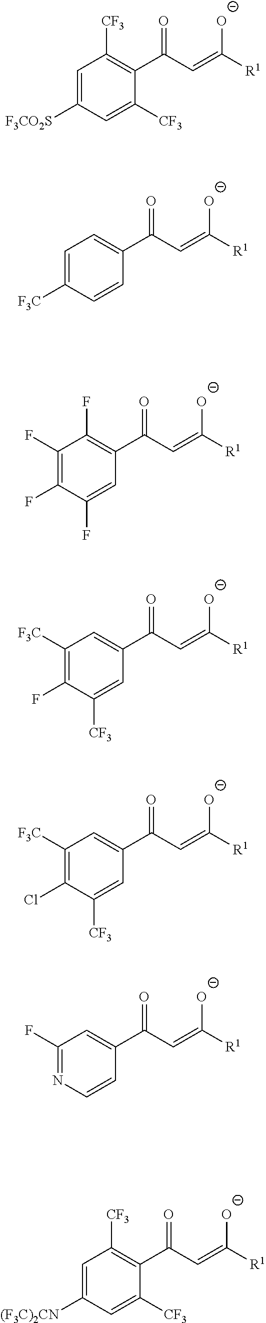 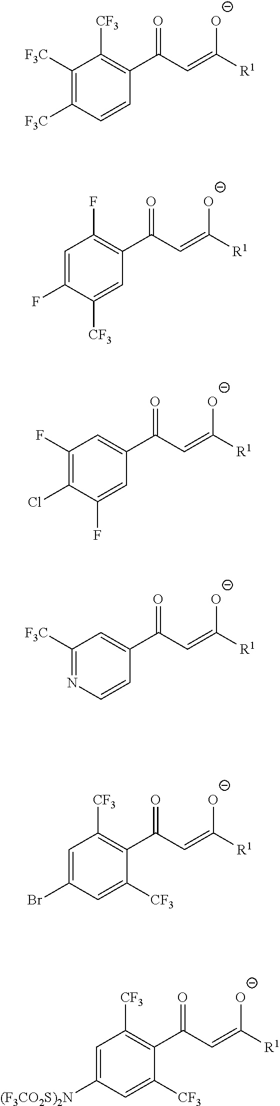 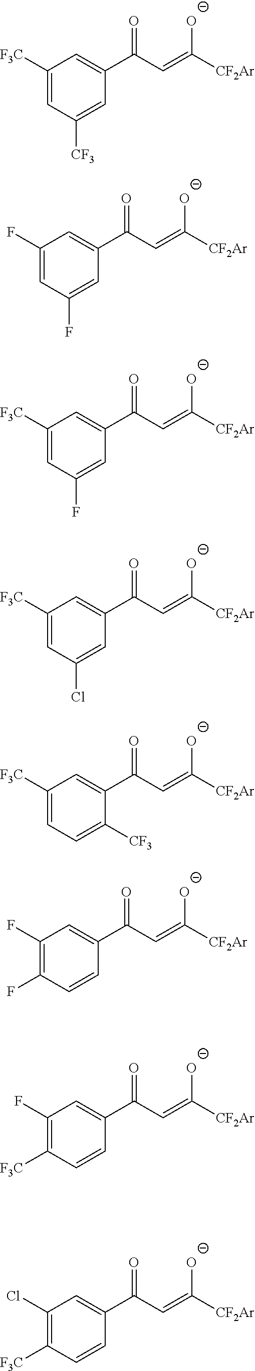 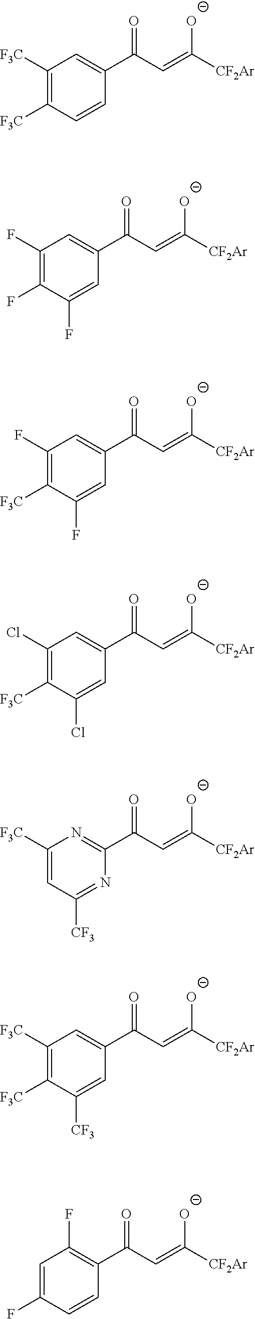 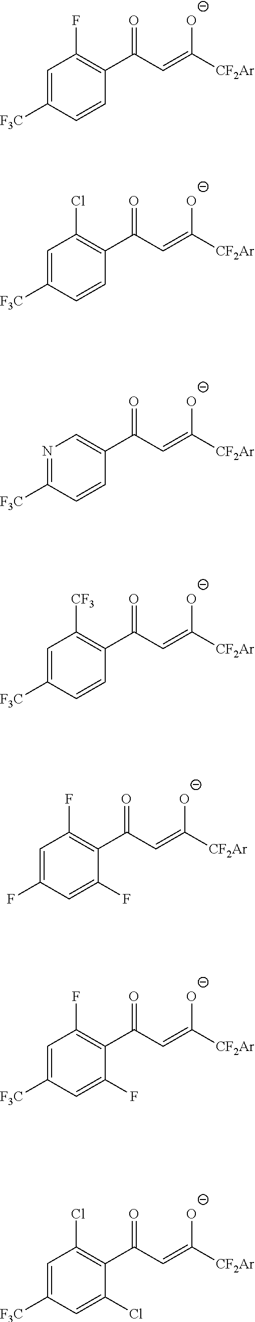 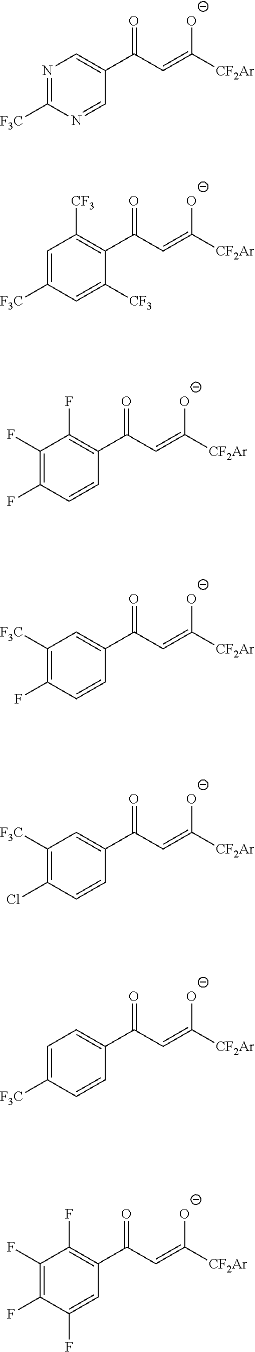 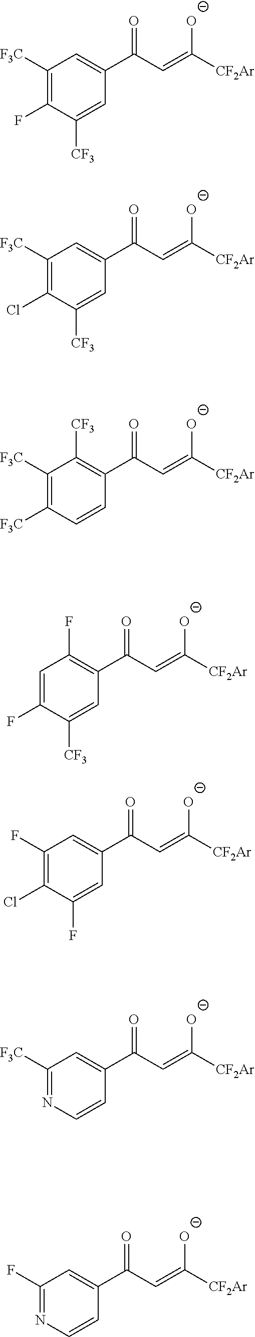 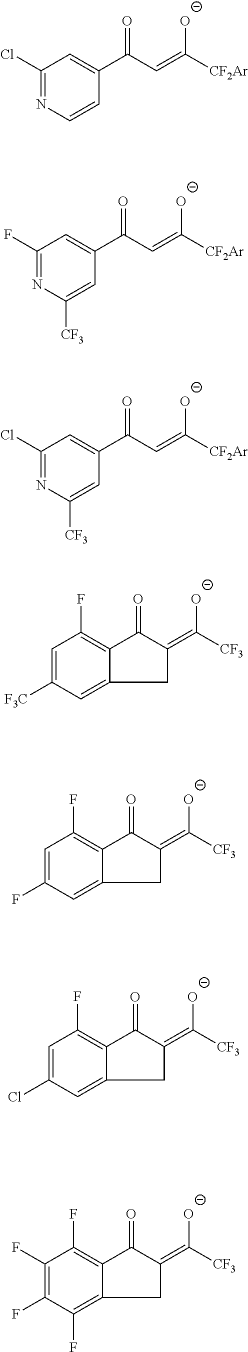 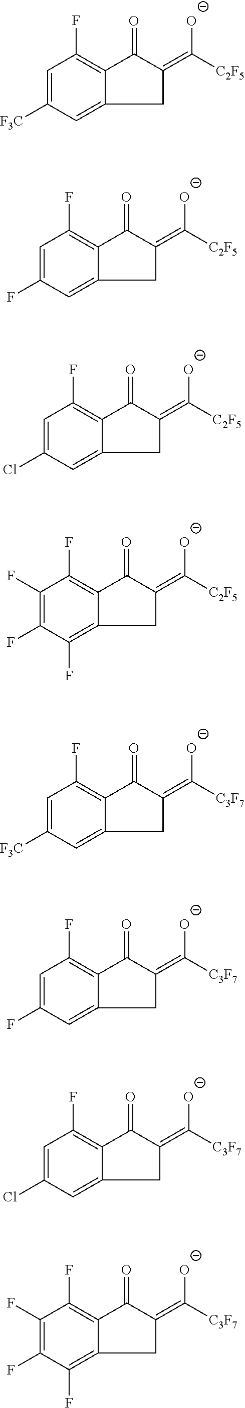 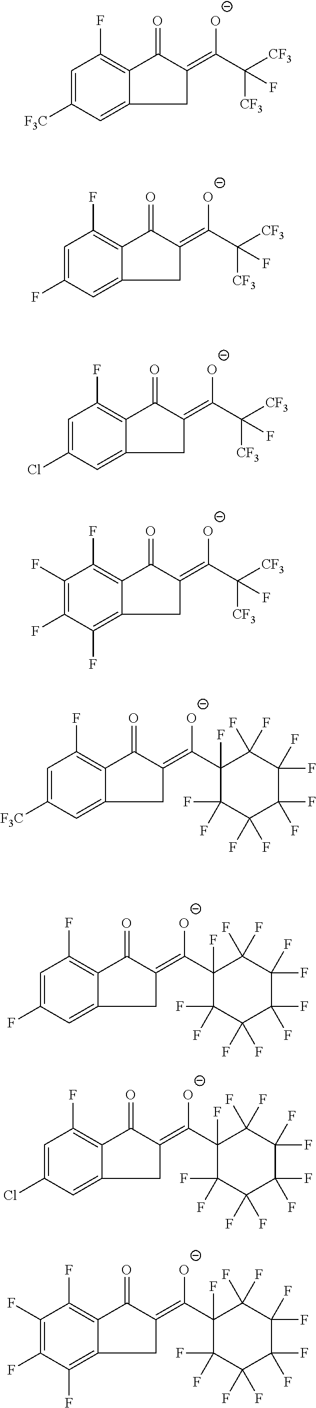 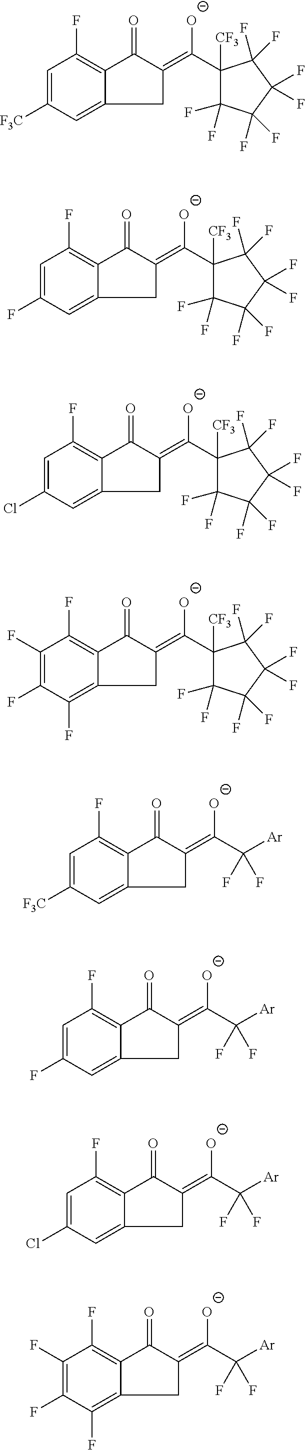 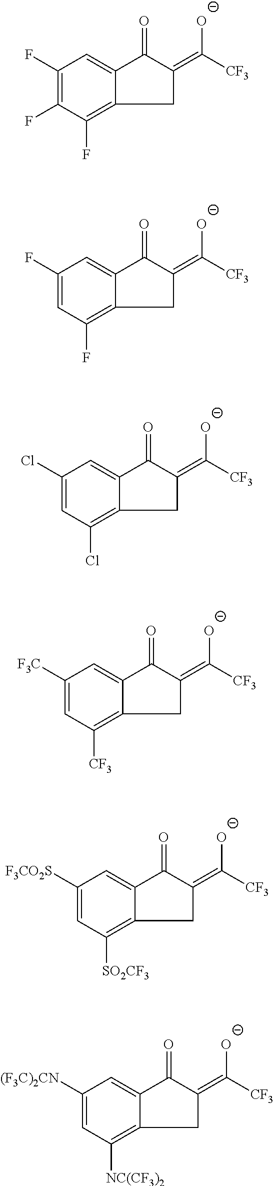 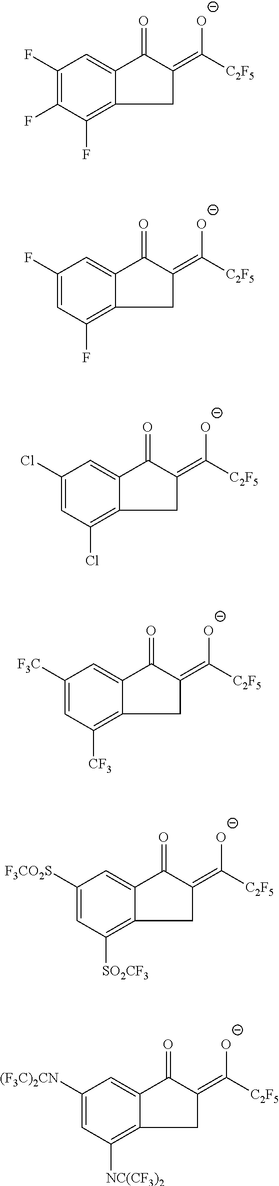 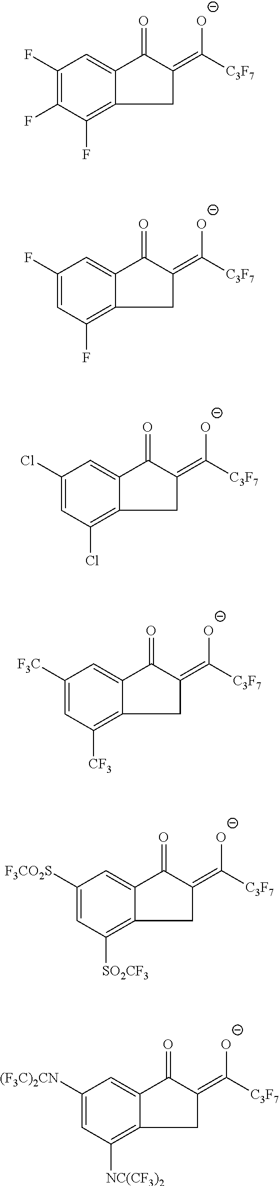 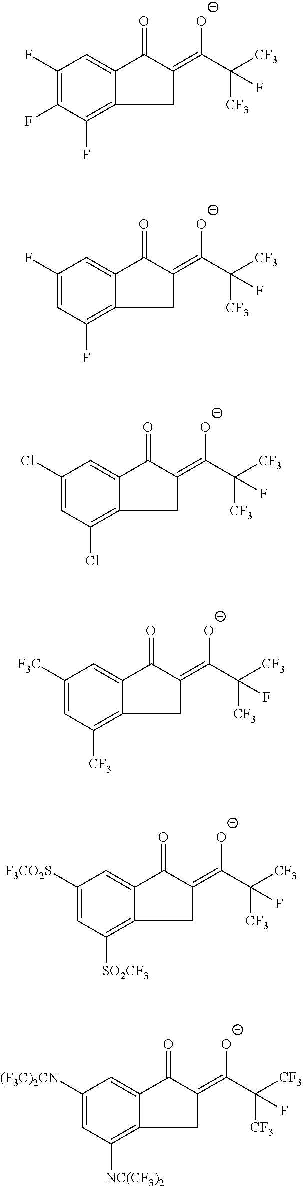 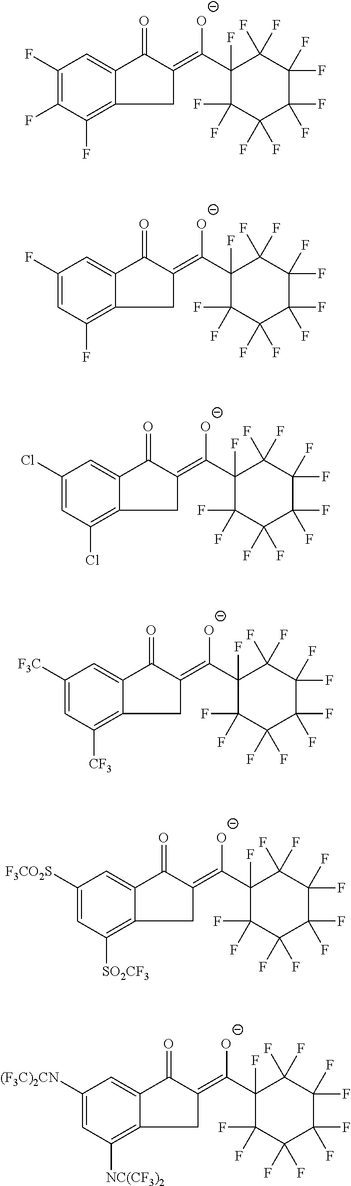 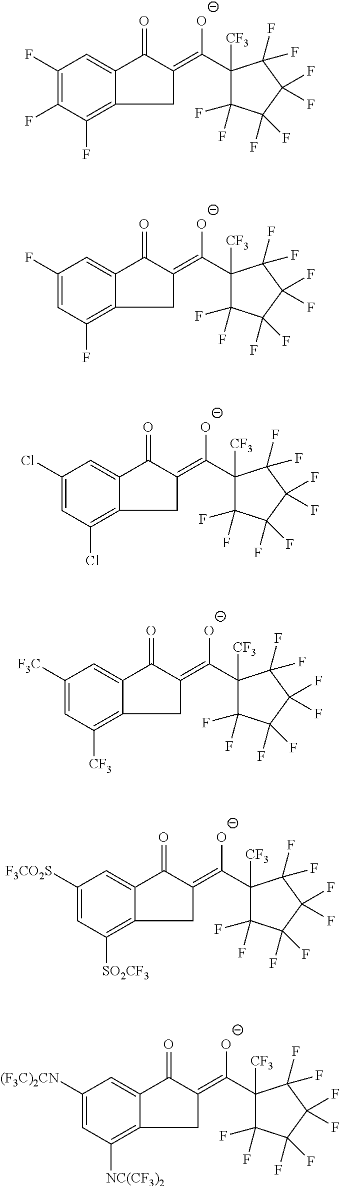 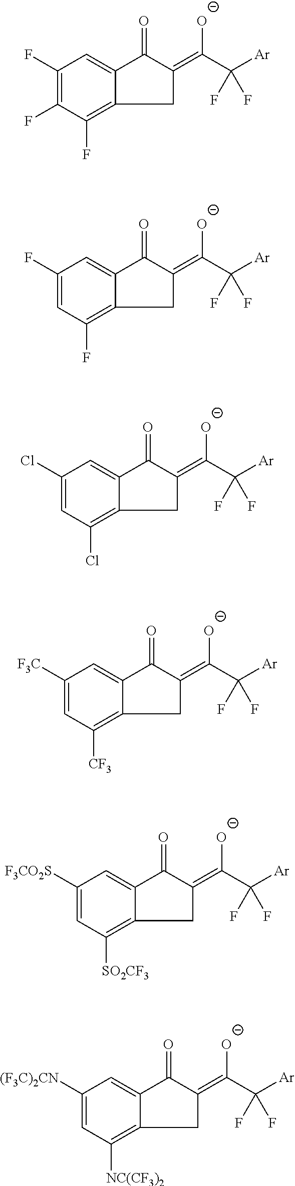 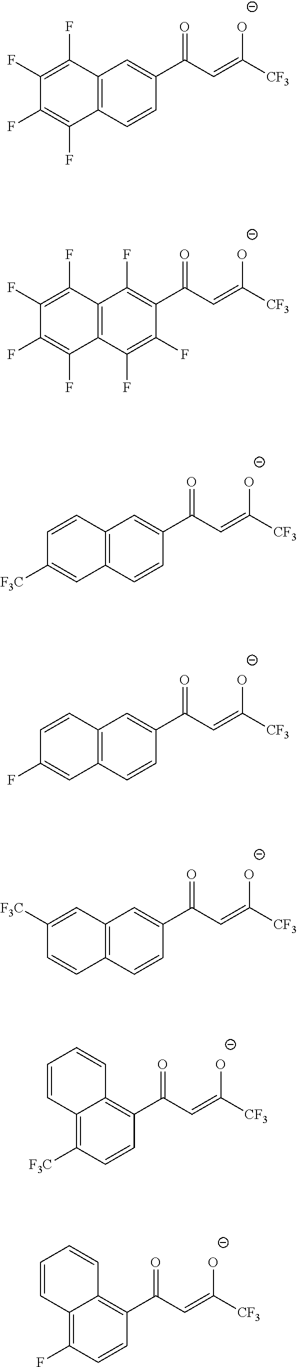 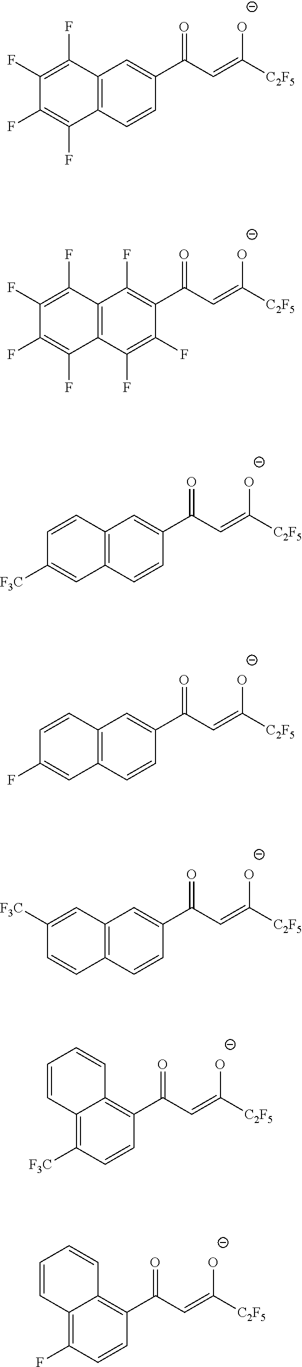 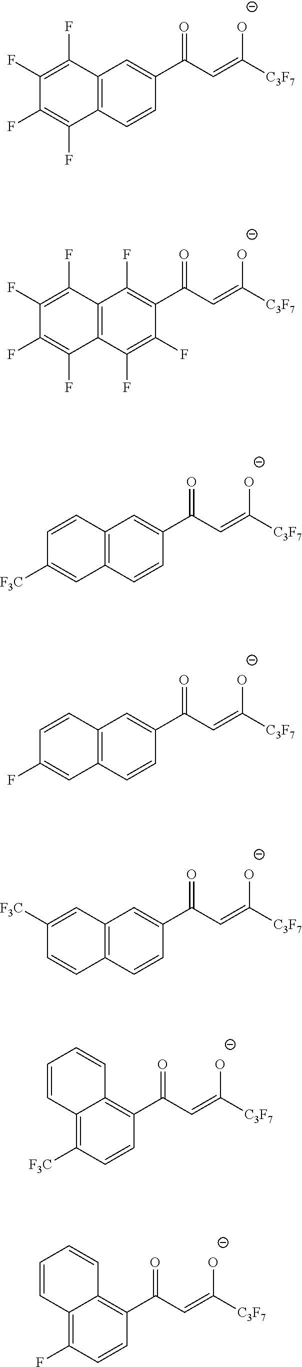 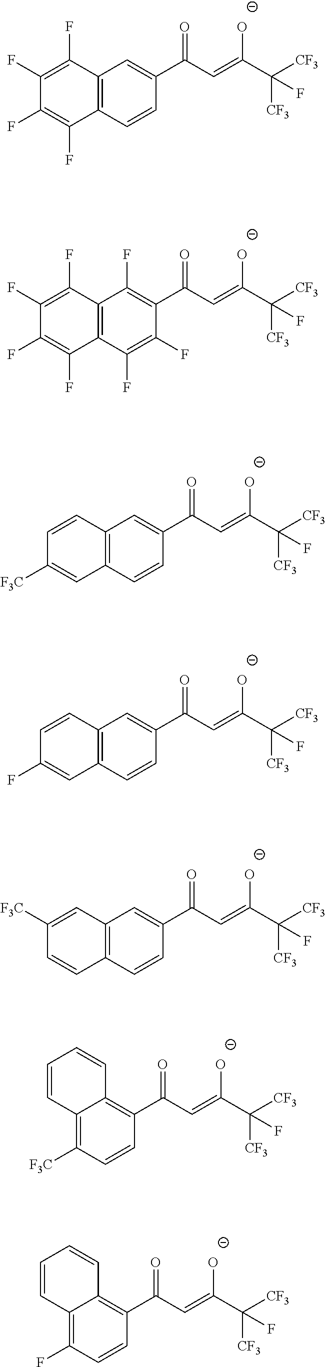 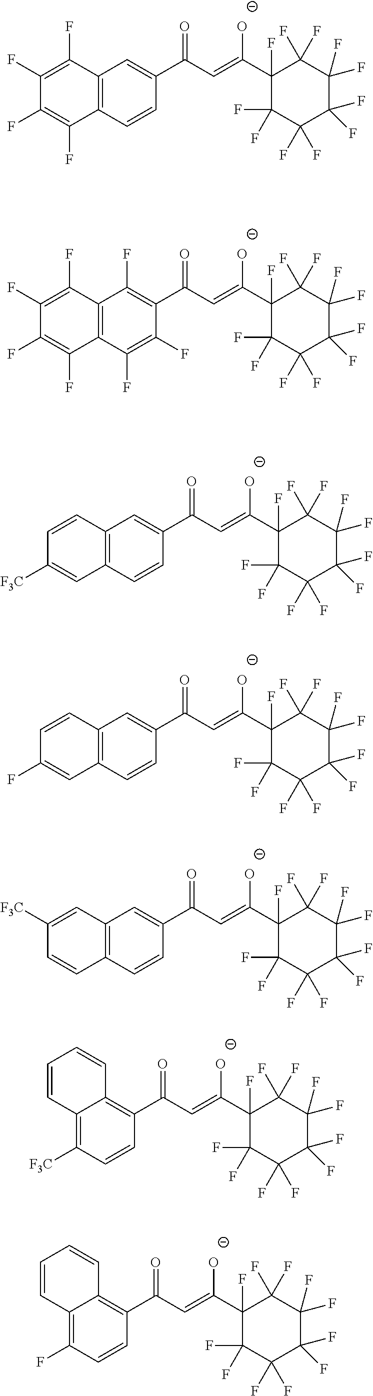 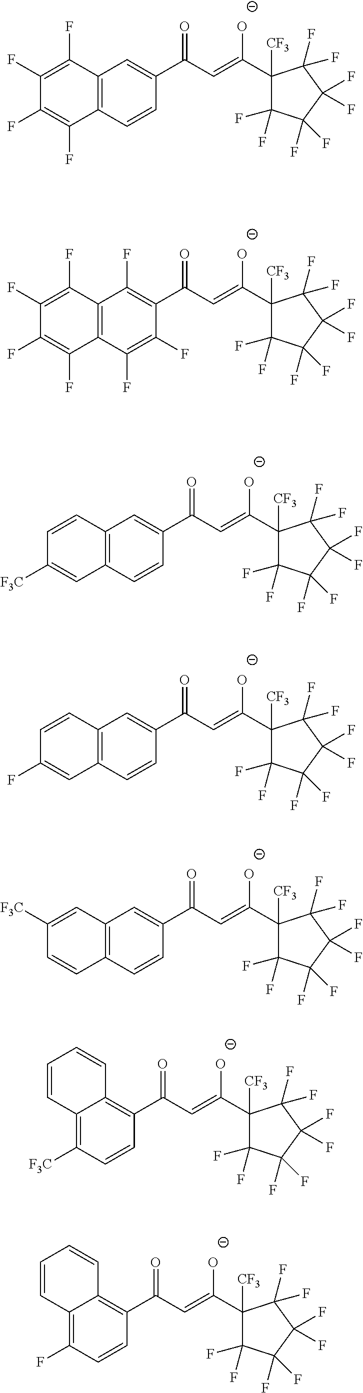 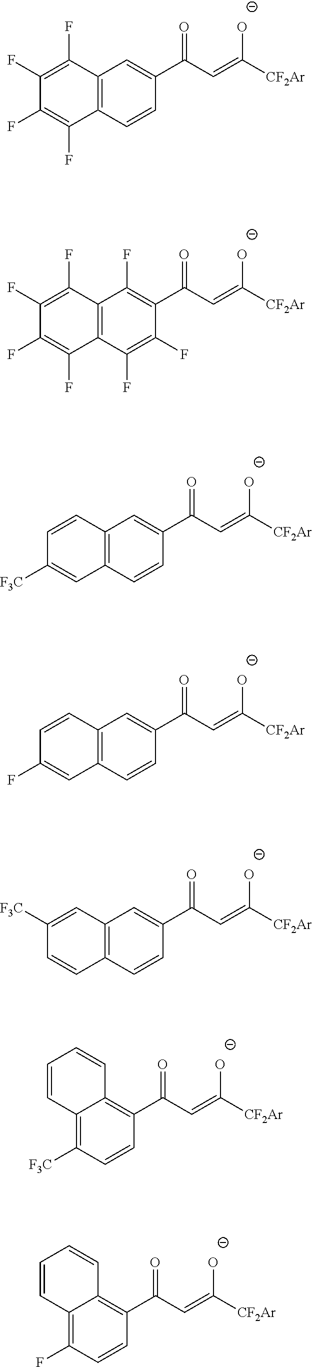 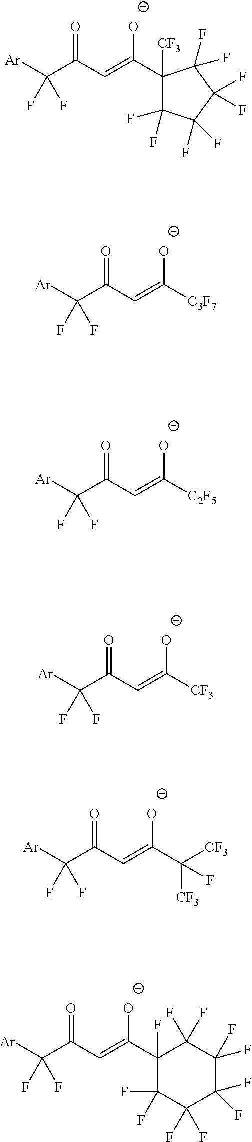 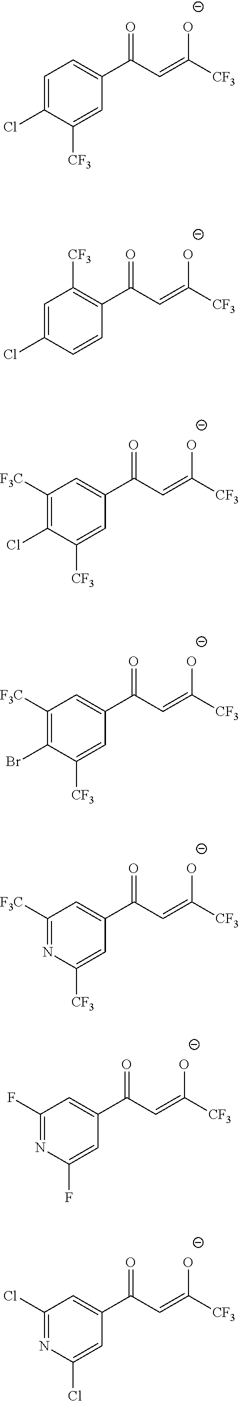 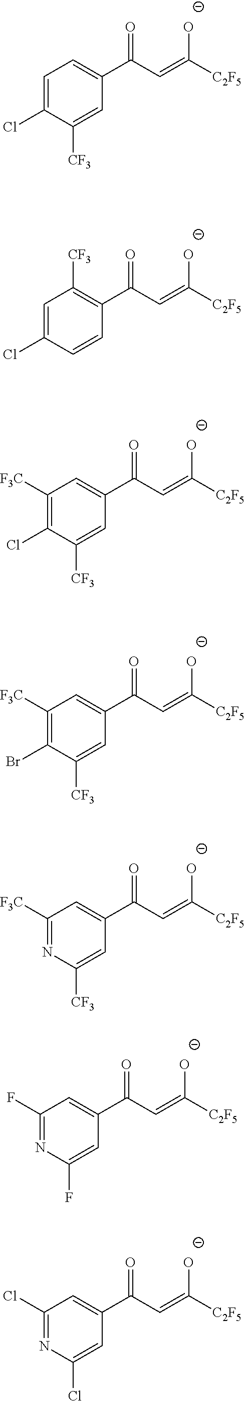 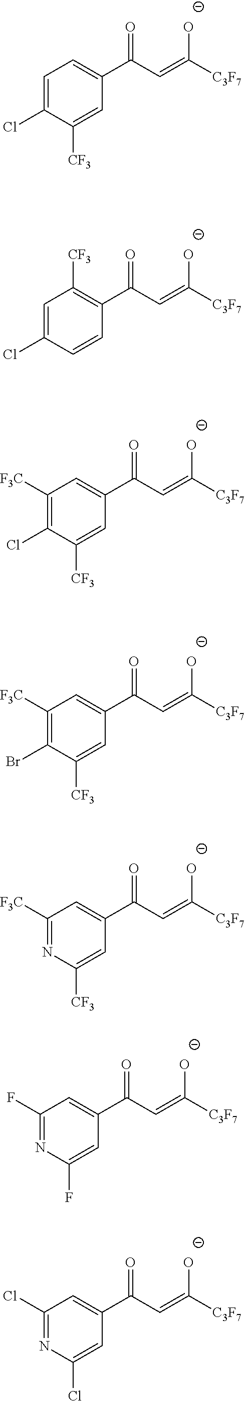 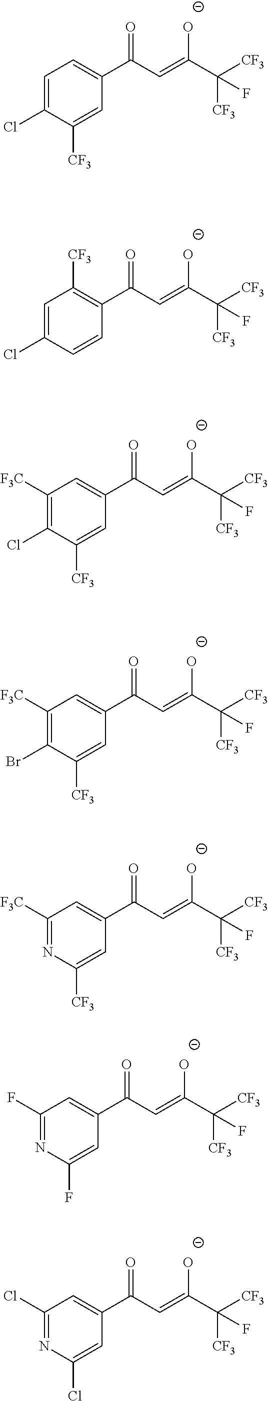 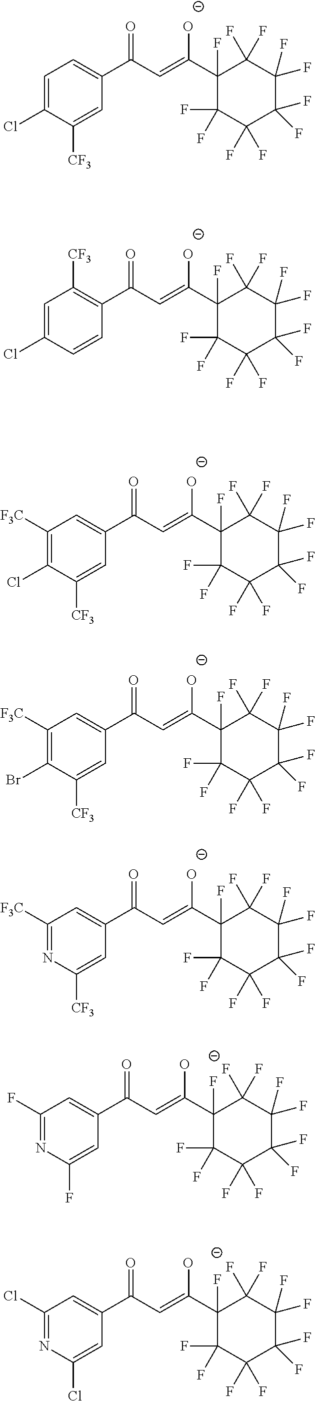 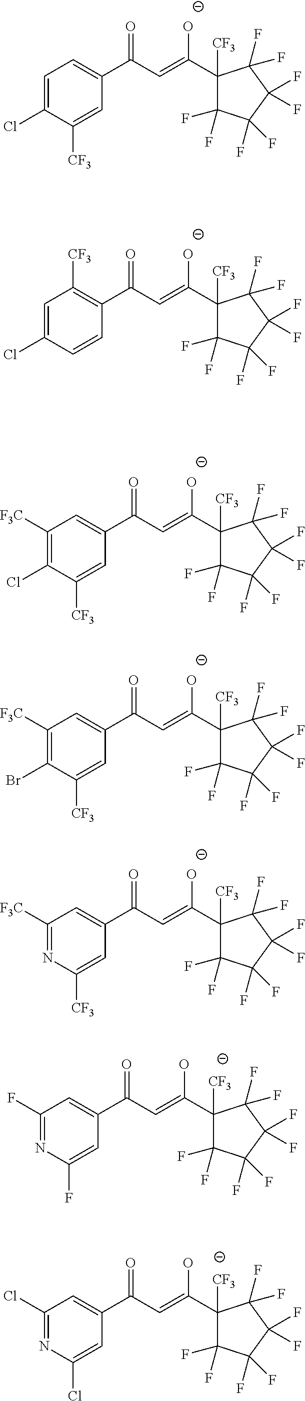 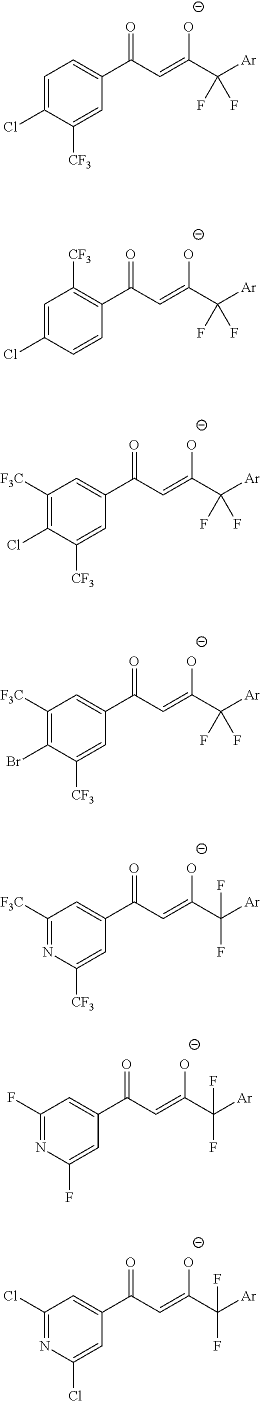 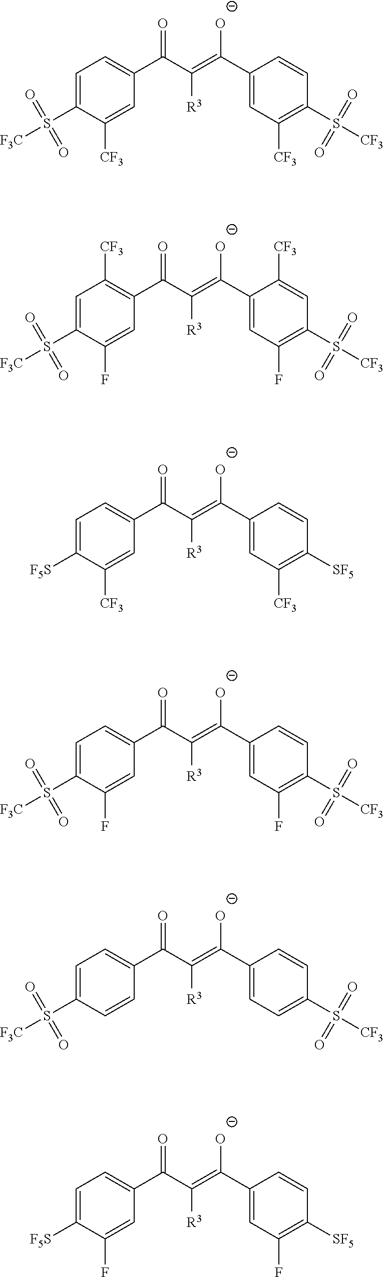 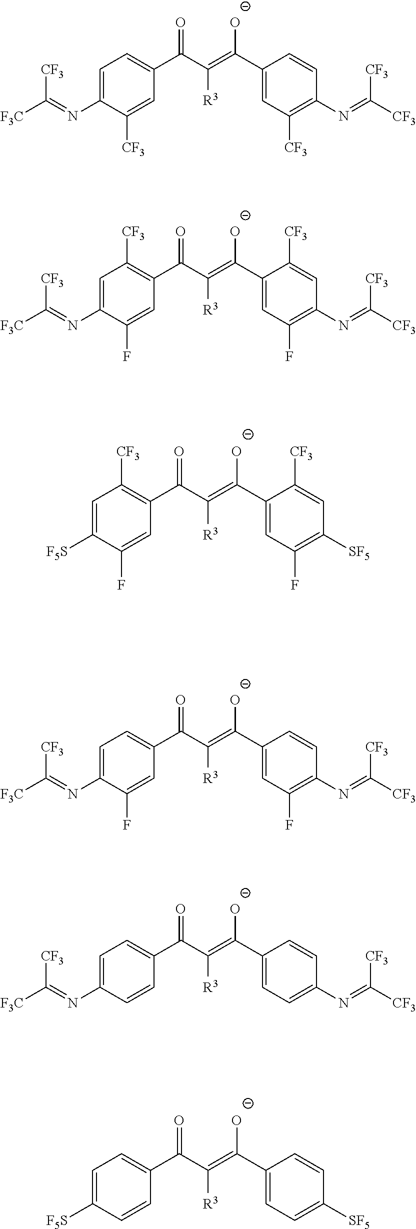 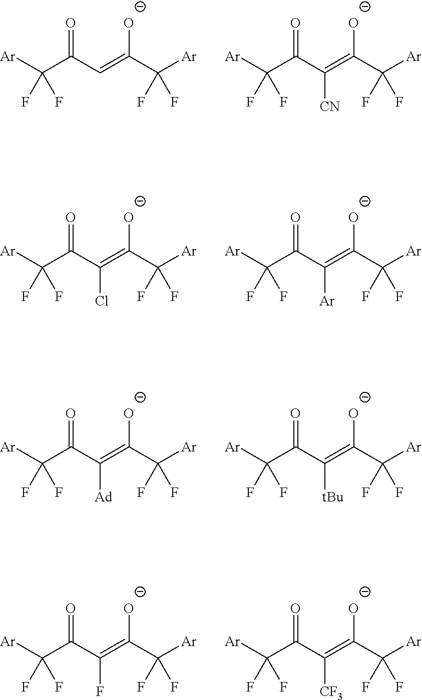 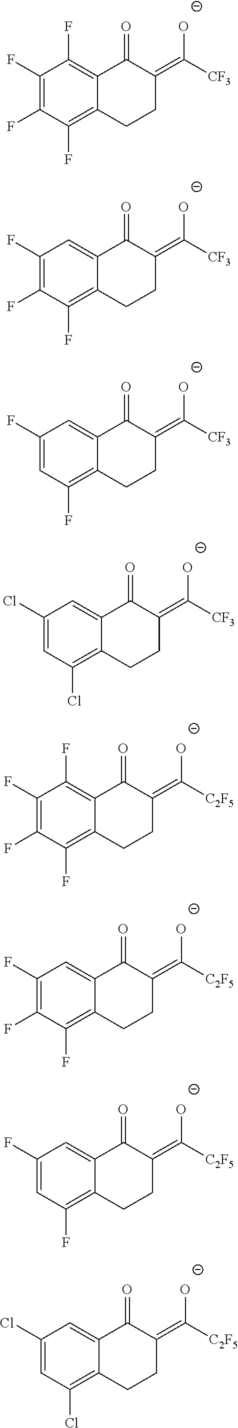 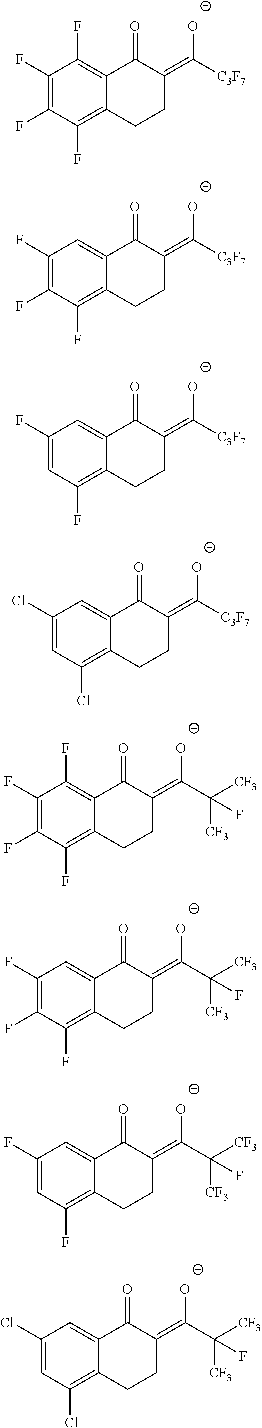 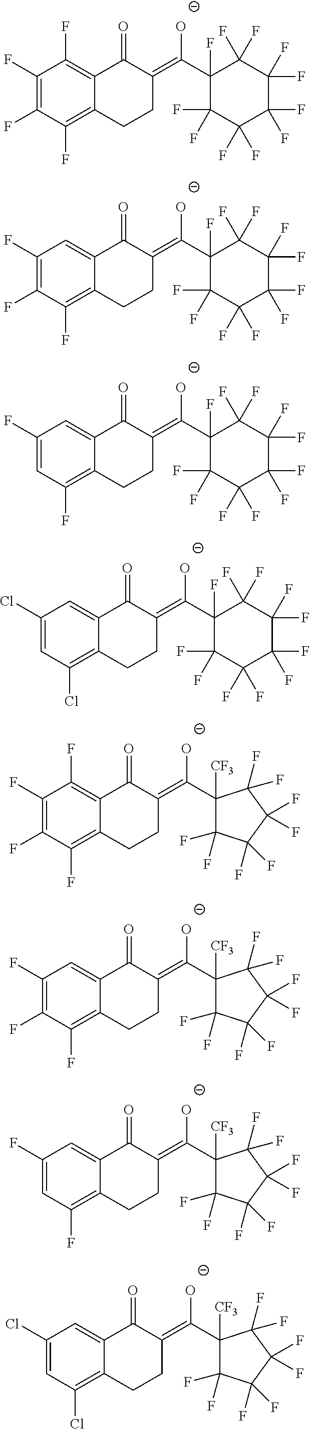 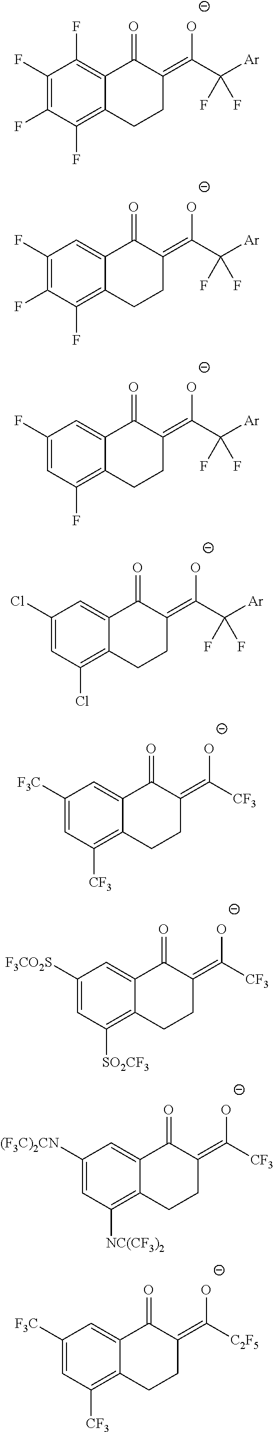 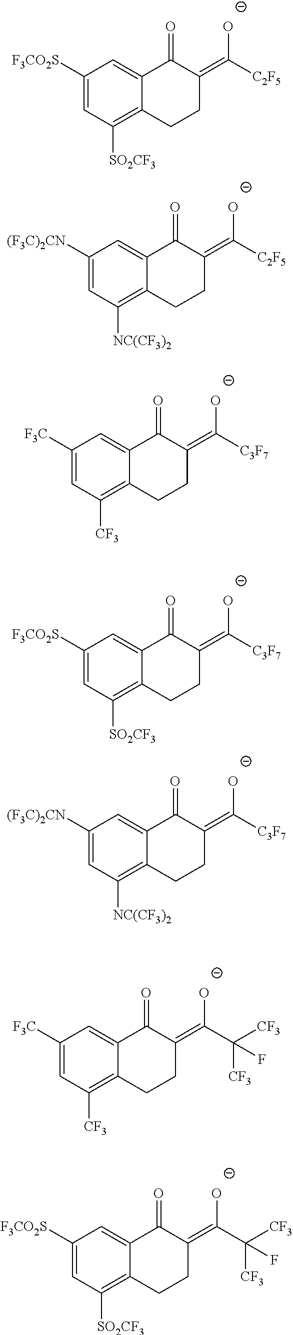 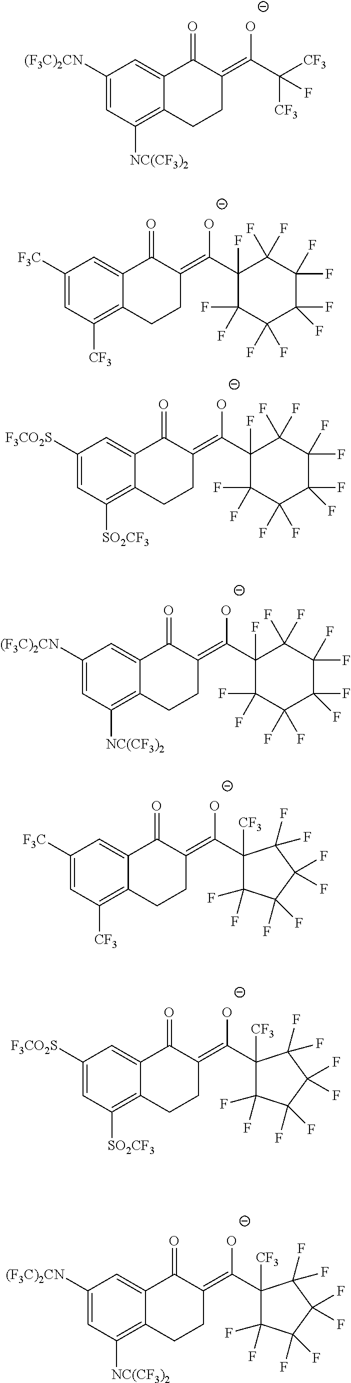 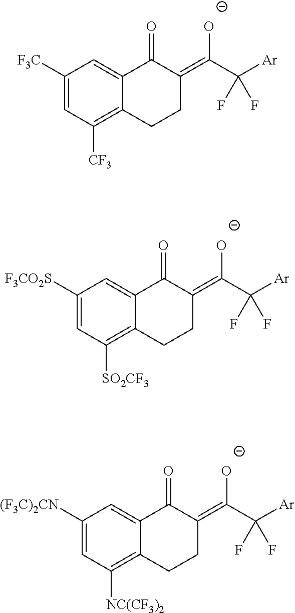 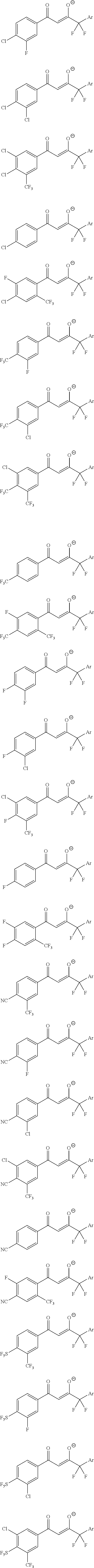 [chemical structure] 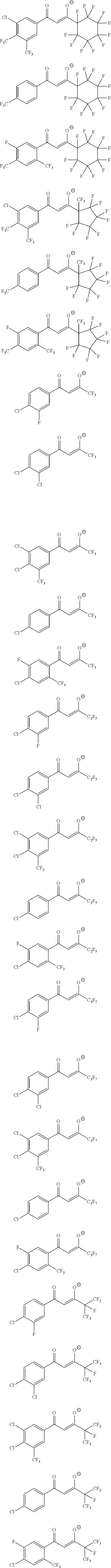 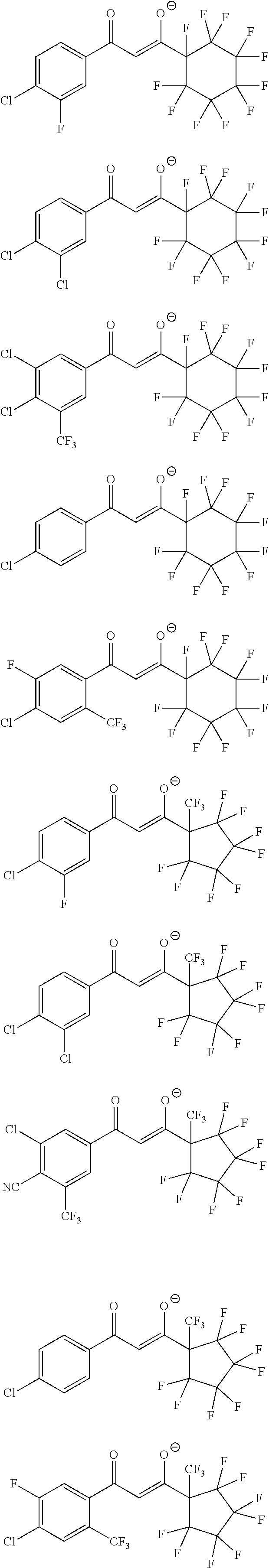 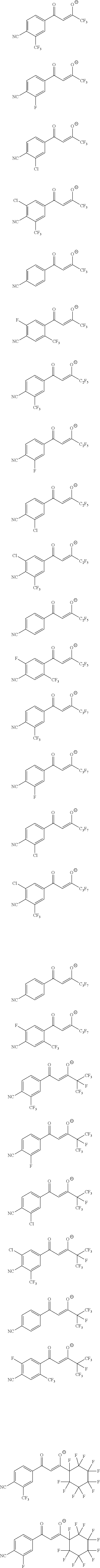 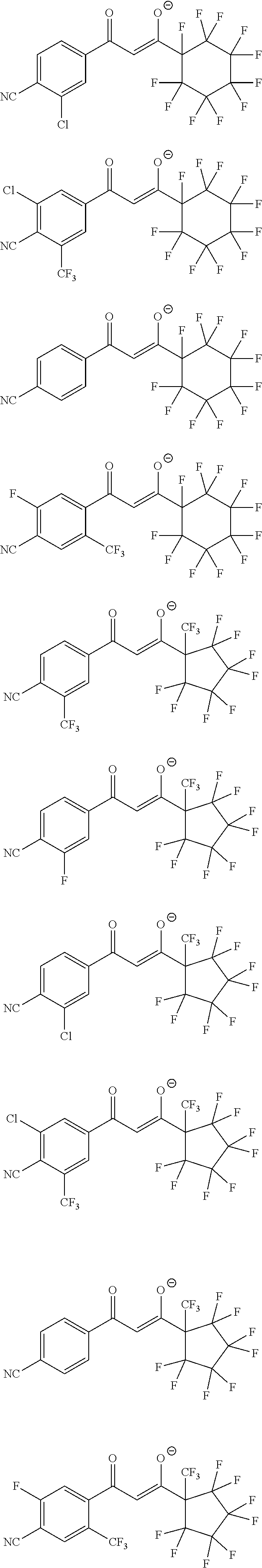 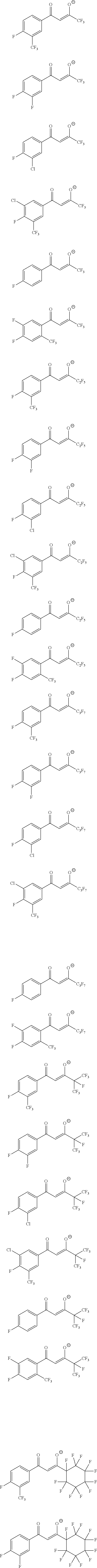 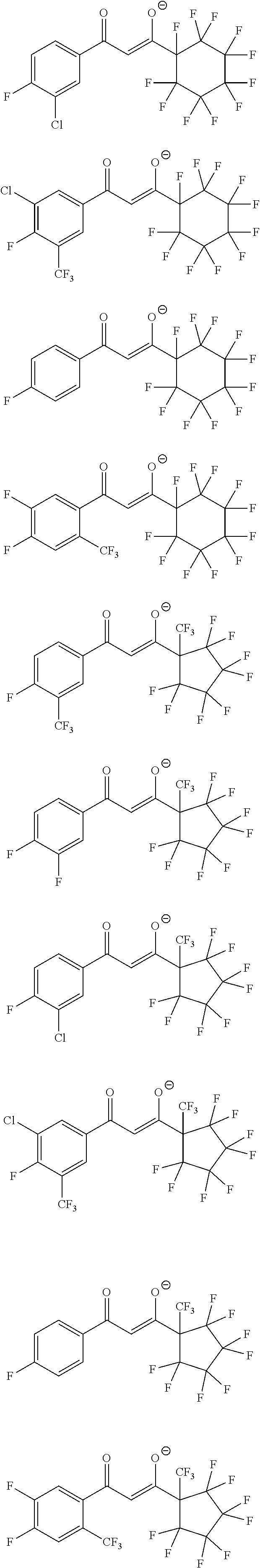 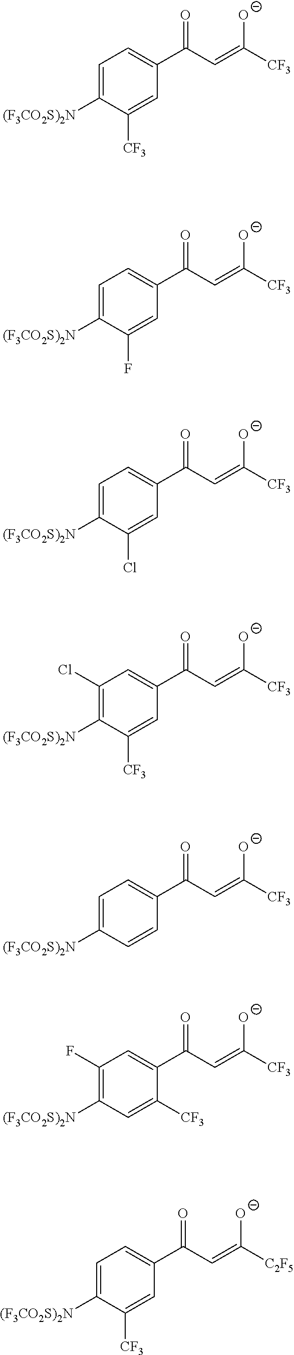 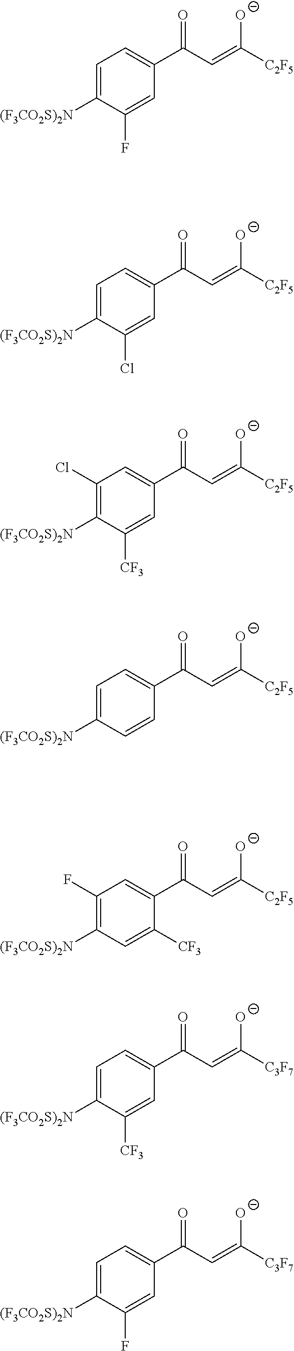 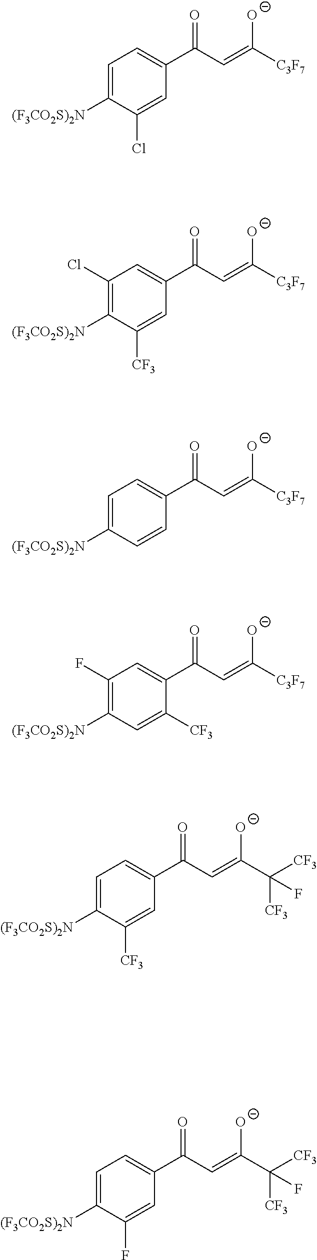 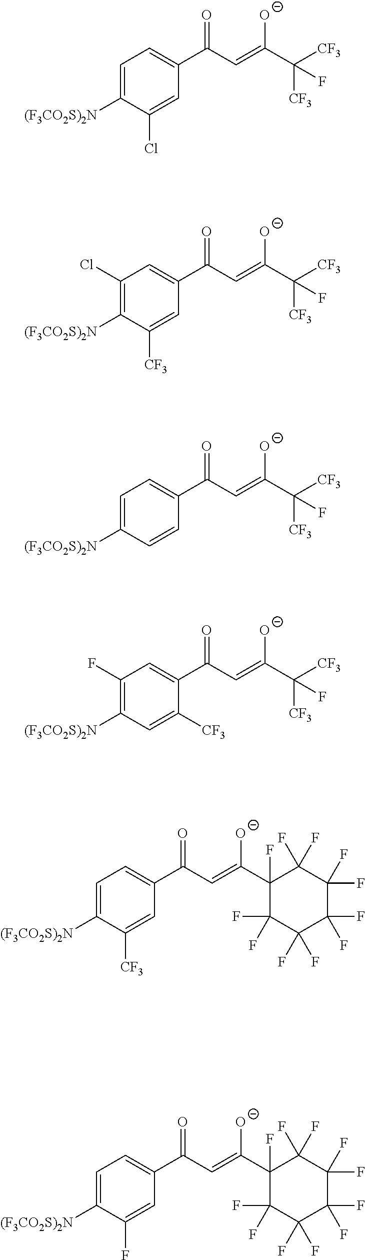 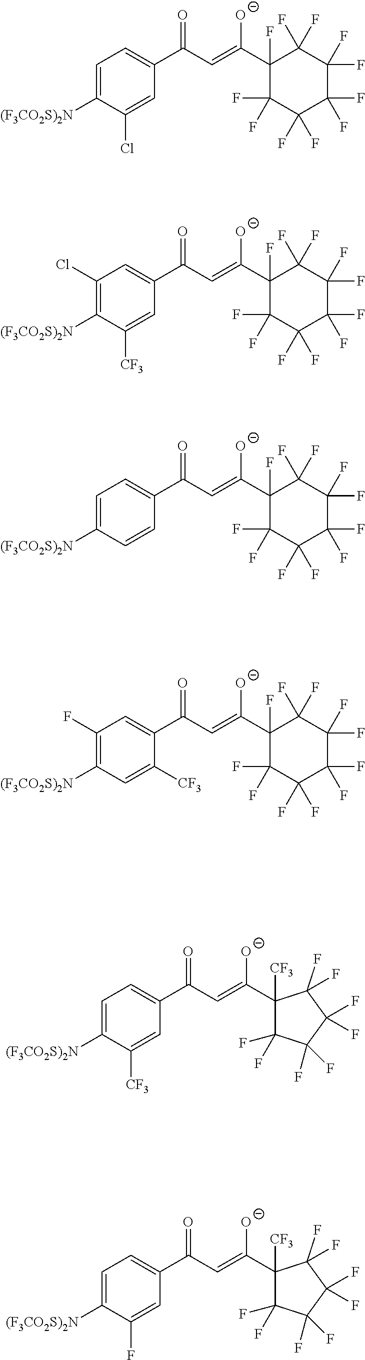 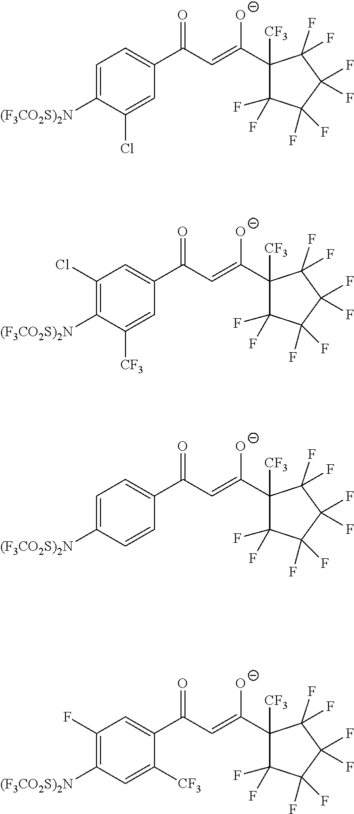 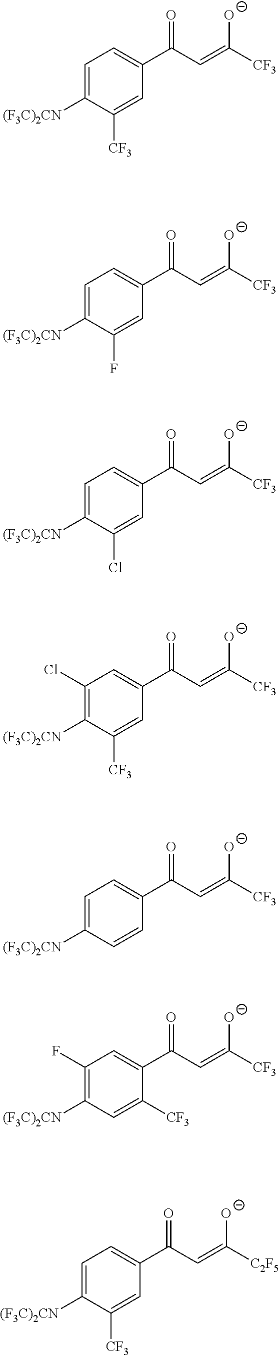 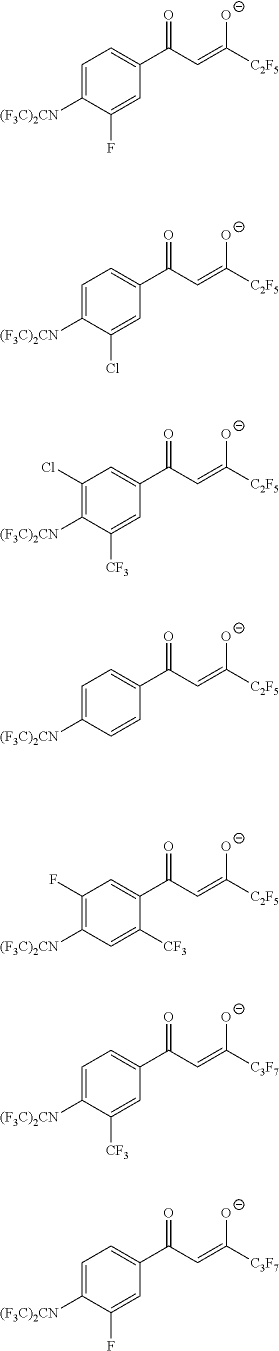 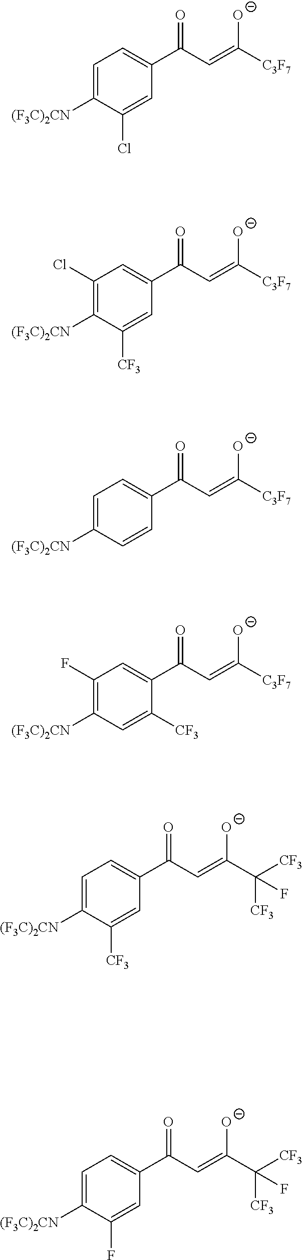 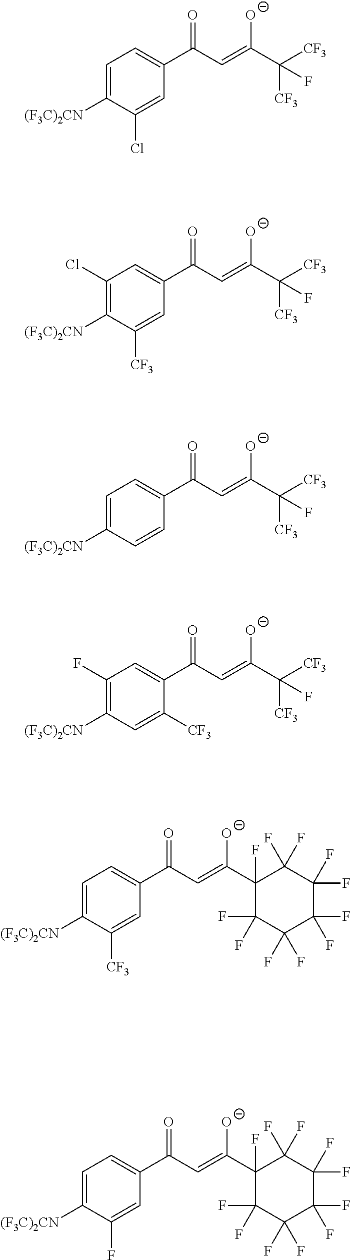 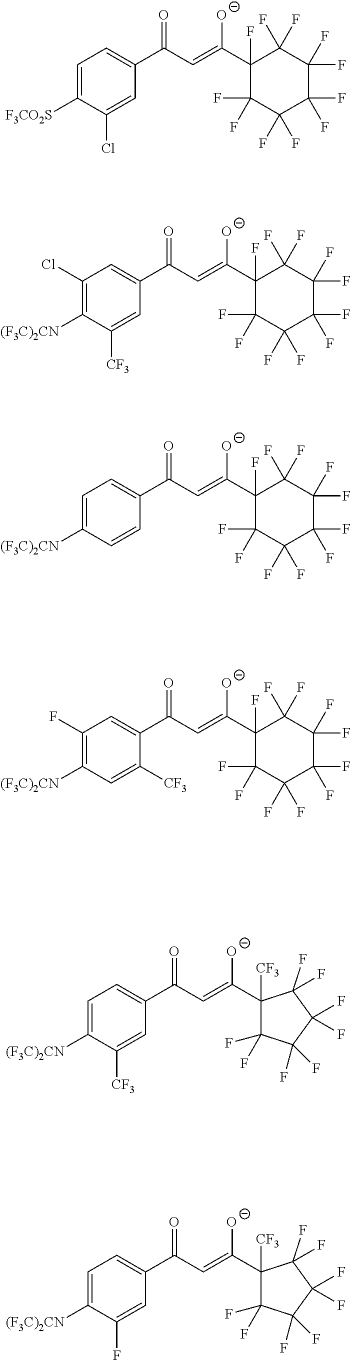 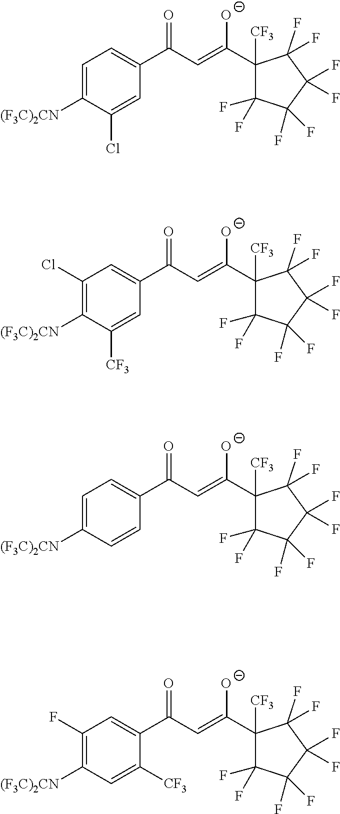 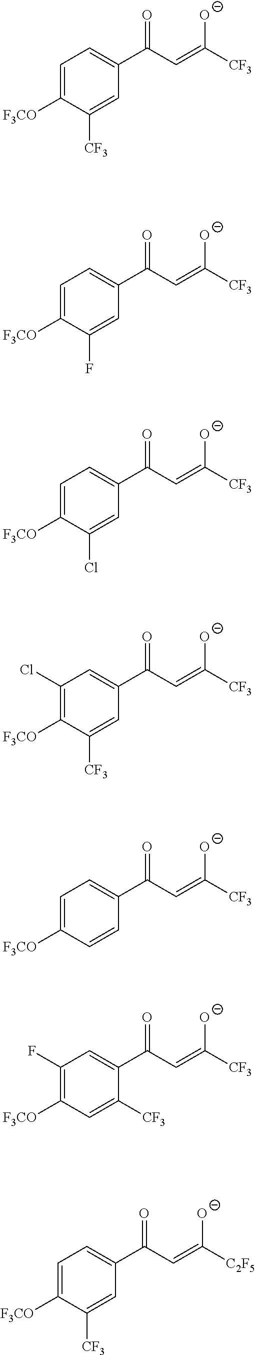 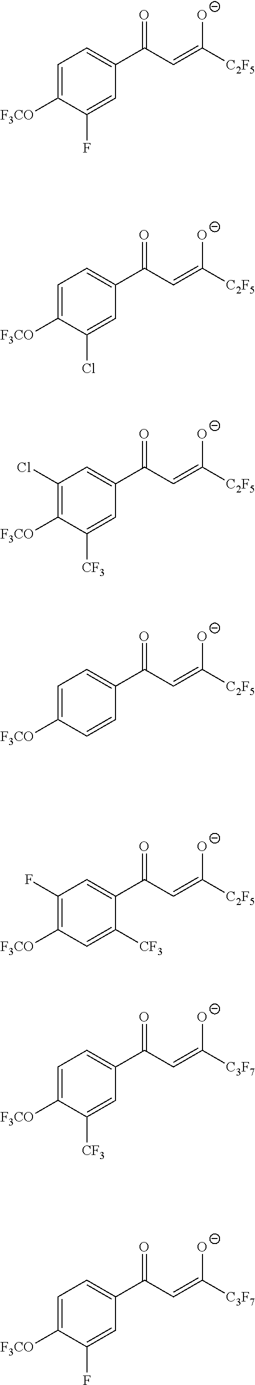 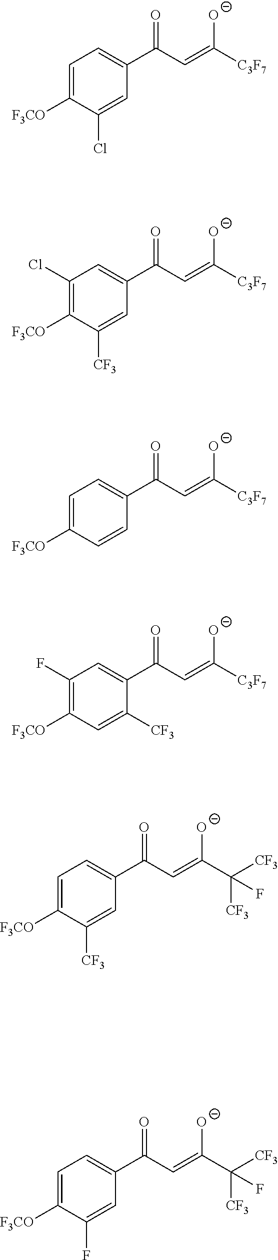 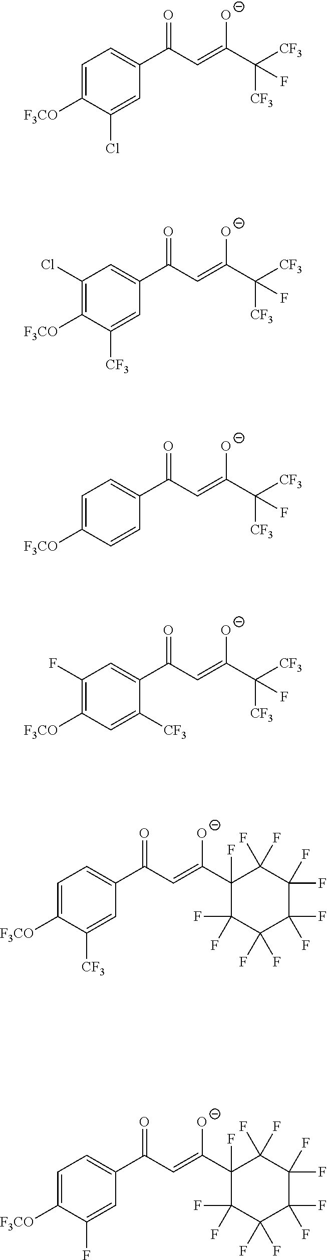 [SMILES: O=C(/C=C(\[O-])C(F)(C(F)(F)F)C(F)(F)F)c1ccc(OC(F)(F)F)c(Cl)c1 ; O=C(/C=C(\[O-])C(F)(C(F)(F)F)C(F)(F)F)c1cc(Cl)c(OC(F)(F)F)c(C(F)(F)F)c1 ; O=C(/C=C(\[O-])C(F)(C(F)(F)F)C(F)(F)F)c1ccc(OC(F)(F)F)cc1 ; O=C(/C=C(\[O-])C(F)(C(F)(F)F)C(F)(F)F)c1cc(F)c(OC(F)(F)F)cc1C(F)(F)F ; O=C(/C=C(\[O-])C1(F)C(F)(F)C(F)(F)C(F)(F)C(F)(F)C1(F)F)c1ccc(OC(F)(F)F)c(C(F)(F)F)c1 ; O=C(/C=C(\[O-])C1(F)C(F)(F)C(F)(F)C(F)(F)C(F)(F)C1(F)F)c1ccc(OC(F)(F)F)c(F)c1] 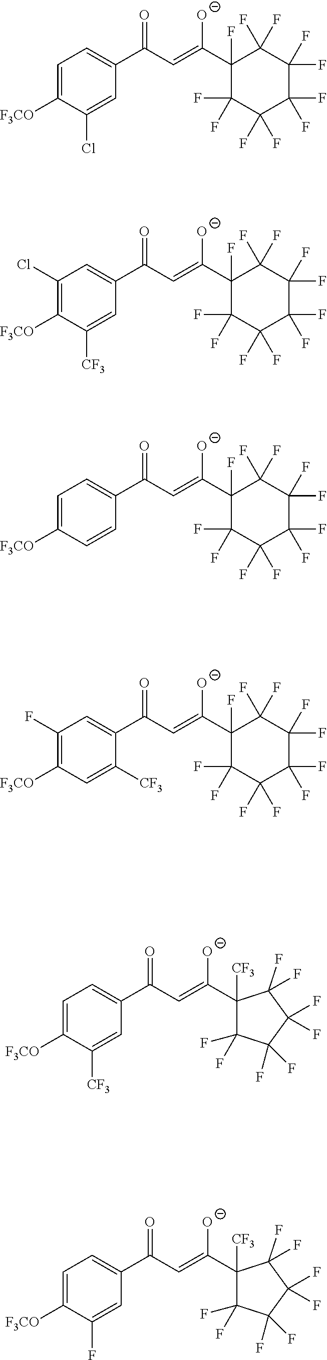 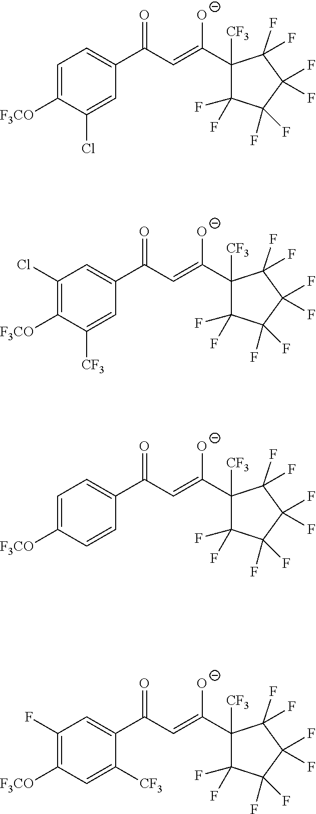 [SMILES: O=C(/C=C(\[O-])C1(C(F)(F)F)C(F)(F)C(F)(F)C(F)(F)C1(F)F)c1ccc(OC(F)(F)F)c(Cl)c1 ; O=C(/C=C(\[O-])C1(C(F)(F)F)C(F)(F)C(F)(F)C(F)(F)C1(F)F)c1cc(Cl)c(OC(F)(F)F)c(C(F)(F)F)c1 ; O=C(/C=C(\[O-])C1(C(F)(F)F)C(F)(F)C(F)(F)C(F)(F)C1(F)F)c1ccc(OC(F)(F)F)cc1 ; O=C(/C=C(\[O-])C1(C(F)(F)F)C(F)(F)C(F)(F)C(F)(F)C1(F)F)c1cc(F)c(OC(F)(F)F)cc1C(F)(F)F] 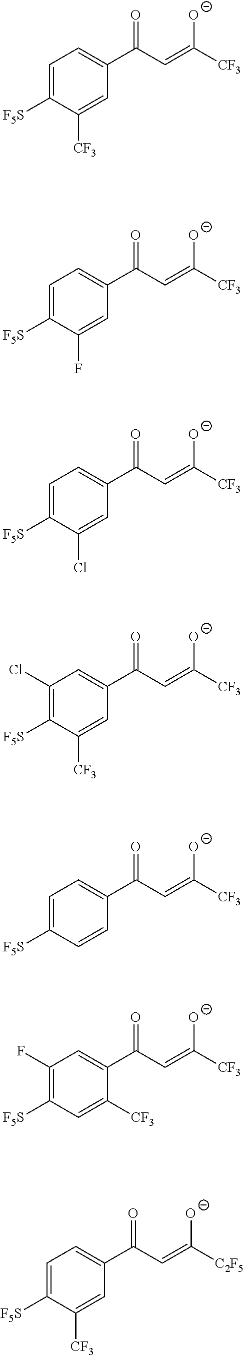 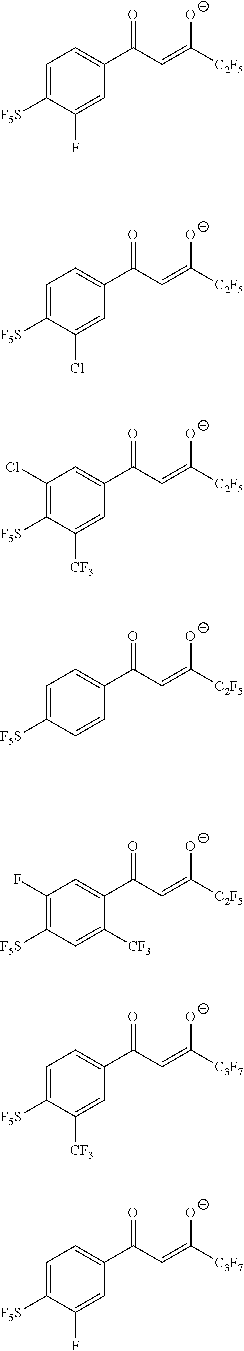 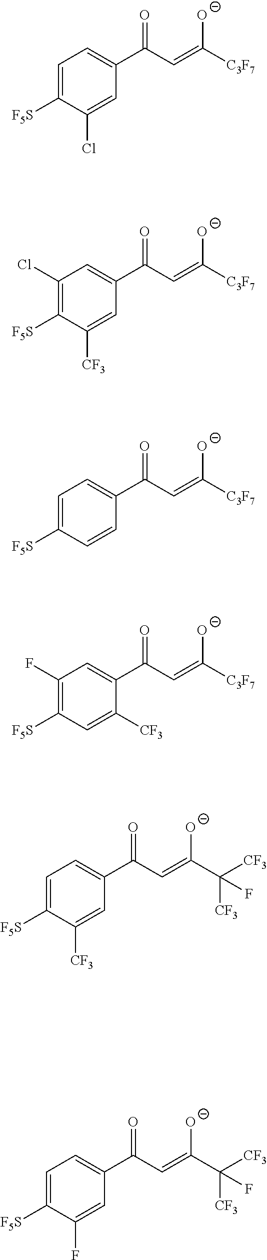 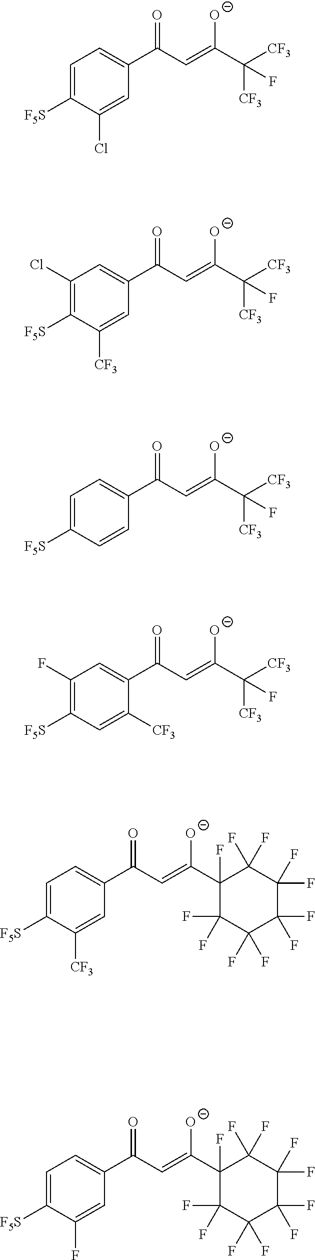 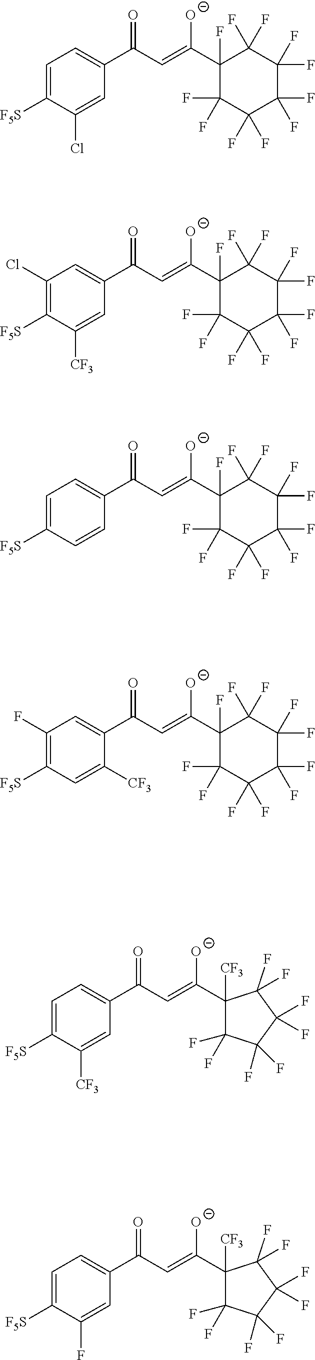 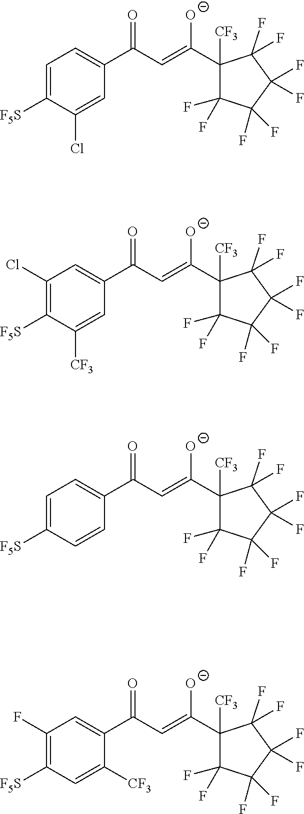 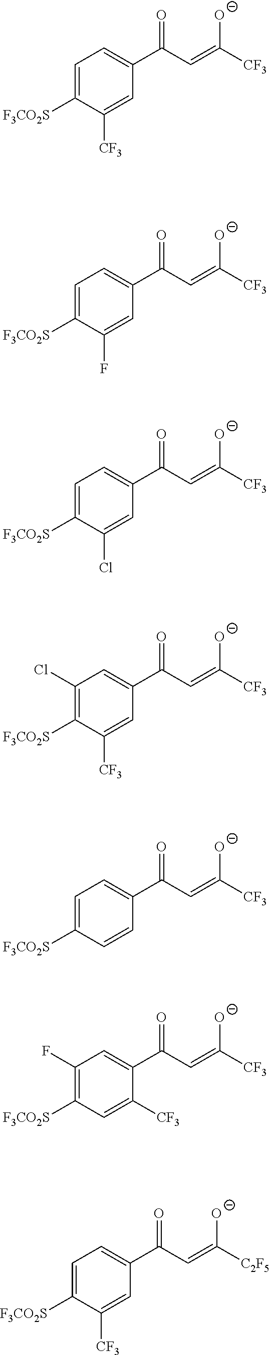 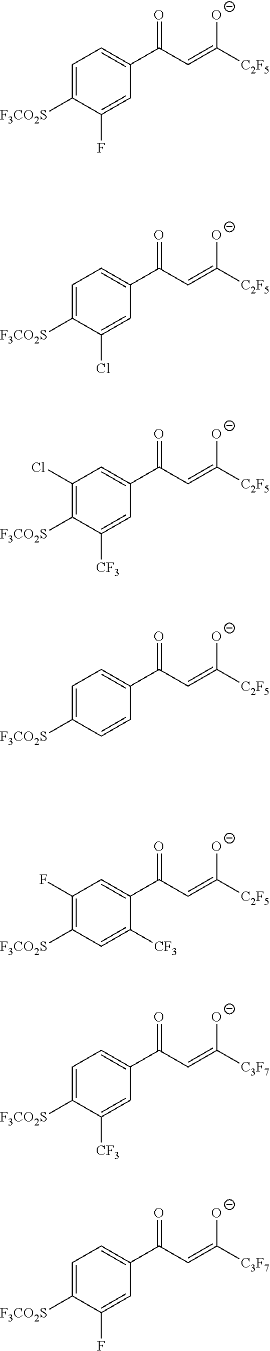 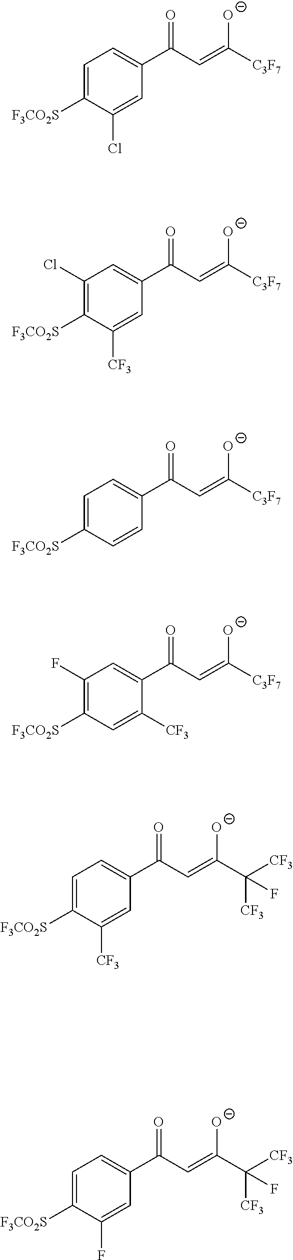 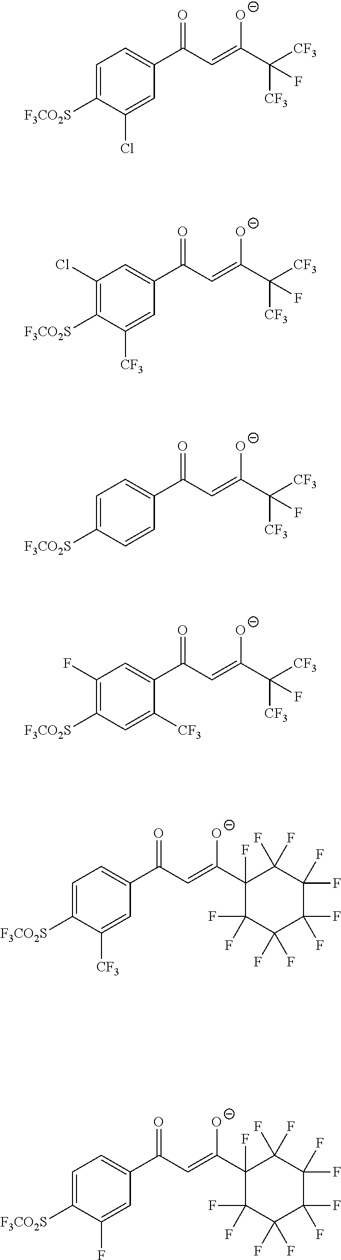 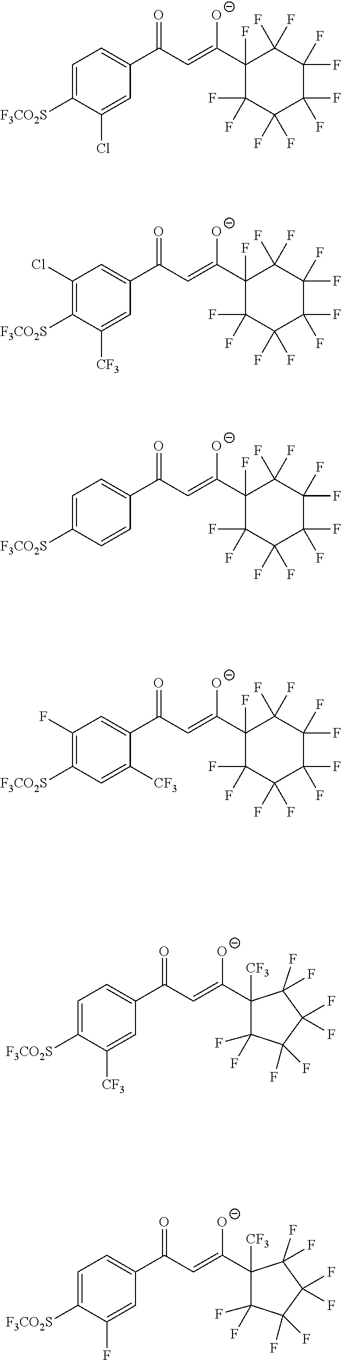 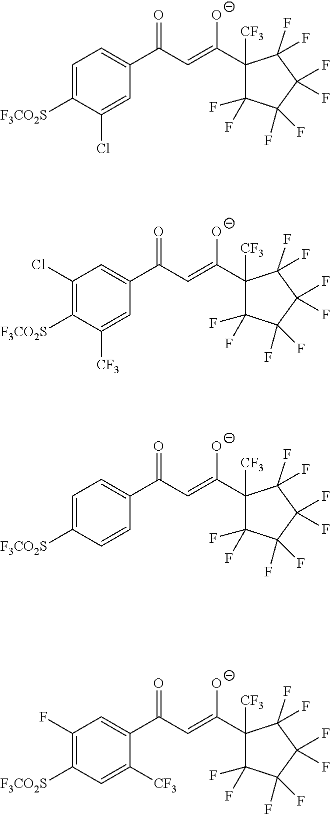 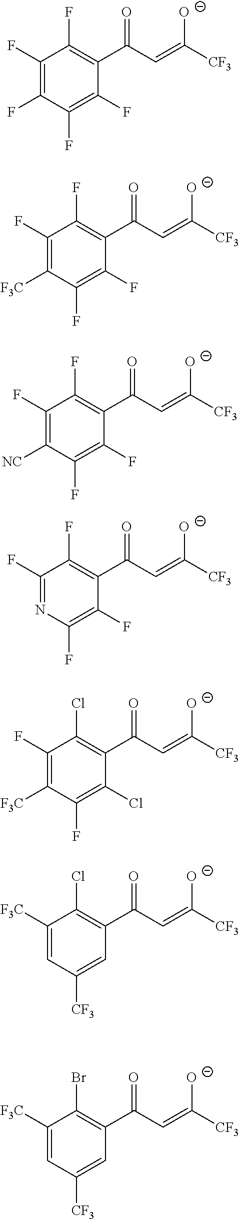 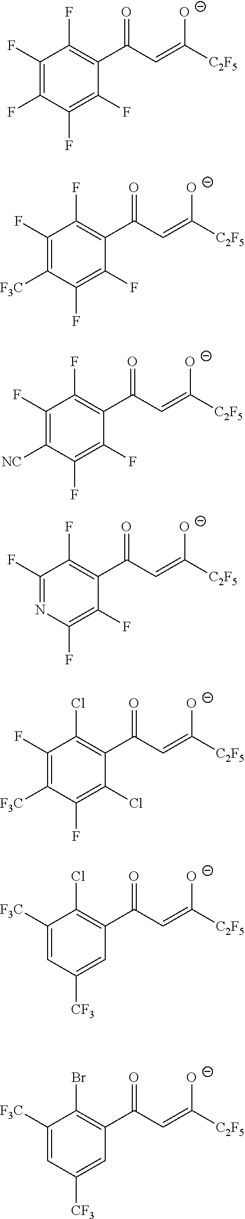 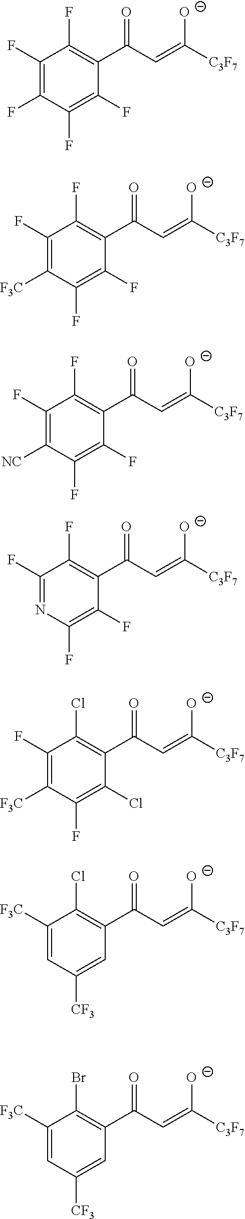 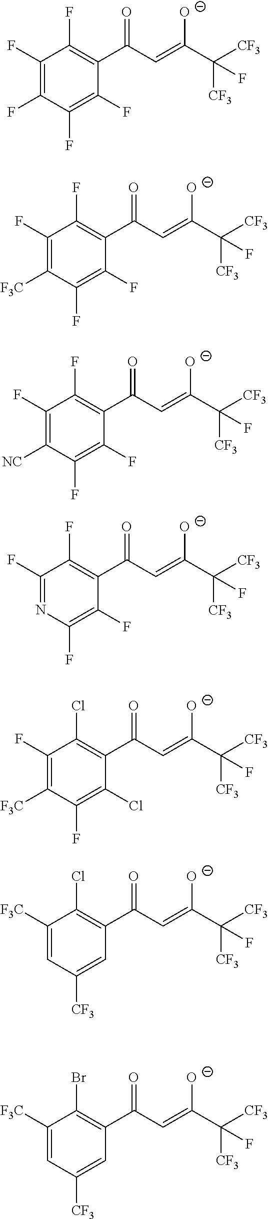 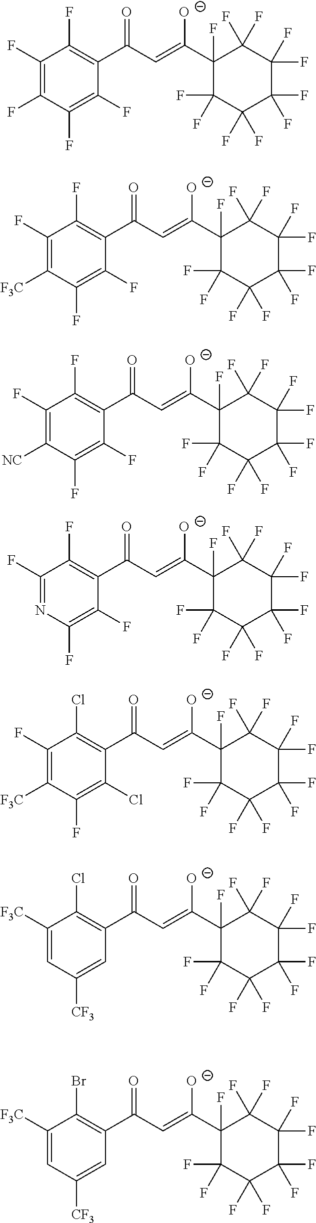 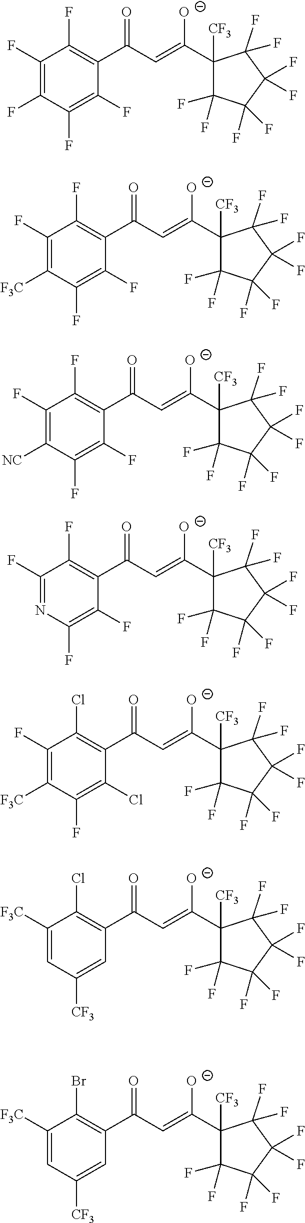 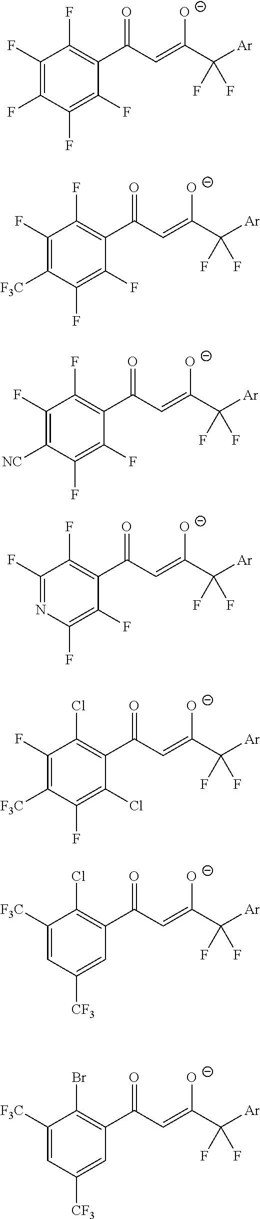 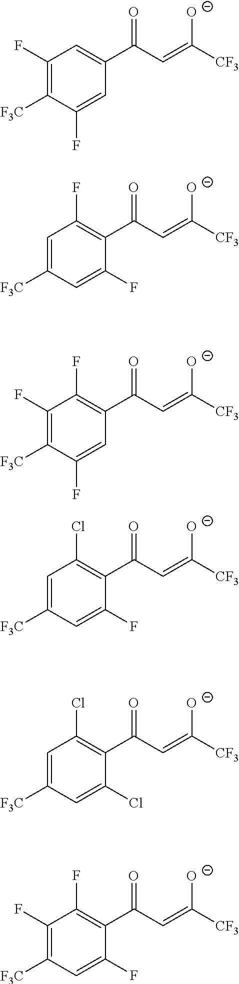 [SMILES: O=C(/C=C(\[O-])C(F)(F)F)c1cc(F)c(C(F)(F)F)c(F)c1 ; O=C(/C=C(\[O-])C(F)(F)F)c1c(F)cc(C(F)(F)F)cc1F ; O=C(/C=C(\[O-])C(F)(F)F)c1cc(F)c(C(F)(F)F)c(F)c1F ; O=C(/C=C(\[O-])C(F)(F)F)c1c(F)cc(C(F)(F)F)cc1Cl ; O=C(/C=C(\[O-])C(F)(F)F)c1c(Cl)cc(C(F)(F)F)cc1Cl ; O=C(/C=C(\[O-])C(F)(F)F)c1c(F)cc(C(F)(F)F)c(F)c1F] 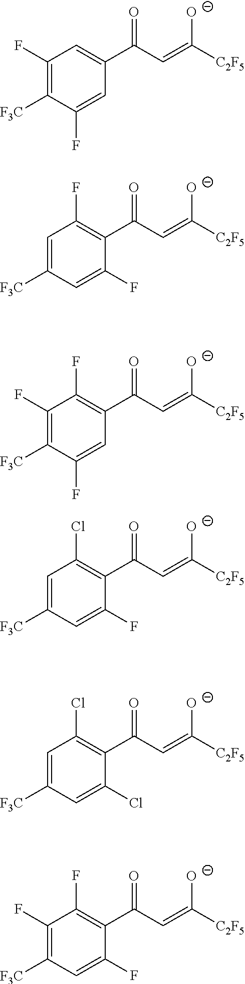 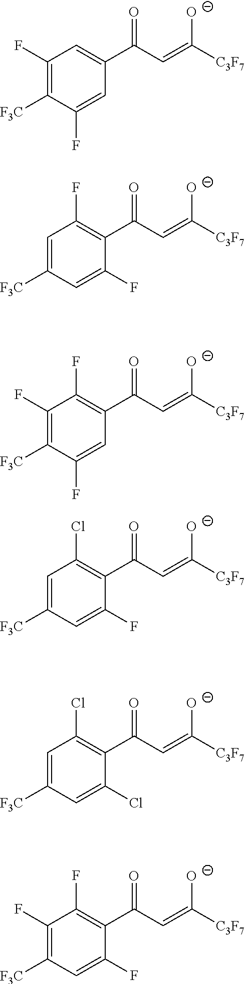 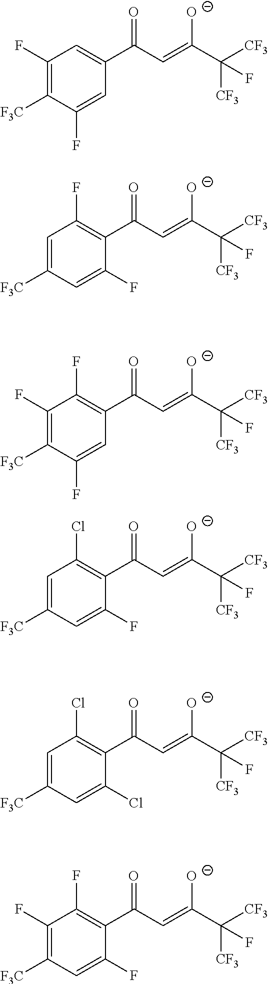 [SMILES: O=C(/C=C(\[O-])C(F)(C(F)(F)F)C(F)(F)F)c1cc(F)c(C(F)(F)F)c(F)c1 ; O=C(/C=C(\[O-])C(F)(C(F)(F)F)C(F)(F)F)c1c(F)cc(C(F)(F)F)cc1F ; O=C(/C=C(\[O-])C(F)(C(F)(F)F)C(F)(F)F)c1cc(F)c(C(F)(F)F)c(F)c1F ; O=C(/C=C(\[O-])C(F)(C(F)(F)F)C(F)(F)F)c1c(F)cc(C(F)(F)F)cc1Cl ; O=C(/C=C(\[O-])C(F)(C(F)(F)F)C(F)(F)F)c1c(Cl)cc(C(F)(F)F)cc1Cl ; O=C(/C=C(\[O-])C(F)(C(F)(F)F)C(F)(F)F)c1c(F)cc(C(F)(F)F)c(F)c1F] 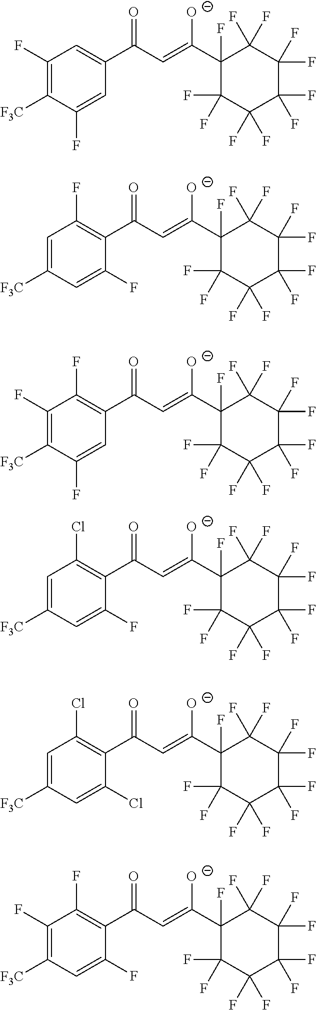 [SMILES: O=C(/C=C(\[O-])C1(F)C(F)(F)C(F)(F)C(F)(F)C(F)(F)C1(F)F)c1cc(F)c(C(F)(F)F)c(F)c1 ; O=C(/C=C(\[O-])C1(F)C(F)(F)C(F)(F)C(F)(F)C(F)(F)C1(F)F)c1c(F)cc(C(F)(F)F)cc1F ; O=C(/C=C(\[O-])C1(F)C(F)(F)C(F)(F)C(F)(F)C(F)(F)C1(F)F)c1cc(F)c(C(F)(F)F)c(F)c1F ; O=C(/C=C(\[O-])C1(F)C(F)(F)C(F)(F)C(F)(F)C(F)(F)C1(F)F)c1c(F)cc(C(F)(F)F)cc1Cl ; O=C(/C=C(\[O-])C1(F)C(F)(F)C(F)(F)C(F)(F)C(F)(F)C1(F)F)c1c(Cl)cc(C(F)(F)F)cc1Cl ; O=C(/C=C(\[O-])C1(F)C(F)(F)C(F)(F)C(F)(F)C(F)(F)C1(F)F)c1c(F)cc(C(F)(F)F)c(F)c1F] 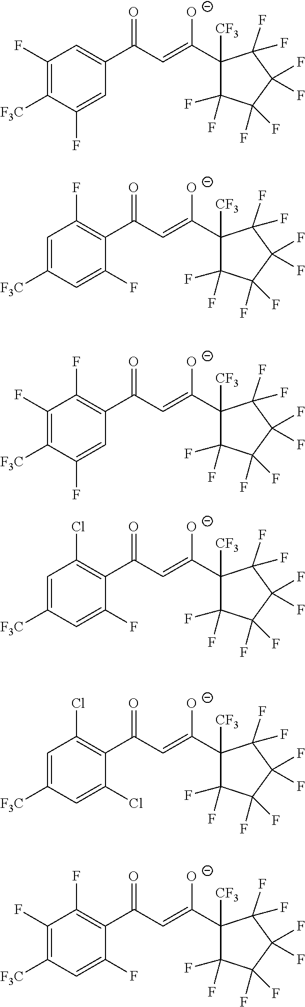 [SMILES: O=C(/C=C(\[O-])C1(C(F)(F)F)C(F)(F)C(F)(F)C(F)(F)C1(F)F)c1cc(F)c(C(F)(F)F)c(F)c1 ; O=C(/C=C(\[O-])C1(C(F)(F)F)C(F)(F)C(F)(F)C(F)(F)C1(F)F)c1c(F)cc(C(F)(F)F)cc1F ; O=C(/C=C(\[O-])C1(C(F)(F)F)C(F)(F)C(F)(F)C(F)(F)C1(F)F)c1cc(F)c(C(F)(F)F)c(F)c1F ; O=C(/C=C(\[O-])C1(C(F)(F)F)C(F)(F)C(F)(F)C(F)(F)C1(F)F)c1c(F)cc(C(F)(F)F)cc1Cl ; O=C(/C=C(\[O-])C1(C(F)(F)F)C(F)(F)C(F)(F)C(F)(F)C1(F)F)c1c(Cl)cc(C(F)(F)F)cc1Cl ; O=C(/C=C(\[O-])C1(C(F)(F)F)C(F)(F)C(F)(F)C(F)(F)C1(F)F)c1c(F)cc(C(F)(F)F)c(F)c1F] 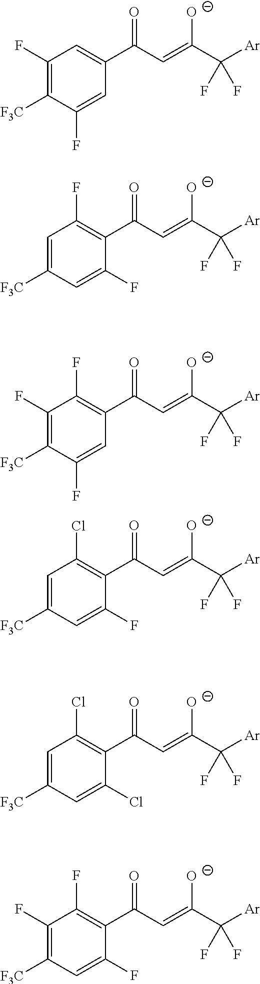 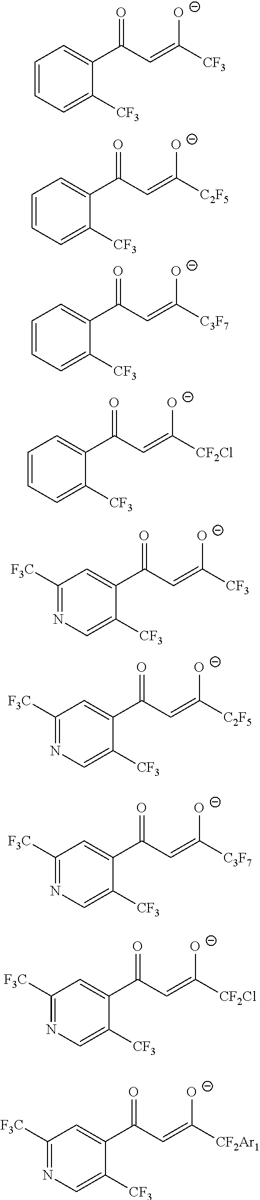 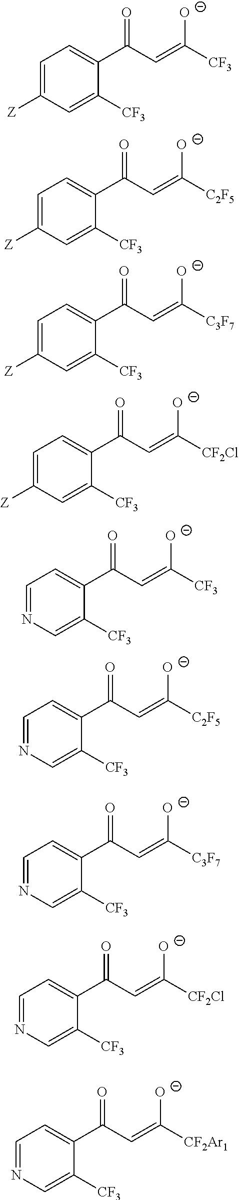 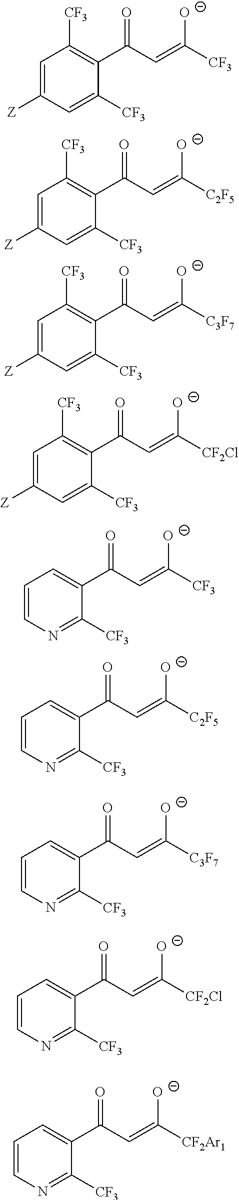 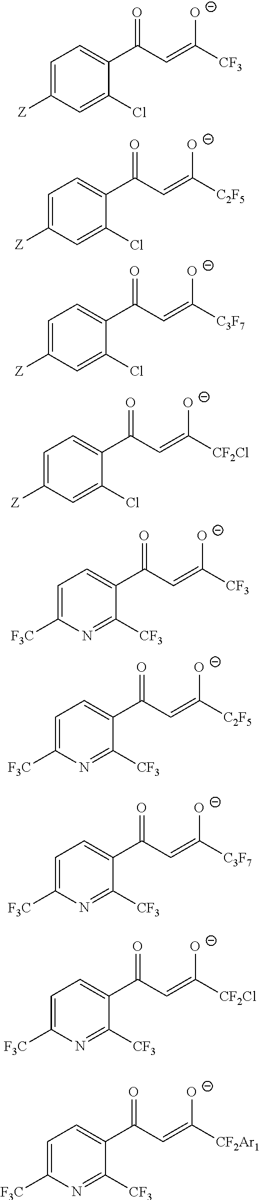 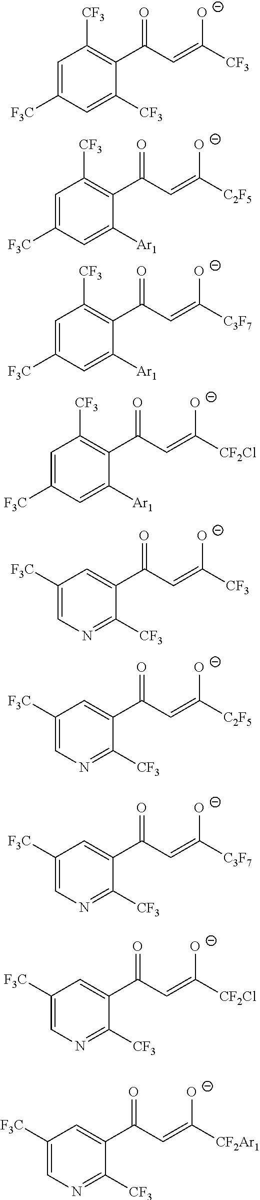 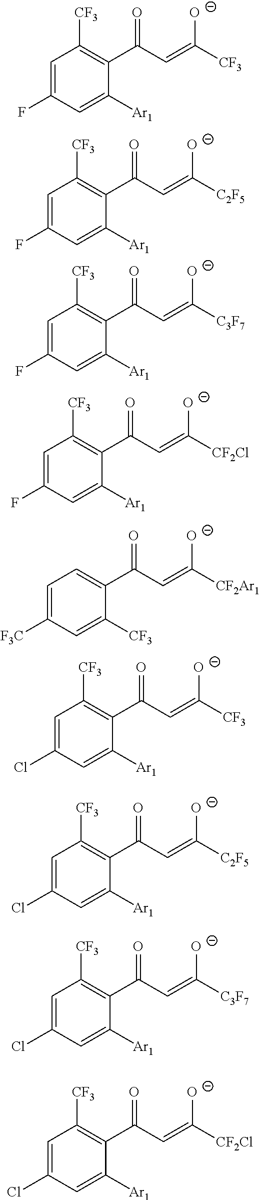 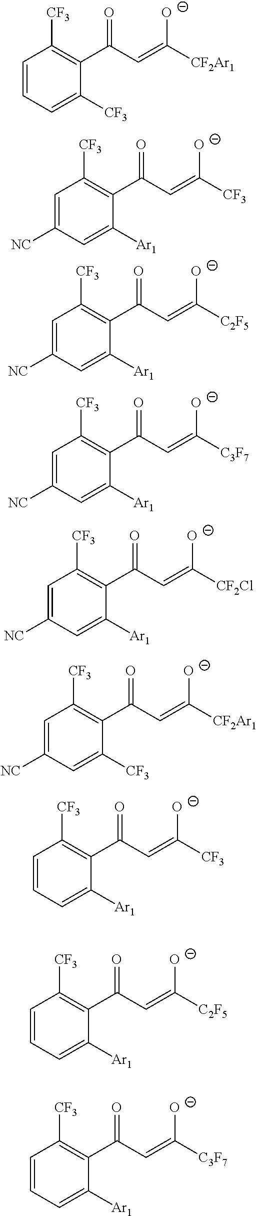 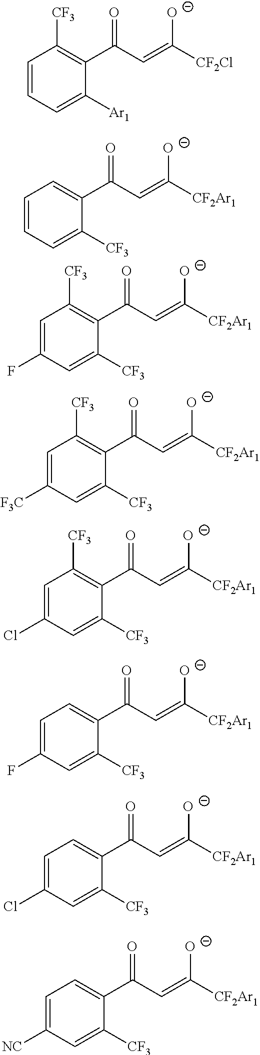 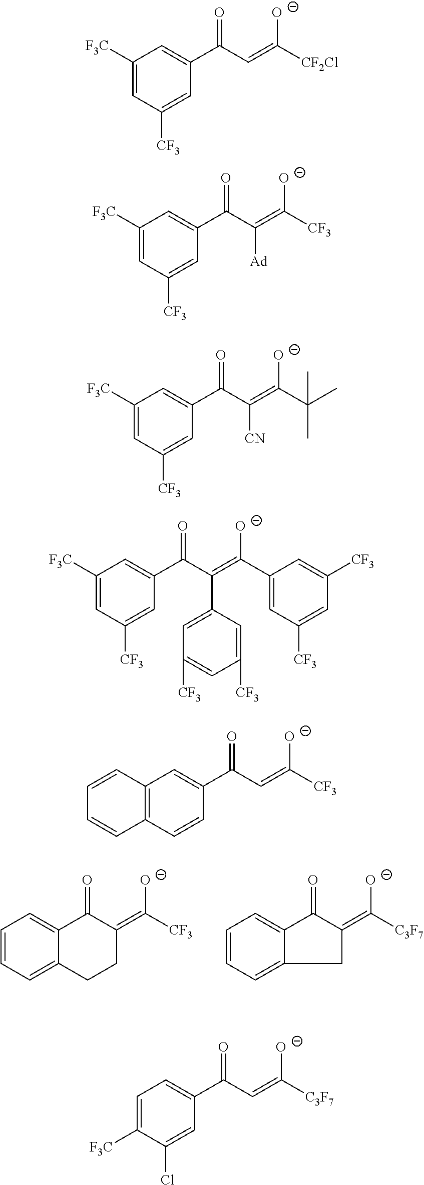
Ad is adamantyl,
R³ is CN, F, Cl, CF₃, Ar, t-butyl, adamantyl,
R¹ is CF₂Cl, CF(CF₃)₂, n-C₃F₇, 2,2,3,3,4,4,5,5-octafluoro-1-(trifluoromethyl)cyclopentyl, and 1,2,2,3,3,4,4,5,5,6,6-undecafluorocyclohexyl,
X is N(SO₂CF₃)₂, SF₅, OCF₃, SCF₃, CF(CF₃)₂,
Z is F, Cl, CF₃, CN, 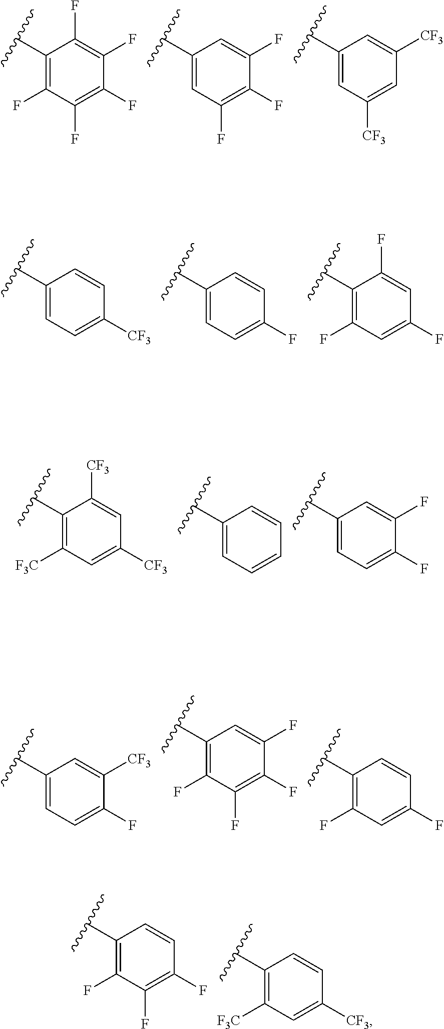
Ar is selected from A1, A2, A3, A4, A5, A6, A7, A8, A9, A10, A11, A12, A13, A14, A15, A16, A17, A18, A19, A20, A21, A22, A23, A24, A25, A26, A27, A28, A29, A30, A31 and A32 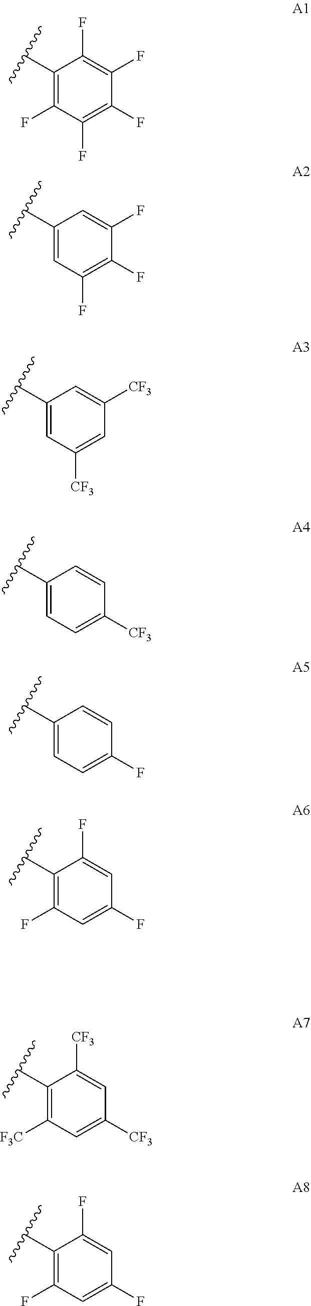 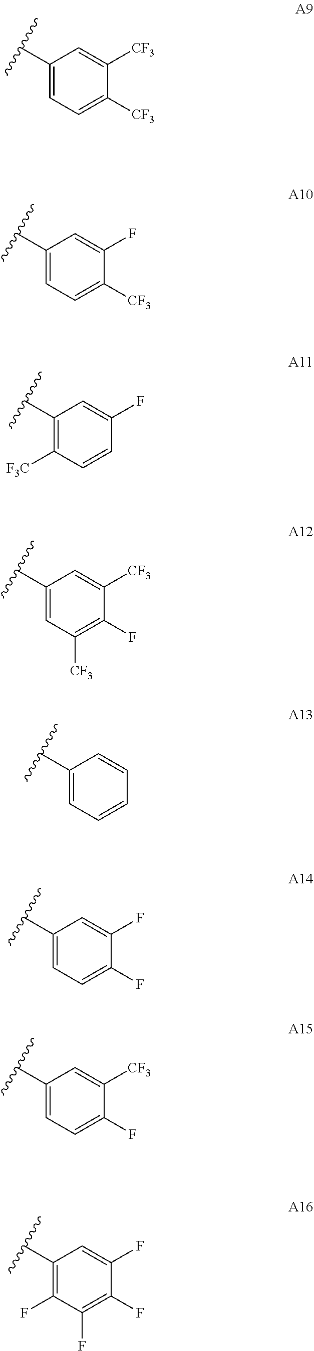 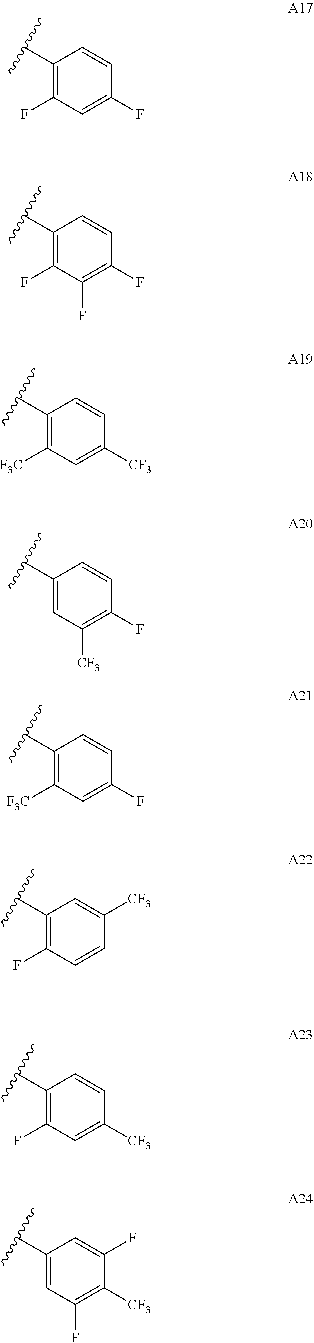 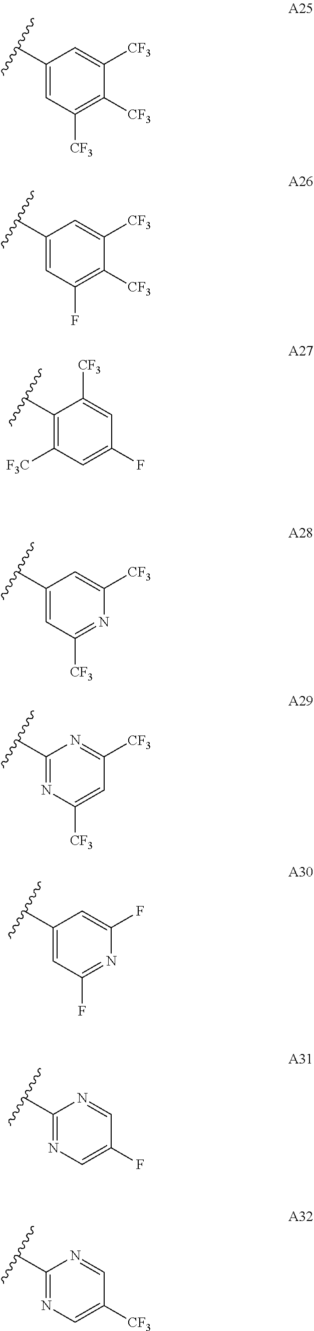
Ar₁ is

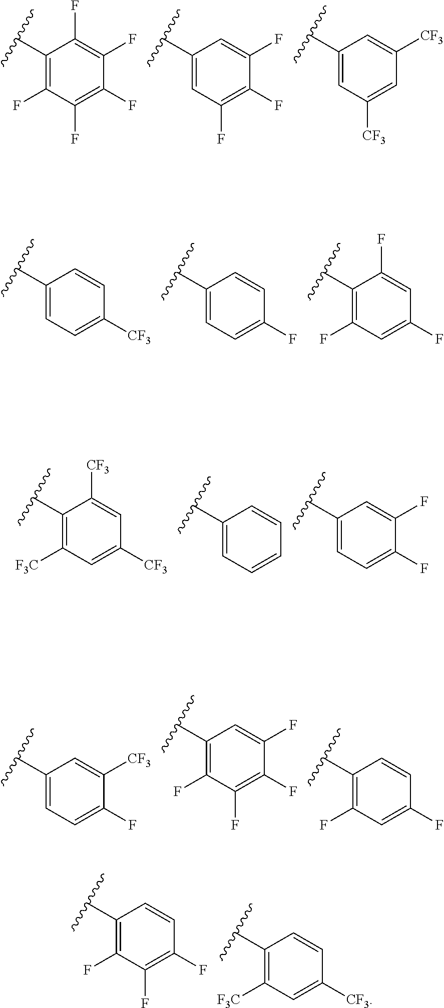

7. Electronic component comprising at least one compound of the general formula (O) 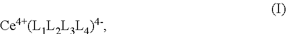
wherein
wherein
L₁; L₂; L₃; and L₄ are independently from each other selected from a bidentate ligand having the general formula (II) 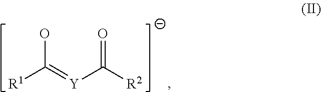
wherein
Y represents N or CR³
R¹ represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
cycloalkyl is unsubstituted or substituted with 1 to 13 radicals selected from fluorine and CF₃,
phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂;
or
R¹ and Y together with the carbon atom of the C—O-group to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R² represents CFR^{a}R^{b}, t-butyl, adamantyl, C₂F₅, n-C₃F₇, C₃-C₇-cycloalkyl, phenyl, naphthyl, pyridyl, pyrimidyl, triazinyl, wherein
cycloalkyl is unsubstituted or substituted with 1 to 13 radicals selected from fluorine and CF₃,
phenyl is substituted by at least one radical selected from NO₂, F, Cl, Br, CN, CF₃, SF₅, OCF₃, SCF₃, SO₂CF₃, N═C(CF₃)₂, and N(SO₂CF₃)₂,
naphthyl is unsubstituted or substituted with 1 to 7 radicals selected from CN, NO₂, F, Cl, Br, C₁-C₄-haloalkyl, OCF₃, SCF₃, SO₂CF₃, N═C(CF₃)₂, SF₅ and N(SO₂CF₃)₂,
pyridyl, pyrimidyl and triazinyl are substituted by at least one radical selected from NO₂, F, Cl, Br, CN, CF₃, SF₅, OCF₃, SCF₃, SO₂CF₃, N═C(CF₃)₂, and N(SO₂CF₃)₂;
or
R² and Y together with the carbon atom of the C—O-group to which they are bonded form a 5 to 7 membered ring which may bear a C═O or C(CH₃)₂ group as ring member and wherein the 5 to 7 membered ring is fused with a benzene group, wherein the fused benzene group is unsubstituted or substituted with 1, 2, 3 or 4 substituents R⁴;
R^{a} and R^{b} independently from each other represent halogen, CF₃, CN and C₆-C₁₄-aryl, which is unsubstituted or substituted with 1, 2, 3, 4 or 5 substituents selected from halogen, C₁-C₄-haloalkyl, C₁-C₄-haloalkoxy, SCF₃, SO₂CF₃, NO₂, N═C(CF₃)₂, SF₅, N(SO₂CF₃)₂ and CN;
R³ represents hydrogen, F, C, CN, CF₃, OCF₃, SCF₃, methyl, ethyl, t-butyl, adamantyl, phenyl or hetaryl having 4 to 5 carbon atoms, wherein hetaryl has 1, 2 or 3 nitrogen atoms as ring members, wherein phenyl and hetaryl are unsubstituted or substituted by 1 or 2 identical or different radicals R⁵;
R⁴ represents CN, NO₂, SF₅, halogen, C₁-C₄-alkyl, C₁-C₄-haloalkyl, C₁-C₄-haloalkoxy, C₁-C₄-haloalkylsulfanyl, C₁-C₄-haloalkylsulfonyl, N═C(CF₃)₂ and N(SO₂CF₃)₂;
R⁵ represents CN, F, Cl, CF₃, OCF₃, SCF₃, SO₂CF₃, NO₂, N═C(CF₃)₂, SF₅, and N(SO₂CF₃)₂;
with the proviso that the electronic components comprising compounds of formula (I.a), 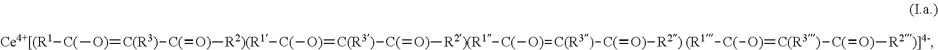
wherein
(R¹, R², R³), (R^{1′}, R^{2′}, R^{3′}), (R^{1″}, R^{2″}, R^{3″}) and (R^{1′″}, R^{2′″}, R^{3′″}) are defined in the table of claim 1 are excluded.

8. Electronic component according to claim 7 in form of an organic light-emitting diode, an organic solar cell, an organic photodetector, a photovoltaic cell, an organic diode or an organic transistor, preferably in form of a thin-film transistor, a Perovskite solar cell.

9. Electronic component according to claim 7, having a layer structure, which comprises 2, 3, 4, 5, 6, 7 or more layers.

10. Electronic component according to claim 7, comprising a hole transport layer and/or a hole injection layer and/or an electron transport layer, which comprises at least of the compound of formula (I).

11. Electronic component according to claim 7, comprising an electron transport layer, which comprises at least of the compound of formula (I).

12. Doped semiconductor matrix material comprising at least one electron donor and at least one compound of the formula (I) as defined in any one of claims 1 to 6, wherein the electron donor is preferably selected from 4,4′,4″-tris(N-(2-naphthyl)-N-phenyl-amino)triphenylamine (2-TNATA), 4,4′,4″-tris(N-3-methylphenyl-N-phenyl-amino)triphenylamine (m-MTDATA), N,N,N′,N′-tetrakis(4-methoxy-phenyl)benzidine (MeO-TPD), (2,2′,7,7′-tetrakis-(N,N-diphenylamino)-9,9′-spirobifluorene (spiro-TTB), N,N′-bis(naphthalene-1-yl)-N,N′-bis(phenyl)-benzidine, N,N′-bis(naphthalene-1-yl)-N,N′-bis(phenyl)-9,9-spiro-bifluorene, 9,9-bis[4-(N,N-bis-biphenyl-4-yl-amino)phenyl]-9H-fluorene, 2,2′-bis[N,N-bis(biphenyl-4-yl)amino]-9,9-spiro-bifluorene, N,N′-((9H-fluorene-9,9-diyl)bis(4,1-phenylene))bis(N-([1,1′-biphenyl]-4-yl)-[1,1′-biphenyl]-4-amine) (BPAPF), N,N′-bis(phenanthrene-9-yl)-N,N′-bis(phenyl)-benzidine, 1,3,5-tris{4-[bis(9,9-dimethyl-fluorene-2-yl)amino]phenyl}benzene, tri(terphenyl-4-yl)amine, N-(4-(6-((9,9-dimethyl-9H-fluorene-2-yl)(6-methoxy-[1,1′-biphenyl]-3-yl)amino)-1,3,3-trimethyl-2,3-dihydro-1H-indene-1-yl)phenyl)-N-(6-methoxy-[1,1′-biphenyl]-3-yl)-9,9-dimethyl-9H-fluorene-2-amine, N-([1,1′-biphenyl]-4-yl)-N-(4-(6-([1,1′-biphenyl]-4-yl(9,9-dimethyl-9H-fluorene-2-yl)amino)-1,3,3-trimethyl-2,3-dihydro-1H-indene-1-yl)phenyl)-9,9-dimethyl-9H-fluorene-2-amine, N,N-di([1,1′-biphenyl]-4-yl)-3-(4-(di([1, 1′-biphenyl]-4-yl)amino)phenyl)-1,1,3-trimethyl-2,3-dihydro-1H-indene-5-amine, N-(4-(6-(bis(9,9-dimethyl-9H-fluorene-2-yl)amino)-1,3,3-trimethyl-2,3-dihydro-1H-inden-1-yl)phenyl)-N-(9,9-dimethyl-9H-fluorene-2-yl)-9,9-dimethyl-9H-fluorene-2-amine, N-(4-(6-(9,9′-spirobi[fluorene]-2-yl(9,9-dimethyl-9H-fluorene-2-yl)amino)-1,3,3-trimethyl-2,3-dihydro-1H-inden-1-yl)phenyl)-N-(9,9-dimethyl-9H-fluoren-2-yl)-9,9′-spirobi[fluorene]-2-amine, N-(4-(6-(dibenzo[b,d]furane-2-yl(9,9-dimethyl-9H-fluorene-2-yl)amino)-1,3,3-trimethyl-2,3-dihydro-1H-indene-1-yl)phenyl)-N-(9,9-dimethyl-9H-fluorene-2-yl)dibenzo[b,d]furan-2-amine, 9-(4-(6-(9H-carbazol-9-yl)-1,3,3-trimethyl-2,3-dihydro-1H-inden-1-yl)phenyl)-9H-carbazole, N-([1,1′-biphenyl]-4-yl)-3-(4-([1,1′-biphenyl]-4-yl(4-methoxyphenyl)amino)phenyl)-N-(4-methoxyphenyl)-1,1,3-trimethyl-2,3-dihydro-1H-inden-5-amine, 3-(4-(bis(6-methoxy-[1,1′-biphenyl]-3-yl)amino)phenyl)-N,N-bis(6-methoxy-[1,1′-biphenyl]-3-yl)-1,1,3-trimethyl-2,3-dihydro-1H-indene-5-amine, N1-([1, 1′-biphenyl]-4-yl)-N1-(4-(6-([1,1′-biphenyl]-4-yl(4-(diphenylamino)phenyl)amino)-1,3,3-trimethyl-2,3-dihydro-1H-inden-1-yl)phenyl)-N4,N4-diphenylbenzene-1,4-diamine, N,N-di([1, 1′-biphenyl]-4-yl)-4′-(6-(4-(di([1,1′-biphenyl]-4-yl)amino)phenyl)-1,3,3-trimethyl-2,3-dihydro-1H-indene-1-yl)-[1,1′-biphenyl]-4-amine, N-(4-(5-(bis(9,9-dimethyl-9H-fluorene-2-yl)amino)-1,3,3-trimethyl-2,3-dihydro-1H-indene-1-yl)phenyl)-N-(9,9-dimethyl-9H-fluorene-2-yl)-9,9-dimethyl-9H-fluorene-2-amine, N-(4-(6-(bis(9,9-dimethyl-9H-fluorene-2-yl)amino)-1,3,3-trimethyl-2,3-dihydro-1H-indene-1-yl)phenyl)-N-(9,9-dimethyl-9H-fluorene-2-yl)-9,9-dimethyl-9H-fluorene-2-amine, N,N′-bis(9,9-dimethyl-fluorene-2-yl)-N,N′-diphenyl-benzidine (BF-DPB), N,N′-((9H-fluorene-9,9-diyl)bis(4,1-phenylene))bis(N-([1,1′-biphenyl]-4-yl)-[1,1′-biphenyl]-4-amine) (BPAPF), N4,N4,N4′,N4′-tetrakis(9,9-dimethyl-9H-fluorene-2-yl)-[1,1′-biphenyl]-4,4′-diamine (TDMFB), N-([1,1′-biphenyl]-2-yl)-N-(9,9-dimethyl-9H-fluorene-2-yl)-9,9′-spirobi[fluorene]-2-amine, (2,7-bis[N,N-bis(4-methoxyphenyl)amino]-9,9-spirobi[9H-fluorene] (spiro-MeO-TPD), a mixture of N-(4-(5-(bis(9,9-dimethyl-9H-fluorene-2-yl)amino)-1,3,3-trimethyl-2,3-dihydro-1H-inden-1-yl)phenyl)-N-(9,9-dimethyl-9H-fluorene-2-yl)-9,9-dimethyl-9H-fluorene-2-amine and N-(4-(6-(bis(9,9-dimethyl-9H-fluorene-2-yl)amino)-1,3,3-trimethyl-2,3-dihydro-1H-inden-1-yl)phenyl)-N-(9,9-dimethyl-9H-fluorene-2-yl)-9,9-dimethyl-9H-fluorene-2-amine, N-([1,1′-biphenyl]-4-yl)-9,9-dimethyl-N-(4-(9-phenyl-9H-carbazol-3-yl)phenyl)-9H-fluorene-2-amine and mixtures thereof.

13. The doped semiconductor matrix material according to claim 12, wherein the molar ratio of matrix molecule to compounds of formula (I) is 10000:1 to 1:1.

14. A method for the production of
an organic semiconductor,
an a redox doping agent in organic semiconductor matrix materials, especially as p-dopant in hole transport layers,
an electron transport material,
an charge injector in a charge injection layer,
a cathode material in organic batteries,
an electrochromic material,
wherein a compound of formula (I) or mixtures thereof, as defined in any one of claims 1 to 6 is employed.

15. A method for the production organic semiconductor or electrochromic material, wherein Ce(III) complex anions obtained by reduction of a compound of formula (I) as defined in any one of claims 1 to 6 or of charge transfer complexes of a compound of formula (I) as defined in any one of claims 1 to 6 with electron donors is employed.
